SEQUENCE LISTING (158 sequences)

SEQ ID NO: 1 - protein, 303 aa, Homo sapiens
  REGION 1..303: note = Description of sequence: human PILRA
MGRPLLLPLLPLLLPPAFLQPSGSTGSGPSYLYGVTQPKHLSASMGGSVEIPFSFYYPWELATAPDVRISWRRGHFHGQSFYSTRPPSIHKDYVNRLFLNWTEGQKSGFLRISNLQKQDQSVYFCRVELDTRSSGRQQWQSIEGTKLSITQAVTTTTQRPSSMTTTWRLSSTTTTTGLRVTQGKRRSDSWHISLETAVGVAVAVTVLGIMILGLICLLRWRRRKGQQRTKATTPAREPFQNTEEPYENIRNEGQNTDPKLNPKDDGIVYASLALSSSTSPRAPPSHRPLKSPQNETLYSVLKA

SEQ ID NO: 2 - protein, 307 aa, Macaca fascicularis
  REGION 1..307: note = Description of sequence: cynomolgus monkey PILRA
MGRPLLLPLLLPLLPLLLPPAFLQPGGSAGSGPSGPYGVTQRKHLSAPMGGSVEIPFSFYHPWELAAAPNMKISWRRGNFHGEFFYRTRPAFIHEDYSNRLLLNWTEGQDRGLLRIWNLRKEDQSVYFCRVELDTRRSGRQRWQSIEGTKLTITQAVTTTTQRPSSMTTTRRPSSATTTAGLRVTQGKRHSDSWHLSLKTAVGVTVAVAVLGIMILGLICLLRWRRRKGQQRTKATTPAKEPFQNTEEPYENIRNEGQNTDPKPNPKDDGIVYASLALSSSTSPRVPPSHHPLKSPQNETLYSVLKV

SEQ ID NO: 3 - protein, 227 aa, Homo sapiens
  REGION 1..227: note = Description of sequence: human PILRB
MGRPLLLPLLLLLQPPAFLQPGGSTGSGPSYLYGVTQPKHLSASMGGSVEIPFSFYYPWELAIVPNVRISWRRGHFHGQSFYSTRPPSIHKDYVNRLFLNWTEGQESGFLRISNLRKEDQSVYFCRVELDTRRSGRQQLQSIKGTKLTITQAVTTTTTWRPSSTTTIAGLRVTESKGHSESWHLSLDTAIRVALAVAVLKTVILGLLCLLLLWWRRRKGSRAPSSDF

SEQ ID NO: 4 - protein, 10 aa, Homo sapiens
  REGION 1..10: note = Description of sequence: CDR-H1
GFTFDDYAMH

SEQ ID NO: 5 - protein, 10 aa, Homo sapiens
  REGION 1..10: note = Description of sequence: CDR-H1
GFTFDDYAIH

SEQ ID NO: 6 - protein, 10 aa, Homo sapiens
  REGION 1..10: note = Description of sequence: CDR-H1
GYTFIGFYIH

SEQ ID NO: 7 - protein, 10 aa, Homo sapiens
  REGION 1..10: note = Description of sequence: CDR-H1 sequence
GYTFTEYYMY

SEQ ID NO: 8 - protein, 10 aa, Homo sapiens
  REGION 1..10: note = Description of sequence: CDR-H1 sequence
GYTFTGHYMH

SEQ ID NO: 9 - protein, 11 aa, Homo sapiens
  REGION 1..11: note = Description of sequence: CDR-H1
GGSISSNNWWS

SEQ ID NO: 10 - protein, 10 aa, Homo sapiens
  REGION 1..10: note = Description of sequence: CDR-H1
GYSFTTYWIA

SEQ ID NO: 11 - protein, 10 aa, Homo sapiens
  REGION 1..10: note = Description of sequence: CDR-H1
GFAFSGYDMS

SEQ ID NO: 12 - protein, 10 aa, Homo sapiens
  REGION 1..10: note = Description of sequence: CDR-H2
GFSWNSGSIG

SEQ ID NO: 13 - protein, 10 aa, Homo sapiens
  REGION 1..10: note = Description of sequence: CDR-H2
GMSWNSGSIG

SEQ ID NO: 14 - protein, 10 aa, Homo sapiens
  REGION 1..10: note = Description of sequence: CDR-H2
WINPESGDTT

SEQ ID NO: 15 - protein, 10 aa, Homo sapiens
  REGION 1..10: note = Description of sequence: CDR-H2
RIDPEDGGTD

SEQ ID NO: 16 - protein, 10 aa, Homo sapiens
  REGION 1..10: note = Description of sequence: CDR-H2
WINPNSGDTD

SEQ ID NO: 17 - protein, 9 aa, Homo sapiens
  REGION 1..9: note = Description of sequence: CDR-H2
EIYHFGTTT

SEQ ID NO: 18 - protein, 10 aa, Homo sapiens
  REGION 1..10: note = Description of sequence: CDR-H2
IIYPGDSDTR

SEQ ID NO: 19 - protein, 10 aa, Homo sapiens
  REGION 1..10: note = Description of sequence: CDR-H2
TISNGGRHTY

SEQ ID NO: 20 - protein, 12 aa, Homo sapiens
  REGION 1..12: note = Description of sequence: CDR-H3
DKSISAAGRFDY

SEQ ID NO: 21 - protein, 12 aa, Homo sapiens
  REGION 1..12: note = Description of sequence: CDR-H3
DKSIGAAGRFDC

SEQ ID NO: 22 - protein, 12 aa, Homo sapiens
  REGION 1..12: note = Description of sequence: CDR-H3
DKSIGAAGRFDS

SEQ ID NO: 23 - protein, 11 aa, Homo sapiens
  REGION 1..11: note = Description of sequence: CDR-H3
GNWNFPDTFDF

SEQ ID NO: 24 - protein, 9 aa, Homo sapiens
  REGION 1..9: note = Description of sequence: CDR-H3
TIRGTVFAF

SEQ ID NO: 25 - protein, 9 aa, Homo sapiens
  REGION 1..9: note = Description of sequence: CDR-H3
TIRGTVFVY

SEQ ID NO: 26 - protein, 10 aa, Homo sapiens
  REGION 1..10: note = Description of sequence: CDR-H3
EGLDGDPFDY

SEQ ID NO: 27 - protein, 11 aa, Homo sapiens
  REGION 1..11: note = Description of sequence: CDR-H3
TLRDFYYYMDV

SEQ ID NO: 28 - protein, 15 aa, Homo sapiens
  REGION 1..15: note = Description of sequence: CDR-H3
TYYYFSGSHWDAFDI

SEQ ID NO: 29 - protein, 11 aa, Homo sapiens
  REGION 1..11: note = Description of sequence: CDR-H3
QKTWDVHAMDY

SEQ ID NO: 30 - protein, 11 aa, Homo sapiens
  REGION 1..11: note = Description of sequence: CDR-L1
QASRRINNYLN

SEQ ID NO: 31 - protein, 11 aa, Homo sapiens
  REGION 1..11: note = Description of sequence: CDR-L1
QASQGINNYLN

SEQ ID NO: 32 - protein, 11 aa, Homo sapiens
  REGION 1..11: note = Description of sequence: CDR-L1
RSSQSISIYLN

SEQ ID NO: 33 - protein, 11 aa, Homo sapiens
  REGION 1..11: note = Description of sequence: CDR-L1
RASEDIFNGLA

SEQ ID NO: 34 - protein, 11 aa, Homo sapiens
  REGION 1..11: note = Description of sequence: CDR-L1
RPSEDIYNGLA

SEQ ID NO: 35 - protein, 16 aa, Homo sapiens
  REGION 1..16: note = Description of sequence: CDR-L1
RSSQSLVHSDGNTYLS

SEQ ID NO: 36 - protein, 10 aa, Homo sapiens
  REGION 1..10: note = Description of sequence: CDR-L1
RTSQNINTYL

SEQ ID NO: 37 - protein, 11 aa, Homo sapiens
  REGION 1..11: note = Description of sequence: CDR-L1
RASQDIRDCLA

SEQ ID NO: 38 - protein, 12 aa, Homo sapiens
  REGION 1..12: note = Description of sequence: CDR-L1
TLSSQHSTYTIE

SEQ ID NO: 39 - protein, 7 aa, Homo sapiens
  REGION 1..7: note = Description of sequence: CDR-L2
DASNLET

SEQ ID NO: 40 - protein, 7 aa, Homo sapiens
  REGION 1..7: note = Description of sequence: CDR-L2
VASSLQS

SEQ ID NO: 41 - protein, 7 aa, Homo sapiens
  REGION 1..7: note = Description of sequence: CDR-L2
NAKTLHT

SEQ ID NO: 42 - protein, 7 aa, Homo sapiens
  REGION 1..7: note = Description of sequence: CDR-L2
NANSLHT

SEQ ID NO: 43 - protein, 7 aa, Homo sapiens
  REGION 1..7: note = Description of sequence: CDR-L2
NISNRFS

SEQ ID NO: 44 - protein, 7 aa, Homo sapiens
  REGION 1..7: note = Description of sequence: CDR-L2
AASSLQS

SEQ ID NO: 45 - protein, 7 aa, Homo sapiens
  REGION 1..7: note = Description of sequence: CDR-L2
AASSFQS

SEQ ID NO: 46 - protein, 11 aa, Homo sapiens
  REGION 1..11: note = Description of sequence: CDR-L2
LKKDGSHSTGD

SEQ ID NO: 47 - protein, 9 aa, Homo sapiens
  REGION 1..9: note = Description of sequence: CDR-L3
QQYDNLPLT

SEQ ID NO: 48 - protein, 9 aa, Homo sapiens
  REGION 1..9: note = Description of sequence: CDR-L3
QQSYSAPFT

SEQ ID NO: 49 - protein, 9 aa, Homo sapiens
  REGION 1..9: note = Description of sequence: CDR-L3
QQYYDYPLT

SEQ ID NO: 50 - protein, 8 aa, Homo sapiens
  REGION 1..8: note = Description of sequence: CDR-L3
IQTTQFST

SEQ ID NO: 51 - protein, 9 aa, Homo sapiens
  REGION 1..9: note = Description of sequence: CDR-L3
QQSFNIPLT

SEQ ID NO: 52 - protein, 9 aa, Homo sapiens
  REGION 1..9: note = Description of sequence: CDR-L3
QQTHSFPYT

SEQ ID NO: 53 - protein, 11 aa, Homo sapiens
  REGION 1..11: note = Description of sequence: CDR-L3
GDTIKEQFVYV

SEQ ID NO: 54 - protein, 121 aa, Homo sapiens
  REGION 1..121: note = Description of sequence: VH
EVQLVESGGGLVQPGRSLRLSCAVSGFTFDDYAMHWVRQAPGKGLEWVSGFSWNSGSIGYPDSVKGRFTISRDNAKNSLYLQMNSLRAEDTAFYYCAKDKSISAAGRFDYWGQGTLVTVSS

SEQ ID NO: 55 - protein, 121 aa, Homo sapiens
  REGION 1..121: note = Description of sequence: VH
EVQLVESGGGLVQPGRSLRLSCAASGFTFDDYAIHWVRQAPGKGLEWVSGMSWNSGSIGYGDSVKGRFTISRDNAKNSLYLQMNSLRAEDTAFYYCAKDKSIGAAGRFDCWGQGTLVTVSS

SEQ ID NO: 56 - protein, 121 aa, Homo sapiens
  REGION 1..121: note = Description of sequence: VH
EVQLVESGGGLVQPGRSLRLSCAASGFTFDDYAIHWVRQAPGKGLEWVSGMSWNSGSIGYGDSVKGRFTISRDNAKNSLYLQMNSLRAEDTAFYYCAKDKSIGAAGRFDSWGQGTLVTVSS

SEQ ID NO: 57 - protein, 120 aa, Homo sapiens
  REGION 1..120: note = Description of sequence: VH
QVQLVQSGAEVKKPGASVRVSCKASGYTFIGFYIHWVRQAPGQGLEWMGWINPESGDTTYAQKFQGRVTMTTDTSINTAYMDLNRLRSDDSAVYFCARGNWNFPDTFDFWGQGTMVIVSS

SEQ ID NO: 58 - protein, 118 aa, Homo sapiens
  REGION 1..118: note = Description of sequence: VH
EVQLQQSGPELQRPGASVKLSCKASGYTFTEYYMYWVKQRPKQGLELIGRIDPEDGGTDYIEKFKNKATLTADTSSNTAYMQLSSLTSEDTATYFCATTIRGTVFAFWGQGTLVTVSS

SEQ ID NO: 59 - protein, 118 aa, Homo sapiens
  REGION 1..118: note = Description of sequence: VH
EVQLQQSGPELQRPGASVKLSCKASGYTFTEYYMYWVKQRPKQGLELMGRIDPEDGGTDYVEKFKNKATLTADTSSNTAYMQLSSLTSEDTATYFCASTIRGTVFVYWGQGTLVTVSS

SEQ ID NO: 60 - protein, 119 aa, Homo sapiens
  REGION 1..119: note = Description of sequence: VH
QVQLVQSGAEVKKPGASVKVSCKASGYTFTGHYMHWVRQAPGQGLEWMGWINPNSGDTDYAQKFQGRVTMTRDTSISTAYMDLNRLRSDDTAVFYCAREGLDGDPFDYWGQGTLVTVSS

SEQ ID NO: 61 - protein, 120 aa, Homo sapiens
  REGION 1..120: note = Description of sequence: VH
QVQLQESGPGLVKPSGTLSLTCAVSGGSISSNNWWSWVRQPPGKGLEWIGEIYHFGTTTYNPSLKSRVTISVDKSKNQFSLKLSSLTAADTAVYYCARTLRDFYYYMDVWGKGTTVTVSS

SEQ ID NO: 62 - protein, 124 aa, Homo sapiens
  REGION 1..124: note = Description of sequence: VH
EVQLVQSGAEIRKPGESLKISCKGSGYSFTTYWIAWVRQMPGKGLEWMGIIYPGDSDTRYSPSFQGQVTISADKSITTAYLQWSSLKASDTGIYYCARTYYYFSGSHWDAFDIWGQGTMVTVSS

SEQ ID NO: 63 - protein, 120 aa, Homo sapiens
  REGION 1..120: note = Description of sequence: VH
EVKLVESGGGLVRPGGSQKLSCAVSGFAFSGYDMSWVRQTPEKRLEWVATISNGGRHTYYPDSVKGRFTISRDNARNTLYLQMSSLRSEDTALYYCARQKTWDVHAMDYWGQGTSVTVSS

SEQ ID NO: 64 - protein, 107 aa, Homo sapiens
  REGION 1..107: note = Description of sequence: VL
DIQMTQSPSSLSASVGDRVTITCQASRRINNYLNWYQQKPGKAPKLLIYDASNLETGVPSRFTGSGSGTDFTLTISSLQPEDIATYYCQQYDNLPLTFGGGTKIKIK

SEQ ID NO: 65 - protein, 107 aa, Homo sapiens
  REGION 1..107: note = Description of sequence: VL
DIQMTQSPSSLSASVGDRVTITCQASRRINNYLNWYQQKPGKAPKLLIYDASNLETGVPSRFTGSGSGTDFTLTISSLQPEDIATYYCQQYDNLPLTFGGGTKVEIK

SEQ ID NO: 66 - protein, 107 aa, Homo sapiens
  REGION 1..107: note = Description of sequence: VL
DIQMTQSPSSLSASVGDRVTITCQASQGINNYLNWYQQKPGKAPKLLIYDASNLETGVPSRFSGSGSGTDFTFTISSLQPEDIATYYCQQYDNLPLTFGGGTKVEIK

SEQ ID NO: 67 - protein, 107 aa, Homo sapiens
  REGION 1..107: note = Description of sequence: VL
DIQMTQSPSSLSASVGDRVTITCRSSQSISIYLNWHQQIPGKAPKLLIYVASSLQSGIPSRFSGRGSGTEFTLTISSLQPEDFATYYCQQSYSAPFTFGPGTKVDIK

SEQ ID NO: 68 - protein, 107 aa, Homo sapiens
  REGION 1..107: note = Description of sequence: VL
DIQMTQSPASLSASLGETVTIECRASEDIFNGLAWYQQKPGKSPHLLIYNAKTLHTGVPSRFSGSGSGSQYSLKINSLQSEDVASYFCQQYYDYPLTFGSGTKLEIK

SEQ ID NO: 69 - protein, 107 aa, Homo sapiens
  REGION 1..107: note = Description of sequence: VL
DIQMTQSPASLSASLGETVTIECRPSEDIYNGLAWYQQKPGESPQLLIYNANSLHTGVPSRFSGSGSGTQYSLKINSLQSEDVASYFCQQYYDYPLTFGSGTKLEIK

SEQ ID NO: 70 - protein, 111 aa, Homo sapiens
  REGION 1..111: note = Description of sequence: VL
EIMLTQTPLSSPVTLGQPASISCRSSQSLVHSDGNTYLSWLQQRPGQPPRLLIYNISNRFSGVPDRFSGRGAGTDFTLKISRVEAEDVGVYYCIQTTQFSTFGQGTKLEIK

SEQ ID NO: 71 - protein, 107 aa, Homo sapiens
  REGION 1..107: note = Description of sequence: VL
DIHMTQSPSSLSASVGDRVTITCRTSQNINTYLNWYQQRPGRAPKLLIYAASSLQSGVPSRFSGSGSGTDFILSISSLQPEDFATYYCQQSFNIPLTFGGGTKVEIK

SEQ ID NO: 72 - protein, 107 aa, Homo sapiens
  REGION 1..107: note = Description of sequence: VL
DIQMTQSPSSVSAYVGDRVTITCRASQDIRDCLAWYQQKPGKAPKFLIYAASSFQSGVPSGFSGSGSGTDFTLTVTSLQPEDSATYYCQQTHSFPYTFGQGTKLEIK

SEQ ID NO: 73 - protein, 115 aa, Homo sapiens
  REGION 1..115: note = Description of sequence: VL
QLVLTQSSSASFSLGASAKLTCTLSSQHSTYTIEWYQQQPLKPPKYVMELKKDGSHSTGDGIPDRFSGSSSGADRYLSISNIQPEDEAIYICGVGDTIKEQFVYVFGGGTKVTVL

SEQ ID NO: 74 - protein, 10 aa, Homo sapiens
  REGION 1..10: note = Description of sequence: CDR-H1
  VARIANT 2: note = X is F or Y
  VARIANT 5: note = X is D or I
  VARIANT 6: note = X is D or G
  VARIANT 7: note = X is Y or F
  VARIANT 8: note = X is A or Y
  VARIANT 9: note = X isr M or I
GXTFXXXXXH

SEQ ID NO: 75-76 - skipped (2 entries)

SEQ ID NO: 77 - protein, 11 aa, Homo sapiens
  REGION 1..11: note = Description of sequence: CDR-L1
  VARIANT 1: note = X is Q or R
  VARIANT 2: note = X is A or S
  VARIANT 4: note = X is R or Q
  VARIANT 5: note = X is R, G or S
  VARIANT 7: note = X is N or S
  VARIANT 8: note = X is N or I
XXSXXIXXYLN

SEQ ID NO: 78 - protein, 7 aa, Homo sapiens
  REGION 1..7: note = Description of sequence: CDR-L2
  VARIANT 1: note = X is D or V
  VARIANT 4: note = X is N or S
  VARIANT 7: note = X is T or S
XASXLZX

SEQ ID NO: 79 - protein, 9 aa, Homo sapiens
  REGION 1..9: note = Description of sequence: CDR-L3
  VARIANT 3: note = X is Y or S
  VARIANT 4: note = X is D or Y
  VARIANT 5: note = X is N or S
  VARIANT 6: note = X is L or A
  VARIANT 8: note = X is L or F
QQXXXXPXT

SEQ ID NO: 80 - protein, 10 aa, Homo sapiens
  REGION 1..10: note = Description of sequence: CDR-H1
  VARIANT 9: note = X is M or I
GFTFDDYAXH

SEQ ID NO: 81 - protein, 10 aa, Homo sapiens
  REGION 1..10: note = Description of sequence: CDR-H2
  VARIANT 2: note = X is F or M
GXSWNSGSIG

SEQ ID NO: 82 - protein, 12 aa, Homo sapiens
  REGION 1..12: note = Description of sequence: CDR-H3
  VARIANT 5: note = X is S or G
  VARIANT 12: note = X is Y or S
DKSIXAAGRFDX

SEQ ID NO: 83 - protein, 11 aa, Homo sapiens
  REGION 1..11: note = Description of sequence: CDR-L1
  VARIANT 4: note = X is R or Q
  VARIANT 5: note = X is R or G
QASXXINNYLN

SEQ ID NO: 84 - protein, 10 aa, Homo sapiens
  REGION 1..10: note = Description of sequence: CDR-H1
  VARIANT 6: note = X is E or G
  VARIANT 7: note = X is Y or H
GYTFTXXYMY

SEQ ID NO: 85 - protein, 10 aa, Homo sapiens
  REGION 1..10: note = Description of sequence: CDR-H2
  VARIANT 1: note = X is R or W
  VARIANT 5: note = X is E or N
  VARIANT 6: note = X is D or S
  VARIANT 8: note = X is G or D
XIBPXXGXTD

SEQ ID NO: 86 - protein, 9 aa, Homo sapiens
  REGION 1..9: note = Description of sequence: CDR-H3
  VARIANT 8: note = X is A or V
  VARIANT 9: note = X is F or Y
TIRGTVFXX

SEQ ID NO: 87 - protein, 11 aa, Homo sapiens
  REGION 1..11: note = Description of sequence: CDR-L1
  VARIANT 2: note = X is A or P
  VARIANT 7: note = X is F or Y
RXSEDIXNGLA

SEQ ID NO: 88 - skipped

SEQ ID NO: 89 - protein, 7 aa, Homo sapiens
  REGION 1..7: note = Description of sequence: CDR-L1
  VARIANT 3: note = X is K or N
  VARIANT 4: note = X is T or S
NAXXLHT

SEQ ID NO: 90 - protein, 5 aa, Homo sapiens
  REGION 1..5: note = Description of sequence: hPILRA
QGKRR

SEQ ID NO: 91 - protein, 8 aa, Homo sapiens
  REGION 1..8: note = Description of sequence: hPILRA
TTQRPSSM

SEQ ID NO: 92 - protein, 4 aa, Homo sapiens
  REGION 1..4: note = Description of sequence: hPILRA
QGKR

SEQ ID NO: 93 - protein, 5 aa, Macaca fascicularis
  REGION 1..5: note = Description of sequence: cynoPILRA
QGKRH

SEQ ID NO: 94 - protein, 217 aa, Homo sapiens
  REGION 1..217: note = Description of sequence: Wild-type human Fc sequence
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 95 - protein, 110 aa, Homo sapiens
  REGION 1..110: note = Description of sequence: CH2 domain sequence
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 96 - protein, 107 aa, Homo sapiens
  REGION 1..107: note = Description of sequence: CH3 domain sequence
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 97 - protein, 15 aa, Homo sapiens
  REGION 1..15: note = Description of sequence: human IgG1 hinge
EPKSCDKTHTCPPCP

SEQ ID NO: 98 - protein, 10 aa, Homo sapiens
  REGION 1..10: note = Description of sequence: human IgG1 partial hinge
DKTHTCPPCP

SEQ ID NO: 99 - protein, 217 aa, Homo sapiens
  REGION 1..217: note = Description of sequence: Fc sequence with LALA
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 100 - protein, 217 aa, Homo sapiens
  REGION 1..217: note = Description of sequence: Fc sequence with LALA PG
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 101 - protein, 217 aa, Homo sapiens
  REGION 1..217: note = Description of sequence: Fc sequence with LALA PS
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 102 - protein, 217 aa, Homo sapiens
  REGION 1..217: note = Description of sequence: Fc sequence with LS
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 103 - protein, 217 aa, Homo sapiens
  REGION 1..217: note = Description of sequence: Fc sequence with LALA LS
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 104 - protein, 217 aa, Homo sapiens
  REGION 1..217: note = Description of sequence: Fc sequence with LALA PG LS
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 105 - protein, 217 aa, Homo sapiens
  REGION 1..217: note = Description of sequence: Fc sequence with LALA PS LS
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALSAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 106 - protein, 193 aa, Homo sapiens
  REGION 1..193: note = Description of sequence: Portion of hPILRA protein-linker-His tag
QPSGSTGSGPSYLYGVTQPKHLSASMGGSVEIPFSFYYPWELATAPDVRISWRRGHFHGQSFYSTRPPSIHKDYVNRLFLNWTEGQKSGFLRISNLQKQDQSVYFCRVELDTRSSGRQQWQSIEGTKLSITQAVTTTTQRPSSMTTTWRLSSTTTTTGLRVTQGKRRSDSWHISLETGGGSGGGSHHHHHHHH

SEQ ID NO: 107 - protein, 193 aa, Homo sapiens
  REGION 1..193: note = Description of sequence: Portion of cynoPILRA protein-linker-His tag
QPGGSAGSGPSGPYGVTQRKHLSAPMGGSVEIPFSFYHPWELAAAPNMKISWRRGNFHGEFFYRTRPAFIHEDYSNRLLLNWTEGQDRGLLRIWNLRKEDQSVYFCRVELDTRRSGRQRWQSIEGTKLTITQAVTTTTQRPSSMTTTRRPSSATTTAGLRVTQGKRHSDSWHLSLKTGGGSGGGSHHHHHHHH

SEQ ID NO: 108 - protein, 185 aa, Homo sapiens
  REGION 1..185: note = Description of sequence: Portion of hPILRB protein-linker-His tag
QPGGSTGSGPSYLYGVTQPKHLSASMGGSVEIPFSFYYPWELAIVPNVRISWRRGHFHGQSFYSTRPPSIHKDYVNRLFLNWTEGQESGFLRISNLRKEDQSVYFCRVELDTRRSGRQQLQSIKGTKLTITQAVTTTTTWRPSSTTTIAGLRVTESKGHSESWHLSLDTGGGSGGGSHHHHHHHH

SEQ ID NO: 109 - protein, 185 aa, Homo sapiens
  REGION 1..185: note = Description of sequence: PILRA-STEM-Truncated-His (hPILRA M1)
QPSGSTGSGPSYLYGVTQPKHLSASMGGSVEIPFSFYYPWELATAPDVRISWRRGHFHGQSFYSTRPPSIHKDYVNRLFLNWTEGQKSGFLRISNLQKQDQSVYFCRVELDTRSSGRQQWQSIEGTKLSITQAVTTTTTWRLSSTTTTTGLRVTQGKRRSDSWHISLETGGGSGGGSHHHHHHHH

SEQ ID NO: 110 - protein, 193 aa, Homo sapiens
  REGION 1..193: note = Description of sequence: PILRA-TAPD63IVPN (hPILRA M2)
QPSGSTGSGPSYLYGVTQPKHLSASMGGSVEIPFSFYYPWELAIVPNVRISWRRGHFHGQSFYSTRPPSIHKDYVNRLFLNWTEGQKSGFLRISNLQKQDQSVYFCRVELDTRSSGRQQWQSIEGTKLSITQAVTTTTQRPSSMTTTWRLSSTTTTTGLRVTQGKRRSDSWHISLETGGGSGGGSHHHHHHHH

SEQ ID NO: 111 - protein, 193 aa, Homo sapiens
  REGION 1..193: note = Description of sequence: PILRA-G78R-His (hPILRA M3)
QPSGSTGSGPSYLYGVTQPKHLSASMGGSVEIPFSFYYPWELATAPDVRISWRRGHFHRQSFYSTRPPSIHKDYVNRLFLNWTEGQKSGFLRISNLQKQDQSVYFCRVELDTRSSGRQQWQSIEGTKLSITQAVTTTTQRPSSMTTTWRLSSTTTTTGLRVTQGKRRSDSWHISLETGGGSGGGSHHHHHHHH

SEQ ID NO: 112 - protein, 193 aa, Homo sapiens
  REGION 1..193: note = Description of sequence: PILRA-K106E-His (hPILRA M4)
QPSGSTGSGPSYLYGVTQPKHLSASMGGSVEIPFSFYYPWELATAPDVRISWRRGHFHGQSFYSTRPPSIHKDYVNRLFLNWTEGQESGFLRISNLQKQDQSVYFCRVELDTRSSGRQQWQSIEGTKLSITQAVTTTTQRPSSMTTTWRLSSTTTTTGLRVTQGKRRSDSWHISLETGGGSGGGSHHHHHHHH

SEQ ID NO: 113 - protein, 193 aa, Homo sapiens
  REGION 1..193: note = Description of sequence: PILRA-QKQ116RKE-His (hPILRA M5)
QPSGSTGSGPSYLYGVTQPKHLSASMGGSVEIPFSFYYPWELATAPDVRISWRRGHFHGQSFYSTRPPSIHKDYVNRLFLNWTEGQKSGFLRISNLRKEDQSVYFCRVELDTRSSGRQQWQSIEGTKLSITQAVTTTTQRPSSMTTTWRLSSTTTTTGLRVTQGKRRSDSWHISLETGGGSGGGSHHHHHHHH

SEQ ID NO: 114 - protein, 193 aa, Homo sapiens
  REGION 1..193: note = Description of sequence: PILRA-S133R-His (hPILRA M6)
QPSGSTGSGPSYLYGVTQPKHLSASMGGSVEIPFSFYYPWELATAPDVRISWRRGHFHGQSFYSTRPPSIHKDYVNRLFLNWTEGQKSGFLRISNLQKQDQSVYFCRVELDTRRSGRQQWQSIEGTKLSITQAVTTTTQRPSSMTTTWRLSSTTTTTGLRVTQGKRRSDSWHISLETGGGSGGGSHHHHHHHH

SEQ ID NO: 115 - protein, 193 aa, Homo sapiens
  REGION 1..193: note = Description of sequence: PILRA-W139L-His (hPILRA M7)
QPSGSTGSGPSYLYGVTQPKHLSASMGGSVEIPFSFYYPWELATAPDVRISWRRGHFHGQSFYSTRPPSIHKDYVNRLFLNWTEGQKSGFLRISNLQKQDQSVYFCRVELDTRSSGRQQLQSIEGTKLSITQAVTTTTQRPSSMTTTWRLSSTTTTTGLRVTQGKRRSDSWHISLETGGGSGGGSHHHHHHHH

SEQ ID NO: 116 - protein, 193 aa, Homo sapiens
  REGION 1..193: note = Description of sequence: PILRA-E143K-His (hPILRA M8)
QPSGSTGSGPSYLYGVTQPKHLSASMGGSVEIPFSFYYPWELATAPDVRISWRRGHFHGQSFYSTRPPSIHKDYVNRLFLNWTEGQKSGFLRISNLQKQDQSVYFCRVELDTRSSGRQQWQSIKGTKLSITQAVTTTTQRPSSMTTTWRLSSTTTTTGLRVTQGKRRSDSWHISLETGGGSGGGSHHHHHHHH

SEQ ID NO: 117 - protein, 193 aa, Homo sapiens
  REGION 1..193: note = Description of sequence: PILRA-L169P-His (hPILRA M9)
QPSGSTGSGPSYLYGVTQPKHLSASMGGSVEIPFSFYYPWELATAPDVRISWRRGHFHGQSFYSTRPPSIHKDYVNRLFLNWTEGQKSGFLRISNLQKQDQSVYFCRVELDTRSSGRQQWQSIEGTKLSITQAVTTTTQRPSSMTTTWRPSSTTTTTGLRVTQGKRRSDSWHISLETGGGSGGGSHHHHHHHH

SEQ ID NO: 118 - protein, 193 aa, Homo sapiens
  REGION 1..193: note = Description of sequence: PILRA-TT175IA-His (hPILRA M10)
QPSGSTGSGPSYLYGVTQPKHLSASMGGSVEIPFSFYYPWELATAPDVRISWRRGHFHGQSFYSTRPPSIHKDYVNRLFLNWTEGQKSGFLRISNLQKQDQSVYFCRVELDTRSSGRQQWQSIEGTKLSITQAVTTTTQRPSSMTTTWRLSSTTTIAGLRVTQGKRRSDSWHISLETGGGSGGGSHHHHHHHH

SEQ ID NO: 119 - protein, 193 aa, Homo sapiens
  REGION 1..193: note = Description of sequence: PILRA-CT-swap-His (hPILRA M11)
QPSGSTGSGPSYLYGVTQPKHLSASMGGSVEIPFSFYYPWELATAPDVRISWRRGHFHGQSFYSTRPPSIHKDYVNRLFLNWTEGQKSGFLRISNLQKQDQSVYFCRVELDTRSSGRQQWQSIEGTKLSITQAVTTTTQRPSSMTTTWRLSSTTTTTGLRVTESKGHSESWHLSLDTGGGSGGGSHHHHHHHH

SEQ ID NO: 120 - protein, 113 aa, Homo sapiens
  REGION 1..113: note = Description of sequence: Heavy chain constant domain 1 (CH1)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCP

SEQ ID NO: 121 - protein, 107 aa, Homo sapiens [sequence omitted]

SEQ ID NO: 122 - protein, 451 aa, Homo sapiens
  REGION 1..451: note = Description of sequence: Heavy chain sequence of Ab.2
EVQLVESGGGLVQPGRSLRLSCAVSGFTFDDYAMHWVRQAPGKGLEWVSGFSWNSGSIGYPDSVKGRFTISRDNAKNSLYLQMNSLRAEDTAFYYCAKDKSISAAGRFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 123 - protein, 214 aa, Homo sapiens
  REGION 1..214: note = Description of sequence: Light chain sequence of Ab.2
DIQMTQSPSSLSASVGDRVTITCQASRRINNYLNWYQQKPGKAPKLLIYDASNLETGVPSRFTGSGSGTDFTLTISSLQPEDIATYYCQQYDNLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 124 - protein, 451 aa, Homo sapiens
  REGION 1..451: note = Description of sequence: Heavy chain sequence of Ab.4
EVQLVESGGGLVQPGRSLRLSCAASGFTFDDYAIHWVRQAPGKGLEWVSGMSWNSGSIGYGDSVKGRFTISRDNAKNSLYLQMNSLRAEDTAFYYCAKDKSIGAAGRFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 125 - protein, 214 aa, Homo sapiens
  REGION 1..214: note = Description of sequence: Light chain sequence of Ab.4
DIQMTQSPSSLSASVGDRVTITCQASQGINNYLNWYQQKPGKAPKLLIYDASNLETGVPSRFSGSGSGTDFTFTISSLQPEDIATYYCQQYDNLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 126 - protein, 450 aa, Homo sapiens
  REGION 1..450: note = Description of sequence: Heavy chain sequence of Ab.5
QVQLVQSGAEVKKPGASVRVSCKASGYTFIGFYIHWVRQAPGQGLEWMGWINPESGDTTYAQKFQGRVTMTTDTSINTAYMDLNRLRSDDSAVYFCARGNWNFPDTFDFWGQGTMVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 127 - protein, 214 aa, Homo sapiens
  REGION 1..214: note = Description of sequence: Light chain sequence of Ab.5
DIQMTQSPSSLSASVGDRVTITCRSSQSISIYLNWHQQIPGKAPKLLIYVASSLQSGIPSRFSGRGSGTEFTLTISSLQPEDFATYYCQQSYSAPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 128 - protein, 450 aa, Homo sapiens
  REGION 1..450: note = Description of sequence: Heavy chain sequence of Reference Antibody #1
QVTLQESGPGILQPSQTLSLTCSFSGFSLSTFGMGVGWIRQPSGKGLEWLAHIWWDDDKYYNPALKSRLTISKDTSKNQVFLKIASVDTADIATYYCARVEDYGNPFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 129 - protein, 214 aa, Homo sapiens
  REGION 1..214: note = Description of sequence: Light chain sequence of Reference Antibody #1
DIQMNQSPSSLSASLGDTITITCHASQNIHVWLNWYQQKPGNIPKLLIYKASNLHTGVPSRFSGSGSGTGFTVTISSLQPEDIATYYCQQGQSYPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 130 - protein, 450 aa, Homo sapiens
  REGION 1..450: note = Description of sequence: Heavy chain sequence of Reference Antibody #2
QVILKESGPGILQSSQTLSLTCSFSGFSLSTFGMGVGWIRQPSGKGLESLAHIWWDDDKFYNPALKSRLTISKDTSKSQVFLKIANVDTADIATYYCTRIEDYGSYFAYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 131 - protein, 214 aa, Homo sapiens
  REGION 1..214: note = Description of sequence: Light chain sequence of Reference Antibody #2
DVQMNQSPSSLSASLGDPITITCHASQNIHVWLNWYQQRPGNIPRLLIYKASNLHTGVPSRFSGSGSGTGFTLTISSLQPEDIATYYCQQGQSYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 132 - protein, 450 aa, Homo sapiens
  REGION 1..450: note = Description of sequence: Heavy chain sequence of Reference Antibody #3
QVTLKESGPGMLQPSQTLSLACSFSGFSLNSFGVAVGWIRQPSGKGLEWLAHIWWDDDKSYNPALKSRLTISKDTSKNQVFLKLANVDTADTATYYCTRIADYGNHFDYWGQGTALTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 133 - protein, 214 aa, Homo sapiens
  REGION 1..214: note = Description of sequence: Light chain sequence of Reference Antibody #3
DIQMNQSPSSLSASLGDTITITCHASQNSHVWLSWYQQKPGNIPKLLIYKASNLHTGVPSRFSGSGSGTGFTLTISGLQPEDIATYYCQQGQTYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 134 - protein, 450 aa, Homo sapiens
  REGION 1..450: note = Description of sequence: Heavy chain sequence of Reference Antibody #4
QVTLKESGPGILQPSQTLSLTCSFSGFSLTTFGMGVGWIRQPSGKGLEWLAHIWWDDDKYYNPALKSRLTISKDISKNQVFLKIANVDTADTATYYCARIEDYGNPFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 135 - protein, 214 aa, Homo sapiens
  REGION 1..214: note = Description of sequence: Light chain sequence of Reference Antibody #4
DIQMNQSPSSLSASLGDTITITCHASQNIHVWLSWYQQKPGNIPKLLIYKASNLHTGVPSRFSGSGSGTGFTLTISSLQPEDIATYYCQQGQSYPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 136 - protein, 303 aa, Homo sapiens
  REGION 1..303: note = Description of sequence: Human PILRA protein (R78)
MGRPLLLPLLPLLLPPAFLQPSGSTGSGPSYLYGVTQPKHLSASMGGSVEIPFSFYYPWELATAPDVRISWRRGHFHRQSFYSTRPPSIHKDYVNRLFLNWTEGQKSGFLRISNLQKQDQSVYFCRVELDTRSSGRQQWQSIEGTKLSITQAVTTTTQRPSSMTTTWRLSSTTTTTGLRVTQGKRRSDSWHISLETAVGVAVAVTVLGIMILGLICLLRWRRRKGQQRTKATTPAREPFQNTEEPYENIRNEGQNTDPKLNPKDDGIVYASLALSSSTSPRAPPSHRPLKSPQNETLYSVLKA

SEQ ID NO: 137 - protein, 118 aa, Homo sapiens
  REGION 1..118: note = Description of sequence: VH
EVQLVQSGAEVKKPGASVKVSCKASGYTFTEYYMYWVRQAPGQGLELIGRIDPEDGGTDYIEKFKNRVTITADTSTSTAYLELSSLRSEDTAVYYCATTIRGTVFAFWGQGTLVTVSS

SEQ ID NO: 138 - protein, 118 aa, Homo sapiens
  REGION 1..118: note = Description of sequence: VH
EVQLVQSGAEVKKPGASVKVSCKASGYTFTEYYMYWVRQAPGQGLELIGRIDPEDGGTDYIEKFKNRVTLTADTSTSTAYLELSSLRSEDTAVYYCATTIRGTVFAFWGQGTLVTVSS

SEQ ID NO: 139 - protein, 118 aa, Homo sapiens
  REGION 1..118: note = Description of sequence: VH
EVQLVQSGAEVKKPGASVKVSCKASGYTFTEYYMYWVRQAPGQGLELIGRIDPEDGGTDYIEKFKNRVTITADTSTSTAYLELSSLRSEDTAVYFCATTIRGTVFAFWGQGTLVTVSS

SEQ ID NO: 140 - protein, 118 aa, Homo sapiens
  REGION 1..118: note = Description of sequence: VH
EVQLVQSGAEVKKPGASVKVSCKASGYTFTEYYMYWVRQAPGQGLELIGRIDPEDGGTDYIEKFKNRSTLTADTSTSTAYLELSSLRSEDTAVYFCATTIRGTVFAFWGQGTLVTVSS

SEQ ID NO: 141 - protein, 118 aa, Homo sapiens
  REGION 1..118: note = Description of sequence: VH
EVQLVQSGAEVKKPGASVKVSCKASGYTFTEYYMYWVRQAPGQGLELIGRIDPEDGGTDYVEKFKNRATITADTSTSTAYLELSSLRSEDTAVYYCASTIRGTVFVYWGQGTLVTVSS

SEQ ID NO: 142 - protein, 118 aa, Homo sapiens
  REGION 1..118: note = Description of sequence: VH
EVQLVQSGAEVKKPGASVKVSCKASGYTFTEYYMYWVRQAPGQGLELIGRIDPEDGGTDYVEKFKNRVTITADTSTSTAYLELSSLRSEDTAVYFCASTIRGTVFVYWGQGTLVTVSS

SEQ ID NO: 143 - protein, 118 aa, Homo sapiens
  REGION 1..118: note = Description of sequence: VH
EVQLVQSGAEVKKPGASVKVSCKASGYTFTEYYMYWVRQAPGQGLELIGRIDPEDGGTDYVEKFKNRATLTADTSTSTAYLELSSLRSEDTAVYFCASTIRGTVFVYWGQGTLVTVSS

SEQ ID NO: 144 - protein, 118 aa, Homo sapiens
  REGION 1..118: note = Description of sequence: VH
EVQLVQSGAEVKKPGASVKVSCKASGYTFTEYYMYWVRQAPGQGLELIGRIDPEDGGTDYVEKFKNRVTATADTSTSTAYLELSSLRSEDTAVYYCASTIRGTVFVYWGQGTLVTVSS

SEQ ID NO: 145 - protein, 107 aa, Homo sapiens
  REGION 1..107: note = Description of sequence: VL
DIQMTQSPSSLSASVGDRVTITCRASEDIFNGLAWYQQKPGKSPKLLIYNAKTLHTGVPSRFSGSGSGSDYTLTISSLQPEDFATYFCQQYYDYPLTFGQGTKVEIK

SEQ ID NO: 146 - protein, 107 aa, Homo sapiens
  REGION 1..107: note = Description of sequence: VL
DIQMTQSPSSLSASVGDRVTITCRPSEDIYNGLAWYQQKPGKAPKLLIYNANSLHTGVPSRFSGSGSGTDYTLTISSLQPEDFATYYCQQYYDYPLTFGQGTKVEIK

SEQ ID NO: 147 - protein, 107 aa, Homo sapiens
  REGION 1..107: note = Description of sequence: VL
DIQMTQSPSSLSASVGDRVTITCRPSEDIYNGLAWYQQKPGKAPKLLIYNANSLHTGVPSRFSGSGSGTDYTLTISSLQPEDFATYFCQQYYDYPLTFGQGTKVEIK

SEQ ID NO: 148 - protein, 107 aa, Homo sapiens
  REGION 1..107: note = Description of sequence: VL
DIQMTQSPSSLSASVGDRVTITCRASEDIFNGLAWYQQKPGKSPKLLIYNAKTLHTGVPSRFSGSGSGTDYTLTISSLQPEDFATYYCQQYYDYPLTFGQGTKVEIK

SEQ ID NO: 149 - protein, 107 aa, Homo sapiens
  REGION 1..107: note = Description of sequence: VL
DIQMTQSPSSLSASVGDRVTITCRPSEDIYNGLAWYQQKPGKSPKLLIYNANSLHTGVPSRFSGSGSGTDYTLTISSLQPEDFATYFCQQYYDYPLTFGQGTKVEIK

SEQ ID NO: 150 - protein, 448 aa, Homo sapiens
  REGION 1..448: note = Description of sequence: Heavy chain sequence of Ab.12
EVQLVQSGAEVKKPGASVKVSCKASGYTFTEYYMYWVRQAPGQGLELIGRIDPEDGGTDYIEKFKNRVTITADTSTSTAYLELSSLRSEDTAVYYCATTIRGTVFAFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 151 - protein, 214 aa, Homo sapiens
  REGION 1..214: note = Description of sequence: Light chain sequence of Ab.12 and Ab.15
DIQMTQSPSSLSASVGDRVTITCRASEDIFNGLAWYQQKPGKSPKLLIYNAKTLHTGVPSRFSGSGSGSDYTLTISSLQPEDFATYFCQQYYDYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 152 - protein, 448 aa, Homo sapiens
  REGION 1..448: note = Description of sequence: Heavy chain sequence of Ab.15
EVQLVQSGAEVKKPGASVKVSCKASGYTFTEYYMYWVRQAPGQGLELIGRIDPEDGGTDYIEKFKNRSTLTADTSTSTAYLELSSLRSEDTAVYFCATTIRGTVFAFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 153 - protein, 448 aa, Homo sapiens
  REGION 1..448: note = Description of sequence: Heavy chain sequence of Ab.23 and Ab.35
EVQLVQSGAEVKKPGASVKVSCKASGYTFTEYYMYWVRQAPGQGLELIGRIDPEDGGTDYVEKFKNRATLTADTSTSTAYLELSSLRSEDTAVYFCASTIRGTVFVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 154 - protein, 214 aa, Homo sapiens
  REGION 1..214: note = Description of sequence: Light chain sequence of Ab.23
DIQMTQSPSSLSASVGDRVTITCRPSEDIYNGLAWYQQKPGKAPKLLIYNANSLHTGVPSRFSGSGSGTDYTLTISSLQPEDFATYYCQQYYDYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 155 - protein, 214 aa, Homo sapiens
  REGION 1..214: note = Description of sequence: Light chain sequence of Ab.35
DIQMTQSPSSLSASVGDRVTITCRPSEDIYNGLAWYQQKPGKSPKLLIYNANSLHTGVPSRFSGSGSGTDYTLTISSLQPEDFATYFCQQYYDYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 156 - protein, 450 aa, Homo sapiens
  REGION 1..450: note = Description of sequence: Heavy chain sequence of PILRB binding positive control antibody (CDRS bold and underlined)
QVQLQESGPGLVKPSGTLSLTCAVSGGSISSNHWWSWVRQPPGKGLEWIGEIYHYGTTDYNPSLQSRVTISVDKSKNQFSLKLTSVTAADTAVYFCARGLRDYYYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 157 - protein, 214 aa, Homo sapiens
  REGION 1..214: note = Description of sequence: Light chain sequence of PILRB binding positive control antibody (CDRS bold and underlined)
DIQMTQSPSSLSASVGDRVTITCRASQTISSYLNWYQQKPGKAPKLLIYAASSLQSGVPSRFSGSGFGTDFTLTISSLQPEDFATYYCQQSYSIPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 158 - protein, 118 aa, Homo sapiens
  REGION 1..118: note = Description of sequence: VH
EVQLVQSGAEVKKPGASVKVSCKASGYTFTEYYMYWVRQAPGQGLELIGRIDPEDGGTDYVEKFKNRVTLTADTSTSTAYLELSSLRSEDTAVYYCASTIRGTVFVYWGQGTLVTVSS

(19) United States
(12) Patent Application Publication
(10) Pub. No.: US 2026/0109763 A1
(43) Pub. Date: Apr. 23, 2026

(54) ANTI-PILRA ANTIBODIES, USES THEREOF, AND RELATED METHODS AND REAGENTS

(71) Applicant: DENALI THERAPEUTICS INC., South San Francisco, CA (US)

(72) Inventors: Claire Da Roza, South San Francisco, CA (US); Do Jin Kim, South San Francisco, CA (US); Kathleen Lisaingo, South San Francisco, CA (US); Madeline MacDonald, South San Francisco, CA (US); Kathryn M. Monroe, South San Francisco, CA (US); Joshua I. Park, South San Francisco, CA (US); Nicholas E. Propson, South San Francisco, CA (US); Hanna Sabelström, South San Francisco, CA (US); Richard Théolis, JR., South San Francisco, CA (US); Tanya N. Weerakkody, South San Francisco, CA (US); Alexander Yang, South San Francisco, CA (US)

(73) Assignee: DENALI THERAPEUTICS INC., South San Francisco, CA (US)

(21) Appl. No.: 18/714,069

(22) PCT Filed: Dec. 16, 2022
(86) PCT No.: PCT/US2022/053245

§ 371 Date: May 28, 2024

Related U.S. Application Data
(60) Provisional application No. 63/290,930, filed on Dec. 17, 2021.

(51) Int. Cl. C07K 16/28; A61K 39/00; A61P 25/28

(52) U.S. Cl. CPC C07K 16/2803; A61P 25/28; A61K 2039/505; C07K 2317/52; C07K 2317/565; C07K 2317/76; C07K 2317/92

(57) ABSTRACT

Provided herein are anti-PILRA antibodies with the highly desirable selectivity: having comparable binding to cynomolgus and human PILRA proteins, but much weaker binding to human PILRB protein, as well as binding to both PILRA G78 and R78 variants. The binding and selectivity profiles of the antibodies described herein allow for them to be used in animal studies (e.g., monkeys) without the need to rely on a surrogate molecule and also when treating subjects with either PILRA variant. Further described herein, for the first time, are biological discoveries related to PILRA and the effects of reducing PILRA signaling in cells.

### CROSS-REFERENCE TO RELATED APPLICATIONS

The present application is a 371 national stage application of International Patent Application No. PCT/US2022/053245, filed Dec. 16, 2022, and claims priority to U.S. Provisional Patent Application No. 63/290,930, filed on Dec. 17, 2021, the disclosures of which are incorporated herein by reference in their entireties for all purposes.

### REFERENCE TO A SEQUENCE LISTING

A Sequence Listing conforming to the rules of WIPO Standard ST.26 is submitted electronically herewith via Patent Center and is hereby incorporated by reference in its entirety. The Sequence Listing file, identified as “632729CORRECTEDSEQLIST.xml”, is 227,418 bytes in size and was created on Oct. 22, 2025.

### BACKGROUND

Paired immunoglobulin-like type 2 receptor alpha (PILRA) is a transmembrane receptor that is expressed on various immune cells, such as microglia and is believed to function in inhibitory cell signaling pathways. A missense variant (G78R) of PILRA is associated with reduced risk of Alzheimer's disease. The G78R variant alters the interaction of residues essential for sialic acid engagement, resulting in reduced binding for several PILRA ligands.

There remains a need for therapeutic agents that modulate PILRA activity.

### BRIEF SUMMARY

Described herein are antibodies that selectively bind to both cynomolgus monkey PILRA (cynoPILRA) and hPILRA, but may have comparatively lower binding to human PILRB (hPILRB). We have identified epitopes that allow for the desired selectivity. This selectivity profile is highly advantageous, but also very challenging, given the high homology between cynoPILRA and hPILRB. Having comparable binding between cyno and human proteins allows for conducting studies in monkeys without having to rely on a surrogate molecule. Binding to PILRB, by contrast, is not desired, because PILRB while having an extracellular domain highly similar to PILRA, has a different intracellular domain, which is expected to have different or even opposing activity.

Furthermore, certain antibodies with this selectivity profile described herein also bind to, and have activity at, both PILRA variant forms (G78 and R78), thus ensuring that they can be used in a variety of populations, given that the frequency of each variant varies highly in different parts of the world.

In addition to developing highly useful antibodies, we have also made significant discoveries related to PILRA biology, including discovery of certain downstream effectors of PILRA signaling, and for the first time, have characterized effects of reducing signaling by the PILRA receptor in microglia. These insights allow, for the first time, linking PILRA ligand blocking to biological effects in cells, which provides novel approaches for both drug discovery as well as measuring biological impacts of known PILRA binders on cells and animals.

In one aspect, the disclosure features an isolated antibody or antigen-binding fragment thereof that specifically binds to a cynomolgus monkey paired immunoglobulin-like type 2 receptor alpha (cynoPILRA), wherein the binding affinity for the cynoPILRA is at least 2-fold (e.g., at least 2-fold, 5-fold, 10-fold, 20-fold, 30-fold, 40-fold, 50-fold, 60-fold, 70-fold, 80-fold, 90-fold, or 100-fold) stronger than the binding affinity for a human paired immunoglobulin-like type 2 receptor beta (hPILRB). In some embodiments, the antibody or antigen-binding fragment thereof also binds to a human paired immunoglobulin-like type 2 receptor alpha (hPILRA).

In another aspect, the disclosure features an isolated antibody or antigen-binding fragment thereof that binds to a human paired immunoglobulin-like type 2 receptor alpha (hPILRA) and a cynomolgus monkey PILRA (cynoPILRA), wherein the binding affinity for the cynoPILRA is within 100-fold (e.g., within 90-fold, 80-fold, 70-fold, 60-fold, 50-fold, 40-fold, 30-fold, 20-fold, 10-fold, 5-fold, or 2-fold) relative to the binding affinity for the hPILRA.

In some embodiments of this aspect, the binding affinity for the cynoPILRA is within 50-fold (e.g., within 45-fold, 40-fold, 35-fold, 30-fold, 25-fold, 20-fold, 15-fold, 10-fold, 5-fold, or 2-fold) relative to the binding affinity for the hPILRA. In some embodiments of this aspect, the binding affinity for the cynoPILRA is within 25-fold (e.g., within 20-fold, 15-fold, 10-fold, 9-fold, 8-fold, 7-fold, 6-fold, 5-fold, 4-fold, 3-fold, or 2-fold) relative to the binding affinity for the hPILRA. In some embodiments, the binding affinity for the cynoPILRA is within 10-fold (e.g., within 9-fold, 8-fold, 7-fold, 6-fold, 5-fold, 4-fold, 3-fold, or 2-fold) relative to the binding affinity for the hPILRA. In certain embodiments, the binding affinity for the cynoPILRA is within 5-fold (e.g., within 4-fold, 3-fold, or 2-fold) relative to the binding affinity for the hPILRA. In particular embodiments, the binding affinity for the cynoPILRA is within 2-fold relative to the binding affinity for the hPILRA.

In some embodiments of this aspect, the antibody or antigen-binding fragment thereof binds to a human paired immunoglobulin-like type 2 receptor beta (hPILRB) with weaker affinity compared to hPILRA and cynoPILRA. In some embodiments, the binding affinity for the hPILRA is at least 10-fold (e.g., at least 20-fold, 40-fold, 60-fold, 80-fold, 100-fold, 120-fold, 140-fold, 160-fold, 180-fold, 200-fold, 220-fold, 240-fold, 260-fold, 280-fold, or 300-fold) stronger than the binding affinity for the hPILRB. In some embodiments, the binding affinity for the hPILRA is at least 25-fold (e.g., at least 30-fold, 35-fold, 40-fold, 45-fold, 50-fold, 55-fold, 60-fold, 80-fold, 100-fold, 120-fold, 140-fold, 160-fold, 180-fold, 200-fold, 220-fold, 240-fold, 260-fold, 280-fold, or 300-fold) stronger than the binding affinity for the hPILRB. In certain embodiments, the binding affinity for the hPILRA is at least 100-fold (e.g., at least 110-fold, 120-fold, 130-fold, 140-fold, 150-fold, 160-fold, 170-fold, 180-fold, 190-fold, 200-fold, 220-fold, 240-fold, 260-fold, 280-fold, or 300-fold) stronger than the binding affinity for the hPILRB.

In some embodiments of the aspects of the disclosure described herein, the binding affinity for the cynoPILRA is at least 10-fold (e.g., at least 20-fold, 40-fold, 60-fold, 80-fold, or 100-fold) stronger than the binding affinity for the hPILRB. In some embodiments, the binding affinity for the cynoPILRA is at least 25-fold (e.g., at least 30-fold, 35-fold, 40-fold, 45-fold, 50-fold, 60-fold, 70-fold, 80-fold, 90-fold, or 100-fold) stronger than the binding affinity for the hPILRB.

In another aspect, the disclosure features an isolated antibody or antigen-binding fragment thereof that binds to a human paired immunoglobulin-like type 2 receptor alpha (hPILRA), wherein the antibody or antigen-binding fragment thereof binds to one or more amino acids at one or more of the following positions: 63, 64, 78, 106, 143, 116-118, and 182-186, wherein the positions are determined with reference to the sequence of SEQ ID NO:1.

In some embodiments, the antibody or antigen-binding fragment thereof binds to one or more amino acids at one or more of the following positions: 78, 106, and 143. In certain embodiments, the antibody or antigen-binding fragment thereof binds to G78, K106, and E143 of SEQ ID NO: 1. In certain embodiments, the antibody or antigen-binding fragment thereof binds to R78, K106, and E143 of SEQ ID NO:136.

In some embodiments, the antibody or antigen-binding fragment thereof binds to one or more amino acids at one or more of the following positions: 63 and 64. In certain embodiments, the antibody or antigen-binding fragment thereof binds to T63 and A64 of SEQ ID NO:1.

In some embodiments, the antibody or antigen-binding fragment thereof binds to one or more amino acids at one or more of the following positions: 106, 116-118, and 182-186. In certain embodiments, the antibody or antigen-binding fragment thereof binds to K106 of SEQ ID NO:1. In certain embodiments, the antibody or antigen-binding fragment thereof binds to Q116, K117, and/or Q118 of SEQ ID NO:1. In certain embodiments, the antibody or antigen-binding fragment thereof binds to Q182, G183, K184, R185, and/or R186 of SEQ ID NO:1.

In some embodiments of the aspects of the disclosure described herein, the antibody or antigen-binding fragment thereof comprises:
(a) a heavy chain CDR1 (CDR-H1) sequence having at least 90% sequence identity (e.g., at least 91%, 92%, 93%, 94%, 95%, 96%, 97%, 98%, or 99% sequence identity) to the amino acid sequence of any one of SEQ ID NOS:4-11, or having up to two amino acid substitutions relative to the amino acid sequence of any one of SEQ ID NOS:4-11;
(b) a heavy chain CDR2 (CDR-H2) sequence having at least 80% sequence identity (e.g., at least 82%, 84%, 86%, 88%, 90%, 91%, 92%, 93%, 94%, 95%, 96%, 97%, 98%, or 99% sequence identity) to the amino acid sequence of any one of SEQ ID NOS:12-19, or having up to two amino acid substitutions relative to the amino acid sequence of any one of SEQ ID NOS:12-19;
(c) a heavy chain CDR3 (CDR-H3) sequence having at least 80% sequence identity (e.g., at least 82%, 84%, 86%, 88%, 90%, 91%, 92%, 93%, 94%, 95%, 96%, 97%, 98%, or 99% sequence identity) to the amino acid sequence of any one of SEQ ID NOS:20-29, or having up to two amino acid substitutions relative to the amino acid sequence of any one of SEQ ID NOS:20-29;
(d) a light chain CDR1 (CDR-L1) sequence having at least 90% sequence identity (e.g., at least 91%, 92%, 93%, 94%, 95%, 96%, 97%, 98%, or 99% sequence identity) to the amino acid sequence of any one of SEQ ID NOS:30-38, or having up to two amino acid substitutions relative to the amino acid sequence of any one of SEQ ID NOS:30-38;
(e) a light chain CDR2 (CDR-L2) sequence having at least 80% sequence identity (e.g., at least 82%, 84%, 86%, 88%, 90%, 91%, 92%, 93%, 94%, 95%, 96%, 97%, 98%, or 99% sequence identity) to the amino acid sequence of SEQ ID NO:39-46, or having up to two amino acid substitutions relative to the amino acid sequence of SEQ ID NO:39-46; and
(f) a light chain CDR3 (CDR-L3) sequence having at least 80% sequence identity (e.g., at least 82%, 84%, 86%, 88%, 90%, 91%, 92%, 93%, 94%, 95%, 96%, 97%, 98%, or 99% sequence identity) to the amino acid sequence of any one of SEQ ID NOS:47-53, or having up to two amino acid substitutions relative to the amino acid sequence of any one of SEQ ID NOS:47-53.

In some embodiments, the amino acid substitutions are conservative substitutions.

In some embodiments, the antibody or antigen-binding fragment comprises:
(i) a CDR-H1 comprising the sequence of SEQ ID NO:4 or one or more conservative substitutions relative to the sequence of SEQ ID NO:4; a CDR-H2 comprising the sequence of SEQ ID NO:12 or one or more conservative substitutions relative to the sequence of SEQ ID NO:12; a CDR-H3 comprising the sequence of SEQ ID NO:20 or one or more conservative substitutions relative to the sequence of SEQ ID NO:20; a CDR-L1 comprising the sequence of SEQ ID NO:30 or one or more conservative substitutions relative to the sequence of SEQ ID NO:30; a CDR-L2 comprising the sequence of SEQ ID NO:39 or one or more conservative substitutions relative to the sequence of SEQ ID NO:39; and a CDR-L3 comprising the sequence of SEQ ID NO:47 or one or more conservative substitutions relative to the sequence of SEQ ID NO:47; or
(ii) a CDR-H1 comprising the sequence of SEQ ID NO:5 or one or more conservative substitutions relative to the sequence of SEQ ID NO:5; a CDR-H2 comprising the sequence of SEQ ID NO:13 or one or more conservative substitutions relative to the sequence of SEQ ID NO:13; a CDR-H3 comprising the sequence of SEQ ID NO:22 or one or more conservative substitutions relative to the sequence of SEQ ID NO:22; a CDR-L1 comprising the sequence of SEQ ID NO:31 or one or more conservative substitutions relative to the sequence of SEQ ID NO:31; a CDR-L2 comprising the sequence of SEQ ID NO:39 or one or more conservative substitutions relative to the sequence of SEQ ID NO:39; and a CDR-L3 comprising the sequence of SEQ ID NO:47 or one or more conservative substitutions relative to the sequence of SEQ ID NO:47; or
(iii) a CDR-H1 comprising the sequence of SEQ ID NO:6 or one or more conservative substitutions relative to the sequence of SEQ ID NO:6; a CDR-H2 comprising the sequence of SEQ ID NO:14 or one or more conservative substitutions relative to the sequence of SEQ ID NO:14; a CDR-H3 comprising the sequence of SEQ ID NO:23 or one or more conservative substitutions relative to the sequence of SEQ ID NO:23; a CDR-L1 comprising the sequence of SEQ ID NO:32 or one or more conservative substitutions relative to the sequence of SEQ ID NO:32; a CDR-L2 comprising the sequence of SEQ ID NO:40 or one or more conservative substitutions relative to the sequence of SEQ ID NO:40; and a CDR-L3 comprising the sequence of SEQ ID NO:48 or one or more conservative substitutions relative to the sequence of SEQ ID NO:48;
(iv) a CDR-H1 comprising the sequence of SEQ ID NO:7 or one or more conservative substitutions relative to the sequence of SEQ ID NO:7; a CDR-H2 comprising the sequence of SEQ ID NO:15 or one or more conservative substitutions relative to the sequence of SEQ ID NO:15; a CDR-H3 comprising the sequence of SEQ ID NO:24 or one or more conservative substitutions relative to the sequence of SEQ ID NO:24; a CDR-L1 comprising the sequence of SEQ ID NO:33 or one or more conservative substitutions relative to the sequence of SEQ ID NO:33; a CDR-L2 comprising the sequence of SEQ ID NO:41 or one or more conservative substitutions relative to the sequence of SEQ ID NO:41; and a CDR-L3 comprising the sequence of SEQ ID NO:49 or one or more conservative substitutions relative to the sequence of SEQ ID NO:49; or
(v) a CDR-H1 comprising the sequence of SEQ ID NO:7 or one or more conservative substitutions relative to the sequence of SEQ ID NO:7; a CDR-H2 comprising the sequence of SEQ ID NO:15 or one or more conservative substitutions relative to the sequence of SEQ ID NO:15; a CDR-H3 comprising the sequence of SEQ ID NO:25 or one or more conservative substitutions relative to the sequence of SEQ ID NO:25; a CDR-L1 comprising the sequence of SEQ ID NO:34 or one or more conservative substitutions relative to the sequence of SEQ ID NO:34; a CDR-L2 comprising the sequence of SEQ ID NO:42 or one or more conservative substitutions relative to the sequence of SEQ ID NO:42; and a CDR-L3 comprising the sequence of SEQ ID NO:49 or one or more conservative substitutions relative to the sequence of SEQ ID NO:49; or
(vi) a CDR-H1 comprising the sequence of SEQ ID NO:8 or one or more conservative substitutions relative to the sequence of SEQ ID NO:8; a CDR-H2 comprising the sequence of SEQ ID NO:16 or one or more conservative substitutions relative to the sequence of SEQ ID NO:16; a CDR-H3 comprising the sequence of SEQ ID NO:26 or one or more conservative substitutions relative to the sequence of SEQ ID NO:26; a CDR-L1 comprising the sequence of SEQ ID NO:35 or one or more conservative substitutions relative to the sequence of SEQ ID NO:35; a CDR-L2 comprising the sequence of SEQ ID NO:43 or one or more conservative substitutions relative to the sequence of SEQ ID NO:43; and a CDR-L3 comprising the sequence of SEQ ID NO:50 or one or more conservative substitutions relative to the sequence of SEQ ID NO:50.

In some embodiments, the antibody or antigen-binding fragment thereof comprises:
(a) a CDR-H1 sequence comprising the sequence of GX₁TFX₂X₃X₄X₅X₆H (SEQ ID NO:74), wherein X₁ is F or Y; X₂ is D or I; X₃ is D or G; X₄ is Y or F; X₅ is A or Y; and X₆ is M or I;
(b) a CDR-H2 sequence comprising the sequence of X₁X₂X₃X₄X₅SGX₆X₇X₈ (SEQ ID NO:75), wherein X₁ is G or W; X₂ is F, M, or I; X₃ is S or N; X₄ is W or P; X₅ is N or E; X₆ is S or D; X₇ is I or T; and X₈ is G or T;
(c) a CDR-H3 sequence comprising the sequence of X₁X₂X₃X₄X₅X₆X₇X₈X₉FDX₁₀ (SEQ ID NO:76), wherein X₁ is D or absent; X₂ is K or G; X₃ is S or N; X₄ is I or W; X₅ is S, G, or N; X₆ is A or F; X₇ is A or P; X₈ is G or D; X₉ is R or T; and X₁₀ is Y, S, or F;
(d) a CDR-L1 sequence comprising the sequence of X₁X₂SX₃X₄IX₅X₆YLN (SEQ ID NO:77), wherein X₁ is Q or R; X₂ is A or S; X₃ is R or Q; X₄ is R, G, or S; X₅ is N or S; and X₆ is N or I;
(e) a CDR-L2 sequence comprising the sequence of X₁ASX₂LX₃X₄ (SEQ ID NO:78), wherein X₁ is D or V; X₂ is N or S; X₃ is E or Q; and X₄ is T or S; and
(f) a CDR-L3 sequence comprising the sequence of QQX₁X₂X₃X₄PX₅T (SEQ ID NO:79), wherein X₁ is Y or S; X₂ is D or Y; X₃ is N or S; X₄ is L or A; and X₅ is L or F.

In some embodiments, the antibody or antigen-binding fragment comprises:
(i) a CDR-H1 comprising the sequence of SEQ ID NO:4; a CDR-H2 comprising the sequence of SEQ ID NO:12; a CDR-H3 comprising the sequence of SEQ ID NO:20; a CDR-L1 comprising the sequence of SEQ ID NO:30; a CDR-L2 comprising the sequence of SEQ ID NO:39; and a CDR-L3 comprising the sequence of SEQ ID NO:47; or
(ii) a CDR-H1 comprising the sequence of SEQ ID NO:5; a CDR-H2 comprising the sequence of SEQ ID NO:13; a CDR-H3 comprising the sequence of SEQ ID NO:22; a CDR-L1 comprising the sequence of SEQ ID NO:31; a CDR-L2 comprising the sequence of SEQ ID NO: 39; and a CDR-L3 comprising the sequence of SEQ ID NO:47; or
(iii) a CDR-H1 comprising the sequence of SEQ ID NO:6; a CDR-H2 comprising the sequence of SEQ ID NO:14; a CDR-H3 comprising the sequence of SEQ ID NO:23; a CDR-L1 comprising the sequence of SEQ ID NO:32; a CDR-L2 comprising the sequence of SEQ ID NO:40; and a CDR-L3 comprising the sequence of SEQ ID NO:48.

In some embodiments, the antibody or antigen-binding fragment thereof comprises:
(i) a CDR-H1 comprising the sequence of SEQ ID NO:7; a CDR-H2 comprising the sequence of SEQ ID NO:15; a CDR-H3 comprising the sequence of SEQ ID NO:24; a CDR-L1 comprising the sequence of SEQ ID NO:33; a CDR-L2 comprising the sequence of SEQ ID NO:41; and a CDR-L3 comprising the sequence of SEQ ID NO:49; or
(ii) a CDR-H1 comprising the sequence of SEQ ID NO:7; a CDR-H2 comprising the sequence of SEQ ID NO:15; a CDR-H3 comprising the sequence of SEQ ID NO:25; a CDR-L1 comprising the sequence of SEQ ID NO:34; a CDR-L2 comprising the sequence of SEQ ID NO:42; and a CDR-L3 comprising the sequence of SEQ ID NO:49; or
(iii) a CDR-H1 comprising the sequence of SEQ ID NO:8; a CDR-H2 comprising the sequence of SEQ ID NO:16; a CDR-H3 comprising the sequence of SEQ ID NO:26; a CDR-L1 comprising the sequence of SEQ ID NO:35; a CDR-L2 comprising the sequence of SEQ ID NO:43; and a CDR-L3 comprising the sequence of SEQ ID NO:50.

In particular embodiments, the antibody or antigen-binding fragment thereof comprises: a CDR-H1 comprising the sequence of SEQ ID NO:7; a CDR-H2 comprising the sequence of SEQ ID NO:15; a CDR-H3 comprising the sequence of SEQ ID NO:24; a CDR-L1 comprising the sequence of SEQ ID NO:33; a CDR-L2 comprising the sequence of SEQ ID NO:41; and a CDR-L3 comprising the sequence of SEQ ID NO:49.

In particular embodiments, the antibody or antigen-binding fragment thereof comprises: a CDR-H1 comprising the sequence of SEQ ID NO:7; a CDR-H2 comprising the sequence of SEQ ID NO:15; a CDR-H3 comprising the sequence of SEQ ID NO:25; a CDR-L1 comprising the sequence of SEQ ID NO:34; a CDR-L2 comprising the sequence of SEQ ID NO:42; and a CDR-L3 comprising the sequence of SEQ ID NO:49.

In some embodiments, the isolated antibody or antigen-binding fragment described herein comprises a heavy chain variable region (V_{H}) sequence that has at least 85% sequence identity (e.g., at least 86%, 87%, 88%, 89%, 90%, 91%, 92%, 93%, 94%, 95%, 96%, 97%, 98%, or 99% sequence identity) to any one of SEQ ID NOS:54-63. In some embodiments, the isolated antibody or antigen-binding fragment described herein comprises a V_{H} sequence has at least 90% sequence identity (e.g., at least 91%, 92%, 93%, 94%, 95%, 96%, 97%, 98%, or 99% sequence identity) to any one of SEQ ID NOS:54-63. In some embodiments, the isolated antibody or antigen-binding fragment described herein comprises a V_{H} sequence that has at least 95% sequence identity (e.g., at least 96%, 97%, 98%, or 99% sequence identity) to any one of SEQ ID NOS:54-63. In some embodiments, the isolated antibody or antigen-binding fragment described herein comprises a V_{H} sequence comprises a sequence of any one of SEQ ID NOS:54-63.

In some embodiments, the isolated antibody or antigen-binding fragment described herein comprises a heavy chain variable region (V_{H}) sequence that has at least 85% sequence identity (e.g., at least 86%, 87%, 88%, 89%, 90%, 91%, 92%, 93%, 94%, 95%, 96%, 97%, 98%, or 99% sequence identity) to any one of SEQ ID NOS:137-144. In some embodiments, the isolated antibody or antigen-binding fragment described herein comprises a V_{H} sequence has at least 90% sequence identity (e.g., at least 91%, 92%, 93%, 94%, 95%, 96%, 97%, 98%, or 99% sequence identity) to any one of SEQ ID NOS:137-144. In some embodiments, the isolated antibody or antigen-binding fragment described herein comprises a V_{H} sequence that has at least 95% sequence identity (e.g., at least 96%, 97%, 98%, or 99% sequence identity) to any one of SEQ ID NOS:137-144. In some embodiments, the isolated antibody or antigen-binding fragment described herein comprises a V_{H} sequence comprises a sequence of any one of SEQ ID NOS:137-144.

In some embodiments, the isolated antibody or antigen-binding fragment described herein comprises a light chain variable region (V_{L}) sequence that has at least 85% sequence identity (e.g., at least 86%, 87%, 88%, 89%, 90%, 91%, 92%, 93%, 94%, 95%, 96%, 97%, 98%, or 99% sequence identity) to any one of SEQ ID NOS:64-73. In some embodiments, the isolated antibody or antigen-binding fragment described herein comprises a V_{L} sequence has at least 90% sequence identity (e.g., at least 91%, 92%, 93%, 94%, 95%, 96%, 97%, 98%, or 99% sequence identity) to any one of SEQ ID NOS:64-73. In some embodiments, the isolated antibody or antigen-binding fragment described herein comprises a V_{L} sequence that has at least 95% sequence identity (e.g., at least 96%, 97%, 98%, or 99% sequence identity) to any one of SEQ ID NOS:64-73. In some embodiments, the isolated antibody or antigen-binding fragment described herein comprises a V_{L} sequence comprises a sequence of any one of SEQ ID NOS:64-73.

In some embodiments, the isolated antibody or antigen-binding fragment described herein comprises a light chain variable region (V_{L}) sequence that has at least 85% sequence identity (e.g., at least 86%, 87%, 88%, 89%, 90%, 91%, 92%, 93%, 94%, 95%, 96%, 97%, 98%, or 99% sequence identity) to any one of SEQ ID NOS:145-149. In some embodiments, the isolated antibody or antigen-binding fragment described herein comprises a V_{L} sequence has at least 90% sequence identity (e.g., at least 91%, 92%, 93%, 94%, 95%, 96%, 97%, 98%, or 99% sequence identity) to any one of SEQ ID NOS:145-149. In some embodiments, the isolated antibody or antigen-binding fragment described herein comprises a V_{L} sequence that has at least 95% sequence identity (e.g., at least 96%, 97%, 98%, or 99% sequence identity) to any one of SEQ ID NOS:145-149. In some embodiments, the isolated antibody or antigen-binding fragment described herein comprises a V_{L} sequence comprises a sequence of any one of SEQ ID NOS:145-149.

In some embodiments, the antibody or antigen-binding fragment comprises:
(i) a V_{H} sequence comprising SEQ ID NO:54 and a V_{L} sequence comprising SEQ ID NO:65; or
(ii) a V_{H} sequence comprising SEQ ID NO:56 and a V_{L} sequence comprising SEQ ID NO:66; or
(iii) a V_{H} sequence comprising SEQ ID NO:57 and a V_{L} sequence comprising SEQ ID NO:67; or
(iv) a V_{H} sequence comprising SEQ ID NO:58 and a V_{L} sequence comprising SEQ ID NO:68; or
(v) a V_{H} sequence comprising SEQ ID NO:59 and a V_{L} sequence comprising SEQ ID NO:69; or
(vi) a V_{H} sequence comprising SEQ ID NO:60 and a V_{L} sequence comprising SEQ ID NO:70.

In some embodiments, the antibody or antigen-binding fragment comprises:
(i) a V_{H} sequence comprising SEQ ID NO:54 and a V_{L} sequence comprising SEQ ID NO:65; or
(ii) a V_{H} sequence comprising SEQ ID NO:56 and a V_{L} sequence comprising SEQ ID NO:66; or
(iii) a V_{H} sequence comprising SEQ ID NO:57 and a V_{L} sequence comprising SEQ ID NO:67.

In some embodiments, the antibody or antigen-binding fragment comprises:
(i) a V_{H} sequence comprising SEQ ID NO:137 and a V_{L} sequence comprising SEQ ID NO:145; or
(ii) a V_{H} sequence comprising SEQ ID NO:140 and a V_{L} sequence comprising SEQ ID NO:145; or
(iii) a V_{H} sequence comprising SEQ ID NO:143 and a V_{L} sequence comprising SEQ ID NO:146; or
(iv) a V_{H} sequence comprising SEQ ID NO:143 and a V_{L} sequence comprising SEQ ID NO:149.

In some embodiments, the antibody comprises two Fc polypeptides forming an Fc domain. In some embodiments, one or both Fc polypeptides comprise a sequence having at least 90% (e.g., at least 91%, 92%, 93%, 94%, 95%, 96%, 97%, 98%, or 99%) identity to the sequence of SEQ ID NO:94.

In some embodiments, the antibody is an IgG1.

In some embodiments, the antibody is a full-length antibody.

In another aspect, the disclosure features an isolated antibody or antigen-binding fragment thereof that binds to a human paired immunoglobulin-like type 2 receptor alpha (hPILRA), wherein the antibody or antigen-binding fragment thereof recognizes an epitope that is the same or substantially the same as the epitope recognized by an antibody clone selected from the group consisting of: antibody clones 1-39 in Table 1.

In some embodiments of this aspect, the antibody or antigen-binding fragment thereof recognizes an epitope that is the same or substantially the same as the epitope recognized by an antibody clone selected from the group consisting of: antibody clones 2, 4, and 5.

In some embodiments of the isolated antibody or antigen-binding fragment thereof described herein, the antibody or antigen-binding fragment thereof antagonizes hPILRA activity. In some embodiments, the antibody or antigen-binding fragment thereof blocks binding of a sialyated protein to hPILRA. In some embodiments, the sialyated protein is a sialyated NPDC1, PANP, HSV-1 gB, COLEC12, C4a, C4b, DAG1, or Clec4g.

In some embodiments of the isolated antibody or antigen-binding fragment thereof described herein, the antibody or antigen-binding fragment thereof enhances or increases phosphorylation of EGFR or STAT3; or inhibits or decreases phosphorylation of STAT1.

In some embodiments of the isolated antibody or antigen-binding fragment thereof described herein, the antibody or antigen-binding fragment thereof enhances cell migration (e.g., microglia migration).

In some embodiments of the isolated antibody or antigen-binding fragment thereof described herein, the antibody or antigen-binding fragment thereof enhances anti-inflammatory gene or protein expression. In some embodiments, the antibody or antigen-binding fragment thereof enhances IL1RN gene expression.

In some embodiments of the isolated antibody or antigen-binding fragment thereof described herein, the antibody or antigen-binding fragment thereof reduces pro-inflammatory cytokine protein expression or secretion. In some embodiments, the antibody or antigen-binding fragment thereof reduces TNF, IL-6, and/or IP-10 expression.

In some embodiments of the isolated antibody or antigen-binding fragment thereof described herein, the antibody or antigen-binding fragment thereof elevates cellular respiration. In some embodiments, the antibody or antigen-binding fragment thereof increases mitochondrial respiration. In some embodiments, the antibody or antigen-binding fragment thereof increases non-mitochondrial respiration.

In some embodiments of the isolated antibody or antigen-binding fragment thereof described herein, the antibody or antigen-binding fragment thereof increases fatty acid metabolism (e.g., increases fatty acid oxidation).

In some embodiments of the isolated antibody or antigen-binding fragment thereof described herein, the antibody or antigen-binding fragment thereof increases ATP production.

In some embodiments of the isolated antibody or antigen-binding fragment thereof described herein, the antibody or antigen-binding fragment thereof does not activate peripheral immune cells. In some embodiments, the antibody or antigen-binding fragment thereof does not activate neutrophils and monocytes.

In some embodiments of the isolated antibody or antigen-binding fragment thereof described herein, the antibody is a monoclonal antibody. In some embodiments, the antibody is a chimeric antibody. In some embodiments, the antibody is a humanized antibody. In some embodiments, the antibody is a fully human antibody. In some embodiments, the antigen-binding fragment is a Fab, a F(ab′)2, a scFv, or a bivalent scFv.

In another aspect, the disclosure features an antibody or antigen-binding fragment thereof that competes with the isolated antibody or antigen-binding fragment thereof described herein for binding to hPILRA.

In another aspect, the disclosure features a pharmaceutical composition comprising the isolated antibody or antigen-binding fragment thereof described herein and a pharmaceutically acceptable carrier.

In another aspect, the disclosure features a polynucleotide comprising a nucleic acid sequence encoding the isolated antibody or antigen-binding fragment thereof described herein. In another aspect, the disclosure features a vector comprising the polynucleotide that comprises a nucleic acid sequence encoding the isolated antibody or antigen-binding fragment thereof described herein. In another aspect, the disclosure features a host cell comprising the polynucleotide that comprises a nucleic acid sequence encoding the isolated antibody or antigen-binding fragment thereof described herein.

In another aspect, the disclosure provides a method for producing an isolated antibody or antigen-binding fragment thereof, comprising culturing a host cell under conditions in which the isolated antibody or antigen-binding fragment thereof encoded by the polynucleotide is expressed.

In another aspect, the disclosure features a kit comprising: the isolated antibody or antigen-binding fragment thereof described herein or the pharmaceutical composition described herein; and instructions for use thereof.

In another aspect, the disclosure features a method of treating a neurodegenerative disease in a subject, comprising administering to the subject the isolated antibody or antigen-binding fragment thereof described herein or the pharmaceutical composition described herein. In some embodiments, the neurodegenerative disease is selected from the group consisting of Alzheimer's disease, primary age-related tauopathy, progressive supranuclear palsy (PSP), frontotemporal dementia, frontotemporal dementia with parkinsonism linked to chromosome 17, argyrophilic grain dementia, amyotrophic lateral sclerosis, amyotrophic lateral sclerosis/parkinsonism-dementia complex of Guam (ALS-PDC), corticobasal degeneration, chronic traumatic encephalopathy, Creutzfeldt-Jakob disease, dementia pugilistica, diffuse neurofibrillary tangles with calcification, Down's syndrome, familial British dementia, familial Danish dementia, Gerstmann-Straussler-Scheinker disease, globular glial tauopathy, Guadeloupean parkinsonism with dementia, Guadelopean PSP, Hallevorden-Spatz disease, hereditary diffuse leukoencephalopathy with spheroids (HDLS), Huntington's disease, inclusion-body myositis, multiple system atrophy, myotonic dystrophy, Nasu-Hakola disease, neurofibrillary tangle-predominant dementia, Niemann-Pick disease type C, pallido-ponto-nigral degeneration, Parkinson's disease, Pick's disease, postencephalitic parkinsonism, prion protein cerebral amyloid angiopathy, progressive subcortical gliosis, subacute sclerosing panencephalitis, and tangle only dementia. In particular embodiments, the neurodegenerative disease is Alzheimer's disease.

In another aspect, the disclosure provides a method for determining whether a molecule has activity at a PILRA protein, the method comprising: (a) contacting a cell that expresses the PILRA protein with the molecule; (b) either prior to, concurrently with, or following step (a), contacting a cell of the same type as in step (a) having lower PILRA expression with the molecule; and (c) measuring one of the following: phosphorylated STAT3 (pSTAT3) level, phosphorylated STAT1 (pSTAT1) level, phosphorylated EGFR (pEGFR) level, cadherin expression, integrin expression, and microglial migration in both cells, wherein a change in the level of one of these measurements between the cells indicates that the molecule has activity at the PILRA protein of step (a).

In some embodiments, the cell of step (a) naturally expresses the PILRA protein. In some embodiments, the cell having lower PILRA expression has the PILRA protein knocked-out. In particular embodiments, the cell is a microglia, such as an iMicroglia. In some embodiments, the cell is a PILRA LoF iMicroglia.

In some embodiments, the cell of step (a) is engineered or modified to express or overexpress the PILRA protein. In some embodiments, the cell having lower PILRA expression naturally expresses the PILRA protein or is not engineered or modified to express the PILRA protein.

In some embodiments, the molecule is from a library of molecules. In certain embodiments, the molecule is known to bind the PILRA protein. In other embodiments, it is unknown whether the molecule binds the PILRA protein. In certain embodiments, the molecule is an antibody, a peptide, an organic small molecule, or a nucleic acid.

In another aspect, the disclosure provides a method for determining whether a molecule that binds a PILRA protein modulates a signaling response or activity in a PILRA-expressing cell, the method comprising: (a) contacting the cell with the molecule; and (b) measuring one of the following: phosphorylated STAT3 (pSTAT3) level, phosphorylated STAT1 (pSTAT1) level, phosphorylated EGFR (pEGFR) level, cadherin expression, integrin expression, and microglial migration, wherein a change in the level of one of the measurements indicates that the molecule modulates the signaling response or activity in the PILRA-expressing cell.

In some embodiments, the change is an increase or decrease in the level of one of the measurements when the molecule contacts the cell, relative to the level in the cell without the molecule. For example, in some embodiments, the change is an increase in pSTAT3 level, e.g., an increase in pSTAT3 Y705 level, and/or pSTAT3 S727 level. In another example, the change is an increase in pEGFR level. In another example, the change is an increase in the expression level and/or cell secretion of a motile protein (e.g., a cadherin, an integrin, such as any of those described herein). In yet another example, the change is an increase in cell (e.g., microglia) migration, which can be measured and quantified using cell migration assays, such as described in Example 4.

In some embodiments, the cell is in an in vitro assay. In other embodiments, the cell is in a mammal. In some embodiments, step (a) comprises administering the molecule to the mamma.

In some embodiments, the cell is a microglia, a myeloid cell, a monocyte, or a neutrophil.

In another aspect, the disclosure provides an engineered human induced pluripotent stem cell (IPSC) or cell line, wherein the IPSC has been modified (i.e., genetically engineered) to express two copies of the gene encoding R78 variant or the G78 variant of a PILRA protein. In some embodiments, the IPSC is modified at the endogenous genomic locus.

In another aspect, the disclosure provides an engineered microglial cell model that is derived from a human induced pluripotent stem cell (IPSC), wherein the IPSC has been modified (i.e., genetically engineered) to express two copies of the gene encoding the R78 variant or the G78 variant of a PILRA protein. In some embodiments, the IPSC is modified at the endogenous genomic locus. In some embodiments, the engineered microglial cell model is derived by directed differentiation.

In another aspect, the disclosure provides a matched pair of cell lines, wherein: (a) the first cell line of the pair is homozygous for the gene encoding the R78 variant of a PILRA protein; and (b) the second cell line of the pair is homozygous for the gene encoding the G78 variant of a PILRA protein, wherein both first and second cell lines of the pair are derived from the same parental cell line, and one or both cell lines have been engineered in the endogenous PILRA gene. In some embodiments, the parental cell line is homozygous for the gene encoding the R78 variant of the PILRA protein. In other embodiments, the parental cell line is homozygous for the gene encoding the G78 variant of the PILRA protein. In some embodiments, the parental cell line is heterozygous for gene encoding the R78 variant and the G78 variant of the PILRA protein.

In some embodiments of the matched pair of cell lines, a third cell line is included that is heterozygous for the gene encoding the G78 variant and the R78 variant of the PILRA protein. In some embodiments, the third cell line is derived from the parental cell line that is homozygous for the gene encoding the R78 variant or the G78 variant of the PILRA protein.

In another aspect, the disclosure provides a method of generating a myeloid cell line, or a stem cell line capable of differentiating into a myeloid cell line (e.g., an IPSC line), with a modified PILRA gene, the method comprising: (a) determining whether an existing myeloid cell line, or an existing stem cell line, is homozygous for the gene encoding the R78 variant of a PILRA protein, homozygous for the gene encoding the G78 variant of a PILRA protein, or heterozygous for the gene encoding the R78 and G78 variants of a PILRA protein; and (b) engineering the cell line by modifying the gene encoding the PILRA protein to produce an engineered cell line that is homozygous for the gene encoding the R78 variant of the PILRA protein or the G78 variant of the PILRA protein, wherein the engineered cell line was not, prior to being engineered, homozygous for the gene that encodes the selected variant.

In another aspect, the disclosure provides a method of generating a matched pair of cell lines, the method comprising: (a) determining whether an existing myeloid cell line, or an existing stem cell line capable of differentiating into a myeloid cell line (e.g., an IPSC line), is homozygous for the gene encoding the R78 variant of a PILRA protein, homozygous for the gene encoding the G78 variant of a PILRA protein, or heterozygous for the gene encoding the R78 and G78 variants of a PILRA protein; and (b) engineering (i) a first cell line by modifying the gene encoding the PILRA protein to produce an engineered cell line that is homozygous for the gene encoding the R78 variant of the PILRA protein, and/or (ii) a second cell line by modifying the gene encoding the PILRA protein to produce an engineered cell line that is homozygous for the gene encoding the G78 variant of the PILRA protein.

In some embodiments, the engineered cell line was not, prior to being engineered, homozygous for the gene that encodes the selected variant.

In certain embodiments, the existing cell line of step (a) is homozygous for the R78 variant of the PILRA protein, and the engineering of step (b) comprises modifying the existing cell line to produce an engineered cell line that is homozygous for the gene encoding the G78 variant of the PILRA protein.

In other embodiments, the existing cell line of step (a) is homozygous for the G78 variant of the PILRA protein, and the engineering of step (b) comprises modifying the existing cell line to produce an engineered cell line that is homozygous for the gene encoding the R78 variant of the PILRA protein.

In some embodiments, the existing cell line of step (a) is heterozygous for the gene encoding the R78 and G78 variants of the PILRA protein, and the engineering of step (b) comprises modifying the existing cell line to produce an engineered cell line that is homozygous for the gene encoding the R78 variant of the PILRA protein, and an engineered cell line that is homozygous for the gene encoding the G78 variant of the PILRA protein.

In another aspect, the disclosure provides a method of generating a matched pair of cell lines from an existing myeloid cell line, or an existing stem cell line capable of differentiating into a myeloid cell line (e.g., an IPSC line), that is heterozygous for gene encoding the R78 and G78 variants of a PILRA protein, the method comprising: (a) engineering the existing cell line to produce a first engineered cell line that is homozygous for the gene encoding the R78 variant of the PILRA protein; and (b) engineering either the cell line generated in step (a) or the existing cell line to produce a second engineered cell line that is homozygous for the gene encoding the G78 variant of the PILRA protein.

In another aspect, the disclosure provides a method of generating a matched pair of cell lines from an existing myeloid cell line, or an existing stem cell line capable of differentiating into a myeloid cell line (e.g., an IPSC line), that is heterozygous for gene encoding the R78 and G78 variants of a PILRA protein, the method comprising: (a) engineering the existing cell line to produce a first engineered cell line that is homozygous for the gene encoding the G78 variant of the PILRA protein; and (b) engineering either the cell line generated in step (a) or the existing cell line to produce a second engineered cell line that is homozygous for the gene encoding the R78 variant of the PILRA protein.

In another aspect, the disclosure provides a method of generating a matched pair of cell lines from an existing myeloid cell line, or an existing stem cell line capable of differentiating into a myeloid cell line (e.g., an ISPC line), that is homozygous for the gene encoding the R78 variant of a PILRA protein, the method comprising: engineering the existing cell line to produce an engineered cell line that is homozygous for gene encoding the G78 variant of the PILRA protein.

In another aspect, the disclosure provides a method of generating a matched pair of cell lines from an existing myeloid cell line, or an existing stem cell line capable of differentiating into a myeloid cell line (e.g., an IPSC line), that is homozygous for the gene encoding the G78 variant of a PILRA protein, the method comprising: engineering the existing cell line to produce an engineered cell line that is homozygous for gene encoding the R78 variant of the PILRA protein.

### BRIEF DESCRIPTION OF THE DRAWINGS

FIGS. 1A-1C: Anti-PILRA antibodies bound to hPILRA expressed on HEK293 cells in a dose-dependent manner.

FIGS. 1D-1F: Anti-PILRA antibodies bound to hPILRA G78 or R78 expressed on HEK293 cells in a dose-dependent manner (FIG. 1D and FIG. 1F). No binding to parental HEK293 cells (FIG. 1E). The data is expressed as median fluorescence intensity fluorescence obtained via FACS assay technology.

FIG. 1G: Anti-PILRA antibodies bound to hPILRA expressed on CHO-K1 cells and did not bind to parental CHO-K1 cells.

FIGS. 1H and 1I: Anti-PILRA antibodies bound to CHO-K1 cells expressing hPILRA G78 (FIG. 1H) in a dose-dependent manner and showed no binding to parental CHO-K1 cells (FIG. 1I). The data is expressed as median fluorescence intensity fluorescence obtained via FACS assay technology.

FIGS. 1J and 1K: Anti-PILRA antibodies bound to human IPSC-derived microglia (FIG. 1J) and did not bind to PILRA LoF human IPSC-derived microglia.

FIGS. 1L and 1M: Anti-PILRA antibodies bound to human IPSC-derived iMicroglia homozygous for PILRA G78 (FIG. 1L) or PILRA R78 (FIG. 1M).

FIG. 2A: Anti-PILRA antibodies bound to CHO-K1 cells expressing cynoPILRA and did not bind to CHO-K1 cells expressing hPILRB or to parental CHO-K1 cells.

FIGS. 2B and 2C: Anti-PILRA antibodies bound to CHO cells expressing cynoPILRA (FIG. 2B) in a dose-dependent manner and did not bind to CHO cells expressing hPILRB (FIG. 2C).

FIG. 2D shows anti-PILRA antibodies did not bind to hPILRB-DAP12 overexpressing HEK293 cells.

FIGS. 2E and 2F: Reference Antibodies bound CHO cells expressing hPILRA but did not bind to CHO cells expressing cyno PILRA or hPILRB.

FIGS. 3A and 3B: Representative SPR sensorgrams of when ligand binding was permitted (FIG. 3A) and when ligand binding was blocked by the antibodies (FIG. 3B).

FIGS. 3C-3F: Sialidase treatment in PILRA G78 HEK cells enhanced anti-PILRA antibody binding.

FIGS. 3G-3J: Sialidase treatment of PILRA R78 HEK cells had minimal effect on anti-PILRA antibody binding.

FIGS. 4A and 4B: PILRA LoF iMicroglia had increased levels of phosphorylated EGFR Y1086 (FIG. 4A) and STAT3 Y705 (FIG. 4B) compared to wild-type human iMicroglia in serum-free media. Graphs show spot intensity expression above background as mean+/−SEM. N=2 technical replicates. P>0.01, 2-way ANOVA.

FIGS. 4C-4E: Anti-PILRA antibodies specifically induced pSTAT3 Y705, pSTAT3 S727, and pEGFR Y1086 in HEK293 cells expressing hPILRA G78. No induction in parental HEK293 cells or by isotype control antibodies. Data is presented as mean fold over background (PBS) or mean+/−SEM fold over isotype control (n=4 technical replicates).

FIG. 4F: Dose-dependent induction of pSTAT3 Y705 in HEK293 cells expressing hPILRA G78. Anti-PILRA antibodies were dose titrated on HEK293 cells expressing hPILRA G78 and pSTAT3 Y705 induction was measured after 30 minutes. Data is presented as mean+/−SEM fold expression over isotype control, n=2 technical replicates.

FIGS. 4G and 4H: Anti-PILRA antibodies dose titrated on human PILRA 78G expressing HEK cells and induced pSTAT3 Y705 (FIG. 4G) or pSTAT3 S727 (FIG. 4H) after 30 minutes.

FIG. 4I: Anti-PILRA antibody-induced pSTAT3 Y705 was partially blocked by 2 hours pre-incubation with mTORC1/2 inhibitor AZD8055 (3.125-50 nM) or mTOR inhibitor Torin1 (31.25-500 nM) in PILRA G7G expressing HEK293 cells. Data is presented as mean+/−SEM fold expression over HEK293 control cells (DMSO vehicle isotype control), n=1-4 technical replicates.

FIG. 4J: Anti-PILRA antibodies blocked induction of pSTAT3 Y705 in HEK293 cells expressing PILRA R78. Data is presented as mean+/−SEM fold expression relative to PILRA G78 expressing HEK293 cells, n=2-3 technical replicates.

FIGS. 4K and 4L: Anti-PILRA antibodies dose titrated on human PILRA 78R expressing HEK293 cells and induced pSTAT3 Y705 (FIG. 4K) or pSTAT3 S727 (FIG. 4L) after 30 minutes.

FIGS. 4M and 4N: PILRA LoF iMicroglia showed lower phosphorylated STAT1 Y701 (FIG. 4M) and total STAT1 (FIG. 4N) levels compared to wild-type human iMicroglia in serum-free media by phospho-kinase profiler and total STAT1 AlphaLisa. Graph in FIG. 4M shows spot intensity expression above background as mean+/−SEM. N=2 technical replicates. Graph in FIG. 4N shows fold expression relative to wild-type as mean+/−SEM. N=4 technical replicates.

FIG. 4O: HEK293 cells expressing PILRA G78 showed higher phosphorylated STAT1 level compared to parental HEK293 cells. Expression is shown as mean+/−SEM. N=3-4 technical replicates.

FIG. 4P: Dosing with anti-PILRA mAb at 100 nM reduced phosphorylated STAT1 Y701 levels in wild-type human iMicroglia, phenocopying PILRA LoF iMicroglia. Expression is shown relative to PBS as mean+/−SEM. N=3 technical replicates.

FIGS. 4Q and 4R: Dosing with anti-PILRA mAb reduced phosphorylated STAT1 Y701 and total STAT1 levels, respectively, in HEK293 cells expressing PILRA G78. There was no reduction in parental HEK293 cells or by isotype control antibody. Graphs show fold expression relative to PBS as mean+/−SEM. N=2-3 technical replicates.

FIG. 5A: PILRA LoF promotes iMicroglial migration to cell-free detection zone 120 hours after stopper removal. Re-expression of PILRA in PILRA LoF iMicroglia (PILRA LoF+OE) brought migration to back to the level observed in wild-type iMicroglia.

FIGS. 5B and 5C: Anti-PILRA antibodies enhanced wild-type iMicroglial migration to cell-free detection zone 120 hours after stopper removal, similar to PILRA LoF iMicroglia cells. PILRA LoF iMicroglia and wild-type iMicroglia were both only dosed with vehicle (PBS). Data is presented as mean+/−SEM, n=1-6 technical replicates.

FIGS. 5D and 5E: PILRA LoF enhanced iMicroglial migration to chemoattractant complement 5a (C5a) (FIG. 5D) and anti-PILRA antibodies enhanced chemotaxis of iMicroglia to C5a, similar to PILRA LoF iMicroglia cells (FIG. 5E).

FIGS. 5F and 5G: Anti-PILRA antibodies enhanced iMicroglial secretion of integrins (FIG. 5F) and cadherins (FIG. 5G) into the supernatant after 4 days of treatment.

FIGS. 6A and 6B: PILRA LoF promoted IL1RN gene expression (FIG. 6A) and stimulated IL1RA cytokine secretion (FIG. 6B) in PILRA LoF iMicroglia compared to wild-type iMicroglia in serum-free media. Data is presented as mean+/−SEM, n=3 technical replicates.

FIG. 6C: Anti-PILRA antibodies stimulated IL1RA cytokine secretion in wild-type iMicroglia in serum-free media, mimicking the phenotype observed in PILRA LoF iMicroglia. Data is presented as mean+/−SEM, n=3 technical replicates.

FIGS. 6D-6F: PILRA LoF suppressed LPS-induced gene expression changes in TNF, IL-6, and CXCL10 in PILRA LoF iMicroglia relative to wild-type iMicroglia. Data is presented as mean+/−SEM, n=3 technical replicates.

FIGS. 6G-6I: PILRA LoF suppressed LPS-induced cytokine expression changes in TNFalpha, IL-6, and IP-10 in PILRA LoF iMicroglia relative to wild-type iMicroglia. Data is presented as mean+/−SEM, n=3 technical replicates.

FIGS. 6J-6O: Anti-PILRA antibodies attenuated LPS-induced IP-10, TNFalpha, and IL-6 cytokine secretion in wild-type iMicroglia, mimicking the phenotype observed in PILRA LoF iMicroglia. Data is presented as mean+/−SEM, n=3 technical replicates.

FIGS. 6P and 6Q: Anti-PILRA antibodies (100 nM) attenuated LPS-induced IP-10 cytokine secretion in homozygous G78 (FIG. 6P) and R78 (FIG. 6Q) PILRA expressing IPSC-derived iMicroglia in serum-free media.

FIGS. 7A and 7B: PILRA LoF iMicroglia displayed elevated maximal respiration and spare mitochondrial capacity. Re-expression of PILRA in PILRA LoF iMicroglia expressing hPILRA (PILRA LoF+OE) restored mitochondrial respiration to wild-type levels. n=5 tech. reps.

FIGS. 7C-7F: Anti-PILRA antibodies increased maximal respiration and spare mitochondrial respiratory capacity in wildtype iMicroglia relative to isotype control. There was no additional impact of antibodies on PILRA LoF iMicroglia, which indicates antibody specificity. n=6 tech. reps.

FIGS. 7G and 7H: Abeta1-42 fibril-induced reduction in non-mitochondrial oxygen consumption rate in wild-type iMicroglia (gray bar in FIG. 7G) was rescued with anti-PILRA antibody (striped gray bar in FIG. 7H). n=6 tech. reps.

FIGS. 7I and 7J: PILRA LoF iMicroglia exhibited higher mitochondrial OXPHOS activity with increased ATP production, and anti-PILRA antibodies recapitulated PILRA LoF in wild-type iMicroglia with enhanced rate ATP generation. n=6 tech. reps.

FIGS. 8A-8D: Anti-PILRA antibody bound ex-vivo to monocytes (FIG. 8A) and neutrophils (FIG. 8B). Anti-PILRA antibody did not bind to B-cells and T-cells (FIGS. 8C and 8D).

FIGS. 8E-8G: Anti-PILRA antibody-treated cells did not show elevated CD25 (FIG. 8E) or HLA-DR (FIGS. 8F and 8G).

FIGS. 8H and 8I: ex vivo human leukocytes did not increase production of proinflammatory cytokines after treatment with aqueous-phase (FIG. 8H) or solid-phase (FIG. 8I) anti-PILRA antibodies at 100 nM for 24 hours.

FIG. 9A: Molecular structure showing hPILRA epitopes of anti-PILRA antibodies.

FIG. 9B: Human PILRA binding epitope bins of anti-PILRA antibodies.

FIG. 10: an alignment of the ECD and stalk region sequences of cynoPILRA, hPILRA, and hPILRB (positions are determined with reference to the sequence of SEQ ID NO:1).

FIG. 11: Reference Antibodies #1-#4 bound to CHO-K1 cells expressing hPILRA G78 and did not bind to CHO-K1 cells expressing hPILRB or cynoPILRA G78 (positions are determined with reference to the sequence of SEQ ID NO:1).

FIGS. 12A and 12B: Anti-PILRA antibody achieved target engagement in brain and plasma at 1 day and 4 days after 50 mg/kg dosing in human PILRA expressing BACtg mice.

FIGS. 12C-12H: Anti-PILRA antibody demonstrated an IgG-like pharmacokinetics in brain, plasma, liver, lung, spleen, and bone marrow at 1 day and 4 days after 50 mg/kg IV administration in human PILRA expressing BACtg mice.

### DETAILED DESCRIPTION

### I. Introduction

PILRA is an inhibitory transmembrane receptor that is expressed on the cell surface of various immune cells, such as microglia, monocytes, macrophages, dendritic cells, and neutrophils. Without being bound to a particular theory, it is believed that upon ligand binding, PILRA acts as an inhibitory receptor by recruiting cytoplasmic phosphatases, such as PTPN6/SHP-1 and PTPN11/SHP-2, via their SH2 domains that block signal transduction through dephosphorylation of signaling molecules. A missense variant (G78R) of PILRA alters the sialic acid binding pocket of PILRA, leading to reduced binding of PILRA to several of its ligands, one of which is the sialyated herpes simplex virus type 1 glycoprotein B (HSV-1 gB). HSV-1 infection has been suggested to be present in some Alzheimer's disease patients. The G78R variant of PILRA is proposed to protect individuals from Alzheimer's disease by antagonizing or reducing PILRA signaling, thereby modifying microglial responses.

As detailed in the Examples section below, antibodies have been generated that specifically bind to human PILRA (hPILRA) and that modulate one or more microglial functions regulated by PILRA. In particular, we have, for the first time, identified antibodies that have highly desirable characteristics. These include antibodies that selectively bind to both cynomolgus monkey (“cyno”) PILRA (cynoPILRA) and hPILRA, but may have comparatively lower binding to human PILRB (hPILRB). This is highly advantageous, but also very challenging, given the high homology between cynoPILRA and hPILRB. Having comparable binding between cyno and human PILRA allows for conducting studies in monkeys without having to employ a surrogate molecule. Binding to PILRB is not desired, because PILRB is thought to have different or opposing activity as compared to PILRA, given differences in their respective intracellular domains. Certain antibodies described herein can bind to cynoPILRA with a binding affinity that is within 100-fold (e.g., within 90-fold, 80-fold, 70-fold, 60-fold, 50-fold, 40-fold, 30-fold, 20-fold, 10-fold, 5-fold, or 2-fold) relative to the binding affinity for hPILRA. The antibodies may also have a binding affinity for hPILRA that is at least 10-fold (e.g., at least 15-fold, 20-fold, 25-fold, 30-fold, 35-fold, 40-fold, 45-fold, 50-fold, 60-fold, 70-fold, 80-fold, 90-fold, or 100-fold) stronger than its binding affinity for hPILRB. In certain embodiments, the antibodies further comprise an Fc polypeptide that may contain (i) mutations that reduce or eliminate effector function and/or (ii) mutations that increase in vivo half-life, e.g., by increasing binding of antibody Fc to Fc neonatal receptor (FcRn).

We have also discovered that antibodies binding to certain amino acid residues of the PILRA sequence can convey desirable properties. These include 63, 64, 78, 106, 143, 116-118, and 182-186. In particular examples, we show that antibodies that bind to an epitope that includes (i) G78, K106, and E143 or (ii) T63 and A64 of hPILRA can also bind cynoPILRA but have reduced binding to hPILRB.

### II. Definitions

As used herein, the singular forms “a,” “an” and “the” include plural referents unless the content clearly dictates otherwise. Thus, for example, reference to “an antibody” optionally includes a combination of two or more such molecules, and the like.

As used herein, the terms “about” and “approximately,” when used to modify an amount specified in a numeric value or range, indicate that the numeric value as well as reasonable deviations from the value known to the skilled person in the art, for example ±20%, ±10%, or ±5%, are within the intended meaning of the recited value.

As used herein, the term “PILRA” refers to a paired immunoglobulin-like type 2 receptor alpha protein that is encoded by the gene PILRA. As used herein, a “PILRA” or “PILRA protein” refers to a native (i.e., wild-type) PILRA protein of any vertebrate, such as but not limited to human, non-human primates (e.g., cynomolgus monkey), rodents (e.g., mice, rat), and other mammals. In some embodiments, a PILRA protein is a human PILRA (hPILRA) protein having the sequence of SEQ ID NO: 1:

MGRPLLLPLLPLLLPPAFLQPSGSTGSGPSYLYGVTQPKHLSASMGGSV
EIPFSFYYPWELATAPDVRISWRRGHFHGQSFYSTRPPSIHKDYVNRLF
LNWTEGQKSGFLRISNLQKQDQSVYFCRVELDTRSSGRQQWQSIEGTKL
SITQAVTTTTQRPSSMTTTWRLSSTTTTTGLRVTQGKRRSDSWHISLET
AVGVAVAVTVLGIMILGLICLLRWRRRKGQQRTKATTPAREPFQNTEEP
YENIRNEGQNTDPKLNPKDDGIVYASLALSSSTSPRAPPSHRPLKSPQN
ETLYSVLKA.

In some embodiments, a PILRA protein is a cynomolgus monkey PILRA (cynoPILRA) protein having the sequence of SEQ ID NO:2:

MGRPLLLPLLLPLLPLLLPPAFLQPGGSAGSGPSGPYGVTQRKHLSAPM
GGSVEIPFSFYHPWELAAAPNMKISWRRGNFHGEFFYRTRPAFIHEDYS
NRLLLNWTEGQDRGLLRIWNLRKEDQSVYFCRVELDTRRSGRQRWQSIE
GTKLTITQAVTTTTQRPSSMTTTRRPSSATTTAGLRVTQGKRHSDSWHL
SLKTAVGVTVAVAVLGIMILGLICLLRWRRRKGQQRTKATTPAKEPFQN
TEEPYENIRNEGQNTDPKPNPKDDGIVYASLALSSSTSPRVPPSHHPLK
SPQNETLYSVLKV.

As used herein, the term “PILRB” refers to a paired immunoglobulin-like type 2 receptor beta protein that is encoded by the gene PILRB. As used herein, a “PILRB” or “PILRB protein” refers to a native (i.e., wild-type) PILRB protein of any vertebrate, such as but not limited to human, non-human primates (e.g., cynomolgus monkey), rodents (e.g., mice, rat), and other mammals. In some embodiments, a PILRB protein is a human PILRB (hPILRB) protein having the sequence of SEQ ID NO:3:

MGRPLLLPLLLLLQPPAFLQPGGSTGSGPSYLYGVTQPKHLSASMGGSV
EIPFSFYYPWELAIVPNVRISWRRGHFHGQSFYSTRPPSIHKDYVNRLF
LNWTEGQESGFLRISNLRKEDQSVYFCRVELDTRRSGRQQLQSIKGTKL
TITQAVTTTTTWRPSSTTTIAGLRVTESKGHSESWHLSLDTAIRVALAV
AVLKTVILGLLCLLLLWWRRRKGSRAPSSDF.

As used herein, the term “anti-PILRA antibody” refers to an antibody that specifically binds to a PILRA protein (e.g., human PILRA).

As used herein, the term “antibody” refers to a protein with an immunoglobulin fold that specifically binds to an antigen via its variable regions. The term encompasses intact polyclonal antibodies, intact monoclonal antibodies, including full-length antibodies as well as single chain antibodies, multispecific antibodies such as bispecific antibodies, monospecific antibodies, monovalent antibodies, chimeric antibodies, humanized antibodies, and human antibodies. The term “antibody,” as used herein, also includes antibody fragments that retain binding specificity via its variable regions, including but not limited to Fab, F(ab′)₂, Fv, scFv, and bivalent scFv. Antibodies can contain light chains that are classified as either kappa or lambda. Antibodies can contain heavy chains that are classified as gamma, mu, alpha, delta, or epsilon, which in turn define the immunoglobulin classes, IgG, IgM, IgA, IgD and IgE, respectively.

As used herein, the term “full-length antibody” generally refers to an immunoglobulin molecule that has four polypeptide chains: two heavy chains and two light chains interconnected by disulfide bonds. Each heavy chain is composed of, from N-terminus to C-terminus, a heavy chain variable region (V_{H}), a CH1 constant domain, a hinge region, a CH2 constant domain, and a CH3 constant domain. Each light chain is composed of, from N-terminus to C-terminus, a light chain variable region (V_{L}) and a CL constant domain. A Fab domain or fragment is formed from V_{H}, CH1, V_{L}, and CL domains. A full-length antibody can also be described as having two Fab domains and an Fc domain, where the Fc domain comprises two Fc polypeptides and each Fc polypeptide can include a CH2 domain, a CH3 domain, and may contain at least part of the hinge region of the antibody.

As used herein, the term “anti-PILRA antigen binding portion” refers to an antigen binding segment or entity that specifically binds to a PILRA protein (e.g., hPILRA and/or cynoPILRA). The terms “antigen-binding portion” and “antigen-binding fragment” are used interchangeably herein and refer to one or more fragments of an antibody that retains the ability to specifically bind to an antigen (e.g., a PILRA protein) via its variable region. Examples of antigen-binding fragments include, but are not limited to, a Fab fragment (a monovalent fragment consisting of the V_{L}, V_{H}, CL and CH1 domains), F(ab′)2 fragment (a bivalent fragment comprising two Fab fragments linked by a disulfide bridge at the hinge region), single chain Fv (scFv), disulfide-linked Fv (dsFv), complementarity determining regions (CDRs), a V_{L} (light chain variable region), and a V_{H} (heavy chain variable region).

The term “variable region” or “variable domain” refers to a domain in an antibody heavy chain or light chain that is derived from a germline Variable (V) gene, Diversity (D) gene, or Joining (J) gene (and not derived from a Constant (Cμ and Cδ) gene segment), and that gives an antibody its specificity for binding to an antigen. Typically, an antibody variable region comprises four conserved “framework” regions interspersed with three hypervariable “complementarity determining regions.”

The term “complementarity determining region” or “CDR” refers to the three hypervariable regions in each chain that interrupt the four framework regions established by the light and heavy chain variable regions. The CDRs are primarily responsible for antibody binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 or CDR-H3 is located in the variable region of the heavy chain of the antibody in which it is found, whereas a V_{L} CDR1 or CDR-L1 is the CDR1 from the variable region of the light chain of the antibody in which it is found.

The “framework regions” or “FRs” of different light or heavy chains are relatively conserved within a species. The framework region of an antibody, that is the combined framework regions of the constituent light and heavy chains, serves to position and align the CDRs in three-dimensional space. Framework sequences can be obtained from public DNA databases or published references that include germline antibody gene sequences. For example, germline DNA sequences for human heavy and light chain variable region genes can be found in the “VBASE2” germline variable gene sequence database for human and mouse sequences.

The amino acid sequences of the CDRs and framework regions can be determined using various well-known definitions in the art, e.g., Kabat, Chothia, international ImMunoGeneTics database (IMGT), AbM, and observed antigen contacts (“Contact”). In some embodiments, CDRs are determined according to the Contact definition. See, MacCallum et al., J. Mol. Biol., 262:732-745 (1996). In some embodiments, CDRs are determined by a combination of Kabat, Chothia, and/or Contact CDR definitions.

The term “epitope” refers to the area or region of an antigen to which the CDRs of an antibody specifically binds and can include a few amino acids or portions of a few amino acids, e.g., 5 or 6, or more, e.g., 20 or more amino acids, or portions of those amino acids. For example, where the target is a protein, the epitope can be comprised of consecutive amino acids (e.g., a linear epitope), or amino acids from different parts of the protein that are brought into proximity by protein folding (e.g., a discontinuous or conformational epitope). In some embodiments, the epitope is phosphorylated at one amino acid (e.g., at a serine or threonine residue).

As used herein, the phrase “recognizes an epitope,” as used with reference to an anti-PILRA antibody, means that the antibody CDRs interact with or specifically bind to the antigen (i.e., the PILRA protein) at that epitope or a portion of the antigen containing that epitope.

A “monoclonal antibody” refers to antibodies produced by a single clone of cells or a single cell line and consisting of or consisting essentially of antibody molecules that are identical in their primary amino acid sequence.

A “polyclonal antibody” refers to an antibody obtained from a heterogeneous population of antibodies in which different antibodies in the population bind to different epitopes of an antigen.

A “chimeric antibody” refers to an antibody molecule in which the constant region, or a portion thereof, is altered, replaced or exchanged so that the antigen-binding site (i.e., variable region, CDR, or portion thereof) is linked to a constant region of a different or altered class, effector function and/or species, or in which the variable region, or a portion thereof, is altered, replaced or exchanged with a variable region having a different or altered antigen specificity (e.g., CDR and framework regions from different species). In some embodiments, a chimeric antibody is a monoclonal antibody comprising a variable region from one source or species (e.g., mouse) and a constant region derived from a second source or species (e.g., human). Methods for producing chimeric antibodies are described in the art.

A “humanized antibody” is a chimeric immunoglobulin derived from a non-human source (e.g., murine) that contains minimal sequences derived from the non-human immunoglobulin outside the CDRs. In general, a humanized antibody will comprise at least one (e.g., two) antigen-binding variable domain(s), in which the CDR regions substantially correspond to those of the non-human immunoglobulin and the framework regions substantially correspond to those of a human immunoglobulin sequence. The humanized antibody can also comprise at least a portion of an immunoglobulin constant region (Fc), typically that of a human immunoglobulin sequence. Methods of antibody humanization are known in the art.

A “human antibody” or a “fully human antibody” is an antibody having human heavy chain and light chain sequences, typically derived from human germline genes. In some embodiments, the antibody is produced by a human cell, by a non-human animal that utilizes human antibody repertoires (e.g., transgenic mice that are genetically engineered to express human antibody sequences), or by phage display platforms.

The term “specifically binds” refers to a molecule (e.g., an antibody or an antigen-binding portion thereof) that binds to an epitope or target with stronger affinity, stronger avidity, and/or greater duration to that epitope or target in a sample than it binds to another epitope or non-target compound (e.g., a structurally different antigen). In some embodiments, an antibody (or an antigen-binding portion thereof) that specifically binds to an epitope or target is an antibody (or an antigen-binding portion thereof) that binds to the epitope or target with at least 1.5-fold stronger affinity than other epitopes or non-target compounds, e.g., at least 1.5-fold, 2.5-fold, 5-fold, 10-fold, 100-fold, 1,000-fold, 10,000-fold, or stronger affinity. The term “specific binding,” “specifically binds to,” or “is specific for” a particular epitope or target, as used herein, can be exhibited, for example, by a molecule having an equilibrium dissociation constant K_{D} for the epitope or target to which it binds of, e.g., 10⁻⁴ M or smaller, e.g., 10⁻⁵ M, 10⁻⁶ M, 10⁻⁷ M, 10⁻⁸ M, 10⁻⁹ M, 10⁻¹⁰ M, 10⁻¹¹ M, or 10⁻¹² M. It will be recognized by one of skill that an antibody that specifically binds to a target (e.g., a PILRA protein (e.g., a hPILRA and/or a cynoPILRA)) from one species may also specifically bind to orthologs of that target.

The term “binding affinity” is used herein to refer to the strength of a non-covalent interaction between two molecules, e.g., between an antibody (or an antigen-binding portion thereof) and an antigen. Thus, for example, the term may refer to 1:1 interactions between an antibody (or an antigen-binding portion thereof) and an antigen, unless otherwise indicated or clear from context. Binding affinity may be quantified by measuring an equilibrium dissociation constant (K_{D}), which refers to the dissociation rate constant (k_{d}, time⁻¹) divided by the association rate constant (kₐ, time⁻¹ M⁻¹). K_{D} can be determined by measurement of the kinetics of complex formation and dissociation, e.g., using Surface Plasmon Resonance (SPR) methods, e.g., a Biacore™ system; kinetic exclusion assays such as KinExA®; and BioLayer interferometry (e.g., using the ForteBio® Octet platform). As used herein, “binding affinity” includes not only formal binding affinities, such as those reflecting 1:1 interactions between an antibody (or an antigen-binding portion thereof) and an antigen, but also apparent affinities for which K_{D} values are calculated that may reflect avid binding.

The term “cross-reacts,” as used herein, refers to the ability of an antibody to bind to an antigen other than the antigen against which the antibody was raised. In some embodiments, cross-reactivity refers to the ability of an antibody to bind to an antigen from another species than the antigen against which the antibody was raised. As a non-limiting example, an anti-PILRA antibody as described herein that is raised against a human PILRA peptide can exhibit cross-reactivity with a PILRA peptide or protein from a different species (e.g., cynomolgus monkey or mouse).

The term “modulate” refers to changing or altering one or more properties of a protein or a cell. Properties of a cell can be altered as a result of altering one or more properties of a protein (e.g., a PILRA protein) of the cell, i.e., by binding to the protein of the cell. Properties of a cell that can be modulated include, but are not limited to, cell growth, migration, survival, signaling, phagocytosis, and biomarker secretion. For example, a molecule that binds to a PILRA protein of a cell can cause one or more downstream signaling responses or activities of the cell as a result of PILRA-binding, thus, the molecule is said to modulate the signaling responses or activities of the cell. In some embodiments, the term “modulate” can refer to an increase or decrease in the signaling response or activity of the cell as a result of PILRA-binding, relative to the signaling response or activity of the cell without PILRA-binding. Examples of changes in signaling responses or activities of a cell as a result of PILRA-binding include, but are not limited to, changes phosphorylated STAT3 (pSTAT3) level, phosphorylated STAT1 (pSTAT1) level, phosphorylated EGFR (pEGFR) level, cadherin expression, integrin expression, and cell (e.g., microglia) migration.

The terms “CH3 domain” and “CH2 domain” as used herein refer to immunoglobulin constant region domain polypeptides. In the context of IgG antibodies, a CH3 domain polypeptide refers to the segment of amino acids from about position 341 to about position 447 as numbered according to the EU numbering scheme, and a CH2 domain polypeptide refers to the segment of amino acids from about position 231 to about position 340 as numbered according to the EU numbering scheme. CH2 and CH3 domain polypeptides may also be numbered by the IMGT (ImMunoGeneTics) numbering scheme in which the CH2 domain numbering is 1-110 and the CH3 domain numbering is 1-107, according to the IMGT Scientific chart numbering (IMGT website). CH2 and CH3 domains are part of the Fc region of an immunoglobulin. In the context of IgG antibodies, an Fc region refers to the segment of amino acids from about position 231 to about position 447 as numbered according to the EU numbering scheme. As used herein, the term “Fc region” may also include at least a part of a hinge region of an antibody. An exemplary partial hinge region sequence is DKTHTCPPCP (SEQ ID NO:98).

The terms “corresponding to,” “determined with reference to,” or “numbered with reference to” when used in the context of the identification of a given amino acid residue in a polypeptide sequence, refers to the position of the residue of a specified reference sequence when the given amino acid sequence is maximally aligned and compared to the reference sequence. Thus, for example, an amino acid residue in a polypeptide “corresponds to” an amino acid in the SEQ ID NO:1 when the residue aligns with the amino acid in SEQ ID NO:1 when optimally aligned to SEQ ID NO:1. The polypeptide that is aligned to the reference sequence need not be the same length as the reference sequence.

As used herein, the term “Fc polypeptide” refers to the C-terminal region of a naturally occurring immunoglobulin heavy chain polypeptide that is characterized by an Ig fold as a structural domain. An Fc polypeptide contains constant region sequences including at least the CH2 domain and/or the CH3 domain and may contain at least part of the hinge region, but does not contain a variable region.

A “modified Fc polypeptide” refers to an Fc polypeptide that has at least one mutation, e.g., a substitution, deletion or insertion, as compared to a wild-type immunoglobulin heavy chain Fc polypeptide sequence, but retains the overall Ig fold or structure of the native Fc polypeptide.

The term “isolated,” as used with reference to a nucleic acid or protein (e.g., antibody), denotes that the nucleic acid or protein is essentially free of other cellular components with which it is associated in the natural state. Purity and homogeneity are typically determined using analytical chemistry techniques such as electrophoresis (e.g., polyacrylamide gel electrophoresis) or chromatography (e.g., high performance liquid chromatography). In some embodiments, an isolated nucleic acid or protein (e.g., antibody) is at least 85% pure, at least 90% pure, at least 95% pure, or at least 99% pure.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Naturally occurring α-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and combinations thereof. Stereoisomers of a naturally occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof “Amino acid analogs” refer to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid. Amino acids may be referred to herein by either their commonly known three letter symbols or by the one-letter symbols recommended by the IUPAC-IUB Biochemical Nomenclature Commission.

The terms “polypeptide” and “peptide” are used interchangeably herein to refer to a polymer of amino acid residues in a single chain. The terms apply to amino acid polymers in which one or more amino acid residue is an artificial chemical mimetic of a corresponding naturally occurring amino acid, as well as to naturally occurring amino acid polymers and non-naturally occurring amino acid polymers. Amino acid polymers may comprise entirely L-amino acids, entirely D-amino acids, or a mixture of L and D amino acids.

The term “protein” as used herein refers to either a polypeptide or a dimer (i.e., two) or multimer (i.e., three or more) of single chain polypeptides. The single chain polypeptides of a protein may be joined by a covalent bond, e.g., a disulfide bond, or non-covalent interactions.

The terms “polynucleotide” and “nucleic acid” interchangeably refer to chains of nucleotides of any length, and include DNA and RNA. The nucleotides can be deoxyribonucleotides, ribonucleotides, modified nucleotides or bases, and/or their analogs, or any substrate that can be incorporated into a chain by DNA or RNA polymerase. A polynucleotide may comprise modified nucleotides, such as methylated nucleotides and their analogs. Examples of polynucleotides contemplated herein include single- and double-stranded DNA, single- and double-stranded RNA, and hybrid molecules having mixtures of single- and double-stranded DNA and RNA.

The terms “conservative substitution” and “conservative mutation” refer to an alteration that results in the substitution of an amino acid with another amino acid that can be categorized as having a similar feature. Examples of categories of conservative amino acid groups defined in this manner can include: a “charged/polar group” including Glu (Glutamic acid or E), Asp (Aspartic acid or D), Asn (Asparagine or N), Gln (Glutamine or Q), Lys (Lysine or K), Arg (Arginine or R), and His (Histidine or H); an “aromatic group” including Phe (Phenylalanine or F), Tyr (Tyrosine or Y), Trp (Tryptophan or W), and (Histidine or H); and an “aliphatic group” including Gly (Glycine or G), Ala (Alanine or A), Val (Valine or V), Leu (Leucine or L), Ile (Isoleucine or I), Met (Methionine or M), Ser (Serine or S), Thr (Threonine or T), and Cys (Cysteine or C). Within each group, subgroups can also be identified. For example, the group of charged or polar amino acids can be sub-divided into sub-groups including: a “positively-charged sub-group” comprising Lys, Arg and His; a “negatively-charged sub-group” comprising Glu and Asp; and a “polar sub-group” comprising Asn and Gln. In another example, the aromatic or cyclic group can be sub-divided into sub-groups including: a “nitrogen ring sub-group” comprising Pro, His and Trp; and a “phenyl sub-group” comprising Phe and Tyr. In another further example, the aliphatic group can be sub-divided into sub-groups, e.g., an “aliphatic non-polar sub-group” comprising Val, Leu, Gly, and Ala; and an “aliphatic slightly-polar sub-group” comprising Met, Ser, Thr, and Cys. Examples of categories of conservative mutations include amino acid substitutions of amino acids within the sub-groups above, such as, but not limited to: Lys for Arg or vice versa, such that a positive charge can be maintained; Glu for Asp or vice versa, such that a negative charge can be maintained; Ser for Thr or vice versa, such that a free —OH can be maintained; and Gln for Asn or vice versa, such that a free —NH₂ can be maintained. In some embodiments, hydrophobic amino acids are substituted for naturally occurring hydrophobic amino acid, e.g., in the active site, to preserve hydrophobicity.

The terms “identical” or percent “identity,” in the context of two or more polypeptide sequences, refer to two or more sequences or subsequences that are the same or have a specified percentage of amino acid residues, e.g., at least 60%, at least 65%, at least 70%, at least 75%, at least 80%, at least 85%, at least 90%, or at least 95% or greater, that are identical over a specified region when compared and aligned for maximum correspondence over a comparison window, or designated region as measured using a sequence comparison algorithm or by manual alignment and visual inspection.

For sequence comparison of polypeptides, typically one amino acid sequence acts as a reference sequence, to which a candidate sequence is compared. Alignment can be performed using various methods available to one of skill in the art, e.g., visual alignment or using publicly available software using known algorithms to achieve maximal alignment. Such programs include the BLAST programs, ALIGN, ALIGN-2 (Genentech, South San Francisco, Calif) or Megalign (DNASTAR). The parameters employed for an alignment to achieve maximal alignment can be determined by one of skill in the art. For sequence comparison of polypeptide sequences for purposes of this application, the BLASTP algorithm standard protein BLAST for aligning two proteins sequence with the default parameters is used.

The terms “subject,” “individual,” and “patient,” as used interchangeably herein, refer to a mammal, including but not limited to humans, non-human primates, rodents (e.g., rats, mice, and guinea pigs), rabbits, cows, pigs, horses, and other mammalian species. In one embodiment, the subject, individual, or patient is a human.

The terms “treating,” “treatment,” and the like are used herein to generally mean obtaining a desired pharmacologic and/or physiologic effect. “Treating” or “treatment” may refer to any indicia of success in the treatment or amelioration of a neurodegenerative disease (e.g., Alzheimer's disease or another neurodegenerative disease described herein), including any objective or subjective parameter such as abatement, remission, improvement in patient survival, increase in survival time or rate, diminishing of symptoms or making the disease more tolerable to the patient, slowing in the rate of degeneration or decline, or improving a patient's physical or mental well-being. The treatment or amelioration of symptoms can be based on objective or subjective parameters. The effect of treatment can be compared to an individual or pool of individuals not receiving the treatment, or to the same patient prior to treatment or at a different time during treatment.

The term “pharmaceutically acceptable excipient” refers to a non-active pharmaceutical ingredient that is biologically or pharmacologically compatible for use in humans or animals, such as, but not limited to a buffer, carrier, or preservative.

As used herein, a “therapeutic amount” or “therapeutically effective amount” of an agent (e.g., an antibody as described herein) is an amount of the agent that treats, alleviates, abates, or reduces the severity of symptoms of a disease in a subject. A “therapeutic amount” of an agent (e.g., an antibody as described herein) may improve patient survival, increase survival time or rate, diminish symptoms, make an injury, disease, or condition (e.g., a neurodegenerative disease) more tolerable, slow the rate of degeneration or decline, or improve a patient's physical or mental well-being.

The term “administer” refers to a method of delivering agents, compounds, or compositions to the desired site of biological action. These methods include, but are not limited to, topical delivery, parenteral delivery, intravenous delivery, intradermal delivery, intramuscular delivery, intrathecal delivery, colonic delivery, rectal delivery, or intraperitoneal delivery. In one embodiment, an antibody as described herein is administered intravenously.

The term “control” or “control value” refers to a reference value or baseline value. Appropriate controls can be determined by one skilled in the art. In some instances, control values can be determined relative to a baseline within the same subject or experiment. In other instances, the control value can be determined relative to a control subject (e.g., a healthy control or a disease control) or an average value in a population of control subjects (e.g., healthy controls or disease controls, e.g., a population of 10, 20, 50, 100, 200, 500, 1000 control subjects or more).

### III. Anti-PILRA Antibodies

In one aspect, antibodies that specifically bind to a paired immunoglobulin-like type 2 receptor alpha (PILRA) protein (e.g., a hPILRA and/or a cynoPILRA protein) are provided. In some embodiments, the antibody specifically binds to a hPILRA protein. In some embodiments, an anti-PILRA antibody is selective for PILRA over other PILRA receptors (e.g., a paired immunoglobulin-like type 2 receptor beta (PILRB)).

In some embodiments, an anti-PILRA antibody is an antibody that comprises one or more complementarity determining region (CDR), heavy chain variable region, and/or light chain variable region sequences as disclosed herein. In some embodiments, an anti-PILRA antibody comprises one or more CDR, heavy chain variable region, and/or light chain variable region sequences as disclosed herein and further comprises one or more functional characteristics as disclosed herein, e.g., an antibody that antagonizes PILRA activity (e.g., blocks binding of a ligand to hPILRA, alters phosphorylation of downstream proteins (e.g., increase phosphorylation of EGFR or STAT3; decrease phosphorylation of STAT1), elevates cellular respiration, fatty acid metabolism (e.g., fatty acid oxidation), and ATP production, enhances cell migration (e.g., microglia migration), increases anti-inflammatory gene or protein expression, and/or reduces cytokine protein expression). In some embodiments, the anti-PILRA antibody comprises Fc polypeptides that comprise one or more modifications as described herein.

In some embodiments, the anti-PILRA antibody is a fully human antibody. In some embodiments, the anti-PILRA antibody is a chimeric antibody. In some embodiments, the anti-PILRA antibody is a humanized and/or affinity matured antibody.

### Anti-PILRA Antibody Sequences

In some embodiments, a heavy chain sequence, or a portion thereof, and/or a light chain sequence, or a portion thereof, is derived from an anti-PILRA antibody described herein (e.g., Clone 2, Clone 4, or Clone 5). The CDR, heavy chain variable region, and light chain variable region amino acid sequences of these clones is set forth in Table 1.

TABLE 1

| Ab | | | | | | | | |
|---|---|---|---|---|---|---|---|---|
| CL | CDR-H1 | CDR-H2 | CDR-H3 | V_{H} | CDR-L1 | CDR-L2 | CDR-L3 | V_{L} |
| 1 | GFTFDD | GFSWNS | DKSISA | EVQLVESGGGLVQPGRSLRLSC | QASRRIN | DASNLET | QQYDNLP | DIQMTQSPSSLSASVGD |
| | YAMH | GSIG | AGRFDY | AVSGFTFDDYAMHWVRQAPG | NYLN | (SEQ ID | LT (SEQ | RVTITCQASRRINNYLN |
| | (SEQ ID NO: 4) | (SEQ ID NO: 12) | (SEQ ID NO: 20) | KGLEWVSGFSWNSGSIGYPDS | (SEQ ID | NO: 39) | ID NO: 47) | WYQQKPGKAPKLLIYD |
| | | | | VKGRFTISRDNAKNSLYLQMN | NO: 30) | | | ASNLETGVPSRFTGSGS |
| | | | | SLRAEDTAFYYCAKDKSISAAG | | | | GTDFTLTISSLQPEDIAT |
| | | | | RFDYWGQGTLVTVSS (SEQ ID | | | | YYCQQYDNLPLTFGGGT |
| | | | | NO: 54) | | | | KIKIK (SEQ ID NO: 64) |
| 2 | GFTFDD | GFSWNS | DKSISA | EVQLVESGGGLVQPGRSLRLSC | QASRRIN | DASNLET | QQYDNLP | DIQMTQSPSSLSASVGD |
| | YAMH | GSIG | AGRFDY | AVSGFTFDDYAMHWVRQAPG | NYLN | (SEQ ID | LT (SEQ | RVTITCQASRRINNYLN |
| | (SEQ ID NO: 4) | (SEQ ID NO: 12) | (SEQ ID NO: 20) | KGLEWVSGFSWNSGSIGYPDS | (SEQ ID | NO: 39) | ID NO: 47) | WYQQKPGKAPKLLIYD |
| | | | | VKGRFTISRDNAKNSLYLQMN | NO: 30) | | | ASNLETGVPSRFTGSGS |
| | | | | SLRAEDTAFYYCAKDKSISAAG | | | | GTDFTLTISSLQPEDIAT |
| | | | | RFDYWGQGTLVTVSS (SEQ ID | | | | YYCQQYDNLPLTFGGGT |
| | | | | NO: 54) | | | | KVEIK (SEQ ID NO: 65) |
| 3 | GFTFDD | GMSWN | DKSIGA | EVQLVESGGGLVQPGRSLRLSC | QASQGIN | DASNLET | QQYDNLP | DIQMTQSPSSLSASVGD |
| | YAIH | SGSIG | AGRFDC | AASGFTFDDYAIHWVRQAPGK | NYLN | (SEQ ID | LT (SEQ | RVTITCQASQGINNYLN |
| | (SEQ ID NO: 5) | (SEQ ID NO: 13) | (SEQ ID NO: 21) | GLEWVSGMSWNSGSIGYGDSV | (SEQ ID | NO: 39) | ID NO: 47) | WYQQKPGKAPKLLIYD |
| | | | | KGRFTISRDNAKNSLYLQMNSL | NO: 31) | | | ASNLETGVPSRFSGSGSG |
| | | | | RAEDTAFYYCAKDKSIGAAGR | | | | TDFTFTISSLQPEDIATYY |
| | | | | FDCWGQGTLVTVSS (SEQ ID | | | | CQQYDNLPLTFGGGTKV |
| | | | | NO: 55) | | | | EIK (SEQ ID NO: 66) |
| 4 | GFTFDD | GMSWN | DKSIGA | EVQLVESGGGLVQPGRSLRLSC | QASQGIN | DASNLET | QQYDNLP | DIQMTQSPSSLSASVGD |
| | YAIH | SGSIG | AGRFDC | AASGFTFDDYAIHWVRQAPGK | NYLN | (SEQ ID | LT (SEQ | RVTITCQASQGINNYLN |
| | (SEQ ID NO: 5) | (SEQ ID NO: 13) | (SEQ ID NO: 21) | GLEWVSGMSWNSGSIGYGDSV | (SEQ ID | NO: 39) | ID NO: 47) | WYQQKPGKAPKLLIYD |
| | | | | KGRFTISRDNAKNSLYLQMNSL | NO: 31) | | | ASNLETGVPSRFSGSGSG |
| | | | | RAEDTAFYYCAKDKSIGAAGR | | | | TDFTFTISSLQPEDIATYY |
| | | | | FDSWGQGTLVTVSS (SEQ ID | | | | CQQYDNLPLTFGGGTKV |
| | | | | NO: 56) | | | | EIK (SEQ ID NO: 66) |
| 5 | GYTFIG | WINPES | GNWNF | QVQLVQSGAEVKKPGASVRVS | RSSQSISI | VASSLQS | QQSYSAP | DIQMTQSPSSLSASVGD |
| | FYIH | GDTT | PDTFDF | CKASGYTFIGFYIHWVRQAPGQ | ID NO: 32) | (SEQ ID | FT (SEQ ID | RVTITCRSSQSISIYLNW |
| | (SEQ ID | (SEQ ID | (SEQ ID | GLEWMGWINPESGDTTYAQKF | YLN (SEQ | NO: 40) | NO: 48) | HQQIPGKAPKLLIYVASS |
| | NO: 6) | NO: 14) | NO: 23) | QGRVTMTTDTSINTAYMDLNR | | | | LQSGIPSRFSGRGSGTEF |
| | | | | LRSDDSAVYFCARGNWNFPDT | | | | TLTISSLQPEDFATYYCQ |
| | | | | FDFWGQGTMVIVSS (SEQ ID | | | | QSYSAPFTFGPGTKVDIK |
| | | | | NO: 57) | | | | (SEQ ID NO: 67) |
| 6 | GYTFTE | RIDPED | TIRGTV | EVQLQQSGPELQRPGASVKLSC | RASEDIFN | NAKTLHT | QQYYDYP | DIQMTQSPASLSASLGET |
| | YYMY (SEQ ID | GGTD | FAF | KASGYTFTEYYMYWVKQRPK | GLA (SEQ | (SEQ ID | LT (SEQ | VTIECRASEDIFNGLAW |
| | NO: 7) | (SEQ ID | (SEQ ID | QGLELIGRIDPEDGGTDYIEKFK | ID NO: 33) | NO: 41) | ID NO: 49) | YSLKINSLQSEDVASYFC |
| | | NO: 15) | NO: 24) | NKATLTADTSSNTAYMQLSSL | | | | TLHTGVPSRFSGSGSGSQ |
| | | | | TSEDTATYFCATTIRGTVFAFW | | | | YQQKPGKSPHLLIYNAK |
| | | | | GQGTLVTVSS (SEQ ID NO: | | | | QQYYDYPLTFGSGTKLE |
| | | | | 58) | | | | IK (SEQ ID NO: 68) |
| 7 | GYTFTE | RIDPED | TIRGTV | EVQLQQSGPELQRPGASVKLSC | RPSEDIYN | NANSLHT | QQYYDYP | DIQMTQSPASLSASLGET |
| | YYMY | GGTD | FVY | KASGYTFTEYYMYWVKQRPK | GLA (SEQ | (SEQ ID | LT (SEQ | VTIECRPSEDIYNGLAW |
| | (SEQ ID | (SEQ ID | (SEQ ID | QGLELMGRIDPEDGGTDYVEK | ID NO: 34) | NO: 42) | ID NO: 49) | YQQKPGESPQLLIYNAN |
| | NO: 7) | NO: 15) | NO: 25) | FKNKATLTADTSSNTAYMQLS | | | | SLHTGVPSRFSGSGSGTQ |
| | | | | SLTSEDTATYFCASTIRGTVFV | | | | YSLKINSLQSEDVASYFC |
| | | | | YWGQGTLVTVSS (SEQ ID | | | | QQYYDYPLTFGSGTKLE |
| | | | | NO: 59) | | | | IK (SEQ ID NO: 69) |
| 8 | GYTFTG | WINPNS | EGLDGD | QVQLVQSGAEVKKPGASVKVS | RSSQSLV | NISNRFS | IQTTQFST | EIMLTQTPLSSPVTLGQP |
| | HYMH | GDTD | PFDY | CKASGYTFTGHYMHWVRQAP | HSDGNTY | SEQ ID | (SEQ ID | ASISCRSSQSLVHSDGNT |
| | (SEQ ID NO: 8) | (SEQ ID NO: 16) | (SEQ ID NO: 26) | GQGLEWMGWINPNSGDTDYA | LS (SEQ ID | NO: 43) | NO: 50) | YLSWLQQRPGQPPRLLI |
| | | | | QKFQGRVTMTRDTSISTAYMD | NO: 35) | | | YNISNRFSGVPDRFSGRG |
| | | | | LNRLRSDDTAVFYCAREGLDG | | | | AGTDFTLKISRVEAEDV |
| | | | | DPFDYWGQGTLVTVSS (SEQ | | | | GVYYCIQTTQFSTFGQG |
| | | | | ID NO: 60) | | | | TKLEIK (SEQ ID NO: 70) |
| 9 | GGSISS | EIYHFG | TLRDFY | QVQLQESGPGLVKPSGTLSLTC | RTSQNINT | AASSLQS | QQSFNIPL | DIHMTQSPSSLSASVGD |
| | NNWWS | TTT | YYMDV | AVSGGSISSNNWWSWVRQPPG | YL | (SEQ ID | T (SEQ ID | RVTITCRTSQNINTYLN |
| | (SEQ ID | (SEQ ID | (SEQ ID | KGLEWIGEIYHFGTTTYNPSLK | (SEQ | NO: 44) | NO: 51) | WYQQRPGRAPKLLIYAA |
| | NO: 9) | NO: 17) | NO: 27) | SRVTISVDKSKNQFSLKLSSLTA | ID NO: 36) | | | SSLQSGVPSRFSGSGSGT |
| | | | | ADTAVYYCARTLRDFYYYMD | | | | DFILSISSLQPEDFATYYC |
| | | | | VWGKGTTVTVSS (SEQ ID | | | | QQSFNIPLTFGGGTKVEI |
| | | | | NO: 61) | | | | K (SEQ ID NO: 71) |
| 10 | GYSFTT | IIYPGDS | TYYYFS | EVQLVQSGAEIRKPGESLKISC | RASQDIR | AASSFQS | QQTHSFP | DIQMTQSPSSVSAYVGD |
| | YWIA | DTR | GSHWD | KGSGYSFTTYWIAWVRQMPGK | DCLA | (SEQ ID | YT (SEQ | RVTITCRASQDIRDCLA |
| | (SEQ ID | (SEQ ID | AFDI | GLEWMGIIYPGDSDTRYSPSFQ | (SEQ ID | NO: 45) | ID NO: 52) | WYQQKPGKAPKFLIYA |
| | NO: 10) | NO: 18) | (SEQ ID | GQVTISADKSITTAYLQWSSLK | NO: 37) | | | ASSFQSGVPSGFSGSGSG |
| | | | NO: 28) | ASDTGIYYCARTYYYFSGSHW | | | | TDFTLTVTSLQPEDSATY |
| | | | | DAFDIWGQGTMVTVSS (SEQ | | | | YCQQTHSFPYTFGQGTK |
| | | | | ID NO: 62) | | | | LEIK (SEQ ID NO: 72) |
| 11 | GFAFSG | TISNGG | QKTWD | EVKLVESGGGLVRPGGSQKLS | TLSSQHST | LKKDGSH | GDTIKEQ | QLVLTQSSSASFSLGASA |
| | YDMS | RHTY | VHAMD | CAVSGFAFSGYDMSWVRQTPE | YTIE (SEQ | STGD | FVYV | KLTCTLSSQHSTYTIEW |
| | (SEQ ID | (SEQ ID | Y (SEQ | KRLEWVATISNGGRHTYYPDS | ID NO: 38) | (SEQ ID | (SEQ ID | YQQQPLKPPKYVMELK |
| | NO: 11) | NO: 19) | ID | VKGRFTISRDNARNTLYLQMSS | | NO: 46) | NO: 53) | KDGSHSTGDGIPDRFSGS |
| | | | NO: 29) | LRSEDTALYYCARQKTWDVH | | | | SSGADRYLSISNIQPEDE |
| | | | | AMDYWGQGTSVTVSS (SEQ ID | | | | AIYICGVGDTIKEQFVYV |
| | | | | NO: 63) | | | | FGGGTKVTVL (SEQ ID |
| | | | | | | | | NO: 73) |
| 12 | GYTFTE | RIDPED | TIRGTV | EVQLVQSGAEVKKPGASVKVS | RASEDIFN | NAKTLHT | QQYYDYP | DIQMTQSPSSLSASVGD |
| | YYMY | GGTD | FAF | CKASGYTFTEYYMYWVRQAP | GLA (SEQ | (SEQ ID | LT (SEQ | RVTITCRASEDIFNGLA |
| | (SEQ ID | (SEQ ID | (SEQ ID | GQGLELIGRIDPEDGGTDYIEKF | ID NO: 33) | NO: 41) | ID NO: 49) | WYQQKPGKSPKLLIYNA |
| | NO: 7) | NO: 15) | NO: 24) | KNRVTITADTSTSTAYLELSSLR | | | | KTLHTGVPSRFSGSGSGS |
| | | | | SEDTAVYYCATTIRGTVFAFW | | | | DYTLTISSLQPEDFATYF |
| | | | | GQGTLVTVSS (SEQ ID NO: | | | | CQQYYDYPLTFGQGTK |
| | | | | 137) | | | | VEIK (SEQ ID NO: 145) |
| 13 | GYTFTE | RIDPED | TIRGTV | EVQLVQSGAEVKKPGASVKVS | RASEDIFN | NAKTLHT | QQYYDYP | DIQMTQSPSSLSASVGD |
| | YYMY | GGTD | FAF | CKASGYTFTEYYMYWVRQAP | GLA (SEQ | (SEQ ID | LT (SEQ | RVTITCRASEDIFNGLA |
| | (SEQ ID | (SEQ ID | (SEQ ID | GQGLELIGRIDPEDGGTDYIEKF | ID NO: 33) | NO: 41) | ID NO: 49) | WYQQKPGKSPKLLIYNA |
| | NO: 7) | NO: 15) | NO: 24) | KNRVTLTADTSTSTAYLELSSL | | | | KTLHTGVPSRFSGSGSGS |
| | | | | RSEDTAVYYCATTIRGTVFAF | | | | DYTLTISSLQPEDFATYF |
| | | | | WGQGTLVTVSS (SEQ ID | | | | CQQYYDYPLTFGQGTK |
| | | | | NO: 138) | | | | VEIK (SEQ ID NO: 145) |
| 14 | GYTFTE | RIDPED | TIRGTV | EVQLVQSGAEVKKPGASVKVS | RASEDIFN | NAKTLHT | QQYYDYP | DIQMTQSPSSLSASVGD |
| | YYMY | GGTD | FAF | CKASGYTFTEYYMYWVRQAP | GLA (SEQ | (SEQ ID | LT (SEQ | RVTITCRASEDIFNGLA |
| | (SEQ ID | (SEQ ID | (SEQ ID | GQGLELIGRIDPEDGGTDYIEKF | ID NO: 33) | NO: 41) | ID NO: 49) | WYQQKPGKSPKLLIYNA |
| | NO: 7) | NO: 15) | NO: 24) | KNRVTITADTSTSTAYLELSSLR | | | | KTLHTGVPSRFSGSGSGS |
| | | | | SEDTAVYFCATTIRGTVFAFWG | | | | DYTLTISSLQPEDFATYF |
| | | | | QGTLVTVSS (SEQ ID NO: | | | | CQQYYDYPLTFGQGTK |
| | | | | 139) | | | | VEIK (SEQ ID NO: 145) |
| 15 | GYTFTE | RIDPED | TIRGTV | EVQLVQSGAEVKKPGASVKVS | RASEDIFN | NAKTLHT | QQYYDYP | DIQMTQSPSSLSASVGD |
| | YYMY | GGTD | FAF | CKASGYTFTEYYMYWVRQAP | GLA (SEQ | (SEQ ID | LT (SEQ | RVTITCRASEDIFNGLA |
| | (SEQ ID | (SEQ ID | (SEQ ID | GQGLELIGRIDPEDGGTDYIEKF | ID NO: 33) | NO: 41) | ID NO: 49) | WYQQKPGKSPKLLIYNA |
| | NO: 7) | NO: 15) | NO: 24) | KNRSTLTADTSTSTAYLELSSL | | | | KTLHTGVPSRFSGSGSGS |
| | | | | RSEDTAVYFCATTIRGTVFAFW | | | | DYTLTISSLQPEDFATYF |
| | | | | GQGTLVTVSS (SEQ ID NO: | | | | CQQYYDYPLTFGQGTK |
| | | | | 140) | | | | VEIK (SEQ ID NO: 145) |
| 16 | GYTFTE | RIDPED | TIRGTV | EVQLVQSGAEVKKPGASVKVS | RASEDIFN | NAKTLHT | QQYYDYP | DIQMTQSPSSLSASVGD |
| | YYMY | GGTD | FVY | CKASGYTFTEYYMYWVRQAP | GLA (SEQ | (SEQ ID | LT (SEQ | RVTITCRASEDIFNGLA |
| | (SEQ ID | (SEQ ID | (SEQ ID | GQGLELIGRIDPEDGGTDYVEK | ID NO: 33) | NO: 41) | ID NO: 49) | WYQQKPGKSPKLLIYNA |
| | NO: 7) | NO: 15) | NO: 25) | FKNRATITADTSTSTAYLELSSL | | | | KTLHTGVPSRFSGSGSGS |
| | | | | RSEDTAVYYCASTIRGTVFVY | | | | DYTLTISSLQPEDFATYF |
| | | | | WGQGTLVTVSS (SEQ ID NO: | | | | CQQYYDYPLTFGQGTK |
| | | | | 141) | | | | VEIK (SEQ ID NO: 145) |
| 17 | GYTFTE | RIDPED | TIRGTV | EVQLVQSGAEVKKPGASVKVS | RASEDIFN | NAKTLHT | QQYYDYP | DIQMTQSPSSLSASVGD |
| | YYMY | GGTD | FVY | CKASGYTFTEYYMYWVRQAP | GLA (SEQ | (SEQ ID | LT (SEQ | RVTITCRASEDIFNGLA |
| | (SEQ ID | (SEQ ID | (SEQ ID | GQGLELIGRIDPEDGGTDYVEK | | | | WYQQKPGKSPKLLIYNA |
| | NO: 7) | NO: 15) | NO: 25) | FKNRVTLTADTSTSTAYLELSS | ID NO: 33) | NO: 41) | ID NO: 49) | KTLHTGVPSRFSGSGSGS |
| | | | | LRSEDTAVYYCASTIRGTVFVY | | | | DYTLTISSLQPEDFATYF |
| | | | | WGQGTLVTVSS (SEQ ID | | | | CQQYYDYPLTFGQGTK |
| | | | | NO: 158) | | | | VEIK (SEQ ID NO: 145) |
| 18 | GYTFTE | RIDPED | TIRGTV | EVQLVQSGAEVKKPGASVKVS | RASEDIFN | NAKTLHT | QQYYDYP | DIQMTQSPSSLSASVGD |
| | YYMY | GGTD | FVY | CKASGYTFTEYYMYWVRQAP | GLA (SEQ | (SEQ ID | LT (SEQ | RVTITCRASEDIFNGLA |
| | (SEQ ID | (SEQ ID | (SEQ ID | GQGLELIGRIDPEDGGTDYVEK | ID NO: 33) | NO: 41) | ID NO: 49) | WYQQKPGKSPKLLIYNA |
| | NO: 7) | NO: 15) | NO: 25) | FKNRVTITADTSTSTAYLELSSL | | | | KTLHTGVPSRFSGSGSGS |
| | | | | RSEDTAVYFCASTIRGTVFVYW | | | | DYTLTISSLQPEDFATYF |
| | | | | GQGTLVTVSS (SEQ ID NO: | | | | CQQYYDYPLTFGQGTK |
| | | | | 142) | | | | VEIK (SEQ ID NO: 145) |
| 19 | GYTFTE | RIDPED | TIRGTV | EVQLVQSGAEVKKPGASVKVS | RASEDIFN | NAKTLHT | QQYYDYP | DIQMTQSPSSLSASVGD |
| | YYMY | GGTD | FVY | CKASGYTFTEYYMYWVRQAP | GLA (SEQ | (SEQ ID | LT (SEQ | RVTITCRASEDIFNGLA |
| | (SEQ ID | (SEQ ID | (SEQ ID | GQGLELIGRIDPEDGGTDYVEK | ID NO: 33) | NO: 41) | ID NO: 49) | WYQQKPGKSPKLLIYNA |
| | NO: 7) | NO: 15) | NO: 25) | FKNRATLTADTSTSTAYLELSS | | | | KTLHTGVPSRFSGSGSGS |
| | | | | LRSEDTAVYFCASTIRGTVFVY | | | | DYTLTISSLQPEDFATYF |
| | | | | WGQGTLVTVSS (SEQ ID NO: | | | | CQQYYDYPLTFGQGTK |
| | | | | 143) | | | | VEIK (SEQ ID NO: 145) |
| 20 | GYTFTE | RIDPED | TIRGTV | EVQLVQSGAEVKKPGASVKVS | RPSEDIYN | NANSLHT | QQYYDYP | DIQMTQSPSSLSASVGD |
| | YYMY | GGTD | FAF | CKASGYTFTEYYMYWVRQAP | GLA (SEQ | (SEQ ID | LT (SEQ | RVTITCRPSEDIYNGLA |
| | (SEQ ID | (SEQ ID | (SEQ ID | GQGLELIGRIDPEDGGTDYIEKF | ID NO: 34) | NO: 42) | ID NO: 49) | WYQQKPGKAPKLLIYN |
| | NO: 7) | NO: 15) | NO: 24) | KNRVTLTADTSTSTAYLELSSL | | | | ANSLHTGVPSRFSGSGS |
| | | | | RSEDTAVYYCATTIRGTVFAF | | | | GTDYTLTISSLQPEDFAT |
| | | | | WGQGTLVTVSS (SEQ ID | | | | YYCQQYYDYPLTFGQG |
| | | | | NO: 138) | | | | TKVEIK (SEQ ID NO: 146) |
| 21 | GYTFTE | RIDPED | TIRGTV | EVQLVQSGAEVKKPGASVKVS | RPSEDIYN | NANSLHT | QQYYDYP | DIQMTQSPSSLSASVGD |
| | YYMY | GGTD | FAF | CKASGYTFTEYYMYWVRQAP | GLA (SEQ | (SEQ ID | LT (SEQ | RVTITCRPSEDIYNGLA |
| | (SEQ ID | (SEQ ID | (SEQ ID | GQGLELIGRIDPEDGGTDYIEKF | ID NO: 34) | NO: 42) | ID NO: 49) | WYQQKPGKAPKLLIYN |
| | NO: 7) | NO: 15) | NO: 24) | KNRSTLTADTSTSTAYLELSSL | | | | ANSLHTGVPSRFSGSGS |
| | | | | RSEDTAVYFCATTIRGTVFAFW | | | | GTDYTLTISSLQPEDFAT |
| | | | | GQGTLVTVSS (SEQ ID NO: | | | | YYCQQYYDYPLTFGQG |
| | | | | 140) | | | | TKVEIK (SEQ ID NO: 146) |
| 22 | GYTFTE | RIDPED | TIRGTV | EVQLVQSGAEVKKPGASVKVS | RPSEDIYN | NANSLHT | QQYYDYP | DIQMTQSPSSLSASVGD |
| | YYMY | GGTD | FVY | CKASGYTFTEYYMYWVRQAP | GLA (SEQ | (SEQ ID | LT (SEQ | RVTITCRPSEDIYNGLA |
| | (SEQ ID | (SEQ ID | (SEQ ID | GQGLELIGRIDPEDGGTDYVEK | ID NO: 34) | NO: 42) | ID NO: 49) | WYQQKPGKAPKLLIYN |
| | NO: 7) | NO: 15) | NO: 25) | FKNRVTLTADTSTSTAYLELSS | | | | ANSLHTGVPSRFSGSGS |
| | | | | LRSEDTAVYYCASTIRGTVFVY | | | | GTDYTLTISSLQPEDFAT |
| | | | | WGQGTLVTVSS (SEQ ID | | | | YYCQQYYDYPLTFGQG |
| | | | | NO: 158) | | | | TKVEIK (SEQ ID NO: 146) |
| 23 | GYTFTE | RIDPED | TIRGTV | EVQLVQSGAEVKKPGASVKVS | RPSEDIYN | NANSLHT | QQYYDYP | DIQMTQSPSSLSASVGD |
| | YYMY | GGTD | FVY | CKASGYTFTEYYMYWVRQAP | GLA (SEQ | (SEQ ID | LT (SEQ | RVTITCRPSEDIYNGLA |
| | (SEQ ID | (SEQ ID | (SEQ ID | GQGLELIGRIDPEDGGTDYVEK | ID NO: 34) | NO: 42) | ID NO: 49) | WYQQKPGKAPKLLIYN |
| | NO: 7) | NO: 15) | NO: 25) | FKNRATLTADTSTSTAYLELSS | | | | ANSLHTGVPSRFSGSGS |
| | | | | LRSEDTAVYFCASTIRGTVFVY | | | | GTDYTLTISSLQPEDFAT |
| | | | | WGQGTLVTVSS (SEQ ID | | | | YYCQQYYDYPLTFGQG |
| | | | | NO: 143) | | | | TKVEIK (SEQ ID NO: 146) |
| 24 | GYTFTE | RIDPED | TIRGTV | EVQLVQSGAEVKKPGASVKVS | RPSEDIYN | NANSLHT | QQYYDYP | DIQMTQSPSSLSASVGD |
| | YYMY | GGTD | FAF | CKASGYTFTEYYMYWVRQAP | GLA (SEQ | (SEQ ID | LT (SEQ | RVTITCRPSEDIYNGLA |
| | (SEQ ID | (SEQ ID | (SEQ ID | GQGLELIGRIDPEDGGTDYIEKF | ID NO: 34) | NO: 42) | ID NO: 49) | WYQQKPGKAPKLLIYN |
| | NO: 7) | NO: 15) | NO: 24) | KNRVTLTADTSTSTAYLELSSL | | | | ANSLHTGVPSRFSGSGS |
| | | | | RSEDTAVYYCATTIRGTVFAF | | | | GTDYTLTISSLQPEDFAT |
| | | | | WGQGTLVTVSS (SEQ ID | | | | YFCQQYYDYPLTFGQGT |
| | | | | NO: 138) | | | | KVEIK (SEQ ID NO: 147) |
| 25 | GYTFTE | RIDPED | TIRGTV | EVQLVQSGAEVKKPGASVKVS | RPSEDIYN | NANSLHT | QQYYDYP | DIQMTQSPSSLSASVGD |
| | YYMY | GGTD | FAF | CKASGYTFTEYYMYWVRQAP | GLA (SEQ | (SEQ ID | LT (SEQ | RVTITCRPSEDIYNGLA |
| | (SEQ ID | (SEQ ID | (SEQ ID | GQGLELIGRIDPEDGGTDYIEKF | ID NO: 34) | NO: 42) | ID NO: 49) | WYQQKPGKAPKLLIYN |
| | NO: 7) | NO: 15) | NO: 24) | KNRSTLTADTSTSTAYLELSSL | | | | ANSLHTGVPSRFSGSGS |
| | | | | RSEDTAVYFCATTIRGTVFAFW | | | | GTDYTLTISSLQPEDFAT |
| | | | | GQGTLVTVSS (SEQ ID NO: | | | | YFCQQYYDYPLTFGQGT |
| | | | | 140) | | | | KVEIK (SEQ ID NO: 147) |
| 26 | GYTFTE | RIDPED | TIRGTV | EVQLVQSGAEVKKPGASVKVS | RPSEDIYN | NANSLHT | QQYYDYP | DIQMTQSPSSLSASVGD |
| | YYMY | GGTD | FVY | CKASGYTFTEYYMYWVRQAP | GLA (SEQ | (SEQ ID | LT (SEQ | RVTITCRPSEDIYNGLA |
| | (SEQ ID | (SEQ ID | (SEQ ID | GQGLELIGRIDPEDGGTDYVEK | ID NO: 34) | NO: 42) | ID NO: 49) | WYQQKPGKAPKLLIYN |
| | NO: 7) | NO: 15) | NO: 25) | FKNRVTLTADTSTSTAYLELSS | | | | ANSLHTGVPSRFSGSGS |
| | | | | LRSEDTAVYYCASTIRGTVFVY | | | | GTDYTLTISSLQPEDFAT |
| | | | | WGQGTLVTVSS (SEQ ID NO: | | | | YFCQQYYDYPLTFGQGT |
| | | | | 158) | | | | KVEIK (SEQ ID NO: 147) |
| 27 | GYTFTE | RIDPED | TIRGTV | EVQLVQSGAEVKKPGASVKVS | RPSEDIYN | NANSLHT | QQYYDYP | DIQMTQSPSSLSASVGD |
| | YYMY | GGTD | FVY | CKASGYTFTEYYMYWVRQAP | GLA (SEQ | (SEQ ID | LT (SEQ | RVTITCRPSEDIYNGLA |
| | (SEQ ID | (SEQ ID | (SEQ ID | GQGLELIGRIDPEDGGTDYVEK | ID NO: 34) | NO: 42) | ID NO: 49) | WYQQKPGKAPKLLIYN |
| | NO: 7) | NO: 15) | NO: 25) | FKNRATLTADTSTSTAYLELSS | | | | ANSLHTGVPSRFSGSGS |
| | | | | LRSEDTAVYFCASTIRGTVFVY | | | | GTDYTLTISSLQPEDFAT |
| | | | | WGQGTLVTVSS (SEQ ID NO: | | | | YFCQQYYDYPLTFGQGT |
| | | | | 143) | | | | KVEIK (SEQ ID NO: 147) |
| 28 | GYTFTE | RIDPED | TIRGTV | EVQLVQSGAEVKKPGASVKVS | RASEDIFN | NAKTLHT | QQYYDYP | DIQMTQSPSSLSASVGD |
| | YYMY | GGTD | FAF | CKASGYTFTEYYMYWVRQAP | GLA (SEQ | (SEQ ID | LT (SEQ | RVTITCRASEDIFNGLA |
| | (SEQ ID | (SEQ ID | (SEQ ID | GQGLELIGRIDPEDGGTDYIEKF | ID NO: 33) | NO: 41) | ID NO: 49) | WYQQKPGKSPKLLIYNA |
| | NO: 7) | NO: 15) | NO: 24) | KNRVTLTADTSTSTAYLELSSL | | | | KTLHTGVPSRFSGSGSG |
| | | | | RSEDTAVYYCATTIRGTVFAF | | | | TDYTLTISSLQPEDFATY |
| | | | | WGQGTLVTVSS (SEQ ID | | | | YCQQYYDYPLTFGQGT |
| | | | | NO: 138) | | | | KVEIK (SEQ ID NO: 148) |
| 29 | GYTFTE | RIDPED | TIRGTV | EVQLVQSGAEVKKPGASVKVS | RASEDIFN | NAKTLHT | QQYYDYP | DIQMTQSPSSLSASVGD |
| | YYMY | GGTD | FAF | CKASGYTFTEYYMYWVRQAP | GLA (SEQ | (SEQ ID | LT (SEQ | RVTITCRASEDIFNGLA |
| | (SEQ ID | (SEQ ID | (SEQ ID | GQGLELIGRIDPEDGGTDYIEKF | ID NO: 33) | NO: 41) | ID NO: 49) | WYQQKPGKSPKLLIYNA |
| | NO: 7) | NO: 15) | NO: 24) | KNRSTLTADTSTSTAYLELSSL | | | | KTLHTGVPSRFSGSGSG |
| | | | | RSEDTAVYFCATTIRGTVFAFW | | | | TDYTLTISSLQPEDFATY |
| | | | | GQGTLVTVSS (SEQ ID NO: | | | | YCQQYYDYPLTFGQGT |
| | | | | 140) | | | | KVEIK (SEQ ID NO: 148) |
| 30 | GYTFTE | RIDPED | TIRGTV | EVQLVQSGAEVKKPGASVKVS | RASEDIFN | NAKTLHT | QQYYDYP | DIQMTQSPSSLSASVGD |
| | YYMY | GGTD | FVY | CKASGYTFTEYYMYWVRQAP | ID NO: 33) | (SEQ ID | LT (SEQ | RVTITCRASEDIFNGLA |
| | (SEQ ID | (SEQ ID | (SEQ ID | GQGLELIGRIDPEDGGTDYVEK | GLA (SEQ | NO: 41) | ID NO: 49) | WYQQKPGKSPKLLIYNA |
| | NO: 7) | NO: 15) | NO: 25) | FKNRVTLTADTSTSTAYLELSS | | | | KTLHTGVPSRFSGSGSG |
| | | | | LRSEDTAVYYCASTIRGTVFVY | | | | TDYTLTISSLQPEDFATY |
| | | | | WGQGTLVTVSS (SEQ ID | | | | YCQQYYDYPLTFGQGT |
| | | | | NO: 158) | | | | KVEIK (SEQ ID NO: 148) |
| 31 | GYTFTE | RIDPED | TIRGTV | EVQLVQSGAEVKKPGASVKVS | RASEDIFN | NAKTLHT | QQYYDYP | DIQMTQSPSSLSASVGD |
| | YYMY | GGTD | FVY | CKASGYTFTEYYMYWVRQAP | GLA (SEQ | (SEQ ID | LT (SEQ | RVTITCRASEDIFNGLA |
| | (SEQ ID | (SEQ ID | (SEQ ID | GQGLELIGRIDPEDGGTDYVEK | ID NO: 33) | NO: 41) | ID NO: 49) | WYQQKPGKSPKLLIYNA |
| | NO: 7) | NO: 15) | NO: 25) | FKNRATLTADTSTSTAYLELSS | | | | KTLHTGVPSRFSGSGSG |
| | | | | LRSEDTAVYFCASTIRGTVFVY | | | | TDYTLTISSLQPEDFATY |
| | | | | WGQGTLVTVSS (SEQ ID NO: | | | | YCQQYYDYPLTFGQGT |
| | | | | 143) | | | | KVEIK (SEQ ID NO: 148) |
| 32 | GYTFTE | RIDPED | TIRGTV | EVQLVQSGAEVKKPGASVKVS | RPSEDIYN | NANSLHT | QQYYDYP | DIQMTQSPSSLSASVGD |
| | YYMY | GGTD | FAF | CKASGYTFTEYYMYWVRQAP | GLA (SEQ | (SEQ ID | LT (SEQ | RVTITCRPSEDIYNGLA |
| | (SEQ ID | (SEQ ID | (SEQ ID | GQGLELIGRIDPEDGGTDYIEKF | ID NO: 34) | NO: 42) | ID NO: 49) | WYQQKPGKSPKLLIYNA |
| | NO: 7) | NO: 15) | NO: 24) | KNRVTLTADTSTSTAYLELSSL | | | | NSLHTGVPSRFSGSGSGT |
| | | | | RSEDTAVYYCATTIRGTVFAF | | | | DYTLTISSLQPEDFATYF |
| | | | | WGQGTLVTVSS (SEQ ID | | | | CQQYYDYPLTFGQGTK |
| | | | | NO: 138) | | | | VEIK (SEQ ID NO: 149) |
| 33 | GYTFTE | RIDPED | TIRGTV | EVQLVQSGAEVKKPGASVKVS | RPSEDIYN | NANSLHT | QQYYDYP | DIQMTQSPSSLSASVGD |
| | YYMY | GGTD | FAF | CKASGYTFTEYYMYWVRQAP | GLA (SEQ | (SEQ ID | LT (SEQ | RVTITCRPSEDIYNGLA |
| | (SEQ ID | (SEQ ID | (SEQ ID | GQGLELIGRIDPEDGGTDYIEKF | ID NO: 34) | NO: 42) | ID NO: 49) | WYQQKPGKSPKLLIYNA |
| | NO: 7) | NO: 15) | NO: 24) | KNRSTLTADTSTSTAYLELSSL | | | | NSLHTGVPSRFSGSGSGT |
| | | | | RSEDTAVYFCATTIRGTVFAFW | | | | DYTLTISSLQPEDFATYF |
| | | | | GQGTLVTVSS (SEQ ID NO: | | | | CQQYYDYPLTFGQGTK |
| | | | | 140) | | | | VEIK (SEQ ID NO: 149) |
| 34 | GYTFTE | RIDPED | TIRGTV | EVQLVQSGAEVKKPGASVKVS | RPSEDIYN | NANSLHT | QQYYDYP | DIQMTQSPSSLSASVGD |
| | YYMY | GGTD | FVY | CKASGYTFTEYYMYWVRQAP | GLA (SEQ | (SEQ ID | LT (SEQ | RVTITCRPSEDIYNGLA |
| | (SEQ ID | (SEQ ID | (SEQ ID | GQGLELIGRIDPEDGGTDYVEK | ID NO: 34) | NO: 42) | ID NO: 49) | WYQQKPGKSPKLLIYNA |
| | NO: 7) | NO: 15) | NO: 25) | FKNRVTLTADTSTSTAYLELSS | | | | NSLHTGVPSRFSGSGSGT |
| | | | | LRSEDTAVYYCASTIRGTVFVY | | | | DYTLTISSLQPEDFATYF |
| | | | | WGQGTLVTVSS (SEQ ID NO: | | | | CQQYYDYPLTFGQGTK |
| | | | | 158) | | | | VEIK (SEQ ID NO: 149) |
| 35 | GYTFTE | RIDPED | TIRGTV | EVQLVQSGAEVKKPGASVKVS | RPSEDIYN | NANSLHT | QQYYDYP | DIQMTQSPSSLSASVGD |
| | YYMY | GGTD | FVY | CKASGYTFTEYYMYWVRQAP | GLA (SEQ | (SEQ ID | LT (SEQ | RVTITCRPSEDIYNGLA |
| | (SEQ ID | (SEQ ID | (SEQ ID | GQGLELIGRIDPEDGGTDYVEK | ID NO: 34) | NO: 42) | ID NO: 49) | WYQQKPGKSPKLLIYNA |
| | NO: 7) | NO: 15) | NO: 25) | FKNRATLTADTSTSTAYLELSS | | | | NSLHTGVPSRFSGSGSGT |
| | | | | LRSEDTAVYFCASTIRGTVFVY | | | | DYTLTISSLQPEDFATYF |
| | | | | WGQGTLVTVSS (SEQ ID | | | | CQQYYDYPLTFGQGTK |
| | | | | NO: 143) | | | | VEIK (SEQ ID NO: 149) |
| 36 | GYTFTE | RIDPED | TIRGTV | EVQLVQSGAEVKKPGASVKVS | RPSEDIYN | NANSLHT | QQYYDYP | DIQMTQSPSSLSASVGD |
| | YYMY | GGTD | FAF | CKASGYTFTEYYMYWVRQAP | GLA (SEQ | (SEQ ID | LT (SEQ | RVTITCRPSEDIYNGLA |
| | (SEQ ID | (SEQ ID | (SEQ ID | GQGLELIGRIDPEDGGTDYIEKF | ID NO: 34) | NO: 42) | ID NO: 49) | WYQQKPGKAPKLLIYN |
| | NO: 7) | NO: 15) | NO: 24) | KNRVTLTADTSTSTAYLELSSL | | | | ANSLHTGVPSRFSGSGS |
| | | | | RSEDTAVYYCATTIRGTVFAF | | | | GTDYTLTISSLQPEDFAT |
| | | | | WGQGTLVTVSS (SEQ ID | | | | YFCQQYYDYPLTFGQGT |
| | | | | NO: 138) | | | | KVEIK (SEQ ID NO: 147) |
| 37 | GYTFTE | RIDPED | TIRGTV | EVQLVQSGAEVKKPGASVKVS | RASEDIFN | NAKTLHT | QQYYDYP | DIQMTQSPSSLSASVGD |
| | YYMY | GGTD | FAF | CKASGYTFTEYYMYWVRQAP | GLA (SEQ | (SEQ ID | LT (SEQ | RVTITCRASEDIFNGLA |
| | (SEQ ID | (SEQ ID | (SEQ ID | GQGLELIGRIDPEDGGTDYIEKF | ID NO: 33) | NO: 41) | ID NO: 49) | WYQQKPGKSPKLLIYNA |
| | NO: 7) | NO: 15) | NO: 24) | KNRVTLTADTSTSTAYLELSSL | | | | KTLHTGVPSRFSGSGSG |
| | | | | RSEDTAVYYCATTIRGTVFAF | | | | TDYTLTISSLQPEDFATY |
| | | | | WGQGTLVTVSS (SEQ ID | | | | YCQQYYDYPLTFGQGT |
| | | | | NO: 138) | | | | KVEIK (SEQ ID NO: 148) |
| 38 | GYTFTE | RIDPED | TIRGTV | EVQLVQSGAEVKKPGASVKVS | RPSEDIYN | NANSLHT | QQYYDYP | DIQMTQSPSSLSASVGD |
| | YYMY | GGTD | FVY | CKASGYTFTEYYMYWVRQAP | GLA (SEQ | (SEQ ID | LT (SEQ | RVTITCRPSEDIYNGLA |
| | (SEQ ID | (SEQ ID | (SEQ ID | GQGLELIGRIDPEDGGTDYVEK | ID NO: 34) | NO: 42) | ID NO: 49) | WYQQKPGKAPKLLIYN |
| | NO: 7) | NO: 15) | NO: 25) | FKNRVTATADTSTSTAYLELSS | | | | ANSLHTGVPSRFSGSGS |
| | | | | LRSEDTAVYYCASTIRGTVFVY | | | | GTDYTLTISSLQPEDFAT |
| | | | | WGQGTLVTVSS (SEQ ID NO: | | | | YFCQQYYDYPLTFGQGT |
| | | | | 144) | | | | KVEIK (SEQ ID NO: 147) |
| 39 | GYTFTE | RIDPED | TIRGTV | EVQLVQSGAEVKKPGASVKVS | RASEDIFN | NAKTLHT | QQYYDYP | DIQMTQSPSSLSASVGD |
| | YYMY | GGTD | FAF | CKASGYTFTEYYMYWVRQAP | GLA (SEQ | (SEQ ID | LT (SEQ | RVTITCRASEDIFNGLA |
| | (SEQ ID | (SEQ ID | (SEQ ID | GQGLELIGRIDPEDGGTDYIEKF | ID NO: 33) | NO: 41) | ID NO: 49) | WYQQKPGKSPKLLIYNA |
| | NO: 7) | NO: 15) | NO: 24) | KNRVTLTADTSTSTAYLELSSL | | | | KTLHTGVPSRFSGSGSGS |
| | | | | RSEDTAVYYCATTIRGTVFAF | | | | DYTLTISSLQPEDFATYF |
| | | | | WGQGTLVTVSS (SEQ ID | | | | CQQYYDYPLTFGQGTK |
| | | | | NO: 138) | | | | VEIK (SEQ ID NO: 145) |

In some embodiments, an anti-PILRA antibody comprises one or more CDRs selected from the group consisting of
(a) a heavy chain CDR1 (CDR-H1) sequence having at least 90% sequence identity (e.g., at least 91%, 92%, 93%, 94%, 95%, 96%, 97%, 98%, or 99% sequence identity) to the amino acid sequence of any one of SEQ ID NOS:4-11, or having up to two amino acid substitutions relative to the amino acid sequence of any one of SEQ ID NOS:4-11;
(b) a heavy chain CDR2 (CDR-H2) sequence having at least 80% sequence identity (e.g., at least 82%, 84%, 86%, 88%, 90%, 91%, 92%, 93%, 94%, 95%, 96%, 97%, 98%, or 99% sequence identity) to the amino acid sequence of any one of SEQ ID NOS:12-19, or having up to two amino acid substitutions relative to the amino acid sequence of any one of SEQ ID NOS:12-19;
(c) a heavy chain CDR3 (CDR-H3) sequence having at least 80% sequence identity (e.g., at least 82%, 84%, 86%, 88%, 90%, 91%, 92%, 93%, 94%, 95%, 96%, 97%, 98%, or 99% sequence identity) to the amino acid sequence of any one of SEQ ID NOS:20-29, or having up to two amino acid substitutions relative to the amino acid sequence of any one of SEQ ID NOS:20-29;
(d) a light chain CDR1 (CDR-L1) sequence having at least 90% sequence identity (e.g., at least 91%, 92%, 93%, 94%, 95%, 96%, 97%, 98%, or 99% sequence identity) to the amino acid sequence of any one of SEQ ID NOS:30-38, or having up to two amino acid substitutions relative to the amino acid sequence of any one of SEQ ID NOS:30-38;
(e) a light chain CDR2 (CDR-L2) sequence having at least 80% sequence identity (e.g., at least 82%, 84%, 86%, 88%, 90%, 91%, 92%, 93%, 94%, 95%, 96%, 97%, 98%, or 99% sequence identity) to the amino acid sequence of SEQ ID NO:39-46, or having up to two amino acid substitutions relative to the amino acid sequence of SEQ ID NO:39-46; and
(f) a light chain CDR3 (CDR-L3) sequence having at least 80% sequence identity (e.g., at least 82%, 84%, 86%, 88%, 90%, 91%, 92%, 93%, 94%, 95%, 96%, 97%, 98%, or 99% sequence identity) to the amino acid sequence of any one of SEQ ID NOS:47-53, or having up to two amino acid substitutions relative to the amino acid sequence of any one of SEQ ID NOS:47-53.

In some embodiments, an anti-PILRA antibody comprises one or more CDRs selected from the group consisting of
(a) a CDR-H1 sequence comprising the amino acid sequence of any one of SEQ ID NOS:4-11;
(b) a CDR-H2 sequence comprising the amino acid sequence of any one of SEQ ID NOS:12-19;
(c) a CDR-H3 sequence comprising the amino acid sequence of any one of SEQ ID NOS:20-29;
(d) a CDR-L1 sequence comprising the amino acid sequence of any one of SEQ ID NOS:30-38;
(e) a CDR-L2 sequence comprising the amino acid sequence of any one of SEQ ID NOS:39-46; and
(f) a CDR-L3 sequence comprising the amino acid sequence of any one of SEQ ID NOS:47-53.

In some embodiments, an anti-PILRA antibody comprises two, three, four, five, or all six of (a)-(f). In some embodiments, an anti-PILRA antibody comprises the CDR-H1 of (a), the CDR-H2 of (b), and the CDR-H3 of (c). In some embodiments, an anti-PILRA antibody comprises the CDR-L1 of (d), the CDR-L2 of (e), and the CDR-L3 of (f). In some embodiments, a CDR having up to two amino acid substitutions has one amino acid substitution (e.g., one conservative substitution) relative to the reference sequence. In some embodiments, a CDR having up to two amino acid substitutions has two amino acid substitutions (e.g., two conservative substitutions) relative to the reference sequence. In some embodiments, the up to two amino acid substitutions are conservative substitutions.

In some embodiments, an anti-PILRA antibody comprises:
(a) a CDR-H1 having at least 90% sequence identity (e.g., at least 91%, 92%, 93%, 94%, 95%, 96%, 97%, 98%, or 99% sequence identity) to the amino acid sequence of SEQ ID NO:4, or having up to two amino acid substitutions (e.g., one or two conservative substitutions) relative to the amino acid sequence of SEQ ID NO:4;
(b) a CDR-H2 having at least 80% sequence identity (e.g., at least 82%, 84%, 86%, 88%, 90%, 91%, 92%, 93%, 94%, 95%, 96%, 97%, 98%, or 99% sequence identity) to the amino acid sequence of SEQ ID NO:12, or having up to two amino acid substitutions (e.g., one or two conservative substitutions) relative to the amino acid sequence of SEQ ID NO:12;
(c) a CDR-H3 having at least 80% sequence identity (e.g., at least 82%, 84%, 86%, 88%, 90%, 91%, 92%, 93%, 94%, 95%, 96%, 97%, 98%, or 99% sequence identity) to the amino acid sequence of SEQ ID NO:20, or having up to two amino acid substitutions (e.g., one or two conservative substitutions) relative to the amino acid sequence of SEQ ID NO:20;
(d) a CDR-L1 having at least 90% sequence identity (e.g., at least 91%, 92%, 93%, 94%, 95%, 96%, 97%, 98%, or 99% sequence identity) to the amino acid sequence of SEQ ID NO:30, or having up to two amino acid substitutions (e.g., one or two conservative substitutions) relative to the amino acid sequence of SEQ ID NO:30;
(e) a CDR-L2 having at least 80% sequence identity (e.g., at least 82%, 84%, 86%, 88%, 90%, 91%, 92%, 93%, 94%, 95%, 96%, 97%, 98%, or 99% sequence identity) to the amino acid sequence of SEQ ID NO:39, or having up to two amino acid substitutions (e.g., one or two conservative substitutions) relative to the amino acid sequence of SEQ ID NO:39; and
(f) a CDR-L3 having at least 80% sequence identity (e.g., at least 82%, 84%, 86%, 88%, 90%, 91%, 92%, 93%, 94%, 95%, 96%, 97%, 98%, or 99% sequence identity) to the amino acid sequence of SEQ ID NO:47, or having up to two amino acid substitutions (e.g., one or two conservative substitutions) relative to the amino acid sequence of SEQ ID NO:47.

In some embodiments, an anti-PILRA antibody comprises:
(a) a CDR-H1 having at least 90% sequence identity (e.g., at least 91%, 92%, 93%, 94%, 95%, 96%, 97%, 98%, or 99% sequence identity) to the amino acid sequence of SEQ ID NO:5, or having up to two amino acid substitutions (e.g., one or two conservative substitutions) relative to the amino acid sequence of SEQ ID NO:5;
(b) a CDR-H2 having at least 80% sequence identity (e.g., at least 82%, 84%, 86%, 88%, 90%, 91%, 92%, 93%, 94%, 95%, 96%, 97%, 98%, or 99% sequence identity) to the amino acid sequence of SEQ ID NO:13, or having up to two amino acid substitutions (e.g., one or two conservative substitutions) relative to the amino acid sequence of SEQ ID NO:13;
(c) a CDR-H3 having at least 80% sequence identity (e.g., at least 82%, 84%, 86%, 88%, 90%, 91%, 92%, 93%, 94%, 95%, 96%, 97%, 98%, or 99% sequence identity) to the amino acid sequence of SEQ ID NO:21, or having up to two amino acid substitutions (e.g., one or two conservative substitutions) relative to the amino acid sequence of SEQ ID NO:21;
(d) a CDR-L1 having at least 90% sequence identity (e.g., at least 91%, 92%, 93%, 94%, 95%, 96%, 97%, 98%, or 99% sequence identity) to the amino acid sequence of SEQ ID NO:31, or having up to two amino acid substitutions (e.g., one or two conservative substitutions) relative to the amino acid sequence of SEQ ID NO:31;
(e) a CDR-L2 having at least 80% sequence identity (e.g., at least 82%, 84%, 86%, 88%, 90%, 91%, 92%, 93%, 94%, 95%, 96%, 97%, 98%, or 99% sequence identity) to the amino acid sequence of SEQ ID NO:39, or having up to two amino acid substitutions (e.g., one or two conservative substitutions) relative to the amino acid sequence of SEQ ID NO:39; and
(f) a CDR-L3 having at least 80% sequence identity (e.g., at least 82%, 84%, 86%, 88%, 90%, 91%, 92%, 93%, 94%, 95%, 96%, 97%, 98%, or 99% sequence identity) to the amino acid sequence of SEQ ID NO:47, or having up to two amino acid substitutions (e.g., one or two conservative substitutions) relative to the amino acid sequence of SEQ ID NO:47.

In some embodiments, an anti-PILRA antibody comprises:
(a) a CDR-H1 having at least 90% sequence identity (e.g., at least 91%, 92%, 93%, 94%, 95%, 96%, 97%, 98%, or 99% sequence identity) to the amino acid sequence of SEQ ID NO:5, or having up to two amino acid substitutions (e.g., one or two conservative substitutions) relative to the amino acid sequence of SEQ ID NO:5;
(b) a CDR-H2 having at least 80% sequence identity (e.g., at least 82%, 84%, 86%, 88%, 90%, 91%, 92%, 93%, 94%, 95%, 96%, 97%, 98%, or 99% sequence identity) to the amino acid sequence of SEQ ID NO:13, or having up to two amino acid substitutions (e.g., one or two conservative substitutions) relative to the amino acid sequence of SEQ ID NO:13;
(c) a CDR-H3 having at least 80% sequence identity (e.g., at least 82%, 84%, 86%, 88%, 90%, 91%, 92%, 93%, 94%, 95%, 96%, 97%, 98%, or 99% sequence identity) to the amino acid sequence of SEQ ID NO:22, or having up to two amino acid substitutions (e.g., one or two conservative substitutions) relative to the amino acid sequence of SEQ ID NO:22;
(d) a CDR-L1 having at least 90% sequence identity (e.g., at least 91%, 92%, 93%, 94%, 95%, 96%, 97%, 98%, or 99% sequence identity) to the amino acid sequence of SEQ ID NO:31, or having up to two amino acid substitutions (e.g., one or two conservative substitutions) relative to the amino acid sequence of SEQ ID NO:31;
(e) a CDR-L2 having at least 80% sequence identity (e.g., at least 82%, 84%, 86%, 88%, 90%, 91%, 92%, 93%, 94%, 95%, 96%, 97%, 98%, or 99% sequence identity) to the amino acid sequence of SEQ ID NO:39, or having up to two amino acid substitutions (e.g., one or two conservative substitutions) relative to the amino acid sequence of SEQ ID NO:39; and
(f) a CDR-L3 having at least 80% sequence identity (e.g., at least 82%, 84%, 86%, 88%, 90%, 91%, 92%, 93%, 94%, 95%, 96%, 97%, 98%, or 99% sequence identity) to the amino acid sequence of SEQ ID NO:47, or having up to two amino acid substitutions (e.g., one or two conservative substitutions) relative to the amino acid sequence of SEQ ID NO:47.

In some embodiments, an anti-PILRA antibody comprises:
(a) a CDR-H1 having at least 90% sequence identity (e.g., at least 91%, 92%, 93%, 94%, 95%, 96%, 97%, 98%, or 99% sequence identity) to the amino acid sequence of SEQ ID NO:6, or having up to two amino acid substitutions (e.g., one or two conservative substitutions) relative to the amino acid sequence of SEQ ID NO:6;
(b) a CDR-H2 having at least 80% sequence identity (e.g., at least 82%, 84%, 86%, 88%, 90%, 91%, 92%, 93%, 94%, 95%, 96%, 97%, 98%, or 99% sequence identity) to the amino acid sequence of SEQ ID NO:14, or having up to two amino acid substitutions (e.g., one or two conservative substitutions) relative to the amino acid sequence of SEQ ID NO:14;
(c) a CDR-H3 having at least 80% sequence identity (e.g., at least 82%, 84%, 86%, 88%, 90%, 91%, 92%, 93%, 94%, 95%, 96%, 97%, 98%, or 99% sequence identity) to the amino acid sequence of SEQ ID NO:23, or having up to two amino acid substitutions (e.g., one or two conservative substitutions) relative to the amino acid sequence of SEQ ID NO:23;
(d) a CDR-L1 having at least 90% sequence identity (e.g., at least 91%, 92%, 93%, 94%, 95%, 96%, 97%, 98%, or 99% sequence identity) to the amino acid sequence of SEQ ID NO:32, or having up to two amino acid substitutions (e.g., one or two conservative substitutions) relative to the amino acid sequence of SEQ ID NO:32;
(e) a CDR-L2 having at least 80% sequence identity (e.g., at least 82%, 84%, 86%, 88%, 90%, 91%, 92%, 93%, 94%, 95%, 96%, 97%, 98%, or 99% sequence identity) to the amino acid sequence of SEQ ID NO:40, or having up to two amino acid substitutions (e.g., one or two conservative substitutions) relative to the amino acid sequence of SEQ ID NO:40; and
(f) a CDR-L3 having at least 80% sequence identity (e.g., at least 82%, 84%, 86%, 88%, 90%, 91%, 92%, 93%, 94%, 95%, 96%, 97%, 98%, or 99% sequence identity) to the amino acid sequence of SEQ ID NO:48, or having up to two amino acid substitutions (e.g., one or two conservative substitutions) relative to the amino acid sequence of SEQ ID NO:48.

In some embodiments, an anti-PILRA antibody comprises:
(a) a CDR-H1 having at least 90% sequence identity (e.g., at least 91%, 92%, 93%, 94%, 95%, 96%, 97%, 98%, or 99% sequence identity) to the amino acid sequence of SEQ ID NO:7, or having up to two amino acid substitutions (e.g., one or two conservative substitutions) relative to the amino acid sequence of SEQ ID NO:7;
(b) a CDR-H2 having at least 80% sequence identity (e.g., at least 82%, 84%, 86%, 88%, 90%, 91%, 92%, 93%, 94%, 95%, 96%, 97%, 98%, or 99% sequence identity) to the amino acid sequence of SEQ ID NO:15, or having up to two amino acid substitutions (e.g., one or two conservative substitutions) relative to the amino acid sequence of SEQ ID NO:15;
(c) a CDR-H3 having at least 80% sequence identity (e.g., at least 82%, 84%, 86%, 88%, 90%, 91%, 92%, 93%, 94%, 95%, 96%, 97%, 98%, or 99% sequence identity) to the amino acid sequence of SEQ ID NO:24, or having up to two amino acid substitutions (e.g., one or two conservative substitutions) relative to the amino acid sequence of SEQ ID NO:24;
(d) a CDR-L1 having at least 90% sequence identity (e.g., at least 91%, 92%, 93%, 94%, 95%, 96%, 97%, 98%, or 99% sequence identity) to the amino acid sequence of SEQ ID NO:33, or having up to two amino acid substitutions (e.g., one or two conservative substitutions) relative to the amino acid sequence of SEQ ID NO:33;
(e) a CDR-L2 having at least 80% sequence identity (e.g., at least 82%, 84%, 86%, 88%, 90%, 91%, 92%, 93%, 94%, 95%, 96%, 97%, 98%, or 99% sequence identity) to the amino acid sequence of SEQ ID NO:41, or having up to two amino acid substitutions (e.g., one or two conservative substitutions) relative to the amino acid sequence of SEQ ID NO:41; and
(f) a CDR-L3 having at least 80% sequence identity (e.g., at least 82%, 84%, 86%, 88%, 90%, 91%, 92%, 93%, 94%, 95%, 96%, 97%, 98%, or 99% sequence identity) to the amino acid sequence of SEQ ID NO:49, or having up to two amino acid substitutions (e.g., one or two conservative substitutions) relative to the amino acid sequence of SEQ ID NO:49.

In some embodiments, an anti-PILRA antibody comprises:
(a) a CDR-H1 having at least 90% sequence identity (e.g., at least 91%, 92%, 93%, 94%, 95%, 96%, 97%, 98%, or 99% sequence identity) to the amino acid sequence of SEQ ID NO:7, or having up to two amino acid substitutions (e.g., one or two conservative substitutions) relative to the amino acid sequence of SEQ ID NO:7;
(b) a CDR-H2 having at least 80% sequence identity (e.g., at least 82%, 84%, 86%, 88%, 90%, 91%, 92%, 93%, 94%, 95%, 96%, 97%, 98%, or 99% sequence identity) to the amino acid sequence of SEQ ID NO:15, or having up to two amino acid substitutions (e.g., one or two conservative substitutions) relative to the amino acid sequence of SEQ ID NO:15;
(c) a CDR-H3 having at least 80% sequence identity (e.g., at least 82%, 84%, 86%, 88%, 90%, 91%, 92%, 93%, 94%, 95%, 96%, 97%, 98%, or 99% sequence identity) to the amino acid sequence of SEQ ID NO:25, or having up to two amino acid substitutions (e.g., one or two conservative substitutions) relative to the amino acid sequence of SEQ ID NO:25;
(d) a CDR-L1 having at least 90% sequence identity (e.g., at least 91%, 92%, 93%, 94%, 95%, 96%, 97%, 98%, or 99% sequence identity) to the amino acid sequence of SEQ ID NO:34, or having up to two amino acid substitutions (e.g., one or two conservative substitutions) relative to the amino acid sequence of SEQ ID NO:34;
(e) a CDR-L2 having at least 80% sequence identity (e.g., at least 82%, 84%, 86%, 88%, 90%, 91%, 92%, 93%, 94%, 95%, 96%, 97%, 98%, or 99% sequence identity) to the amino acid sequence of SEQ ID NO:42, or having up to two amino acid substitutions (e.g., one or two conservative substitutions) relative to the amino acid sequence of SEQ ID NO:42; and
(f) a CDR-L3 having at least 80% sequence identity (e.g., at least 82%, 84%, 86%, 88%, 90%, 91%, 92%, 93%, 94%, 95%, 96%, 97%, 98%, or 99% sequence identity) to the amino acid sequence of SEQ ID NO:49, or having up to two amino acid substitutions (e.g., one or two conservative substitutions) relative to the amino acid sequence of SEQ ID NO:49.

In some embodiments, an anti-PILRA antibody comprises:
(a) a CDR-H1 having at least 90% sequence identity (e.g., at least 91%, 92%, 93%, 94%, 95%, 96%, 97%, 98%, or 99% sequence identity) to the amino acid sequence of SEQ ID NO:8, or having up to two amino acid substitutions (e.g., one or two conservative substitutions) relative to the amino acid sequence of SEQ ID NO:8;
(b) a CDR-H2 having at least 80% sequence identity (e.g., at least 82%, 84%, 86%, 88%, 90%, 91%, 92%, 93%, 94%, 95%, 96%, 97%, 98%, or 99% sequence identity) to the amino acid sequence of SEQ ID NO:16, or having up to two amino acid substitutions (e.g., one or two conservative substitutions) relative to the amino acid sequence of SEQ ID NO:16;
(c) a CDR-H3 having at least 80% sequence identity (e.g., at least 82%, 84%, 86%, 88%, 90%, 91%, 92%, 93%, 94%, 95%, 96%, 97%, 98%, or 99% sequence identity) to the amino acid sequence of SEQ ID NO:26, or having up to two amino acid substitutions (e.g., one or two conservative substitutions) relative to the amino acid sequence of SEQ ID NO:26;
(d) a CDR-L1 having at least 90% sequence identity (e.g., at least 91%, 92%, 93%, 94%, 95%, 96%, 97%, 98%, or 99% sequence identity) to the amino acid sequence of SEQ ID NO:35, or having up to two amino acid substitutions (e.g., one or two conservative substitutions) relative to the amino acid sequence of SEQ ID NO:35;
(e) a CDR-L2 having at least 80% sequence identity (e.g., at least 82%, 84%, 86%, 88%, 90%, 91%, 92%, 93%, 94%, 95%, 96%, 97%, 98%, or 99% sequence identity) to the amino acid sequence of SEQ ID NO:43, or having up to two amino acid substitutions (e.g., one or two conservative substitutions) relative to the amino acid sequence of SEQ ID NO:43; and
(f) a CDR-L3 having at least 80% sequence identity (e.g., at least 82%, 84%, 86%, 88%, 90%, 91%, 92%, 93%, 94%, 95%, 96%, 97%, 98%, or 99% sequence identity) to the amino acid sequence of SEQ ID NO:50, or having up to two amino acid substitutions (e.g., one or two conservative substitutions) relative to the amino acid sequence of SEQ ID NO:50.

In some embodiments, an anti-PILRA antibody comprises:
(a) a CDR-H1 having at least 90% sequence identity (e.g., at least 91%, 92%, 93%, 94%, 95%, 96%, 97%, 98%, or 99% sequence identity) to the amino acid sequence of SEQ ID NO:9, or having up to two amino acid substitutions (e.g., one or two conservative substitutions) relative to the amino acid sequence of SEQ ID NO:9;
(b) a CDR-H2 having at least 80% sequence identity (e.g., at least 82%, 84%, 86%, 88%, 90%, 91%, 92%, 93%, 94%, 95%, 96%, 97%, 98%, or 99% sequence identity) to the amino acid sequence of SEQ ID NO:17, or having up to two amino acid substitutions (e.g., one or two conservative substitutions) relative to the amino acid sequence of SEQ ID NO:17;
(c) a CDR-H3 having at least 80% sequence identity (e.g., at least 82%, 84%, 86%, 88%, 90%, 91%, 92%, 93%, 94%, 95%, 96%, 97%, 98%, or 99% sequence identity) to the amino acid sequence of SEQ ID NO:27, or having up to two amino acid substitutions (e.g., one or two conservative substitutions) relative to the amino acid sequence of SEQ ID NO:27;
(d) a CDR-L1 having at least 90% sequence identity (e.g., at least 91%, 92%, 93%, 94%, 95%, 96%, 97%, 98%, or 99% sequence identity) to the amino acid sequence of SEQ ID NO:36, or having up to two amino acid substitutions (e.g., one or two conservative substitutions) relative to the amino acid sequence of SEQ ID NO:36;
(e) a CDR-L2 having at least 80% sequence identity (e.g., at least 82%, 84%, 86%, 88%, 90%, 91%, 92%, 93%, 94%, 95%, 96%, 97%, 98%, or 99% sequence identity) to the amino acid sequence of SEQ ID NO:44, or having up to two amino acid substitutions (e.g., one or two conservative substitutions) relative to the amino acid sequence of SEQ ID NO:44; and
(f) a CDR-L3 having at least 80% sequence identity (e.g., at least 82%, 84%, 86%, 88%, 90%, 91%, 92%, 93%, 94%, 95%, 96%, 97%, 98%, or 99% sequence identity) to the amino acid sequence of SEQ ID NO:51, or having up to two amino acid substitutions (e.g., one or two conservative substitutions) relative to the amino acid sequence of SEQ ID NO:51.

In some embodiments, an anti-PILRA antibody comprises:
(a) a CDR-H1 having at least 90% sequence identity (e.g., at least 91%, 92%, 93%, 94%, 95%, 96%, 97%, 98%, or 99% sequence identity) to the amino acid sequence of SEQ ID NO:10, or having up to two amino acid substitutions (e.g., one or two conservative substitutions) relative to the amino acid sequence of SEQ ID NO:10;
(b) a CDR-H2 having at least 80% sequence identity (e.g., at least 82%, 84%, 86%, 88%, 90%, 91%, 92%, 93%, 94%, 95%, 96%, 97%, 98%, or 99% sequence identity) to the amino acid sequence of SEQ ID NO:18, or having up to two amino acid substitutions (e.g., one or two conservative substitutions) relative to the amino acid sequence of SEQ ID NO:18;
(c) a CDR-H3 having at least 80% sequence identity (e.g., at least 82%, 84%, 86%, 88%, 90%, 91%, 92%, 93%, 94%, 95%, 96%, 97%, 98%, or 99% sequence identity) to the amino acid sequence of SEQ ID NO:28, or having up to two amino acid substitutions (e.g., one or two conservative substitutions) relative to the amino acid sequence of SEQ ID NO:28;
(d) a CDR-L1 having at least 90% sequence identity (e.g., at least 91%, 92%, 93%, 94%, 95%, 96%, 97%, 98%, or 99% sequence identity) to the amino acid sequence of SEQ ID NO:37, or having up to two amino acid substitutions (e.g., one or two conservative substitutions) relative to the amino acid sequence of SEQ ID NO:37;
(e) a CDR-L2 having at least 80% sequence identity (e.g., at least 82%, 84%, 86%, 88%, 90%, 91%, 92%, 93%, 94%, 95%, 96%, 97%, 98%, or 99% sequence identity) to the amino acid sequence of SEQ ID NO:45, or having up to two amino acid substitutions (e.g., one or two conservative substitutions) relative to the amino acid sequence of SEQ ID NO:45; and
(f) a CDR-L3 having at least 80% sequence identity (e.g., at least 82%, 84%, 86%, 88%, 90%, 91%, 92%, 93%, 94%, 95%, 96%, 97%, 98%, or 99% sequence identity) to the amino acid sequence of SEQ ID NO:52, or having up to two amino acid substitutions (e.g., one or two conservative substitutions) relative to the amino acid sequence of SEQ ID NO:52.

In some embodiments, an anti-PILRA antibody comprises:
(a) a CDR-H1 having at least 90% sequence identity (e.g., at least 91%, 92%, 93%, 94%, 95%, 96%, 97%, 98%, or 99% sequence identity) to the amino acid sequence of SEQ ID NO: 11, or having up to two amino acid substitutions (e.g., one or two conservative substitutions) relative to the amino acid sequence of SEQ ID NO:11;
(b) a CDR-H2 having at least 80% sequence identity (e.g., at least 82%, 84%, 86%, 88%, 90%, 91%, 92%, 93%, 94%, 95%, 96%, 97%, 98%, or 99% sequence identity) to the amino acid sequence of SEQ ID NO:19, or having up to two amino acid substitutions (e.g., one or two conservative substitutions) relative to the amino acid sequence of SEQ ID NO:19;
(c) a CDR-H3 having at least 80% sequence identity (e.g., at least 82%, 84%, 86%, 88%, 90%, 91%, 92%, 93%, 94%, 95%, 96%, 97%, 98%, or 99% sequence identity) to the amino acid sequence of SEQ ID NO:29, or having up to two amino acid substitutions (e.g., one or two conservative substitutions) relative to the amino acid sequence of SEQ ID NO:29;
(d) a CDR-L1 having at least 90% sequence identity (e.g., at least 91%, 92%, 93%, 94%, 95%, 96%, 97%, 98%, or 99% sequence identity) to the amino acid sequence of SEQ ID NO:38, or having up to two amino acid substitutions (e.g., one or two conservative substitutions) relative to the amino acid sequence of SEQ ID NO:38;
(e) a CDR-L2 having at least 80% sequence identity (e.g., at least 82%, 84%, 86%, 88%, 90%, 91%, 92%, 93%, 94%, 95%, 96%, 97%, 98%, or 99% sequence identity) to the amino acid sequence of SEQ ID NO:46, or having up to two amino acid substitutions (e.g., one or two conservative substitutions) relative to the amino acid sequence of SEQ ID NO:46; and
(f) a CDR-L3 having at least 80% sequence identity (e.g., at least 82%, 84%, 86%, 88%, 90%, 91%, 92%, 93%, 94%, 95%, 96%, 97%, 98%, or 99% sequence identity) to the amino acid sequence of SEQ ID NO:53, or having up to two amino acid substitutions (e.g., one or two conservative substitutions) relative to the amino acid sequence of SEQ ID NO:53.

In some embodiments, an anti-PILRA antibody comprises:
(a) a CDR-H1 comprising the amino acid sequence of SEQ ID NO:4, a CDR-H2 comprising the amino acid sequence of SEQ ID NO:12, a CDR-H3 comprising the amino acid sequence of SEQ ID NO:20, a CDR-L1 comprising the amino acid sequence of SEQ ID NO:30, a CDR-L2 comprising the amino acid sequence of SEQ ID NO:39, and a CDR-L3 comprising the amino acid sequence of SEQ ID NO:47; or
(b) a CDR-H1 comprising the amino acid sequence of SEQ ID NO:5, a CDR-H2 comprising the amino acid sequence of SEQ ID NO:13, a CDR-H3 comprising the amino acid sequence of SEQ ID NO:21, a CDR-L1 comprising the amino acid sequence of SEQ ID NO:31, a CDR-L2 comprising the amino acid sequence of SEQ ID NO:39, and a CDR-L3 comprising the amino acid sequence of SEQ ID NO:47; or
(c) a CDR-H1 comprising the amino acid sequence of SEQ ID NO:5, a CDR-H2 comprising the amino acid sequence of SEQ ID NO:13, a CDR-H3 comprising the amino acid sequence of SEQ ID NO:22, a CDR-L1 comprising the amino acid sequence of SEQ ID NO:31, a CDR-L2 comprising the amino acid sequence of SEQ ID NO:39, and a CDR-L3 comprising the amino acid sequence of SEQ ID NO:47; or
(d) a CDR-H1 comprising the amino acid sequence of SEQ ID NO:6, a CDR-H2 comprising the amino acid sequence of SEQ ID NO:14, a CDR-H3 comprising the amino acid sequence of SEQ ID NO:23, a CDR-L1 comprising the amino acid sequence of SEQ ID NO:32, a CDR-L2 comprising the amino acid sequence of SEQ ID NO:40, and a CDR-L3 comprising the amino acid sequence of SEQ ID NO:48; or
(e) a CDR-H1 comprising the amino acid sequence of SEQ ID NO:7, a CDR-H2 comprising the amino acid sequence of SEQ ID NO:15, a CDR-H3 comprising the amino acid sequence of SEQ ID NO:24, a CDR-L1 comprising the amino acid sequence of SEQ ID NO:33, a CDR-L2 comprising the amino acid sequence of SEQ ID NO:41, and a CDR-L3 comprising the amino acid sequence of SEQ ID NO:49; or
(f) a CDR-H1 comprising the amino acid sequence of SEQ ID NO:7, a CDR-H2 comprising the amino acid sequence of SEQ ID NO:15, a CDR-H3 comprising the amino acid sequence of SEQ ID NO:25, a CDR-L1 comprising the amino acid sequence of SEQ ID NO:34, a CDR-L2 comprising the amino acid sequence of SEQ ID NO:42, and a CDR-L3 comprising the amino acid sequence of SEQ ID NO:49; or
(g) a CDR-H1 comprising the amino acid sequence of SEQ ID NO:8, a CDR-H2 comprising the amino acid sequence of SEQ ID NO:16, a CDR-H3 comprising the amino acid sequence of SEQ ID NO:26, a CDR-L1 comprising the amino acid sequence of SEQ ID NO:35, a CDR-L2 comprising the amino acid sequence of SEQ ID NO:43, and a CDR-L3 comprising the amino acid sequence of SEQ ID NO:50; or
(h) a CDR-H1 comprising the amino acid sequence of SEQ ID NO:9, a CDR-H2 comprising the amino acid sequence of SEQ ID NO:17, a CDR-H3 comprising the amino acid sequence of SEQ ID NO:27, a CDR-L1 comprising the amino acid sequence of SEQ ID NO:36, a CDR-L2 comprising the amino acid sequence of SEQ ID NO:44, and a CDR-L3 comprising the amino acid sequence of SEQ ID NO:51; or
(i) a CDR-H1 comprising the amino acid sequence of SEQ ID NO:10, a CDR-H2 comprising the amino acid sequence of SEQ ID NO:18, a CDR-H3 comprising the amino acid sequence of SEQ ID NO:28, a CDR-L1 comprising the amino acid sequence of SEQ ID NO:37, a CDR-L2 comprising the amino acid sequence of SEQ ID NO:45, and a CDR-L3 comprising the amino acid sequence of SEQ ID NO:52; or
(j) a CDR-H1 comprising the amino acid sequence of SEQ ID NO:11, a CDR-H2 comprising the amino acid sequence of SEQ ID NO:19, a CDR-H3 comprising the amino acid sequence of SEQ ID NO:29, a CDR-L1 comprising the amino acid sequence of SEQ ID NO:38, a CDR-L2 comprising the amino acid sequence of SEQ ID NO:46, and a CDR-L3 comprising the amino acid sequence of SEQ ID NO:53.

In some embodiments, an anti-PILRA antibody comprises a heavy chain variable region comprising an amino acid sequence that has at least 85%, 90%, 91%, 92%, 93%, 94%, 95%, 96%, 97%, 98%, or 99% sequence identity to any one of SEQ ID NOS:54-63. In some embodiments, an anti-PILRA comprises a heavy chain variable region comprising the amino acid sequence of any one of SEQ ID NOS:54-63.

In some embodiments, an anti-PILRA antibody comprises a heavy chain variable region comprising an amino acid sequence that has at least 85%, 90%, 91%, 92%, 93%, 94%, 95%, 96%, 97%, 98%, or 99% sequence identity to any one of SEQ ID NOS:137-144 and 158. In some embodiments, an anti-PILRA comprises a heavy chain variable region comprising the amino acid sequence of any one of SEQ ID NOS:137-144 and 158.

In some embodiments, an anti-PILRA antibody comprises a light chain variable region comprising an amino acid sequence that has at least 85%, 90%, 91%, 92%, 93%, 94%, 95%, 96%, 97%, 98%, or 99% sequence identity to any one of SEQ ID NOS:64-73. In some embodiments, an anti-PILRA antibody comprises a light chain variable region comprising the amino acid sequence of any one of SEQ ID NOS:64-73.

In some embodiments, an anti-PILRA antibody comprises: a heavy chain variable region comprising an amino acid sequence that has at least 85%, 90%, 91%, 92%, 93%, 94%, 95%, 96%, 97%, 98%, or 99% sequence identity to any one of SEQ ID NOS:54-63, a light chain variable region comprising an amino acid sequence that has at least 85%, 90%, 91%, 92%, 93%, 94%, 95%, 96%, 97%, 98%, or 99% sequence identity to any one of SEQ ID NOS:64-73. In some embodiments, an anti-PILRA comprises: a heavy chain variable region comprising the amino acid sequence of any one of SEQ ID NOS:54-63, and a light chain variable region comprising the amino acid sequence of any one of SEQ ID NOS:64-73.

In some embodiments, an anti-PILRA antibody comprises: a heavy chain variable region comprising an amino acid sequence that has at least 85%, 90%, 91%, 92%, 93%, 94%, 95%, 96%, 97%, 98%, or 99% sequence identity to any one of SEQ ID NOS:137-144, a light chain variable region comprising an amino acid sequence that has at least 85%, 90%, 91%, 92%, 93%, 94%, 95%, 96%, 97%, 98%, or 99% sequence identity to any one of SEQ ID NOS:145-149. In some embodiments, an anti-PILRA comprises: a heavy chain variable region comprising the amino acid sequence of any one of SEQ ID NOS:137-144, and a light chain variable region comprising the amino acid sequence of any one of SEQ ID NOS:145-149.

In some embodiments, an anti-PILRA antibody comprises:
(a) a V_{H} sequence that has at least 85%, 90%, 91%, 92%, 93%, 94%, 95%, 96%, 97%, 98%, or 99% sequence identity to SEQ ID NO:54 and a V_{L} sequence has at least 85%, 90%, 91%, 92%, 93%, 94%, 95%, 96%, 97%, 98%, or 99% sequence identity to SEQ ID NO:64; or
(b) a V_{H} sequence that has at least 85%, 90%, 91%, 92%, 93%, 94%, 95%, 96%, 97%, 98%, or 99% sequence identity to SEQ ID NO:54 and a V_{L} sequence has at least 85%, 90%, 91%, 92%, 93%, 94%, 95%, 96%, 97%, 98%, or 99% sequence identity to SEQ ID NO:65; or
(c) a V_{H} sequence that has at least 85%, 90%, 91%, 92%, 93%, 94%, 95%, 96%, 97%, 98%, or 99% sequence identity to SEQ ID NO:55 and a V_{L} sequence has at least 85%, 90%, 91%, 92%, 93%, 94%, 95%, 96%, 97%, 98%, or 99% sequence identity to SEQ ID NO:66; or
(d) a V_{H} sequence that has at least 85%, 90%, 91%, 92%, 93%, 94%, 95%, 96%, 97%, 98%, or 99% sequence identity to SEQ ID NO:56 and a V_{L} sequence has at least 85%, 90%, 91%, 92%, 93%, 94%, 95%, 96%, 97%, 98%, or 99% sequence identity to SEQ ID NO:66; or
(e) a V_{H} sequence that has at least 85%, 90%, 91%, 92%, 93%, 94%, 95%, 96%, 97%, 98%, or 99% sequence identity to SEQ ID NO:57 and a V_{L} sequence has at least 85%, 90%, 91%, 92%, 93%, 94%, 95%, 96%, 97%, 98%, or 99% sequence identity to SEQ ID NO:67; or
(f) a V_{H} sequence that has at least 85%, 90%, 91%, 92%, 93%, 94%, 95%, 96%, 97%, 98%, or 99% sequence identity to SEQ ID NO:58 and a V_{L} sequence has at least 85%, 90%, 91%, 92%, 93%, 94%, 95%, 96%, 97%, 98%, or 99% sequence identity to SEQ ID NO:68; or
(g) a V_{H} sequence that has at least 85%, 90%, 91%, 92%, 93%, 94%, 95%, 96%, 97%, 98%, or 99% sequence identity to SEQ ID NO:59 and a V_{L} sequence has at least 85%, 90%, 91%, 92%, 93%, 94%, 95%, 96%, 97%, 98%, or 99% sequence identity to SEQ ID NO:69; or
(h) a V_{H} sequence that has at least 85%, 90%, 91%, 92%, 93%, 94%, 95%, 96%, 97%, 98%, or 99% sequence identity to SEQ ID NO:60 and a V_{L} sequence has at least 85%, 90%, 91%, 92%, 93%, 94%, 95%, 96%, 97%, 98%, or 99% sequence identity to SEQ ID NO:70; or
(i) a V_{H} sequence that has at least 85%, 90%, 91%, 92%, 93%, 94%, 95%, 96%, 97%, 98%, or 99% sequence identity to SEQ ID NO:61 and a V_{L} sequence has at least 85%, 90%, 91%, 92%, 93%, 94%, 95%, 96%, 97%, 98%, or 99% sequence identity to SEQ ID NO:71; or
(j) a V_{H} sequence that has at least 85%, 90%, 91%, 92%, 93%, 94%, 95%, 96%, 97%, 98%, or 99% sequence identity to SEQ ID NO:62 and a V_{L} sequence has at least 85%, 90%, 91%, 92%, 93%, 94%, 95%, 96%, 97%, 98%, or 99% sequence identity to SEQ ID NO:72; or
(k) a V_{H} sequence that has at least 85%, 90%, 91%, 92%, 93%, 94%, 95%, 96%, 97%, 98%, or 99% sequence identity to SEQ ID NO:63 and a V_{L} sequence has at least 85%, 90%, 91%, 92%, 93%, 94%, 95%, 96%, 97%, 98%, or 99% sequence identity to SEQ ID NO:73.

In some embodiments, an anti-PILRA antibody comprises:
(a) a CDR-H1, a CDR-H2, a CDR-H3 comprising the amino acid sequences of SEQ ID NOS:4, 12, and 20, respectively, and a V_{H} sequence that has at least 85%, 90%, 91%, 92%, 93%, 94%, 95%, 96%, 97%, 98%, or 99% sequence identity to SEQ ID NO:54 and a CDR-L1, a CDR-L2, a CDR-L3 comprising the amino acid sequences of SEQ ID NOS:30, 39, and 47, respectively, and a V_{L} sequence has at least 85%, 90%, 91%, 92%, 93%, 94%, 95%, 96%, 97%, 98%, or 99% sequence identity to SEQ ID NO:64; or
(b) a CDR-H1, a CDR-H2, a CDR-H3 comprising the amino acid sequences of SEQ ID NOS:4, 12, and 20, respectively, and a V_{H} sequence that has at least 85%, 90%, 91%, 92%, 93%, 94%, 95%, 96%, 97%, 98%, or 99% sequence identity to SEQ ID NO:54 and a CDR-L1, a CDR-L2, a CDR-L3 comprising the amino acid sequences of SEQ ID NOS:30, 39, and 47, respectively, and a V_{L} sequence has at least 85%, 90%, 91%, 92%, 93%, 94%, 95%, 96%, 97%, 98%, or 99% sequence identity to SEQ ID NO:65; or
(c) a CDR-H1, a CDR-H2, a CDR-H3 comprising the amino acid sequences of SEQ ID NOS:5, 13, and 21, respectively, and a V_{H} sequence that has at least 85%, 90%, 91%, 92%, 93%, 94%, 95%, 96%, 97%, 98%, or 99% sequence identity to SEQ ID NO:55 and a CDR-L1, a CDR-L2, a CDR-L3 comprising the amino acid sequences of SEQ ID NOS:31, 39, and 47, respectively, and a V_{L} sequence has at least 85%, 90%, 91%, 92%, 93%, 94%, 95%, 96%, 97%, 98%, or 99% sequence identity to SEQ ID NO:66; or
(d) a CDR-H1, a CDR-H2, a CDR-H3 comprising the amino acid sequences of SEQ ID NOS:5, 13, and 22, respectively, and a V_{H} sequence that has at least 85%, 90%, 91%, 92%, 93%, 94%, 95%, 96%, 97%, 98%, or 99% sequence identity to SEQ ID NO:56 and a CDR-L1, a CDR-L2, a CDR-L3 comprising the amino acid sequences of SEQ ID NOS:31, 39, and 47, respectively, and a V_{L} sequence has at least 85%, 90%, 91%, 92%, 93%, 94%, 95%, 96%, 97%, 98%, or 99% sequence identity to SEQ ID NO:66; or
(e) a CDR-H1, a CDR-H2, a CDR-H3 comprising the amino acid sequences of SEQ ID NOS:6, 14, and 23, respectively, and a V_{H} sequence that has at least 85%, 90%, 91%, 92%, 93%, 94%, 95%, 96%, 97%, 98%, or 99% sequence identity to SEQ ID NO:57 and a CDR-L1, a CDR-L2, a CDR-L3 comprising the amino acid sequences of SEQ ID NOS:32, 40, and 48, respectively, and a V_{L} sequence has at least 85%, 90%, 91%, 92%, 93%, 94%, 95%, 96%, 97%, 98%, or 99% sequence identity to SEQ ID NO:67; or
(f) a CDR-H1, a CDR-H2, a CDR-H3 comprising the amino acid sequences of SEQ ID NOS:7, 15, and 24, respectively, and a V_{H} sequence that has at least 85%, 90%, 91%, 92%, 93%, 94%, 95%, 96%, 97%, 98%, or 99% sequence identity to SEQ ID NO:58 and a CDR-L1, a CDR-L2, a CDR-L3 comprising the amino acid sequences of SEQ ID NOS:33, 41, and 49, respectively, and a V_{L} sequence has at least 85%, 90%, 91%, 92%, 93%, 94%, 95%, 96%, 97%, 98%, or 99% sequence identity to SEQ ID NO:68; or
(g) a CDR-H1, a CDR-H2, a CDR-H3 comprising the amino acid sequences of SEQ ID NOS:7, 15, and 25, respectively, and a V_{H} sequence that has at least 85%, 90%, 91%, 92%, 93%, 94%, 95%, 96%, 97%, 98%, or 99% sequence identity to SEQ ID NO:59 and a CDR-L1, a CDR-L2, a CDR-L3 comprising the amino acid sequences of SEQ ID NOS:34, 42, and 49, respectively, and a V_{L} sequence has at least 85%, 90%, 91%, 92%, 93%, 94%, 95%, 96%, 97%, 98%, or 99% sequence identity to SEQ ID NO:69; or
(h) a CDR-H1, a CDR-H2, a CDR-H3 comprising the amino acid sequences of SEQ ID NOS:8, 16, and 26, respectively, and a V_{H} sequence that has at least 85%, 90%, 91%, 92%, 93%, 94%, 95%, 96%, 97%, 98%, or 99% sequence identity to SEQ ID NO:60 and a CDR-L1, a CDR-L2, a CDR-L3 comprising the amino acid sequences of SEQ ID NOS:35, 43, and 50, respectively, and a V_{L} sequence has at least 85%, 90%, 91%, 92%, 93%, 94%, 95%, 96%, 97%, 98%, or 99% sequence identity to SEQ ID NO:70; or
(i) a CDR-H1, a CDR-H2, a CDR-H3 comprising the amino acid sequences of SEQ ID NOS:9, 17, and 27, respectively, and a V_{H} sequence that has at least 85%, 90%, 91%, 92%, 93%, 94%, 95%, 96%, 97%, 98%, or 99% sequence identity to SEQ ID NO:61 and a CDR-L1, a CDR-L2, a CDR-L3 comprising the amino acid sequences of SEQ ID NOS:36, 44, and 51, respectively, and a V_{L} sequence has at least 85%, 90%, 91%, 92%, 93%, 94%, 95%, 96%, 97%, 98%, or 99% sequence identity to SEQ ID NO:71; or
(j) a CDR-H1, a CDR-H2, a CDR-H3 comprising the amino acid sequences of SEQ ID NOS:10, 18, and 28, respectively, and a V_{H} sequence that has at least 85%, 90%, 91%, 92%, 93%, 94%, 95%, 96%, 97%, 98%, or 99% sequence identity to SEQ ID NO:62 and a CDR-L1, a CDR-L2, a CDR-L3 comprising the amino acid sequences of SEQ ID NOS:37, 45, and 52, respectively, and a V_{L} sequence has at least 85%, 90%, 91%, 92%, 93%, 94%, 95%, 96%, 97%, 98%, or 99% sequence identity to SEQ ID NO:72; or
(k) a CDR-H1, a CDR-H2, a CDR-H3 comprising the amino acid sequences of SEQ ID NOS:11, 19, and 29, respectively, and a V_{H} sequence that has at least 85%, 90%, 91%, 92%, 93%, 94%, 95%, 96%, 97%, 98%, or 99% sequence identity to SEQ ID NO:63 and a CDR-L1, a CDR-L2, a CDR-L3 comprising the amino acid sequences of SEQ ID NOS:38, 46, and 53, respectively, and a V_{L} sequence has at least 85%, 90%, 91%, 92%, 93%, 94%, 95%, 96%, 97%, 98%, or 99% sequence identity to SEQ ID NO:73.

### Clone 2

In some embodiments, an anti-PILRA antibody comprises a CDR-H1 sequence comprising the amino acid sequence of SEQ ID NO:4, a CDR-H2 sequence comprising the amino acid sequence of SEQ ID NO:12, a CDR-H3 sequence comprising the amino acid sequence of SEQ ID NO:20, a CDR-L1 sequence comprising the amino acid sequence of SEQ ID NO:30, a CDR-L2 sequence comprising the amino acid sequence of SEQ ID NO:39, and a CDR-L3 sequence comprising the amino acid sequence of SEQ ID NO:47.

In some embodiments, an anti-PILRA antibody comprises a heavy chain variable region comprising an amino acid sequence that has at least 85% sequence identity (e.g., at least 90%, 91%, 92%, 93%, 94%, 95%, 96%, 97%, 98%, or 99% sequence identity) to SEQ ID NO:54. In some embodiments, an anti-PILRA antibody comprises a heavy chain variable region comprising the amino acid sequence of SEQ ID NO:54.

In some embodiments, an anti-PILRA antibody comprises a light chain variable region comprising an amino acid sequence that has at least 85% sequence identity (e.g., at least 90%, 91%, 92%, 93%, 94%, 95%, 96%, 97%, 98%, or 99% sequence identity) to SEQ ID NO:65. In some embodiments, an anti-PILRA antibody comprises a light chain variable region comprising the amino acid sequence of SEQ ID NO:65.

In some embodiments, an anti-PILRA antibody comprises a heavy chain variable region comprising an amino acid sequence that has at least 85% sequence identity (e.g., at least 90%, 91%, 92%, 93%, 94%, 95%, 96%, 97%, 98%, or 99% sequence identity) to SEQ ID NO:54 and a light chain variable region comprising an amino acid sequence that has at least 85% sequence identity (e.g., at least 90%, 91%, 92%, 93%, 94%, 95%, 96%, 97%, 98%, or 99% sequence identity) to SEQ ID NO:65. In some embodiments, an anti-PILRA antibody comprises a heavy chain variable region comprising the amino acid sequence of SEQ ID NO:54 and a light chain variable region comprising the amino acid sequence of SEQ ID NO:65.

In some embodiments, an anti-PILRA antibody comprises a heavy chain variable region that comprises a heavy chain CDR1-3 comprising the amino acid sequences of SEQ ID NOS:4, 12, and 20, respectively, and that has at least 85% sequence identity (e.g., at least 90%, 91%, 92%, 93%, 94%, 95%, 96%, 97%, 98%, or 99% sequence identity) to SEQ ID NO:54, and a light chain variable region that comprises a light chain CDR1-3 comprising the amino acid sequences of SEQ ID NOS:30, 39, and 47, respectively, and that has at least 85% sequence identity (e.g., at least 90%, 91%, 92%, 93%, 94%, 95%, 96%, 97%, 98%, or 99% sequence identity) to SEQ ID NO:65.

In some embodiments, an anti-PILRA antibody comprises two heavy chains that each comprises a heavy chain CDR1-3 comprising the amino acid sequences of SEQ ID NOS:4, 12, and 20, respectively, and has at least 85% sequence identity (e.g., at least 90%, 91%, 92%, 93%, 94%, 95%, 96%, 97%, 98%, or 99% sequence identity) to SEQ ID NO:122, and two light chains that each comprises a light chain CDR1-3 comprising the amino acid sequences of SEQ ID NOS:30, 39, and 47, respectively, and has at least 85% sequence identity (e.g., at least 90%, 91%, 92%, 93%, 94%, 95%, 96%, 97%, 98%, or 99% sequence identity) to SEQ ID NO:123.

### Clone 4

In some embodiments, an anti-PILRA antibody comprises a CDR-H1 sequence comprising the amino acid sequence of SEQ ID NO:5, a CDR-H2 sequence comprising the amino acid sequence of SEQ ID NO:13, a CDR-H3 sequence comprising the amino acid sequence of SEQ ID NO:22, a CDR-L1 sequence comprising the amino acid sequence of SEQ ID NO:31, a CDR-L2 sequence comprising the amino acid sequence of SEQ ID NO:39, and a CDR-L3 sequence comprising the amino acid sequence of SEQ ID NO:47.

In some embodiments, an anti-PILRA antibody comprises a heavy chain variable region comprising an amino acid sequence that has at least 85% sequence identity (e.g., at least 90%, 91%, 92%, 93%, 94%, 95%, 96%, 97%, 98%, or 99% sequence identity) to SEQ ID NO:56. In some embodiments, an anti-PILRA antibody comprises a heavy chain variable region comprising the amino acid sequence of SEQ ID NO:56.

In some embodiments, an anti-PILRA antibody comprises a light chain variable region comprising an amino acid sequence that has at least 85% sequence identity (e.g., at least 90%, 91%, 92%, 93%, 94%, 95%, 96%, 97%, 98%, or 99% sequence identity) to SEQ ID NO:66. In some embodiments, an anti-PILRA antibody comprises a light chain variable region comprising the amino acid sequence of SEQ ID NO:66.

In some embodiments, an anti-PILRA antibody comprises a heavy chain variable region comprising an amino acid sequence that has at least 85% sequence identity (e.g., at least 90%, 91%, 92%, 93%, 94%, 95%, 96%, 97%, 98%, or 99% sequence identity) to SEQ ID NO:56 and a light chain variable region comprising an amino acid sequence that has at least 85% sequence identity (e.g., at least 90%, 91%, 92%, 93%, 94%, 95%, 96%, 97%, 98%, or 99% sequence identity) to SEQ ID NO:66. In some embodiments, an anti-PILRA antibody comprises a heavy chain variable region comprising the amino acid sequence of SEQ ID NO:56 and a light chain variable region comprising the amino acid sequence of SEQ ID NO:66.

In some embodiments, an anti-PILRA antibody comprises a heavy chain variable region that comprises a heavy chain CDR1-3 comprising the amino acid sequences of SEQ ID NOS:5, 13, and 22, respectively, and that has at least 85% sequence identity (e.g., at least 90%, 91%, 92%, 93%, 94%, 95%, 96%, 97%, 98%, or 99% sequence identity) to SEQ ID NO:56, and a light chain variable region that comprises a light chain CDR1-3 comprising the amino acid sequences of SEQ ID NOS:31, 39, and 47, respectively, and that has at least 85% sequence identity (e.g., at least 90%, 91%, 92%, 93%, 94%, 95%, 96%, 97%, 98%, or 99% sequence identity) to SEQ ID NO:66.

In some embodiments, an anti-PILRA antibody comprises two heavy chains that each comprises a heavy chain CDR1-3 comprising the amino acid sequences of SEQ ID NOS:5, 13, and 22, respectively, and has at least 85% sequence identity (e.g., at least 90%, 91%, 92%, 93%, 94%, 95%, 96%, 97%, 98%, or 99% sequence identity) to SEQ ID NO:124, and two light chains that each comprises a light chain CDR1-3 comprising the amino acid sequences of SEQ ID NOS:31, 39, and 47, respectively, and has at least 85% sequence identity (e.g., at least 90%, 91%, 92%, 93%, 94%, 95%, 96%, 97%, 98%, or 99% sequence identity) to SEQ ID NO:125.

### Clone 5

In some embodiments, an anti-PILRA antibody comprises a CDR-H1 sequence comprising the amino acid sequence of SEQ ID NO:6, a CDR-H2 sequence comprising the amino acid sequence of SEQ ID NO:14, a CDR-H3 sequence comprising the amino acid sequence of SEQ ID NO:23, a CDR-L1 sequence comprising the amino acid sequence of SEQ ID NO:32, a CDR-L2 sequence comprising the amino acid sequence of SEQ ID NO:40, and a CDR-L3 sequence comprising the amino acid sequence of SEQ ID NO:48.

In some embodiments, an anti-PILRA antibody comprises a heavy chain variable region comprising an amino acid sequence that has at least 85% sequence identity (e.g., at least 90%, 91%, 92%, 93%, 94%, 95%, 96%, 97%, 98%, or 99% sequence identity) to SEQ ID NO:57. In some embodiments, an anti-PILRA antibody comprises a heavy chain variable region comprising the amino acid sequence of SEQ ID NO:57.

In some embodiments, an anti-PILRA antibody comprises a light chain variable region comprising an amino acid sequence that has at least 85% sequence identity (e.g., at least 90%, 91%, 92%, 93%, 94%, 95%, 96%, 97%, 98%, or 99% sequence identity) to SEQ ID NO:67. In some embodiments, an anti-PILRA antibody comprises a light chain variable region comprising the amino acid sequence of SEQ ID NO:67.

In some embodiments, an anti-PILRA antibody comprises a heavy chain variable region comprising an amino acid sequence that has at least 85% sequence identity (e.g., at least 90%, 91%, 92%, 93%, 94%, 95%, 96%, 97%, 98%, or 99% sequence identity) to SEQ ID NO:57 and a light chain variable region comprising an amino acid sequence that has at least 85% sequence identity (e.g., at least 90%, 91%, 92%, 93%, 94%, 95%, 96%, 97%, 98%, or 99% sequence identity) to SEQ ID NO:67. In some embodiments, an anti-PILRA antibody comprises a heavy chain variable region comprising the amino acid sequence of SEQ ID NO:57 and a light chain variable region comprising the amino acid sequence of SEQ ID NO:67.

In some embodiments, an anti-PILRA antibody comprises a heavy chain variable region that comprises a heavy chain CDR1-3 comprising the amino acid sequences of SEQ ID NOS:6, 14, and 23, respectively, and that has at least 85% sequence identity (e.g., at least 90%, 91%, 92%, 93%, 94%, 95%, 96%, 97%, 98%, or 99% sequence identity) to SEQ ID NO:57, and a light chain variable region that comprises a light chain CDR1-3 comprising the amino acid sequences of SEQ ID NOS:32, 40, and 48, respectively, and that has at least 85% sequence identity (e.g., at least 90%, 91%, 92%, 93%, 94%, 95%, 96%, 97%, 98%, or 99% sequence identity) to SEQ ID NO:67.

In some embodiments, an anti-PILRA antibody comprises two heavy chains that each comprises a heavy chain CDR1-3 comprising the amino acid sequences of SEQ ID NOS:6, 14, and 23, respectively, and has at least 85% sequence identity (e.g., at least 90%, 91%, 92%, 93%, 94%, 95%, 96%, 97%, 98%, or 99% sequence identity) to SEQ ID NO:126, and two light chains that each comprises a light chain CDR1-3 comprising the amino acid sequences of SEQ ID NOS:32, 40, and 48, respectively, and has at least 85% sequence identity (e.g., at least 90%, 91%, 92%, 93%, 94%, 95%, 96%, 97%, 98%, or 99% sequence identity) to SEQ ID NO:127.

### Clone 12

In some embodiments, an anti-PILRA antibody comprises a CDR-H1 sequence comprising the amino acid sequence of SEQ ID NO:7, a CDR-H2 sequence comprising the amino acid sequence of SEQ ID NO:15, a CDR-H3 sequence comprising the amino acid sequence of SEQ ID NO:24, a CDR-L1 sequence comprising the amino acid sequence of SEQ ID NO:33, a CDR-L2 sequence comprising the amino acid sequence of SEQ ID NO:41, and a CDR-L3 sequence comprising the amino acid sequence of SEQ ID NO:49.

In some embodiments, an anti-PILRA antibody comprises a heavy chain variable region comprising an amino acid sequence that has at least 85% sequence identity (e.g., at least 90%, 91%, 92%, 93%, 94%, 95%, 96%, 97%, 98%, or 99% sequence identity) to SEQ ID NO: 137. In some embodiments, an anti-PILRA antibody comprises a heavy chain variable region comprising the amino acid sequence of SEQ ID NO:137.

In some embodiments, an anti-PILRA antibody comprises a light chain variable region comprising an amino acid sequence that has at least 85% sequence identity (e.g., at least 90%, 91%, 92%, 93%, 94%, 95%, 96%, 97%, 98%, or 99% sequence identity) to SEQ ID NO: 145. In some embodiments, an anti-PILRA antibody comprises a light chain variable region comprising the amino acid sequence of SEQ ID NO:145.

In some embodiments, an anti-PILRA antibody comprises a heavy chain variable region comprising an amino acid sequence that has at least 85% sequence identity (e.g., at least 90%, 91%, 92%, 93%, 94%, 95%, 96%, 97%, 98%, or 99% sequence identity) to SEQ ID NO: 137 and a light chain variable region comprising an amino acid sequence that has at least 85% sequence identity (e.g., at least 90%, 91%, 92%, 93%, 94%, 95%, 96%, 97%, 98%, or 99% sequence identity) to SEQ ID NO:145. In some embodiments, an anti-PILRA antibody comprises a heavy chain variable region comprising the amino acid sequence of SEQ ID NO: 137 and a light chain variable region comprising the amino acid sequence of SEQ ID NO:145.

In some embodiments, an anti-PILRA antibody comprises a heavy chain variable region that comprises a heavy chain CDR1-3 comprising the amino acid sequences of SEQ ID NOS:7, 15, and 24, respectively, and that has at least 85% sequence identity (e.g., at least 90%, 91%, 92%, 93%, 94%, 95%, 96%, 97%, 98%, or 99% sequence identity) to SEQ ID NO:137, and a light chain variable region that comprises a light chain CDR1-3 comprising the amino acid sequences of SEQ ID NOS:33, 41, and 49, respectively, and that has at least 85% sequence identity (e.g., at least 90%, 91%, 92%, 93%, 94%, 95%, 96%, 97%, 98%, or 99% sequence identity) to SEQ ID NO:145.

In some embodiments, an anti-PILRA antibody comprises two heavy chains that each comprises a heavy chain CDR1-3 comprising the amino acid sequences of SEQ ID NOS:7, 15, and 24, respectively, and has at least 85% sequence identity (e.g., at least 90%, 91%, 92%, 93%, 94%, 95%, 96%, 97%, 98%, or 99% sequence identity) to SEQ ID NO:150, and two light chains that each comprises a light chain CDR1-3 comprising the amino acid sequences of SEQ ID NOS:33, 41, and 49, respectively, and has at least 85% sequence identity (e.g., at least 90%, 91%, 92%, 93%, 94%, 95%, 96%, 97%, 98%, or 99% sequence identity) to SEQ ID NO:151.

### Clone 15

In some embodiments, an anti-PILRA antibody comprises a CDR-H1 sequence comprising the amino acid sequence of SEQ ID NO:7, a CDR-H2 sequence comprising the amino acid sequence of SEQ ID NO:15, a CDR-H3 sequence comprising the amino acid sequence of SEQ ID NO:24, a CDR-L1 sequence comprising the amino acid sequence of SEQ ID NO:33, a CDR-L2 sequence comprising the amino acid sequence of SEQ ID NO:41, and a CDR-L3 sequence comprising the amino acid sequence of SEQ ID NO:49.

In some embodiments, an anti-PILRA antibody comprises a heavy chain variable region comprising an amino acid sequence that has at least 85% sequence identity (e.g., at least 90%, 91%, 92%, 93%, 94%, 95%, 96%, 97%, 98%, or 99% sequence identity) to SEQ ID NO: 140. In some embodiments, an anti-PILRA antibody comprises a heavy chain variable region comprising the amino acid sequence of SEQ ID NO:140.

In some embodiments, an anti-PILRA antibody comprises a light chain variable region comprising an amino acid sequence that has at least 85% sequence identity (e.g., at least 90%, 91%, 92%, 93%, 94%, 95%, 96%, 97%, 98%, or 99% sequence identity) to SEQ ID NO: 145. In some embodiments, an anti-PILRA antibody comprises a light chain variable region comprising the amino acid sequence of SEQ ID NO:145.

In some embodiments, an anti-PILRA antibody comprises a heavy chain variable region comprising an amino acid sequence that has at least 85% sequence identity (e.g., at least 90%, 91%, 92%, 93%, 94%, 95%, 96%, 97%, 98%, or 99% sequence identity) to SEQ ID NO: 140 and a light chain variable region comprising an amino acid sequence that has at least 85% sequence identity (e.g., at least 90%, 91%, 92%, 93%, 94%, 95%, 96%, 97%, 98%, or 99% sequence identity) to SEQ ID NO:145. In some embodiments, an anti-PILRA antibody comprises a heavy chain variable region comprising the amino acid sequence of SEQ ID NO: 140 and a light chain variable region comprising the amino acid sequence of SEQ ID NO: 145.

In some embodiments, an anti-PILRA antibody comprises a heavy chain variable region that comprises a heavy chain CDR1-3 comprising the amino acid sequences of SEQ ID NOS:7, 15, and 24, respectively, and that has at least 85% sequence identity (e.g., at least 90%, 91%, 92%, 93%, 94%, 95%, 96%, 97%, 98%, or 99% sequence identity) to SEQ ID NO:140, and a light chain variable region that comprises a light chain CDR1-3 comprising the amino acid sequences of SEQ ID NOS:33, 41, and 49, respectively, and that has at least 85% sequence identity (e.g., at least 90%, 91%, 92%, 93%, 94%, 95%, 96%, 97%, 98%, or 99% sequence identity) to SEQ ID NO:145.

In some embodiments, an anti-PILRA antibody comprises two heavy chains that each comprises a heavy chain CDR1-3 comprising the amino acid sequences of SEQ ID NOS:7, 15, and 24, respectively, and has at least 85% sequence identity (e.g., at least 90%, 91%, 92%, 93%, 94%, 95%, 96%, 97%, 98%, or 99% sequence identity) to SEQ ID NO:152, and two light chains that each comprises a light chain CDR1-3 comprising the amino acid sequences of SEQ ID NOS:33, 41, and 49, respectively, and has at least 85% sequence identity (e.g., at least 90%, 91%, 92%, 93%, 94%, 95%, 96%, 97%, 98%, or 99% sequence identity) to SEQ ID NO:151.

### Clone 23

In some embodiments, an anti-PILRA antibody comprises a CDR-H1 sequence comprising the amino acid sequence of SEQ ID NO:7, a CDR-H2 sequence comprising the amino acid sequence of SEQ ID NO:15, a CDR-H3 sequence comprising the amino acid sequence of SEQ ID NO:25, a CDR-L1 sequence comprising the amino acid sequence of SEQ ID NO:34, a CDR-L2 sequence comprising the amino acid sequence of SEQ ID NO:42, and a CDR-L3 sequence comprising the amino acid sequence of SEQ ID NO:49.

In some embodiments, an anti-PILRA antibody comprises a heavy chain variable region comprising an amino acid sequence that has at least 85% sequence identity (e.g., at least 90%, 91%, 92%, 93%, 94%, 95%, 96%, 97%, 98%, or 99% sequence identity) to SEQ ID NO: 143. In some embodiments, an anti-PILRA antibody comprises a heavy chain variable region comprising the amino acid sequence of SEQ ID NO:143.

In some embodiments, an anti-PILRA antibody comprises a light chain variable region comprising an amino acid sequence that has at least 85% sequence identity (e.g., at least 90%, 91%, 92%, 93%, 94%, 95%, 96%, 97%, 98%, or 99% sequence identity) to SEQ ID NO: 146. In some embodiments, an anti-PILRA antibody comprises a light chain variable region comprising the amino acid sequence of SEQ ID NO:146.

In some embodiments, an anti-PILRA antibody comprises a heavy chain variable region comprising an amino acid sequence that has at least 85% sequence identity (e.g., at least 90%, 91%, 92%, 93%, 94%, 95%, 96%, 97%, 98%, or 99% sequence identity) to SEQ ID NO: 143 and a light chain variable region comprising an amino acid sequence that has at least 85% sequence identity (e.g., at least 90%, 91%, 92%, 93%, 94%, 95%, 96%, 97%, 98%, or 99% sequence identity) to SEQ ID NO:146. In some embodiments, an anti-PILRA antibody comprises a heavy chain variable region comprising the amino acid sequence of SEQ ID NO:143 and a light chain variable region comprising the amino acid sequence of SEQ ID NO:146.

In some embodiments, an anti-PILRA antibody comprises a heavy chain variable region that comprises a heavy chain CDR1-3 comprising the amino acid sequences of SEQ ID NOS:7, 15, and 25, respectively, and that has at least 85% sequence identity (e.g., at least 90%, 91%, 92%, 93%, 94%, 95%, 96%, 97%, 98%, or 99% sequence identity) to SEQ ID NO:143, and a light chain variable region that comprises a light chain CDR1-3 comprising the amino acid sequences of SEQ ID NOS:34, 42, and 49, respectively, and that has at least 85% sequence identity (e.g., at least 90%, 91%, 92%, 93%, 94%, 95%, 96%, 97%, 98%, or 99% sequence identity) to SEQ ID NO:146.

In some embodiments, an anti-PILRA antibody comprises two heavy chains that each comprises a heavy chain CDR1-3 comprising the amino acid sequences of SEQ ID NOS:7, 15, and 25, respectively, and has at least 85% sequence identity (e.g., at least 90%, 91%, 92%, 93%, 94%, 95%, 96%, 97%, 98%, or 99% sequence identity) to SEQ ID NO:153, and two light chains that each comprises a light chain CDR1-3 comprising the amino acid sequences of SEQ ID NOS:34, 42, and 49, respectively, and has at least 85% sequence identity (e.g., at least 90%, 91%, 92%, 93%, 94%, 95%, 96%, 97%, 98%, or 99% sequence identity) to SEQ ID NO:154.

### Clone 35

In some embodiments, an anti-PILRA antibody comprises a CDR-H1 sequence comprising the amino acid sequence of SEQ ID NO:7, a CDR-H2 sequence comprising the amino acid sequence of SEQ ID NO:15, a CDR-H3 sequence comprising the amino acid sequence of SEQ ID NO:25, a CDR-L1 sequence comprising the amino acid sequence of SEQ ID NO:34, a CDR-L2 sequence comprising the amino acid sequence of SEQ ID NO:42, and a CDR-L3 sequence comprising the amino acid sequence of SEQ ID NO:49.

In some embodiments, an anti-PILRA antibody comprises a heavy chain variable region comprising an amino acid sequence that has at least 85% sequence identity (e.g., at least 90%, 91%, 92%, 93%, 94%, 95%, 96%, 97%, 98%, or 99% sequence identity) to SEQ ID NO: 143. In some embodiments, an anti-PILRA antibody comprises a heavy chain variable region comprising the amino acid sequence of SEQ ID NO:143.

In some embodiments, an anti-PILRA antibody comprises a light chain variable region comprising an amino acid sequence that has at least 85% sequence identity (e.g., at least 90%, 91%, 92%, 93%, 94%, 95%, 96%, 97%, 98%, or 99% sequence identity) to SEQ ID NO: 149. In some embodiments, an anti-PILRA antibody comprises a light chain variable region comprising the amino acid sequence of SEQ ID NO:149.

In some embodiments, an anti-PILRA antibody comprises a heavy chain variable region comprising an amino acid sequence that has at least 85% sequence identity (e.g., at least 90%, 91%, 92%, 93%, 94%, 95%, 96%, 97%, 98%, or 99% sequence identity) to SEQ ID NO: 143 and a light chain variable region comprising an amino acid sequence that has at least 85% sequence identity (e.g., at least 90%, 91%, 92%, 93%, 94%, 95%, 96%, 97%, 98%, or 99% sequence identity) to SEQ ID NO:149. In some embodiments, an anti-PILRA antibody comprises a heavy chain variable region comprising the amino acid sequence of SEQ ID NO: 143 and a light chain variable region comprising the amino acid sequence of SEQ ID NO:149.

In some embodiments, an anti-PILRA antibody comprises a heavy chain variable region that comprises a heavy chain CDR1-3 comprising the amino acid sequences of SEQ ID NOS:7, 15, and 25, respectively, and that has at least 85% sequence identity (e.g., at least 90%, 91%, 92%, 93%, 94%, 95%, 96%, 97%, 98%, or 99% sequence identity) to SEQ ID NO:143, and a light chain variable region that comprises a light chain CDR1-3 comprising the amino acid sequences of SEQ ID NOS:34, 42, and 49, respectively, and that has at least 85% sequence identity (e.g., at least 90%, 91%, 92%, 93%, 94%, 95%, 96%, 97%, 98%, or 99% sequence identity) to SEQ ID NO:149.

In some embodiments, an anti-PILRA antibody comprises two heavy chains that each comprises a heavy chain CDR1-3 comprising the amino acid sequences of SEQ ID NOS:7, 15, and 25, respectively, and has at least 85% sequence identity (e.g., at least 90%, 91%, 92%, 93%, 94%, 95%, 96%, 97%, 98%, or 99% sequence identity) to SEQ ID NO:153, and two light chains that each comprises a light chain CDR1-3 comprising the amino acid sequences of SEQ ID NOS:34, 42, and 49, respectively, and has at least 85% sequence identity (e.g., at least 90%, 91%, 92%, 93%, 94%, 95%, 96%, 97%, 98%, or 99% sequence identity) to SEQ ID NO:155.

### Consensus Sequences

In some embodiments, an anti-PILRA antibody comprises one or more sequences that are encompassed by a consensus sequence disclosed herein. As a non-limiting example, consensus sequences can be identified by aligning heavy chain or light chain sequences (e.g., CDRs) for antibodies that are from the same (or similar) germlines. In some embodiments, consensus sequences may be generated from antibodies that contain sequences that are of the same (or similar) length and/or have at least one highly similar CDR (e.g., a highly similar CDR3). In some embodiments, such sequences in these antibodies may be aligned and compared to identify conserved amino acids or motifs (i.e., where alteration in sequences may alter protein function) and/or regions where variation occurs the sequences (i.e., where variation of sequence is not likely to significantly affect protein function). Alternatively, consensus sequences can be identified by aligning heavy chain or light chain sequences (e.g., CDRs) for antibodies that bind to the same or similar (e.g., overlapping) epitopes to determine conserved amino acids or motifs (i.e., where alteration in sequences may alter protein function) and regions where variation occurs in alignment of sequences (i.e., where variation of sequence is not likely to significantly affect protein function). In some embodiments, one or more consensus sequences can be identified for antibodies that recognize the same or similar epitope as an anti-PILRA antibody as disclosed herein. It will be appreciated that, when selecting an amino acid to insert at a position marked by an “X” in a consensus sequence, in some embodiments the amino acid is selected from those amino acids found at the corresponding position in the aligned sequences.

### Clones 2, 4, and 5

In some embodiments, an anti-PILRA antibody comprises:
(a) a CDR-H1 sequence comprising the sequence of GX₁TFX₂X₃X₄X₅X₆H (SEQ ID NO:74), wherein X₁ is F or Y; X₂ is D or I; X₃ is D or G; X₄ is Y or F; X₅ is A or Y; and X₆ is M or I;
(b) a CDR-H2 sequence comprising the sequence of X₁X₂X₃X₄X₅SGX₆X₇X₈ (SEQ ID NO:75), wherein X₁ is G or W; X₂ is F, M, or I; X₃ is S or N; X₄ is W or P; X₅ is N or E; X₆ is S or D; X₇ is I or T; and X₈ is G or T;
(c) a CDR-H3 sequence comprising the sequence of X₁X₂X₃X₄X₅X₆X₇X₈X₉FDX₁₀ (SEQ ID NO:76), wherein X₁ is D or absent; X₂ is K or G; X₃ is S or N; X₄ is I or W; X₅ is S, G, or N; X₆ is A or F; X₇ is A or P; X₈ is G or D; X₉ is R or T; and X₁₀ is Y, S, or F;
(d) a CDR-L1 sequence comprising the sequence of X₁X₂SX₃X₄IX₅X₆YLN (SEQ ID NO:77), wherein X₁ is Q or R; X₂ is A or S; X₃ is R or Q; X₄ is R, G, or S; X₅ is N or S; and X₆ is N or I;
(e) a CDR-L2 sequence comprising the sequence of X₁ASX₂LX₃X₄ (SEQ ID NO:78), wherein X₁ is D or V; X₂ is N or S; X₃ is E or Q; and X₄ is T or S; and
(f) a CDR-L3 sequence comprising the sequence of QQX₁X₂X₃X₄PX₅T (SEQ ID NO:79), wherein X₁ is Y or S; X₂ is D or Y; X₃ is N or S; X₄ is L or A; and X₅ is L or F.

In particular embodiments, an anti-PILRA antibody comprises:
(a) a CDR-H1 sequence comprising the sequence of GFTFDDYAX₁H (SEQ ID NO:80), wherein X₁ is M or I, or GYTFIGFYIH (SEQ ID NO:6);
(b) a CDR-H2 sequence comprising the sequence of GX₁SWNSGSIG (SEQ ID NO:81), wherein X₁ is F or M, or WINPESGDTT (SEQ ID NO:14);
(c) a CDR-H3 sequence comprising the sequence of DKSIX₁AAGRFDX₂ (SEQ ID NO:82), wherein X₁ is S or G; and X₂ is Y or S, or GNWNFPDTFDF (SEQ ID NO:23);
(d) a CDR-L1 sequence comprising the sequence of QASX₁X₂INNYLN (SEQ ID NO:83), wherein X₁ is R or Q; and X₂ is R or G, or RSSQSISIYLN (SEQ ID NO:32);
(e) a CDR-L2 sequence comprising the sequence of DASNLET (SEQ ID NO:39) or VASSLQS (SEQ ID NO:40); and
(f) a CDR-L3 sequence comprising the sequence of QQYDNLPLT (SEQ ID NO:47) or QQSYSAPFT (SEQ ID NO:48).

In particular embodiments, an anti-PILRA antibody comprises:
(a) a CDR-H1 sequence comprising the sequence of GFTFDDYAX₁H (SEQ ID NO:80), wherein X₁ is M or I;
(b) a CDR-H2 sequence comprising the sequence of GX₁SWNSGSIG (SEQ ID NO:81), wherein X₁ is F or M;
(c) a CDR-H3 sequence comprising the sequence of DKSIX₁AAGRFDX₂ (SEQ ID NO:82), wherein X₁ is S or G; and X₂ is Y or S;
(d) a CDR-L1 sequence comprising the sequence of QASX₁X₂INNYLN (SEQ ID NO:83), wherein X₁ is R or Q; and X₂ is R or G;
(e) a CDR-L2 sequence comprising the sequence of DASNLET (SEQ ID NO: 39); and
(f) a CDR-L3 sequence comprising the sequence of QQYDNLPLT (SEQ ID NO:47).

### Clones 6, 7, and 8

In some embodiments, an anti-PILRA antibody comprises:
(a) a CDR-H1 sequence comprising the sequence of GYTFTX₁X₂YMY (SEQ ID NO:84), wherein X₁ is E or G; and X₂ is Y or H;
(b) a CDR-H2 sequence comprising the sequence of X₁IX₂PX₃X₄GX₅TD (SEQ ID NO:85), wherein X₁ is R or W; X₂ is D or N; X₃ is E or N; X₄ is D or S; and X₅ is G or D;
(c) a CDR-H3 sequence comprising the sequence of TIRGTVFX₁X₂ (SEQ ID NO:86), wherein X₁ is A or V; and X₂ is F or Y, or EGLDGDPFDY (SEQ ID NO:26)
(d) a CDR-L1 sequence comprising the sequence of RX₁SEDIX₂NGLA (SEQ ID NO:87), wherein X₁ is A or P; and X₂ is F or Y, or RSSQSLVHSDGNTYLS (SEQ ID NO:35);
(e) a CDR-L2 sequence comprising the sequence of NX₁X₂X₃X₄X₅X₆ (SEQ ID NO:88), wherein X₁ is A or I; X₂ is K, N, or S; X₃ is T, S, or N; X₄ is L or R; X₅ is H or F; and X₆ is T or S; and
(f) a CDR-L3 sequence comprising the sequence of QQYYDYPLT (SEQ ID NO:49) or IQTTQFST (SEQ ID NO:50).

In particular embodiments, an anti-PILRA antibody comprises:
(a) a CDR-H1 sequence comprising the sequence of GYTFTEYYMY (SEQ ID NO:7) or GYTFTGHYMH (SEQ ID NO:8);
(b) a CDR-H2 sequence comprising the sequence of RIDPEDGGTD (SEQ ID NO:15) or WINPNSGDTD (SEQ ID NO: 16);
(c) a CDR-H3 sequence comprising the sequence of TIRGTVFX₁X₂ (SEQ ID NO:86), wherein X₁ is A or V; and X₂ is F or Y, or EGLDGDPFDY (SEQ ID NO:26);
(d) a CDR-L1 sequence comprising the sequence of RX₁SEDIX₂NGLA (SEQ ID NO:87), wherein X₁ is A or P; and X₂ is F or Y, or RSSQSLVHSDGNTYLS (SEQ ID NO:35);
(e) a CDR-L2 sequence comprising the sequence of NAX₁X₂LHT (SEQ ID NO:89), wherein X₁ is K or N; and X₂ is T or S, or NISNRFS (SEQ ID NO:43); and
(f) a CDR-L3 sequence comprising the sequence of QQYYDYPLT (SEQ ID NO:49) or IQTTQFST (SEQ ID NO:50).

In particular embodiments, an anti-PILRA antibody comprises:
(a) a CDR-H1 sequence comprising the sequence of GYTFTEYYMY (SEQ ID NO:7);
(b) a CDR-H2 sequence comprising the sequence of RIDPEDGGTD (SEQ ID NO:15);
(c) a CDR-H3 sequence comprising the sequence of TIRGTVFX₁X₂ (SEQ ID NO:86), wherein X₁ is A or V; and X₂ is F or Y;
(d) a CDR-L1 sequence comprising the sequence of RX₁SEDIX₂NGLA (SEQ ID NO:87), wherein X₁ is A or P; and X₂ is F or Y;
(e) a CDR-L2 sequence comprising the sequence of NAX₁X₂LHT (SEQ ID NO:89), wherein X₁ is K or N; and X₂ is T or S; and
(f) a CDR-L3 sequence comprising the sequence of QQYYDYPLT (SEQ ID NO:49).

### Binding Characteristics of Anti-PILRA Antibodies

In some embodiments, an antibody as described herein that specifically binds to a PILRA protein (e.g., a hPILRA protein) binds to PILRA that is expressed on a cell (e.g., a cell line that endogenously expresses PILRA, such as immune cells, or a cell line that has been engineered to express PILRA, e.g., as described in the Examples section below). In some embodiments, an antibody that specifically binds to a PILRA protein as described herein binds to purified or recombinant PILRA protein of a portion thereof, or to a chimeric protein comprising PILRA or a portion thereof.

In some embodiments, some embodiments, an antibody that specifically binds to human PILRA protein exhibits cross-reactivity with one or more other PILRA proteins of another species. In some embodiments, an antibody that specifically binds to human PILRA protein exhibits cross-reactivity with a cynomolgus monkey (“cyno”) PILRA protein (cynoPILRA).

Methods for analyzing binding affinity, binding kinetics, and cross-reactivity are known in the art. These methods include, but are not limited to, solid-phase binding assays (e.g., ELISA assay), immunoprecipitation, surface plasmon resonance (e.g., Biacore™ (GE Healthcare, Piscataway, NJ)), kinetic exclusion assays (e.g., KinExA®), flow cytometry, fluorescence-activated cell sorting (FACS), BioLayer interferometry (e.g., Octet™ (FortéBio, Inc., Menlo Park, CA)), and western blot analysis. In some embodiments, ELISA is used to determine binding affinity and/or cross-reactivity. Methods for performing ELISA assays are known in the art, and are also described in the Examples section below. In some embodiments, surface plasmon resonance (SPR) is used to determine binding affinity, binding kinetics, and/or cross-reactivity. In some embodiments, kinetic exclusion assays are used to determine binding affinity, binding kinetics, and/or cross-reactivity. In some embodiments, BioLayer interferometry assays are used to determine binding affinity, binding kinetics, and/or cross-reactivity.

In some embodiments, an anti-PILRA antibody described herein specifically binds to a cynomolgus monkey paired immunoglobulin-like type 2 receptor alpha (cynoPILRA), wherein the binding affinity for the cynoPILRA is at least 2-fold (e.g., at least 2-fold, 5-fold, 10-fold, 20-fold, 30-fold, 40-fold, 50-fold, 60-fold, 70-fold, 80-fold, 90-fold, or 100-fold) stronger than the binding affinity for a human paired immunoglobulin-like type 2 receptor beta (hPILRB).

As described herein, in some embodiments, an anti-PILRA antibody described herein exhibits cross-reactivity with both hPILRA and cynoPILRA. In some embodiments, an anti-PILRA antibody described herein binds to both hPILRA and cynoPILRA.

In certain embodiments, the binding affinity of the anti-PILRA antibody for the cynoPILRA is within 100-fold (e.g., within 100-fold, 90-fold, 80-fold, 70-fold, 60-fold, 50-fold, 40-fold, 30-fold, 20-fold, 10-fold, 9-fold, 8-fold, 7-fold, 6-fold, 5-fold, 4-fold, 3-fold, 2-fold, or 1.5 fold) relative to the binding affinity for the hPILRA. In particular embodiments, the anti-PILRA antibody binds to the hPILRA with a binding affinity of between 0.1 nM and 500 nM (e.g., between 0.1 nM and 400 nM, between 0.1 nM and 300 nM, between 0.1 nM and 200 nM, or between 0.1 nM and 100 nM). In particular embodiments, the anti-PILRA antibody binds to the hPILRA with a binding affinity of between 0.1 nM and 100 nM (e.g., between 0.1 nM and 90 nM, between 0.1 nM and 80 nM, between 0.1 nM and 70 nM, between 0.1 nM and 60 nM, between 0.1 nM and 50 nM, between 0.1 nM and 40 nM, between 0.1 nM and 30 nM, between 0.1 nM and 20 nM, between 0.1 nM and 10 nM, between 0.1 nM and 5 nM, between 0.1 nM and 1 nM, between 1 nM and 100 nM, between 5 nM and 100 nM, between 10 nM and 100 nM, between 20 nM and 100 nM, between 30 nM and 100 nM, between 40 nM and 100 nM, between 50 nM and 100 nM, between 60 nM and 100 nM, between 70 nM and 100 nM, between 80 nM and 100 nM, or between 90 nM and 100 nM).

In some embodiments, an anti-PILRA antibody described herein selectively binds to hPILRA and/or cynoPILRA over hPILRB. In particular embodiments, the binding affinity of the antibody for the hPILRA is at least 10-fold (e.g., at least 10-fold, 20-fold, 40-fold, 60-fold, 80-fold, 100-fold, 120-fold, 140-fold, 160-fold, 180-fold, 200-fold, 220-fold, 240-fold, 260-fold, 280-fold, or 300-fold) stronger than the binding affinity for the hPILRB.

### Epitopes Recognized by Anti-PILRA Antibodies

In some embodiments, an anti-PILRA antibody recognizes an epitope of human PILRA that is the same or substantially the same as the epitope recognized by an antibody clone as described herein. As used herein, the term “substantially the same,” as used with reference to an epitope recognized by an antibody clone as described herein, means that the anti-PILRA antibody recognizes an epitope that is identical, within, or nearly identical to (e.g., has at least 90% sequence identity (e.g., at least 91%, 92%, 93%, 94%, 95%, 96%, 97%, 98%, or 99% sequence identity) to, or has one, two, or three amino acid substitutions, e.g., conservative substitutions, relative to), or has substantial overlap with (e.g., at least 50%, 60%, 70%, 80%, 90%, or 95% overlap with) the epitope recognized by the antibody clone as described herein.

In some embodiments, an anti-PILRA antibody recognizes an epitope of human PILRA that is the same or substantially the same as the epitope recognized by an antibody clone selected from the group consisting of Clone 2 and Clones 4-8 (e.g., Clones 2, 4, and 5) and variants of the same.

In some embodiments, an anti-PILRA antibody recognizes an epitope of human PILRA within the extracellular domain (ECD) of PILRA, e.g., the ECD comprising amino acids 20 to 143 of SEQ ID NO:1. In some embodiments, an anti-PILRA antibody binds to human PILRA at an epitope within the stalk region of PILRA. In some embodiments, an anti-PILRA antibody is an antagonist that inhibits PILRA signaling.

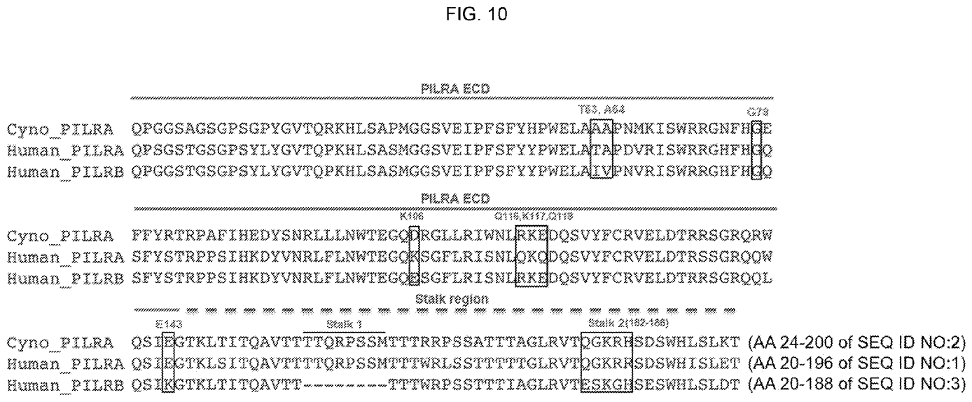

In some embodiments, an anti-PILRA antibody binds to one or more amino acids at one or more of the following positions: 63, 64, 78, 106, 143, 116-118, and 182-186, wherein the positions are determined with reference to SEQ ID NO:1. In particular embodiments, an anti-PILRA antibody described herein binds to one or more amino acids at one or more of the following positions in SEQ ID NO:1: 63, 64, 78, 106, 143, 116-118, and 182-186. FIG. 10 shows an alignment of the ECD and stalk region sequences of cynoPILRA, hPILRA, and hPILRB. In certain embodiments, the anti-PILRA antibody binds to one or more amino acids at one or more of the following positions: 78, 106, and 143 of hPILRA. In particular embodiments, the anti-PILRA antibody binds to G78, K106, and/or E143 of SEQ ID NO:1. In some embodiments, the anti-PILRA antibody binds to G78 of SEQ ID NO:1. In some embodiments, the anti-PILRA antibody binds to R78 of SEQ ID NO:136. In some embodiments, the anti-PILRA antibody binds to K106 of SEQ ID NO:1. In some embodiments, the anti-PILRA antibody binds to E143 of SEQ ID NO:1. In particular embodiments, the anti-PILRA antibody binds to G78, K106, and E143 of SEQ ID NO:1. In particular embodiments, the anti-PILRA antibody binds to R78, K106, and E143 of SEQ ID NO:136.

In certain embodiments, the anti-PILRA antibody binds to one or more amino acids at one or more of the following positions: 63 and 64 of hPILRA. In particular embodiments, the anti-PILRA antibody binds to T63 and/or A64 of SEQ ID NO:1. In some embodiments, the anti-PILRA antibody binds to T63 of SEQ ID NO:1. In some embodiments, the anti-PILRA antibody binds to A64 of SEQ ID NO: 1. In particular embodiments, the anti-PILRA antibody binds to T63 and A64 of SEQ ID NO: 1.

In certain embodiments, the anti-PILRA antibody binds to one or more amino acids at one or more of the following positions: 106 and 116-118 of hPILRA. In some embodiments, the anti-PILRA antibody binds to Q116, K117, and/or Q118 of SEQ ID NO:1 (e.g., Q116, K117, and Q118).

In some embodiments, an anti-PILRA antibody recognizes an epitope within stalk 2 region of hPILRA, e.g., QGKRR (SEQ ID NO:90) from positions 182-186 of SEQ ID NO:1. In some embodiments, an anti-PILRA antibody recognizes an epitope comprising 1, 2, 3, or 4 amino acids within residues 182-186 of SEQ ID NO:1. In some embodiments, an anti-PILRA antibody recognizes an epitope comprising 2, 3, or 4 contiguous amino acids within residues 182-186 of SEQ ID NO:1. In some embodiments, an anti-PILRA antibody recognizes an epitope comprising all five amino acids within residues 182-186 of SEQ ID NO:1. In some embodiments, the anti-PILRA antibody binds to Q182, G183, K184, R185, and/or R186 of SEQ ID NO:1 (e.g., Q182, G183, K184, R185, and R186).

In some embodiments, an anti-PILRA antibody recognizes an epitope within stalk 1 region of hPILRA, e.g., TTQRPSSM (SEQ ID NO:91) from positions 156-163 of SEQ ID NO:1. In some embodiments, an anti-PILRA antibody recognizes an epitope comprising 1, 2, 3, 4, 5, 6, or 7 amino acids within residues 156-163 of SEQ ID NO:1. In some embodiments, an anti-PILRA antibody recognizes an epitope comprising 2, 3, 4, 5, 6, or 7 contiguous amino acids within residues 156-163 of SEQ ID NO:1. In some embodiments, an anti-PILRA antibody recognizes an epitope comprising all eight amino acids within residues 156-163 of SEQ ID NO:1. In some embodiments, the anti-PILRA antibody binds to T156, T157, Q158, R159, P160, S161, S162, and/or M163 of SEQ ID NO:1 (e.g., T156, T157, Q158, R159, P160, S161, S162, and M163).

### Cross-Reactivity

In certain embodiments, an anti-PILRA antibody recognizes one or more epitopes that are conserved between hPILRA and cynoPILRA. In some embodiments, an anti-PILRA antibody binds to one or more amino acids at one or more of the following positions in hPILRA and/or in cynoPILRA: 64, 78, 139, 143, 156-163, and 182-185, wherein the positions are determined with reference to SEQ ID NO:1. In particular embodiments, an anti-PILRA antibody binds to one or more amino acids at one or more of the following positions in hPILRA and in cynoPILRA: 64, 78, 139, 143, 156-163, and 182-185, wherein the positions are determined with reference to SEQ ID NO:1. In particular embodiments, the anti-PILRA antibody binds to A64, G78, W139, E143, T156, T157, Q158, R159, P160, S161, S162, M163, Q182, G183, K184, and/or R185 of hPILRA having the sequence of SEQ ID NO:1 and A68, G82, W143, E147, T160, T161, Q162, R163, P164, S165, S166, M167, Q186, G187, K188, and/or R189 of cynoPILRA having the sequence of SEQ ID NO:2.

In particular embodiments, the anti-PILRA antibody binds to A64 of hPILRA having the sequence of SEQ ID NO:1 and A68 of cynoPILRA having the sequence of SEQ ID NO:2. In particular embodiments, the anti-PILRA antibody binds to G78 of hPILRA having the sequence of SEQ ID NO:1 and G82 of cynoPILRA having the sequence of SEQ ID NO:2. In particular embodiments, the anti-PILRA antibody binds to R78 of hPILRA having the sequence of SEQ ID NO:136 and G82 of cynoPILRA having the sequence of SEQ ID NO:2. In particular embodiments, the anti-PILRA antibody binds to W139 of hPILRA having the sequence of SEQ ID NO:1 and W143 of cynoPILRA having the sequence of SEQ ID NO:2. In particular embodiments, the anti-PILRA antibody binds to E143 of hPILRA having the sequence of SEQ ID NO:1 and E147 of cynoPILRA having the sequence of SEQ ID NO:2. In particular embodiments, the anti-PILRA antibody binds to the same one or more amino acids within TTQRPSSM (SEQ ID NO:91) of both hPILRA (e.g., positions 156-163 of SEQ ID NO:1) and cynoPILRA (e.g., positions 160 to 167 of SEQ ID NO:2). In particular embodiments, the anti-PILRA antibody binds to the same one or more amino acids within QGKR (SEQ ID NO:92) of both hPILRA (e.g., positions 182-185 of SEQ ID NO: 1) and cynoPILRA (e.g., positions 186 to 189 of SEQ ID NO:2).

In particular embodiments, the anti-PILRA antibody binds to G78, K106, E143 of hPILRA having the sequence of SEQ ID NO:1 and G82, D110, E147 of cynoPILRA having the sequence of SEQ ID NO:2. In particular embodiments, the anti-PILRA antibody binds to R78, K106, E143 of hPILRA having the sequence of SEQ ID NO:136 and G82, D110, E147 of cynoPILRA having the sequence of SEQ ID NO:2. In particular embodiments, the anti-PILRA antibody binds to T63 and A64 of hPILRA having the sequence of SEQ ID NO:1 and A67 and A68 of cynoPILRA having the sequence of SEQ ID NO:2. In particular embodiments, the anti-PILRA antibody binds to one or more positions within QGKRR (SEQ ID NO:90) of hPILRA (e.g., positions 182-186 of SEQ ID NO:1) and the same corresponding positions with QGKRH (SEQ ID NO:93) of cynoPILRA (e.g., positions 186 to 190 of SEQ ID NO:2).

### Functional Characteristics of Anti-PILRA Antibodies

In some embodiments, an anti-PILRA antibody (e.g., an antibody having one or more CDR, heavy chain variable region, and/or light chain variable region sequences as disclosed) functions in one or more activities as disclosed herein. For example, in some embodiments, an anti-PILRA antibody antagonize or reduce PILRA activity, i.e., PILRA activity induced by a ligand.

In certain embodiments, an anti-PILRA antibody blocks the binding of a ligand to hPILRA. In particular embodiments, an anti-PILRA antibody blocks the binding of a sialyated protein to hPILRA, e.g., a sialyated form of any of the following proteins: neural proliferation differentiation and control protein 1 (NPDC1), PILRA-associated neural protein (PANP; PIANP), herpes simplex virus type 1 glycoprotein B (HSV-1 gB), collectin-12 (COLEC12), complement component 4A (C4a), complement component 4B (C4b), dystroglycan 1 (dystrophin-associated glycoprotein 1; DAG1), and c-type lectin domain family member G (Clec4g).

Further, in some embodiments, an anti-PILRA antibody alters phosphorylation of one or more downstream proteins, e.g., increases phosphorylation of EGFR or STAT3, or decreases phosphorylation of STAT1. In some embodiments, an anti-PILRA antibody induces or increases phosphorylation of one or more downstream proteins (e.g., EGFR or STAT3) if the level of downstream protein phosphorylation in a sample treated with the anti-PILRA antibody is increased by at least 10%, at least 20%, at least 30%, at least 40%, at least 50%, at least 60%, at least 70%, at least 80%, at least 90% or more as compared to a control value. In some embodiments, an anti-PILRA antibody induces phosphorylation of one or more downstream proteins (e.g., EGFR or STAT3) if the level of downstream protein phosphorylation in a sample treated with the anti-PILRA antibody is increased by at least 2-fold, 3-fold, 4-fold, 5-fold, 6-fold, 7-fold, 8-fold, 9-fold, 10-fold, or more as compared to a control value. In some embodiments, an anti-PILRA antibody decreases phosphorylation of one or more downstream proteins (e.g., STAT1) if the level of downstream protein phosphorylation in a sample treated with the anti-PILRA antibody is decreased by at least 10%, at least 20%, at least 30%, at least 40%, at least 50%, at least 60%, at least 70%, at least 80%, at least 90% or more as compared to a control value. In some embodiments, an anti-PILRA antibody decreases phosphorylation of one or more downstream proteins (e.g., STAT1) if the level of downstream protein phosphorylation in a sample treated with the anti-PILRA antibody is decreased by at least 2-fold, 3-fold, 4-fold, 5-fold, 6-fold, 7-fold, 8-fold, 9-fold, 10-fold, or more as compared to a control value.

In some embodiments, the control value is the level of downstream protein phosphorylation in an untreated sample (e.g., a sample comprising a PILRA-expressing cell that has not been treated with an anti-PILRA antibody, or a sample from a subject that has not been treated with an anti-PILRA antibody), or a sample that has been treated with a PILRA ligand but not an anti-PILRA antibody, or a sample treated with an appropriate non-PILRA-binding antibody.

For detecting and/or quantifying phosphorylation in a sample, in some embodiments, an immunoassay is used. In some embodiments, the immunoassay is an enzyme immunoassay (EIA), enzyme multiplied immunoassay (EMIA), enzyme-linked immunosorbent assay (ELISA), microparticle enzyme immunoassay (MEIA), immunohistochemistry (IHC), immunocytochemistry, capillary electrophoresis immunoassay (CEIA), radioimmunoassay (RIA), immunofluorescence, chemiluminescence immunoassay (CL), or electrochemiluminescence immunoassay (ECL). In some embodiments, phosphorylation is detected and/or quantified using an immunoassay that utilizes an amplified luminescent proximity homogenous assay (AlphaLISA®, PerkinElmer Inc.).

In some embodiments, phosphorylation is measured using a sample that comprises one or more cells, e.g., one or more PILRA-expressing cells (e.g., a cell line that endogenously expresses PILRA, such as human IPSC-derived microglia, or a cell line that has been engineered to express PILRA, e.g., as described in the Examples section below). In some embodiments, the sample comprises a fluid, e.g., blood, plasma, serum, urine, or cerebrospinal fluid. In some embodiments, the sample comprises tissue (e.g., lung, brain, kidney, spleen, nervous tissue, or skeletal muscle) or cells from such tissue. In some embodiments, the sample comprises endogenous fluid, tissue, or cells (e.g., from a human or non-human subject).

Further, in some embodiments, an anti-PILRA antibody increases anti-inflammatory gene or protein expression. For example, an anti-PILRA antibody enhances IL1RN gene expression. In some embodiments, an anti-PILRA antibody enhances anti-inflammatory gene or protein expression if the level of anti-inflammatory gene or protein expression in a sample treated with the anti-PILRA antibody is increased by at least 10%, at least 20%, at least 30%, at least 40%, at least 50%, at least 60%, at least 70%, at least 80%, at least 90% or more as compared to a control value. In other embodiments, an anti-PILRA antibody reduces pro-inflammatory cytokine protein expression or secretion. For example, an anti-PILRA antibody reduces TNF, IL-6, and/or IP-10 expression. In some embodiments, an anti-PILRA antibody reduces cytokine protein expression if the level of cytokine protein expression in a sample treated with the anti-PILRA antibody is reduced by at least 10%, at least 20%, at least 30%, at least 40%, at least 50%, at least 60%, at least 70%, at least 80%, at least 90% or more as compared to a control value.

Further, in some embodiments, an anti-PILRA antibody enhances cell migration and/or cell function (e.g., for microglia, including IPSC-derived microglia and disease-associated microglia). Disease-associated microglia and methods of detecting disease-associated microglia are described in Keren-Shaul et al., Cell, 2017, 169:1276-1290. In some embodiments, an anti-PILRA antibody enhances cell migration of one or more cell types (e.g., microglia, monocytes, or neutrophils). In some embodiments, an anti-PILRA antibody enhances cell function (e.g., ATP production, fatty acid metabolism, and/or cellular respiration) of one or more cell types (e.g., microglia, monocytes, or neutrophils). In some embodiments, an anti-PILRA antibody enhances the cell migration and/or cell function of microglia. In some embodiments, an anti-PILRA antibody enhances the cell migration and/or cell function of disease-associated microglia.

In some embodiments, an anti-PILRA antibody enhances cell migration and/or cell function if the level of activity in a sample treated with the anti-PILRA antibody is increased by at least 10%, at least 20%, at least 30%, at least 40%, at least 50%, at least 60%, at least 70%, at least 80%, at least 90% or more as compared to a control value. In some embodiments, an anti-PILRA antibody enhances cell migration and/or cell function if the level of activity in a sample treated with the anti-PILRA antibody is increased by at least 2-fold, 3-fold, 4-fold, 5-fold, 6-fold, 7-fold, 8-fold, 9-fold, 10-fold, or more as compared to a control value. In some embodiments, the control value is the level of activity (e.g., migration or function) in an untreated sample (e.g., a sample that has not been treated with an anti-PILRA antibody), a sample that has been treated with a PILRA ligand but not an anti-PILRA antibody, or a sample treated with an appropriate non-PILRA-binding antibody.

In some embodiments, cell migration is measured using a chemotaxis assay. Chemotaxis assays are known in the art. In some embodiments, the cell migration assay (e.g., chemotaxis assay) is performed on a sample comprising cells that endogenously express PILRA, such as human IPSC-derived microglia. In some embodiments, the cell migration assay (e.g., chemotaxis assay) is performed on a sample comprising cells that have been engineered to express PILRA. In some embodiments, the cell migration assay is performed on a sample comprising cells in which PILRA has been deleted or rendered functionally inactive. In some embodiments, cell migration is measured using a chemotaxis assay as described in the Examples section below.

In some embodiments, cell function is measured using a functional assay that is appropriate for that cell. In some embodiments, an anti-PILRA antibody increases fatty acid metabolism (e.g., fatty acid oxidation). In some embodiments, an anti-PILRA antibody enhances cellular ATP production. In some embodiments, an anti-PILRA antibody enhances cellular respiration (e.g., mitochondrial or non-mitochondrial respiration). Changes in cellular ATP production and/or respiration can be evaluated using one or more assays, e.g., as described in the Examples section below.

### IV. Fc Polypeptides and Modifications Thereof

In some aspects, an anti-PILRA antibody comprises two Fc polypeptides, one or both of which may each comprise independently selected modifications (e.g., mutations) or may be a wild-type Fc polypeptide, e.g., a human IgG1 Fc polypeptide. In some embodiments, one or both Fc polypeptides in an anti-PILRA antibody described herein can comprise a sequence having at least 90% (e.g., 90%, 92%, 94%, 96%, 97%, 98%, 99%, or 100%) identity to the sequence of a wild-type Fc polypeptide (e.g., SEQ ID NO:94). In some embodiments, one Fc polypeptide in an anti-PILRA antibody described herein can be a wild-type Fc polypeptide (e.g., SEQ ID NO:94), while the other Fc polypeptide can have at least one amino acid modification relative to a wild-type Fc polypeptide (e.g., SEQ ID NO:94). In some embodiments, both Fc polypeptides in an anti-PILRA antibody described herein can be a wild-type Fc polypeptide (e.g., SEQ ID NO:94). In some embodiments, both Fc polypeptides in an anti-PILRA antibody described herein can have at least one amino acid modification relative to a wild-type Fc polypeptide (e.g., SEQ ID NO:94). Non-limiting examples of mutations that can be introduced into one or both Fc polypeptides include, e.g., mutations to increase serum stability, to modulate effector function, to influence glycosylation, and/or to reduce immunogenicity in humans.

### Fc Polypeptide Modifications for Modulating Effector Function

In some embodiments, one or both Fc polypeptides present in an antibody described herein may comprise modifications that reduce effector function, i.e., having a reduced ability to induce certain biological functions upon binding to an Fc receptor expressed on an effector cell that mediates the effector function. Examples of antibody effector functions include, but are not limited to, C1q binding and complement dependent cytotoxicity (CDC), Fc receptor binding, antibody-dependent cell-mediated cytotoxicity (ADCC), antibody-dependent cell-mediated phagocytosis (ADCP), down-regulation of cell surface receptors (e.g., B cell receptor), and B-cell activation. Effector functions may vary with the antibody class. For example, native human IgG1 and IgG3 antibodies can elicit ADCC and CDC activities upon binding to an appropriate Fc receptor present on an immune system cell; and native human IgG1, IgG2, IgG3, and IgG4 can elicit ADCP functions upon binding to the appropriate Fc receptor present on an immune cell.

In some embodiments, one or both Fc polypeptides in an Fc polypeptide dimer can comprise modifications that reduce or eliminate effector function. Illustrative Fc polypeptide mutations that reduce effector function include, but are not limited to, substitutions in a CH2 domain, e.g., at positions 234 and 235 and/or at position 329, according to the EU numbering scheme. For example, in some embodiments, one or both Fc polypeptides comprise Ala residues at positions 234 and 235 (also referred to as “LALA” herein). In some embodiments, one or both Fc polypeptides comprise Gly residue at position 329 (also referred to as “P329G” or “PG” herein) or Ser residue at position 329 (also referred to as “P329S” or “PS” herein). In some embodiments, one or both Fc polypeptides comprise Ala residues at positions 234 and 235, and Gly residue at position 329 (also referred to as “LALA PG” herein). In some embodiments, one or both Fc polypeptides comprise Ala residues at positions 234 and 235, and Ser residue at position 329 (also referred to as “LALA PS” herein).

Additional Fc polypeptide mutations that modulate an effector function include, but are not limited to, the following: position 329 may have a mutation in which proline is substituted with a glycine or arginine or an amino acid residue large enough to destroy the Fc/Fcγ receptor interface that is formed between proline 329 of the Fc and tryptophan residues Trp 87 and Trp 110 of FcγRIII. Additional illustrative substitutions include S228P, E233P, L235E, N297A, N297D, and P331S, according to the EU numbering scheme. Multiple substitutions may also be present, e.g., L234A and L235A of a human IgG1 Fc region; L234A, L235A, and P329G of a human IgG1 Fc region; L234A, L235A, and P329S of a human IgG1 Fc region; S228P and L235E of a human IgG4 Fc region; L234A and G237A of a human IgG1 Fc region; L234A, L235A, and G237A of a human IgG1 Fc region; V234A and G237A of a human IgG2 Fc region; L235A, G237A, and E318A of a human IgG4 Fc region; and S228P and L236E of a human IgG4 Fc region, according to the EU numbering scheme. In some embodiments, one or both Fc polypeptides may have one or more amino acid substitutions that modulate ADCC, e.g., substitutions at positions 298, 333, and/or 334, according to the EU numbering scheme.

### Fc Polypeptide Modifications for Extending Serum Half-Life

In some embodiments, modifications to enhance serum half-life can be introduced into any Fc polypeptides described herein. For example, in some embodiments, one or both Fc polypeptides in an Fc polypeptide dimer can comprise M428L and N434S substitutions (also referred to as LS substitutions), as numbered according to the EU numbering scheme. Alternatively, one or both Fc polypeptides in an Fc polypeptide dimer can have an N434S or N434A substitution. Alternatively, one or both Fc polypeptides in an Fc polypeptide dimer can have an M428L substitution. In other embodiments, one or both Fc polypeptides in an Fc polypeptide dimer can comprise M252Y, S254T, and T256E substitutions.

In some embodiments, one or both of the Fc polypeptides can have its C-terminal lysine removed (e.g., the Lys residue at position 447 of the Fc polypeptide, according to EU numbering). The C-terminal lysine residue is highly conserved in immunoglobulins across many species and may be fully or partially removed by the cellular machinery during protein production. In some embodiments, removal of the C-terminal lysines in the Fc polypeptides can improve the stability of the proteins.

In some embodiments, a hinge region (e.g., SEQ ID NO:97) or a portion thereof (e.g., SEQ ID NO:98) can be joined to an Fc polypeptide or a modified Fc polypeptide described herein. The hinge region can be from any immunoglobulin subclass or isotype. An illustrative immunoglobulin hinge is an IgG hinge region, such as an IgG1 hinge region, e.g., human IgG1 hinge amino acid sequence EPKSCDKTHTCPPCP (SEQ ID NO:97) or a portion thereof (e.g., DKTHTCPPCP; SEQ ID NO:98). In some embodiments, the hinge region is at the N-terminal region of the Fc polypeptide.

### V. Cell Lines and Methods of Engineering

Provided herein are also cells and cell lines that are homozygous for the gene encoding the G78 variant of the PILRA protein, homozygous for the gene encoding the R78 variant of the PILRA protein, or heterozygous for gene encoding the G78 variant and R78 variant of the PILRA protein. The disclosure provides an engineered human induced pluripotent stem cell (IPSC) or cell line that has been modified (i.e., genetically engineered) to express two copies of (i.e., homozygous for) the gene encoding R78 variant or the G78 variant of a PILRA protein. In some embodiments, the IPSC is modified at the endogenous genomic locus.

The disclosure also provides an engineered microglial cell or cell line that is derived from a human induced pluripotent stem cell (IPSC) that has been modified (i.e., genetically engineered) to express two copies of (i.e., homozygous for) the gene encoding the R78 variant or the G78 variant of a PILRA protein. An engineered microglial cell or cell line can also be derived from a human induced pluripotent stem cell (IPSC) that has been modified (i.e., genetically engineered) to express one copy of the gene encoding the R78 variant and one copy of the gene encoding the G78 variant of a PILRA protein (i.e., heterozygous for the gene encoding the R78 and G78 variants). In some embodiments, the IPSC is modified at the endogenous genomic locus. In some embodiments, the engineered microglial cell or cell line is derived by directed differentiation.

Also provided herein are two cell lines serving as a matched pair of cell lines (e.g., an IPSC line or microglia derived therefrom), in which one cell line expresses the G78 variant of the PILRA protein and the other cell line expresses the R78 variant of the PILRA protein. The disclosure provides a matched pair of cell lines, wherein: (a) the first cell line of the pair is homozygous for the gene encoding the R78 variant of a PILRA protein; and (b) the second cell line of the pair is homozygous for the gene encoding the G78 variant of a PILRA protein, in which both first and second cell lines of the pair are derived from the same parental cell line, and one or both cell lines have been engineered in the endogenous PILRA gene. In particular embodiments of the matched pair of cell lines, the parental cell line used to generate the matched pair of cell lines can be homozygous for the gene encoding the R78 variant of the PILRA protein, which means that only the cell line in the pair that is homozygous for the gene encoding the G78 variant of the PILRA protein needs to be generated from the parental cell line. In other embodiments of the matched pair of cell lines, the parental cell line used to generate the matched pair of cell lines can be homozygous for the gene encoding the G78 variant of the PILRA protein, which means that only the cell line in the pair that is homozygous for the gene encoding the R78 variant of the PILRA protein needs to be generated from the parental cell line. In yet other embodiments, the parental cell line is heterozygous for gene encoding the R78 variant and the G78 variant of the PILRA protein (i.e., one allele encoding the G78 variant and the other allele encoding the R78 variant). In this case, both cell lines in the matched pair need to be generated from the parental cell line.

In some embodiments of the matched pair of cell lines, a third cell line is included that is heterozygous for the gene encoding the G78 variant and the R78 variant of the PILRA protein. In some embodiments, the third cell line is derived from the parental cell line that is homozygous for the gene encoding the R78 variant or the G78 variant of the PILRA protein.

The disclosure also provides methods of generating a myeloid cell line, or a stem cell line capable of differentiating into a myeloid cell line (e.g., an IPSC line or microglia derived therefrom), with a modified PILRA gene, the method comprising: (a) determining whether an existing myeloid cell line, or an existing stem cell line, is homozygous for the gene encoding the R78 variant of a PILRA protein, homozygous for the gene encoding the G78 variant of a PILRA protein, or heterozygous for the gene encoding the R78 and G78 variants of a PILRA protein; and (b) engineering the cell line by modifying the gene encoding the PILRA protein to produce an engineered cell line that is homozygous for the gene encoding the R78 variant of the PILRA protein or the G78 variant of the PILRA protein, wherein the engineered cell line was not, prior to being engineered, homozygous for the gene that encodes the selected variant. In other words, depending on the existing cell line, the existing cell line may or may not need to be modified to generate the selected variant in the desired cell line.

The disclosure also provides methods of generating a matched pair of cell lines (e.g., an IPSC line or microglia derived therefrom), the method comprising: (a) determining whether an existing myeloid cell line, or an existing stem cell line capable of differentiating into a myeloid cell line, is homozygous for the gene encoding the R78 variant of a PILRA protein, homozygous for the gene encoding the G78 variant of a PILRA protein, or heterozygous for the gene encoding the R78 and G78 variants of a PILRA protein; and (b) engineering (i) a first cell line by modifying the gene encoding the PILRA protein to produce an engineered cell line that is homozygous for the gene encoding the R78 variant of the PILRA protein, and/or (ii) a second cell line by modifying the gene encoding the PILRA protein to produce an engineered cell line that is homozygous for the gene encoding the G78 variant of the PILRA protein. In some embodiments, the engineered cell line was not, prior to being engineered, homozygous for the gene that encodes the selected variant.

In particular embodiments, the existing cell line of step (a) is homozygous for the R78 variant of the PILRA protein, and the engineering of step (b) comprises modifying the existing cell line to produce an engineered cell line that is homozygous for the gene encoding the G78 variant of the PILRA protein. In particular embodiments, the existing cell line of step (a) is homozygous for the G78 variant of the PILRA protein, and the engineering of step (b) comprises modifying the existing cell line to produce an engineered cell line that is homozygous for the gene encoding the R78 variant of the PILRA protein. In other embodiments, the existing cell line of step (a) is heterozygous for the gene encoding the R78 and G78 variants of the PILRA protein, and the engineering of step (b) comprises modifying the existing cell line to produce an engineered cell line that is homozygous for the gene encoding the R78 variant of the PILRA protein, and an engineered cell line that is homozygous for the gene encoding the G78 variant of the PILRA protein.

Engineered cells or cell lines with modifications at an endogenous genomic locus (e.g., a PILRA gene locus) can be generated using a variety of methods and techniques, for example, the CRIPSR/Cas9 system, a zinc finger nuclease (ZFN), a Tale-effector domain nuclease (TALEN), and a transposon-mediated system. These methods typically comprise administering to the cell one or more polynucleotides encoding one or more nucleases such that the nuclease mediates modification of the endogenous gene by cleaving the DNA to create 5′ and 3′ cut ends in the DNA strand. In the presence of a donor sequence that is flanked by a left and a right homology arms that are substantially homologous to a sequence extending 5′ from the 5′ end and a sequence extending 3′ from the 3′ end, the donor is integrated into the endogenous gene targeted by the nuclease via homology-directed repair (HDR). In some embodiments, the modification at the endogenous genomic locus is conducted using the CRISPR/Cas9 system. For example, a nucleic acid sequence encoding a heterologous gene encoding a PILRA with the desired variant is introduced into the endogenous PILRA genomic locus of the cell to be modified, which results in that the naturally occurring sequence that encodes the endogenous PILRA is replaced by the heterologous gene.

### CRISPR

In some embodiments, the introduction or knock-in of a heterologous gene encoding a PILRA with the desired variant is performed using the CRIPSR/Cas9 system. The CRISPR/Cas9 system includes a Cas9 protein and at least one to two ribonucleic acids that are capable of directing the Cas9 protein to and hybridizing to a target motif in the endogenous PILRA gene that is to be replaced. These ribonucleic acids are commonly referred to as the “single guide RNA” or “sgRNA.” The Cas9 protein then cleaves the target motif, which results in a double-strand break or a single-strand break. In the presence of a donor DNA that comprises the heterologous PILRA gene sequence flanked by two homology arms, the donor DNA is inserted into the target DNA, replacing the endogenous gene.

The Cas9 protein used in the disclosure can be a naturally occurring Cas9 protein or a functional derivative thereof. A “functional derivative” of a native sequence polypeptide is a compound having a qualitative biological property in common with a native sequence polypeptide. “Functional derivatives” include, but are not limited to, fragments of a native sequence and derivatives of a native sequence polypeptide and its fragments, provided that they have a biological activity in common with a corresponding native sequence polypeptide. A biological activity contemplated herein is the ability of the functional derivative of Cas9 to hydrolyze a DNA substrate into fragments. Suitable functional derivatives of a Cas9 polypeptide or a fragment thereof include but are not limited to mutants, fusions, covalent modifications of Cas9 protein or a fragment thereof.

In some embodiments, the Cas9 protein is from Streptococcus pyogenes. Cas9 contains 2 endonuclease domains, including a RuvC-like domain which cleaves target DNA that is noncomplementary to the sgRNA, and an HNH nuclease domain which cleave target DNA complementary to sgRNA. The double-stranded endonuclease activity of Cas9 also requires that a short conserved sequence (2-5 nucleotides), known as a protospacer-associated motif (PAM), follows immediately 3′ of a target motif in the target sequence. In some embodiments, the PAM motif is an NGG motif. A donor DNA is introduced to the reaction. In one example, the donor DNA comprises the heterologous PILRA gene of the desired variant that is between a left homology arm and a right homology arm.

The sgRNAs can be selected depending on the particular CRISPR/Cas9 system employed and the sequence of the target polynucleotide. In some embodiments, the one to two ribonucleic acids are designed to hybridize to a target motif immediately adjacent to a deoxyribonucleic acid motif recognized by the Cas9 protein. In some embodiments, each of the one to two ribonucleic acids are designed to hybridize to target motifs immediately adjacent to deoxyribonucleic acid motifs recognized by the Cas9 protein, wherein the target motifs flank the genomic sequence to be replaced. Guide RNAs can be designed using software that is readily available, for example, at http://crispr.mit.edu.

In some embodiments, the donor DNA as disclosed herein comprises a nucleotide sequence that encodes the amino acid sequence of a hPILRA G78 variant. In some embodiments, the donor DNA as disclosed herein comprises a nucleotide sequence that encodes the amino acid sequence of a hPILRA R78 variant. The donor DNA as disclosed herein further comprises a left homology arm and a right homology arm that flank the nucleotide sequence and are designed to overlap the 5′ and 3′ exon sequences relative to the cleave site by the Cas9 protein. The homology arms may extend beyond the 5′ and 3′ exon sequences, and each of the homology arms may be at least 20, 30, 40, 50, 100, or 150 nucleotides in length. One of skilled in the art can readily determine the optimal length of the homology arm required for the experiment.

In some embodiments, the sgRNAs can also be selected to minimize hybridization with nucleic acid sequences other than the target polynucleotide sequence. In some embodiments, the one to two ribonucleic acids are designed to hybridize to a target motif that contains at least two mismatches when compared with all other genomic nucleotide sequences in the cell to minimize off-target effects of the CRISPR/Cas9 system. Those skilled in the art will appreciate that a variety of techniques can be used to select suitable target motifs for minimizing off-target effects (e.g., bioinformatics analyses).

### Zinc Finger Nuclease (ZFN)

In some embodiments, the introduction or knock-in of a heterologous gene encoding a PILRA with the desired variant is performed using a ZFN. ZFNs are fusion proteins that comprise a non-specific cleavage domain (N) of FokI endonuclease and a zinc finger protein (ZFP). A pair of ZNFs are involved to recognize a specific locus in a target gene: one that recognizes the sequence upstream and the other that recognizes the sequence downstream of the site to be modified. The nuclease portion of the ZFN cuts at the specific locus. The donor DNA can then be inserted into the specific locus. Methods of using the ZFNs are well known, for example, as disclosed in U.S. Pat. No. 9,045,763 and also in Durai et al., “Zinc Finger Nucleases: Custom-Designed Molecular Scissors for Genome Engineering of Plant and Mammalian cells,” Nucleic Acid Research, 33 (18):5978-5990 (2005), the disclosures of which are incorporated by reference in their entirety.

### Transcription Activator-Like Effector Nucleases (TALENs)

In some embodiments, the introduction or knock-in of a heterologous gene encoding a PILRA with the desired variant is performed using TALENs. TALENs are similar to ZFNs in that they bind as a pair around a genomic site and direct the same non-specific nuclease, FokI, to cleave the genome at a specific site, but instead of recognizing DNA triplets, each domain recognizes a single nucleotide. Methods of using the ZFNs are also well known, for example, as disclosed in U.S. Pat. No. 9,005,973 and also Christian et al., “Targeting DNA Double-Strand Breaks with TAL Effector Nucleases,” Genetics, 186(2): 757-761 (2010), the disclosures of which are incorporated by reference in their entirety.

The disclosure also provides methods of generating a matched pair of cell lines from an existing myeloid cell line (e.g., an IPSC line or microglia derived therefrom), or an existing stem cell line capable of differentiating into a myeloid cell line, that is heterozygous for gene encoding the R78 and G78 variants of a PILRA protein, the method comprising: (a) engineering the existing cell line to produce a first engineered cell line that is homozygous for the gene encoding the R78 variant of the PILRA protein; and (b) engineering either the cell line generated in step (a) or the existing cell line to produce a second engineered cell line that is homozygous for the gene encoding the G78 variant of the PILRA protein. As described herein, the CRIPSR/Cas9 system can be used to generate the matched pair of cell lines using a donor DNA that comprises a nucleotide sequence that encodes the amino acid sequence of a hPILRA R78 variant or G78 variant.

In another aspect, the disclosure provides a method of generating a matched pair of cell lines from an existing myeloid cell line, or an existing stem cell line capable of differentiating into a myeloid cell (e.g., an IPSC line or microglia derived therefrom), that is heterozygous for gene encoding the R78 and G78 variants of a PILRA protein, the method comprising: (a) engineering the existing cell line to produce a first engineered cell line that is homozygous for the gene encoding the G78 variant of the PILRA protein; and (b) engineering either the cell line generated in step (a) or the existing cell line to produce a second engineered cell line that is homozygous for the gene encoding the R78 variant of the PILRA protein. As described herein, the CRIPSR/Cas9 system can be used to generate the matched pair of cell lines using a donor DNA that comprises a nucleotide sequence that encodes the amino acid sequence of a hPILRA R78 variant or G78 variant.

The disclosure also provides methods of generating a matched pair of cell lines from an existing myeloid cell line, or an existing stem cell line capable of differentiating into a myeloid cell line (e.g., an IPSC line or microglia derived therefrom), that is homozygous for the gene encoding the R78 variant of a PILRA protein, the method comprising: engineering the existing cell line to produce an engineered cell line that is homozygous for gene encoding the G78 variant of the PILRA protein. As described herein, the CRIPSR/Cas9 system can be used to generate the matched pair of cell lines using a donor DNA that comprises a nucleotide sequence that encodes the amino acid sequence of a hPILRA G78 variant.

The disclosure also provides methods of generating a matched pair of cell lines from an existing myeloid cell line, or an existing stem cell line capable of differentiating into a myeloid cell line (e.g., an IPSC line or microglia derived therefrom), that is homozygous for the gene encoding the G78 variant of a PILRA protein, the method comprising: engineering the existing cell line to produce an engineered cell line that is homozygous for gene encoding the R78 variant of the PILRA protein. As described herein, the CRIPSR/Cas9 system can be used to generate the matched pair of cell lines using a donor DNA that comprises a nucleotide sequence that encodes the amino acid sequence of a hPILRA R78 variant.

In some embodiments, the engineered cell, cell line, or cell model described herein is derived by directed differentiation.

### VI. Methods of Screening

The disclosure also provides methods for screening and identifying molecules that that bind to and/or modulate expression or activity of a PILRA protein, especially molecules that antagonize or reduce PILRA activity (i.e., molecules that block binding of a ligand to hPILRA). In some embodiments, one or more downstream signaling responses that is related to PILRA binding and/or activation can be measured to identify PILRA-binding molecules. For example, a molecule that binds to a PILRA protein of a cell can cause one or more downstream signaling responses or activities of the cell as a result of PILRA-binding. In some embodiments, a molecule that binds to a PILRA protein can cause an increase or decrease in the signaling response or activity of the cell as a result of PILRA-binding, relative to the signaling response or activity of the cell without PILRA-binding. Examples of changes in signaling responses or activities of a cell as a result of PILRA-binding include, but are not limited to, changes phosphorylated STAT3 (pSTAT3) level, phosphorylated STAT1 (pSTAT1) level, phosphorylated EGFR (pEGFR) level, cadherin expression, integrin expression, and cell (e.g., microglia) migration. In particular embodiments, molecules that bind to PILRA and antagonize or reduce PILRA activity can cause a downstream signaling response, such as an increase in pSTAT3 (e.g., pSTAT3 Y705, or pSTAT3 S727) level, an increase in pEGFR level, an increase in the expression level and/or cell secretion of a protein (e.g., cadherin, integrin), and/or an increase in cell (e.g., microglia) migration. Examples of other downstream signaling responses that can be caused by molecules that bind to PILRA and antagonize or reduce PILRA activity can be, for example, elevated cellular respiration, elevated fatty acid metabolism (e.g., fatty acid oxidation), elevated ATP production, increased anti inflammatory gene or protein expression, and/or reduced cytokine protein expression.

Provided herein are methods for screening to determine whether a molecule has activity at a PILRA protein, the method comprising: (a) contacting a cell that expresses the PILRA protein with the molecule; (b) either prior to, concurrently with, or following step (a), contacting a cell of the same type as in step (a) having lower PILRA expression with the molecule; and (c) measuring one of the following: phosphorylated STAT3 (pSTAT3) level, phosphorylated STAT1 (pSTAT1) level, phosphorylated EGFR (pEGFR) level, cadherin expression, integrin expression, and microglial migration in both cells. In some embodiments, a change in the level of one of these measurements between the cells indicates that the molecule has activity at the PILRA protein of step (a).

In certain embodiments of the methods for screening, the cell of step (a) naturally expresses the PILRA protein. In some embodiments, the cell having lower PILRA expression has the PILRA protein knocked-out or silenced. In particular embodiments, the cell can be a microglia. In certain embodiments, the cell is an iMicroglia (e.g., a PILRA LoF iMicroglia).

In certain embodiments of the methods for screening, the cell of step (a) is engineered or modified to express or overexpress the PILRA protein. In some embodiments, the cell having lower PILRA expression naturally expresses the PILRA protein or is not engineered or modified to express the PILRA protein.

In some embodiments, a library of molecules can be screened using the methods described herein. In certain cases, the molecule is known to bind the PILRA protein. In other cases, it is unknown whether the molecule binds the PILRA protein. Examples of molecules that can be screened to determine whether the molecule has any activity at the PILRA protein include, but are not limited to, antibodies, peptides, organic small molecules, or nucleic acids.

Also provided herein are methods for determining whether a molecule that binds a PILRA protein modulates a signaling response or activity in a PILRA-expressing cell, the method comprising: (a) contacting the cell with the molecule; and (b) measuring one of the following: phosphorylated STAT3 (pSTAT3) level, phosphorylated STAT1 (pSTAT1) level, phosphorylated EGFR (pEGFR) level, cadherin expression, integrin expression, and cell (e.g., microglia) migration. In some embodiments, a change in the level of one of the measurements indicates that the molecule modulates the signaling response or activity in the PILRA-expressing cell. In certain embodiments, the change is an increase or decrease in the level of one of the measurements when the molecule contacts the cell, relative to the level in the cell without the molecule, in particular, the changes described elsewhere in the application. In particular embodiments of these methods, the cell is in an in vitro assay. In other embodiments, the cell is in a mammal (i.e., in vivo methods).

In some embodiments, when the methods are used in vivo, and step (a) comprises administering the molecule to a mammal.

In some embodiments, a PILRA-expressing cell can be a microglia, a myeloid cell, a monocyte, or a neutrophil.

### Screening Assays

Screening assays to identify molecules that that bind to and/or modulate expression or activity of a PILRA protein can be carried out by standard methods. The screening methods may involve high-throughput techniques. In addition, these screening techniques may be carried out in cultured cells or in organisms such as mice, worms, flies, or yeast.

Any number of methods is available for carrying out such screening assays. According to one approach, candidate molecules are added at varying concentrations to the culture medium of PILRA-expressing cells. If downstream signaling such as phospho-STAT3 (pSTAT3) induction is used as a measurement of whether the molecule binds and/or modulate expression or activity of the PILRA protein, pSTAT3 levels can be measured in a cell that expresses the PILRA protein and compared to the pSTAT3 level in a corresponding cell that expresses a lower level of PILRA (e.g., a PILRA knockout). In other cases, the pSTAT3 level can be measured before and after adding the molecule to the cell. These levels of pSTAT3 can be compared.

In another approach, cellular secretions of proteins such as integrins and cadherins can also be measured to determine whether the molecule binds and/or modulate expression or activity of the PILRA protein, as it is demonstrated in the examples that anti-PILRA antibodies enhanced iMicroglial secretion of these proteins. Standard laboratory techniques can be used to isolate these proteins from the cell and detection of these proteins can be performed using, e.g., mass spectrometry, Western blot, and Proteome profiler kit; Human Soluble Receptor Array Kit—Non-Hematopoietic Panel (R&D ARY012).

In other embodiments, a candidate molecule that binds to a PILRA protein may be identified using a chromatography-based technique. For example, recombinant PILRA may be purified by standard techniques from cells engineered to express PILRA and may be immobilized on a column. A solution of candidate molecules is then passed through the column, and a molecule specific for PILRA is identified on the basis of its ability to bind to the polypeptide and be immobilized on the column. To isolate the molecule, the column is washed to remove non-specifically bound molecules, and the molecule of interest is then released from the column and collected. Molecules isolated by this method (or any other appropriate method) may, if desired, be further purified (e.g., by high performance liquid chromatography).

### Test Molecules

In general, potential molecules can be identified from large libraries of both natural product or synthetic (or semi-synthetic) extracts or chemical libraries according to methods known in the art. Those skilled in the field of drug discovery and development will understand that the precise source of test extracts or compounds is not critical to the screening procedure(s) of the disclosure. Accordingly, virtually any number of chemical extracts or molecules can be screened using the methods described herein. Examples of such extracts or molecules include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and polynucleotide-based compounds. Synthetic compound libraries are commercially available. Alternatively, libraries of natural compounds in the form of bacterial, fungal, plant, and animal extracts are commercially available. In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

When a crude extract is found to have an activity, further fractionation of the positive lead extract is necessary to isolate chemical constituents responsible for the observed effect. Thus, the goal of the extraction, fractionation, and purification process is the characterization and identification of a chemical entity within the crude extract having the desired activity. Methods of fractionation and purification of such heterogenous extracts are known in the art. If desired, molecules shown to be useful can be chemically modified according to methods known in the art.

### VII. Measuring Activity of PILRA-Binding Molecules in a Cell or Animal

Also provided herein are methods for measuring the binding and/or activity of a molecule that binds to a PILRA protein (e.g., hPILRA G78 or R78). In some embodiments, the molecule antagonizes or reduces PILRA activity (i.e., molecules that block binding of a ligand to hPILRA). Various measurements can be made to determine the binding and/or activity of a PILRA-binding molecule and its effects on the cell or animal. For example, we have demonstrated herein that the induction of phosphorylated STAT3 is a cellular downstream signaling response that is PILRA-dependent and happens when PILRA is antagonized.

In some embodiments, to measure the binding and/or activity of a PILRA-binding molecule, after cells were incubated with the PILRA-binding molecule, phosphorylated STAT3 (e.g., pSTAT3 Y705 and/or pSTAT3 S727) level can be measured using, for example, a Proteome Profiler Human Phospho-Kinase Array Kit (e.g., ARY003C, R&D Systems). In other embodiments, to measure phosphorylated protein levels, after the cells were incubated with the PILRA-binding molecule, the cells can be fixed and the phosphorylated protein can be detected using standard immunocytochemistry protocol. Cells can then be imaged with a confocal microscope and images can be analyzed using a software to calculate mean fluorescent spot area and intensity per cell to determine phosphorylated protein level.

Other cellular responses that are dependent on PILRA binding (i.e., antagonizing) include, e.g., an increase in phosphorylated EGFR (e.g., pEGFR Y1086) level, which can also be measured using a phosphor-kinase array kit or immunocytochemistry, as mentioned above.

In some embodiments, measuring the phosphorylation level of STAT3 and/or EGFR upon PILRA-binding by the molecules can be used to rank antagonistic effects of the molecules. For example, a PILRA-binding molecule whose binding resulted in the highest level of pSTAT3 can be determined to have the most antagonistic activity at the PILRA protein.

Other measurements that can be made to determine the binding and/or activity of a PILRA-binding molecule and its effects on the cell or animal include, for example, measuring cell migration, which is another cellular downstream signaling response that is PILRA-dependent and happens when PILRA is antagonized. As described in, e.g., Example 4, measurement and quantification of cell migration can be performed using a cell migration assay where a rubber stopper can be used to create a cell-free detection zone. The rubber stopper can then be removed upon addition of the PILRA-binding molecule, and a cell stain such as NucBlue or DAPI can be added. The cells can be imaged using microscopy and the images can be analyzed using a software to quantify nuclear labeling of the cells that migrated to the detection zone. Further, as PILRA-binding molecules that antagonize PILRA also enhance the cell secretion of motile proteins, quantification of such motile proteins in the cell supernatant after addition of PILRA-binding molecule to the cells can also be performed. For example, soluble analytes in the supernatants can be analyzed with a proteome profiler kit, such as Human Soluble Receptor Array Kit—Non-Hematopoietic Panel (e.g., R&D ARY012). Examples of motile proteins that can be quantified in this manner include, but are not limited to, cadherins and integrins.

Measuring the binding and/or activity of a PILRA-binding molecule can be performed in a cell or an animal (e.g., mice, monkeys). For in vivo studies, an animal, e.g., an animal expressing a PILRA protein (e.g., PILRA G78 or R78) can be administered a PILRA-binding molecule via any mode of administration available (e.g., IV, IP, oral, nasal, or transdermal administration). The appropriate cells, tissues, and/or fluid samples can be isolated from the animal to measure and quantify one or more of the PILRA-dependent downstream signaling responses described herein, such as pSTAT3 level, pEGFR level, amount of motile proteins (e.g., cadherins, integrins). In some embodiments, the cell or animal is homozygous for the gene encoding PILRA G78. In some embodiments, the cell or animal is homozygous for the gene encoding PILRA R78. In some embodiments, the cell or animal is heterozygous for the gene encoding PILRA G78 and R78 variants.

### VIII. Preparation of Antibodies

In some embodiments, antibodies are prepared by immunizing an animal or animals (e.g., mice, rabbits, or rats) with an antigen or a mixture of antigens for the induction of an antibody response. In some embodiments, the antigen or mixture of antigens is administered in conjugation with an adjuvant (e.g., Freund's adjuvant). After an initial immunization, one or more subsequent booster injections of the antigen or antigens may be administered to improve antibody production. Following immunization, antigen-specific B cells are harvested, e.g., from the spleen and/or lymphoid tissue. For generating monoclonal antibodies, the B cells are fused with myeloma cells, which are subsequently screened for antigen specificity. Methods of preparing antibodies are also described in the Examples section below.

The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Alternatively, phage or yeast display technology can be used to identify antibodies and Fab fragments that specifically bind to selected antigens. Antibodies can also be made bispecific, i.e., able to recognize two different antigens. Antibodies can also be heteroconjugates, e.g., two covalently joined antibodies, or immunotoxins.

Antibodies can be produced using any number of expression systems, including prokaryotic and eukaryotic expression systems. In some embodiments, the expression system is a mammalian cell expression, such as a hybridoma, or a CHO cell expression system. Many such systems are widely available from commercial suppliers. In embodiments in which an antibody comprises both a V_{H} and V_{L} region, the V_{H} and V_{L} regions may be expressed using a single vector, e.g., in a di-cistronic expression unit, or under the control of different promoters. In other embodiments, the V_{H} and V_{region} may be expressed using separate vectors. A V_{H} or V_{L} region as described herein may optionally comprise a methionine at the N-terminus.

In some embodiments, the antibody is a chimeric antibody. Methods for making chimeric antibodies are known in the art. For example, chimeric antibodies can be made in which the antigen binding region (heavy chain variable region and light chain variable region) from one species, such as a mouse, is fused to the effector region (constant domain) of another species, such as a human. As another example, “class switched” chimeric antibodies can be made in which the effector region of an antibody is substituted with an effector region of a different immunoglobulin class or subclass.

In some embodiments, the antibody is a humanized antibody. Generally, a non-human antibody is humanized in order to reduce its immunogenicity. Humanized antibodies typically comprise one or more variable regions (e.g., CDRs) or portions thereof that are non-human (e.g., derived from a mouse variable region sequence), and possibly some framework regions or portions thereof that are non-human, and further comprise one or more constant regions that are derived from human antibody sequences. Methods for humanizing non-human antibodies are known in the art. Transgenic mice, or other organisms such as other mammals, can be used to express humanized or human antibodies. Other methods of humanizing antibodies include, for example, variable domain resurfacing, CDR grafting, grafting specificity-determining residues (SDR), guided selection, and framework shuffling.

As an alternative to humanization, fully human antibodies can be generated. As a non-limiting example, transgenic animals (e.g., mice) can be produced that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. As another example, human antibodies can be produced by hybridoma-based methods, such as by using primary human B cells for generating cell lines producing human monoclonal antibodies.

Human antibodies can also be produced using phage display or yeast display technology. In phage display, repertoires of variable heavy chain and variable light chain genes are amplified and expressed in phage display vectors. In some embodiments, the antibody library is a natural repertoire amplified from a human source. In some embodiments, the antibody library is a synthetic library made by cloning heavy chain and light chain sequences and recombining to generate a large pool of antibodies with different antigenic specificity. Phage typically display antibody fragments (e.g., Fab fragments or scFv fragments), which are then screened for binding to an antigen of interest.

In some embodiments, antibody fragments (such as a Fab, a Fab′, a F(ab′)₂, a scFv, a V_{H}, or a V_{HH}) are generated. Various techniques have been developed for the production of antibody fragments. Traditionally, these fragments were derived via proteolytic digestion of intact antibodies. However, these fragments can now be produced directly using recombinant host cells. For example, antibody fragments can be isolated from antibody phage libraries. Alternatively, Fab′-SH fragments can be directly recovered from E. coli cells and chemically coupled to form F(ab′)₂ fragments. According to another approach, F(ab′)₂ fragments can be isolated directly from recombinant host cell culture. Other techniques for the production of antibody fragments will be apparent to those skilled in the art.

In some embodiments, an antibody or an antibody fragment is conjugated to another molecule, e.g., polyethylene glycol (PEGylation) or serum albumin, to provide an extended half-life in vivo.

### IX. Nucleic Acids, Vectors, and Host Cells

In some embodiments, the anti-PILRA antibodies as disclosed herein are prepared using recombinant methods. Accordingly, in some aspects, the disclosure provides isolated nucleic acids comprising a nucleic acid sequence encoding any of the anti-PILRA antibodies as described herein (e.g., any one or more of the CDRs, heavy chain variable regions, and light chain variable regions described herein); vectors comprising such nucleic acids; and host cells into which the nucleic acids are introduced that are used to replicate the antibody-encoding nucleic acids and/or to express the antibodies.

In some embodiments, a polynucleotide (e.g., an isolated polynucleotide) comprises a nucleotide sequence encoding an antibody as described herein. In some embodiments, the polynucleotide comprises a nucleotide sequence encoding one or more amino acid sequences (e.g., CDR, heavy chain, or light chain sequences) disclosed in Table 1. In some embodiments, the polynucleotide comprises a nucleotide sequence encoding an amino acid sequence having at least 85% sequence identity (e.g., at least 85%, at least 90%, at least 91%, at least 92%, at least 93%, at least 94%, at least 95%, at least 96%, at least 97%, at least 98%, or at least 99% sequence identity) to a sequence (e.g., a CDR, heavy chain, or light chain sequence) disclosed in Table 1. In some embodiments, a polynucleotide as described herein is operably linked to a heterologous nucleic acid, e.g., a heterologous promoter.

Suitable vectors containing polynucleotides encoding antibodies of the present disclosure, or fragments thereof, include cloning vectors and expression vectors. While the cloning vector selected may vary according to the host cell intended to be used, useful cloning vectors generally have the ability to self-replicate, may possess a single target for a particular restriction endonuclease, and/or may carry genes for a marker that can be used in selecting clones containing the vector. Examples include plasmids and bacterial viruses, e.g., pUC18, pUC19, Bluescript (e.g., pBS SK+) and its derivatives, mpl8, mpl9, pBR322, pMB9, ColE1, pCR1, RP4, phage DNAs, and shuttle vectors such as pSA3 and pAT28. These and many other cloning vectors are available from commercial vendors such as BioRad, Strategene, and Invitrogen.

Expression vectors generally are replicable polynucleotide constructs that contain a nucleic acid of the present disclosure. The expression vector may replicate in the host cells either as episomes or as an integral part of the chromosomal DNA. Suitable expression vectors include but are not limited to plasmids, viral vectors, including adenoviruses, adeno-associated viruses, retroviruses, and any other vector.

Suitable host cells for cloning or expressing a polynucleotide or vector as described herein include prokaryotic or eukaryotic cells. In some embodiments, the host cell is prokaryotic. In some embodiments, the host cell is eukaryotic, e.g., Chinese Hamster Ovary (CHO) cells or lymphoid cells. In some embodiments, the host cell is a human cell, e.g., a Human Embryonic Kidney (HEK) cell.

In another aspect, methods of making an anti-PILRA antibody as described herein are provided. In some embodiments, the method includes culturing a host cell as described herein (e.g., a host cell expressing a polynucleotide or vector as described herein) under conditions suitable for expression of the antibody. In some embodiments, the antibody is subsequently recovered from the host cell (or host cell culture medium).

### X. Therapeutic Methods Using Anti-PILRA Antibodies

In another aspect, therapeutic methods using an anti-PILRA antibody as disclosed herein (e.g., an anti-PILRA antibody as described in Section III above) are provided. In some embodiments, methods of treating a neurodegenerative disease are provided. In some embodiments, methods of modulating one or more PILRA activities (e.g., in a subject having a neurodegenerative disease) are provided.

In some embodiments, methods of treating a neurodegenerative disease are provided. In some embodiments, the neurodegenerative disease is selected from the group consisting of Alzheimer's disease, primary age-related tauopathy, progressive supranuclear palsy (PSP), frontotemporal dementia, frontotemporal dementia with parkinsonism linked to chromosome 17, argyrophilic grain dementia, amyotrophic lateral sclerosis, amyotrophic lateral sclerosis/parkinsonism-dementia complex of Guam (ALS-PDC), corticobasal degeneration, chronic traumatic encephalopathy, Creutzfeldt-Jakob disease, dementia pugilistica, diffuse neurofibrillary tangles with calcification, Down's syndrome, familial British dementia, familial Danish dementia, Gerstmann-Straussler-Scheinker disease, globular glial tauopathy, Guadeloupean parkinsonism with dementia, Guadelopean PSP, Hallevorden-Spatz disease, hereditary diffuse leukoencephalopathy with spheroids (HDLS), Huntington's disease, inclusion-body myositis, multiple system atrophy, myotonic dystrophy, Nasu-Hakola disease, neurofibrillary tangle-predominant dementia, Niemann-Pick disease type C, pallido-ponto-nigral degeneration, Parkinson's disease, Pick's disease, postencephalitic parkinsonism, prion protein cerebral amyloid angiopathy, progressive subcortical gliosis, subacute sclerosing panencephalitis, and tangle only dementia. In some embodiments, the neurodegenerative disease is Alzheimer's disease. In some embodiments, the neurodegenerative disease is Nasu-Hakola disease. In some embodiments, the neurodegenerative disease is frontotemporal dementia. In some embodiments, the neurodegenerative disease is Parkinson's disease. In some embodiments, the method comprises administering to the subject an isolated antibody or an antigen-binding fragment thereof that specifically binds to a hPILRA protein, e.g., an anti-PILRA antibody as described herein, or a pharmaceutical composition comprising an anti-PILRA antibody as described herein.

In some embodiments, an anti-PILRA antibody (or antigen-binding portion or pharmaceutical composition thereof) as described herein is used in treating a neurodegenerative disease that is characterized by PILRA activity. In some embodiments, the neurodegenerative disease that is characterized by PILRA activity is Alzheimer's disease.

In some embodiments, methods of modulating one or more PILRA activities in a subject (e.g., a subject having a neurodegenerative disease) are provided. In some embodiments, the method comprises antagonizing or reducing PILRA activity, e.g., blocking binding of a ligand to hPILRA, altering phosphorylation of one or more downstream proteins (e.g., increases phosphorylation of EGFR or STAT3; decreases phosphorylation of STAT1), elevating cellular respiration, fatty acid metabolism (e.g., fatty acid oxidation), and ATP production, enhancing cell migration, increasing anti-inflammatory gene or protein expression, and/or reducing cytokine protein expression. Thus, in another aspect, methods of antagonizing PILRA activity, e.g., in a subject having a neurodegenerative disease, are provided. In some embodiments, the method of modulating one or more PILRA activities in a subject comprises administering to the subject an isolated antibody or an antigen-binding portion thereof that specifically binds to a hPILRA protein, e.g., an anti-PILRA antibody as describe herein, or a pharmaceutical composition comprising an anti-PILRA antibody as described herein.

In some embodiments, the subject to be treated is a human, e.g., a human adult or a human child.

In some embodiments, methods of reducing plaque accumulation in a subject having a neurodegenerative disease are provided. In some embodiments, the method comprises administering to the subject an antibody or pharmaceutical composition as described herein. In some embodiments, the subject has Alzheimer's disease. In some embodiments, the subject is an animal model of a neurodegenerative disease (e.g., a 5XFAD or APP/PS1 mouse model). In some embodiments, plaque accumulation is measured by amyloid plaque imaging and/or Tau imaging, e.g., using positron emission tomography (PET) scanning. In some embodiments, administration of an anti-PILRA antibody reduces plaque accumulation by at least 20%, at least 30%, at least 40%, at least 50%, at least 60%, at least 70%, at least 80%, or at least 90% as compared to a baseline value (e.g., the level of plaque accumulation in the subject prior to administration of the anti-PILRA antibody).

In some embodiments, an anti-PILRA antibody is administered to a subject at a therapeutically effective amount or dose. The dosages, however, may be varied according to several factors, including the chosen route of administration, the formulation of the composition, patient response, the severity of the condition, the subject's weight, and the judgment of the prescribing physician. The dosage can be increased or decreased over time, as required by an individual patient. In certain instances, a patient initially is given a low dose, which is then increased to an efficacious dosage tolerable to the patient. Determination of an effective amount is well within the capability of those skilled in the art.

The route of administration of an anti-PILRA antibody as described herein can be oral, intraperitoneal, transdermal, subcutaneous, intravenous, intramuscular, intrathecal, inhalational, topical, intralesional, rectal, intrabronchial, nasal, transmucosal, intestinal, ocular or otic delivery, or any other methods known in the art. In some embodiments, the antibody is administered orally, intravenously, or intraperitoneally.

In some embodiments, the anti-PILRA antibody (and optionally another therapeutic agent) is administered to the subject over an extended period of time, e.g., for at least 30, 40, 50, 60, 70, 80, 90, 100, 150, 200, 250, 300, 350 days or longer.

### XI. Pharmaceutical Compositions and Kits

In another aspect, pharmaceutical compositions and kits comprising an antibody that specifically binds to a hPILRA protein are provided. In some embodiments, the pharmaceutical compositions and kits are for use in treating a neurodegenerative disease. In some embodiments, the pharmaceutical compositions and kits are for use in modulating (e.g., enhancing or inhibiting) one or more PILRA activities, e.g., EGFR, STAT3, and/or STAT1 phosphorylation.

### Pharmaceutical Compositions

In some embodiments, pharmaceutical compositions comprising an anti-PILRA antibody or an antigen-binding fragment thereof are provided. In some embodiments, the anti-PILRA antibody is an antibody as described in Section III above or an antigen-binding fragment thereof.

In some embodiments, a pharmaceutical composition comprises an anti-PILRA antibody as described herein and further comprises one or more pharmaceutically acceptable carriers and/or excipients. A pharmaceutically acceptable carrier includes any solvents, dispersion media, or coatings that are physiologically compatible and that does not interfere with or otherwise inhibit the activity of the active agent. Various pharmaceutically acceptable excipients are well-known in the art.

In some embodiments, the carrier is suitable for intravenous, intramuscular, oral, intraperitoneal, intrathecal, transdermal, topical, or subcutaneous administration. Pharmaceutically acceptable carriers can contain one or more physiologically acceptable compound(s) that act, for example, to stabilize the composition or to increase or decrease the absorption of the active agent(s). Physiologically acceptable compounds can include, for example, carbohydrates, such as glucose, sucrose, or dextrans, antioxidants, such as ascorbic acid or glutathione, chelating agents, low molecular weight proteins, compositions that reduce the clearance or hydrolysis of the active agents, or excipients or other stabilizers and/or buffers. Other pharmaceutically acceptable carriers and their formulations are well-known in the art.

The pharmaceutical compositions described herein can be manufactured in a manner that is known to those of skill in the art, e.g., by means of conventional mixing, dissolving, granulating, dragee-making, emulsifying, encapsulating, entrapping or lyophilizing processes. The following methods and excipients are merely exemplary and are in no way limiting.

For oral administration, an anti-PILRA antibody can be formulated by combining it with pharmaceutically acceptable carriers that are well known in the art. Such carriers enable the compounds to be formulated as tablets, pills, dragees, capsules, emulsions, lipophilic and hydrophilic suspensions, liquids, gels, syrups, slurries, suspensions and the like, for oral ingestion by a patient to be treated. Pharmaceutical preparations for oral use can be obtained by mixing the compounds with a solid excipient, optionally grinding a resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents can be added, such as a cross-linked polyvinyl pyrrolidone, agar, or alginic acid or a salt thereof such as sodium alginate.

An anti-PILRA antibody can be formulated for parenteral administration by injection, e.g., by bolus injection or continuous infusion. For injection, the compound or compounds can be formulated into preparations by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives. In some embodiments, compounds can be formulated in aqueous solutions, e.g., in physiologically compatible buffers such as Hanks's solution, Ringer's solution, or physiological saline buffer. Formulations for injection can be presented in unit dosage form, e.g., in ampules or in multi-dose containers, with an added preservative. The compositions can take such forms as suspensions, solutions or emulsions in oily or aqueous vehicles, and can contain formulatory agents such as suspending, stabilizing and/or dispersing agents.

Typically, a pharmaceutical composition for use in in vivo administration is sterile. Sterilization can be accomplished according to methods known in the art, e.g., heat sterilization, steam sterilization, sterile filtration, or irradiation.

Dosages and desired drug concentration of pharmaceutical compositions of the disclosure may vary depending on the particular use envisioned. The determination of the appropriate dosage or route of administration is well within the skill of one in the art. Suitable dosages are also described in above.

### Kits

In some embodiments, kits comprising an anti-PILRA antibody are provided. In some embodiments, the anti-PILRA antibody is an antibody as described in Section III above or an antigen-binding fragment thereof.

In some embodiments, the kit further comprises one or more additional therapeutic agents. For example, in some embodiments, the kit comprises an anti-PILRA antibody as described herein and further comprises one or more additional therapeutic agents for use in the treatment of a neurodegenerative disease, e.g., Alzheimer's disease. In some embodiments, the therapeutic agent is an agent for use in treating a cognitive or behavioral symptom of a neurodegenerative disease (e.g., an antidepressant, a dopamine agonist, or an anti-psychotic). In some embodiments, the therapeutic agent is a neuroprotective agent (e.g., carbidopa/levodopa, an anticholinergic agent, a dopaminergic agent, a monoamine oxidase B (MAO-B) inhibitor, a catechol-O-methyl transferase (COMT) inhibitor, a glutamatergic agent, a histone deacetylase (HDAC) inhibitor, a cannabinoid, a caspase inhibitor, melatonin, an anti-inflammatory agent, a hormone (e.g., estrogen or progesterone), or a vitamin).

In some embodiments, the kit comprises an anti-PILRA antibody as described herein and further comprises one or more reagents for measuring anti-PILRA antibody induced activity (e.g., for measuring EGFR, STAT3, and/or STAT1 phosphorylation).

In some embodiments, the kit further comprises instructional materials containing directions (i.e., protocols) for the practice of the methods described herein (e.g., instructions for using the kit for a therapeutic method as described above). While the instructional materials typically comprise written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD-ROM), and the like. Such media may include addresses to internet sites that provide such instructional materials.

### EXAMPLES

The present disclosure will be described in greater detail by way of specific examples. The following examples are offered for illustrative purposes only, and are not intended to limit the disclosure in any manner.

### Example 1—Evaluation of PILRA and PILRB Binding in IPSC-Derived Microglia, HEK293, and CHO-K1 Cells

### PILRA mAb Binding to HEK293, CHO-K1, and Human IPSC-Derived Microglia

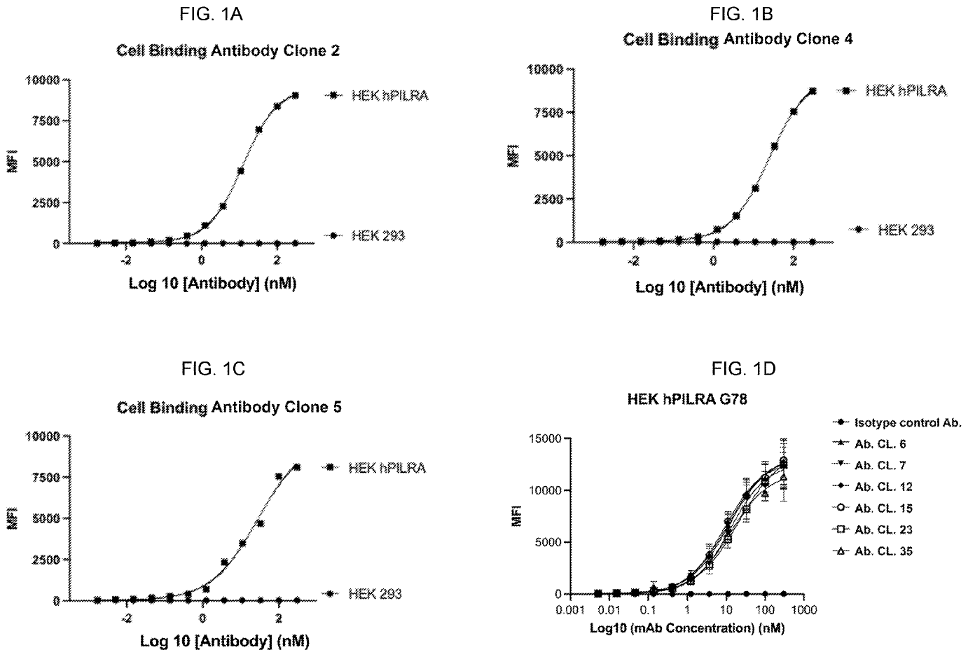

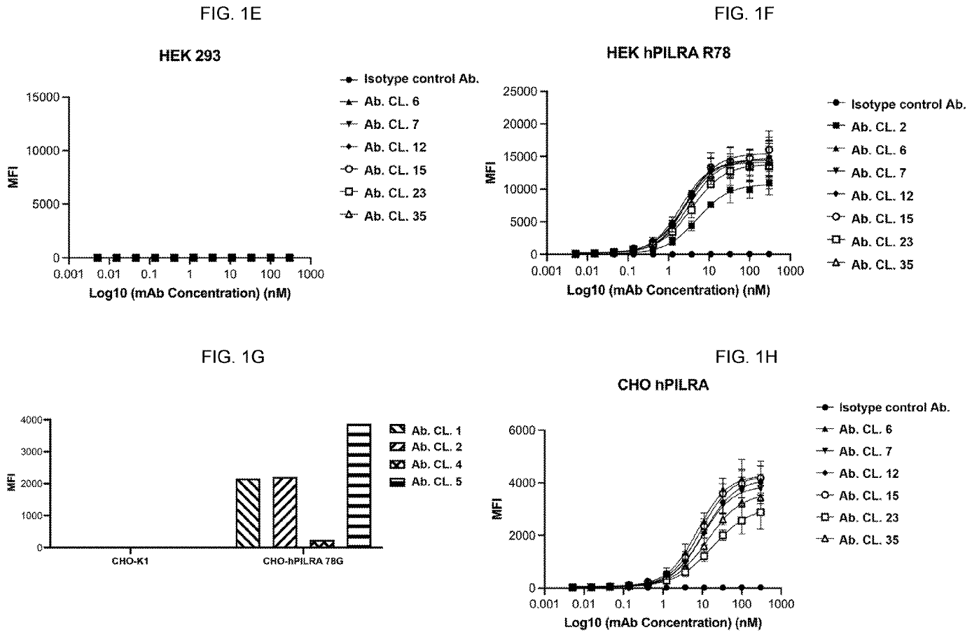

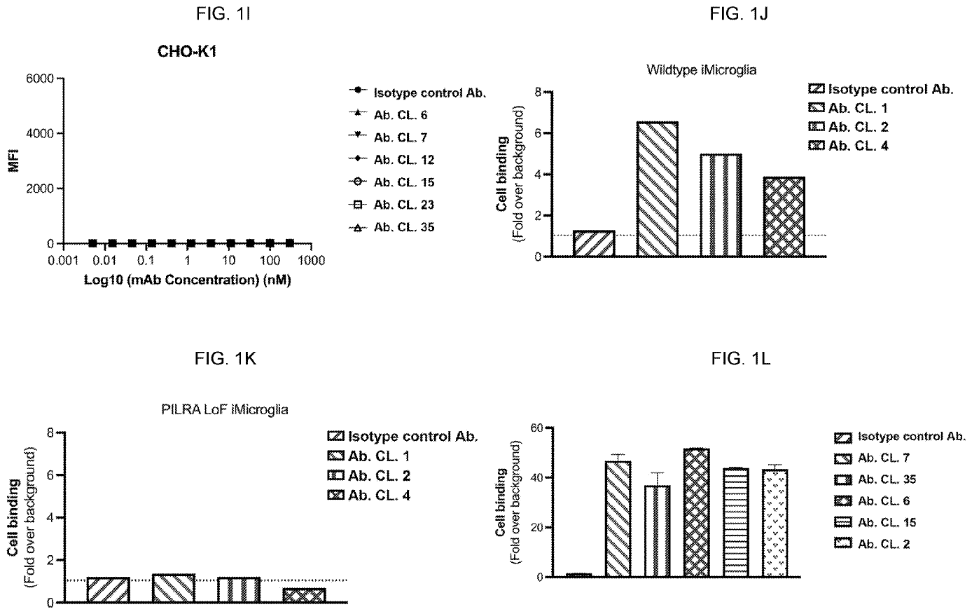

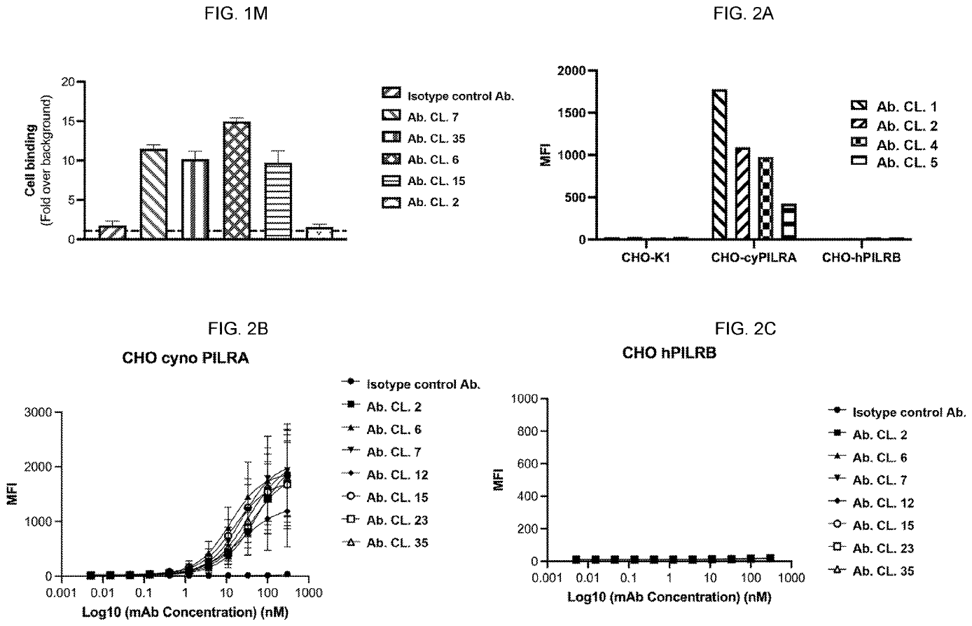

Parental HEK293 cells were labeled with NucBlue Live ReadyProbes Reagent for 30 mins. Parental HEK293 and hPILRA-expressing HEK293 cells were mixed, washed, and incubated with various concentrations of anti-PILRA or isotype control antibodies for 30 minutes on ice in FACS diluent (PBS 0.2% BSA and 1 nM EDTA). Cells were washed twice with FACS diluent, incubated with Alexa Fluor 647-conjugated anti-human IgG for 30 minutes on ice and wash once. Antibody binding to the cells was detected by FACS and median fluorescence intensity (MFI) was derived from data analysis performed with the FLOJO software (FIGS. 1A-1C).

CHO-K1 and hPILRA-expressing CHO-K1 cells were incubated with anti-PILRA or isotype control antibodies at a single concentration of 100 nM for 30 minutes on ice. Cells were washed twice with FACS diluent, then incubated with Alexa Fluor 647-conjugated anti-human IgG for 30 minutes on ice and washed once in FACS diluent. Antibody binding to the cells was detected by FACS and MFI was derived from data analysis performed with the FLOJO software (FIG. 1G). FIGS. 1A-1C and 1G together demonstrate binding of anti-PILRA antibodies to hPILRA expressed on HEK293 and CHO-K1 cells. The lack of binding of anti-PILRA antibodies to parental HEK293 and CHO-K1 cells also demonstrates specificity of binding.

Human microglia derived from wild-type IPSCs (human iMicroglia; PILRA R78/G78 heterozygous) or PILRA loss-of-function (LoF) IPSCs (human PILRA LoF iMicroglia) were dosed with 100 nM biotinylated anti-PILRA or isotype control antibodies for 45 minutes on ice. Cells were washed with PBS followed by 30 minutes incubation with Alexa Fluor 488-conjugated streptavidin for 30 minutes on ice. Cells were imaged using confocal microscopy after several PBS washes. Harmony Software was used to calculate mean fluorescent spot area per cell. Data is presented as fold expression over background signal in Alexa Fluor 488-conjugated streptavidin-only treated control wells (FIGS. 1J and 1K). FIGS. 1J and 1K together demonstrate binding of anti-PILRA antibodies to human iMicroglia, a CNS-relevant cell type, with endogenous cell surface levels of hPILRA. Further, the lack of binding of anti-PILRA antibodies to human PILRA LoF iMicroglia demonstrated the specificity of binding to hPILRA. Furthermore, the lack of binding of the isotype control antibody to both human iMicroglia and PILRA LoF iMicroglia demonstrated the lack of non-specific antibody binding to a CNS-relevant cell type.

PILRA mAb Binding to CHO Cells Expressing cynoPILRA or hPILRB

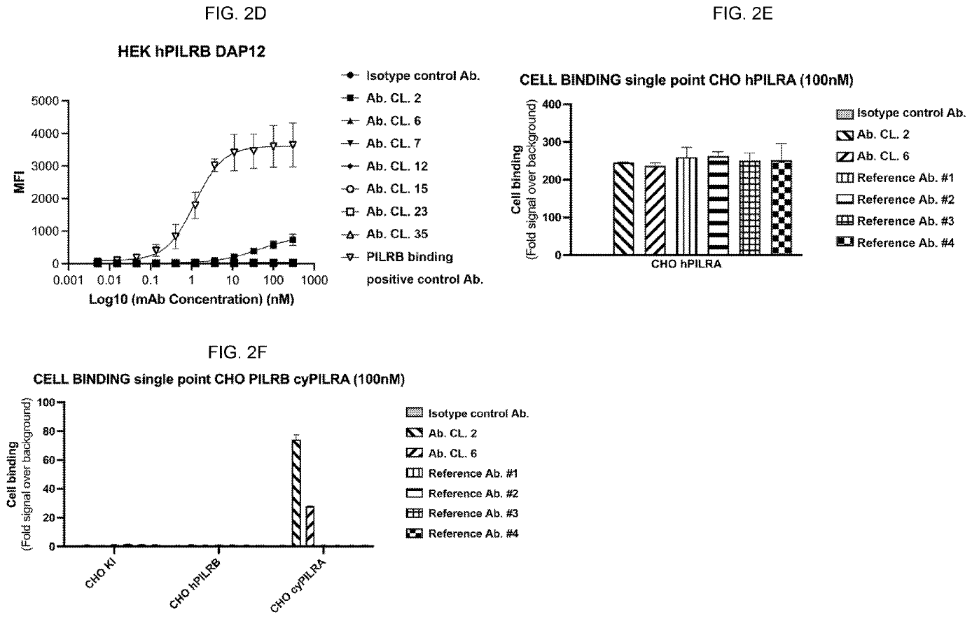

CHO-K1, cynoPILRA-expressing CHO-K1, and hPILRB-expressing CHO-K1 cells were incubated with anti-PILRA or isotype control antibodies at a single concentration of 100 nM for 30 minutes on ice. Cells were washed twice with FACS diluent, then incubated with Alexa Fluor 647-conjugated anti human IgG for 30 minutes on ice and washed once in FACS diluent. Antibody binding to the cells was detected by FACS and MFI was derived from data analysis performed with the FLOJO software. As shown in FIG. 2A, anti-PILRA antibodies bound to CHO-K1 cells expressing cynoPILRA, but there was no binding to CHO-K1 cells expressing hPILRB or to parental CHO-K1 cells. The binding of anti-PILRA antibodies to cynoPILRA expressed on CHO-K1 cells demonstrated the antibodies' cell surface target engagement and cyno cross-reactivity, which is a unique binding property that enables antibody safety assessment and TE/PK/PD studies in cynomolgus monkeys.

Overall, the anti-PILRA antibodies bound to hPILRA-expressing HEK293 and CHO-K1 cells, as well as cynoPILRA-expressing CHO-K1 cells, demonstrating binding specificity and cyno cross-reactivity. The antibodies also bound to human iMicroglia that express hPILRA at endogenous cell surface level and did not bind to PILRA LoF iMicroglia.

### Example 2—Characterization of Anti-PILRA Antibodies

### Binding Properties

The binding affinities of anti-PILRA antibodies to hPILRA, hPILRB, and cynoPILRA extracellular domain (ECD) were measured by SPR using a Biacore 8K instrument (Table 2). Antibodies were captured on Biacore™ Series S CM5 sensor chips immobilized with mouse anti-human Fab (human Fab capture kit from GE Healthcare) followed by injections of serial 3-fold dilutions of recombinant ECD reagents at a flow rate of 30 μL/min. Each sample was analyzed using a 3-minute association followed by a 10-minute dissociation. After each injection, the sensor chip was regenerated using a 5 0 mM glycine pH2.0 regeneration buffer. A 1:1 Languir model of simultaneous fitting of kₒₙ and k_{off} was used for kinetics analysis.

### Epitope Mapping

PILRA binding epitopes of anti-PILRA antibodies were identified by SPR using a Biacore 8K instrument. Anti-PILRA antibodies were captured on Biacore™ Series S CM5 sensor chips immobilized with mouse anti-human Fab (human Fab capture kit from GE Healthcare) followed by injections of single point PILRA to PILRB mutant variants at 1 μM concentration. For epitope binning, 1 μM recombinant human PILRA was injected for 300 seconds on a Biacore™ Series S CM5 sensor chip immobilized with anti-PILRA antibodies in each channel. Binding of secondary anti-PILRA antibody was monitored by subsequent injection of a single anti-PILRA antibody at each cycle.

### Ligand Blocking to Human PILRA

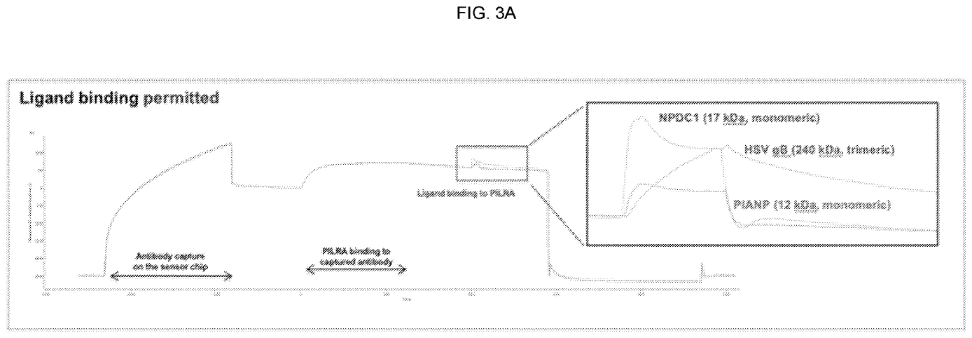

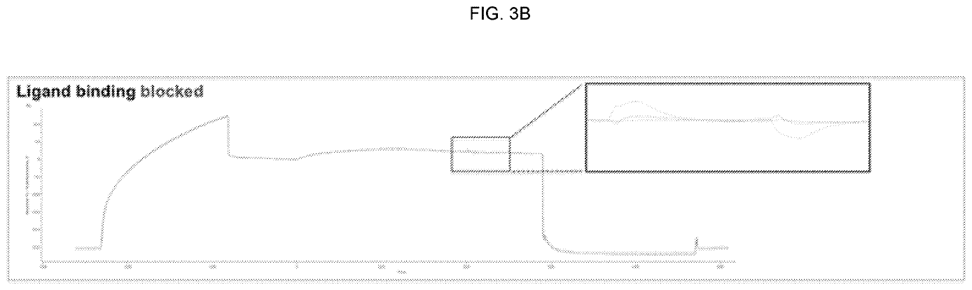

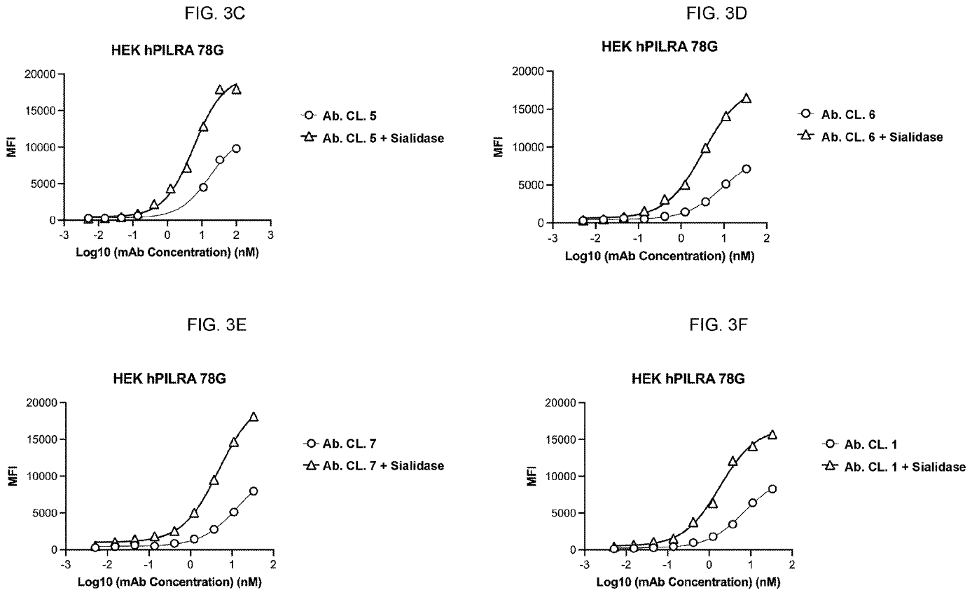

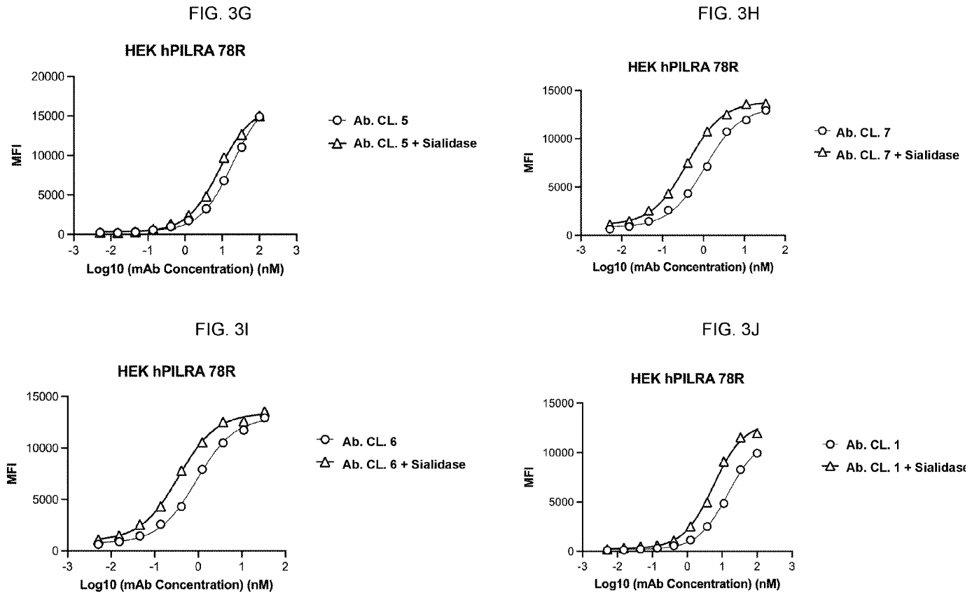

Ligand blocking characteristics of anti-PILRA antibodies were evaluated by SPR using a Biacore 8K instrument (FIGS. 3A and 3B). Anti-PILRA antibodies were captured on Biacore™ Series S CM5 sensor chips immobilized with mouse anti-human Fab (human Fab capture kit from GE Healthcare) followed by injections of 300 nM recombinant hPILRA ECD. hPILRA-ligand interactions were monitored by subsequent injection of recombinant PILRA ligands: hNPDC1(35-181), hPANP(76-178), and HSV gB(23-279), which are known sialyated ligands of PILRA. Blockage of the ligands to bind to hPILRA demonstrated that the antibodies were able to antagonize hPILRA.

Table 2 below shows the binding affinities of anti-PILRA antibodies to hPILRA G78, hPILRA R78, hPILRB, and cynoPILRA, the C50 binding values measured in HEK293 cells expressing hPILRA G78, the antibodies' binding epitopes of hPILRA, as well as whether the antibodies blocked various tested ligands.

TABLE 2

| Anti- | SPR Binding Affinities | | | | | | | Cell Binding | Human | |
|---|---|---|---|---|---|---|---|---|---|---|
| PILRA | Human | | Human | | | | | EC50 | PILRA | |
| Antibody | PILRA | | PILRA | | Human | Cyno | | (HEK293 | Binding | Ligand |
| Clone | G78 | | R78 | | PILRB | PILRA | | hPILRA G78) | Epitope | Blocking |
| 1 | 2.3 | nM | | | No | 910 | pM | 13.76 nM | G78, K106, | All tested |
| | | | | | binding | | | | E143 | ligands |
| 2 | 690 | pM | 270 | nM | No | 1.7 | nM | 12.51 nM | G78, K106, | All |
| | | | | | binding | | | | E143 | tested ligands |
| 3 | 1.7 | nM | | | No | 3.0 | nM | 14.09 nM | G78, K106, | All |
| | | | | | binding | | | | E143 | tested ligands |
| 4 | 8.3 | nM | | | No | 7.4 | nM | 24.88 nM | G78, K106, | All |
| | | | | | binding | | | | E143 | tested ligands |
| 5 | 660 | pM | | | No | 19 | nM | 32.52 nM | T63, A64 | All |
| | | | | | binding | | | | | tested ligands |
| 6 | 720 | pM | 9.6 | nM | 90 nM | 5.6 | nM | 9.30 nM | G78, K106, | All |
| | | | | | | | | | E143 | tested ligands |
| 7 | 1.2 | nM | 17 | nM | 250 nM | 4.8 | nM | 13.0 nM | G78, K106, | All |
| | | | | | | | | | E143 | tested ligands |
| 8 | 11 | nM | | | No | 59 | nM | 535.9 nM | T63, A64 | All |
| | | | | | binding | | | | | tested ligands |
| 9 | 1.6 | nM | | | No | No | | 1.283 nM | K106 | Blocks gB |
| | | | | | binding | binding | | | | only |
| 10 | 61 | nM | | | No | No | | 1.284 nM | 116-118 | Blocks gB |
| | | | | | binding | binding | | | | only |
| 11 | 140 | nM | | | No | 110 | nM | 10.50 nM | Stalk 2 | No |

| | | | | | |
|---|---|---|---|---|---|
| | binding | | | | |
| Reference | No | No | 639 pM | 116-118 | Blocks gB |
| Antibody | binding | binding | | | only |
| #1 | | | | | |
| Reference | No | No | 1.33 nM | 116-118 | Blocks gB |
| Antibody | binding | binding | | | only |
| #2 | | | | | |
| Reference | No | No | 1.56 nM | 116-118 | Blocks gB |
| Antibody | binding | binding | | | only |
| #3 | | | | | |
| Reference | No | No | 2.07 nM | 116-118 | Blocks gB |
| Antibody | binding | binding | | | only |
| #4 | | | | | |

As shown above, our anti-PILRA antibodies all demonstrated strong binding to hPILRA (both G78 and R78 variants) and cynoPILRA, while showed no binding or very weak binding to hPILRB. Further, 8 of the 11 antibodies blocked all tested ligands, while clone 9 and clone 10 blocked HSV gB(23-279). However, all four Reference Antibodies showed no binding to cynoPILRA and only blocked HSV gB(23-279).

### Example 3—Anti-PILRA Antibody-Induced Signaling Pathways

Evaluation of Phosphokinase Protein Activity in Human iMicroglia and PILRA LoF iMicroglia—EGFR and STAT3 Y705

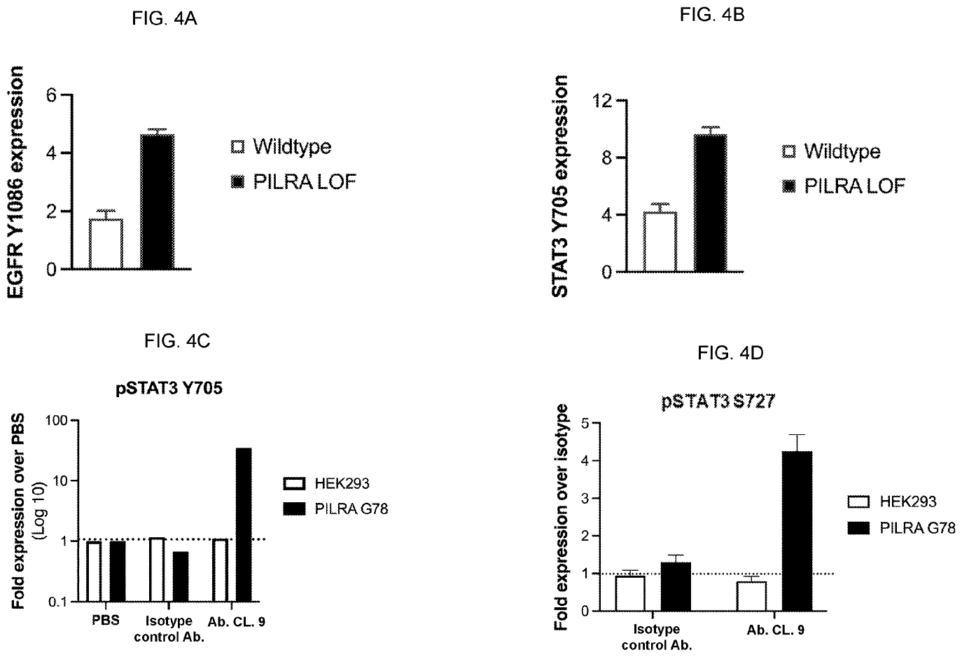

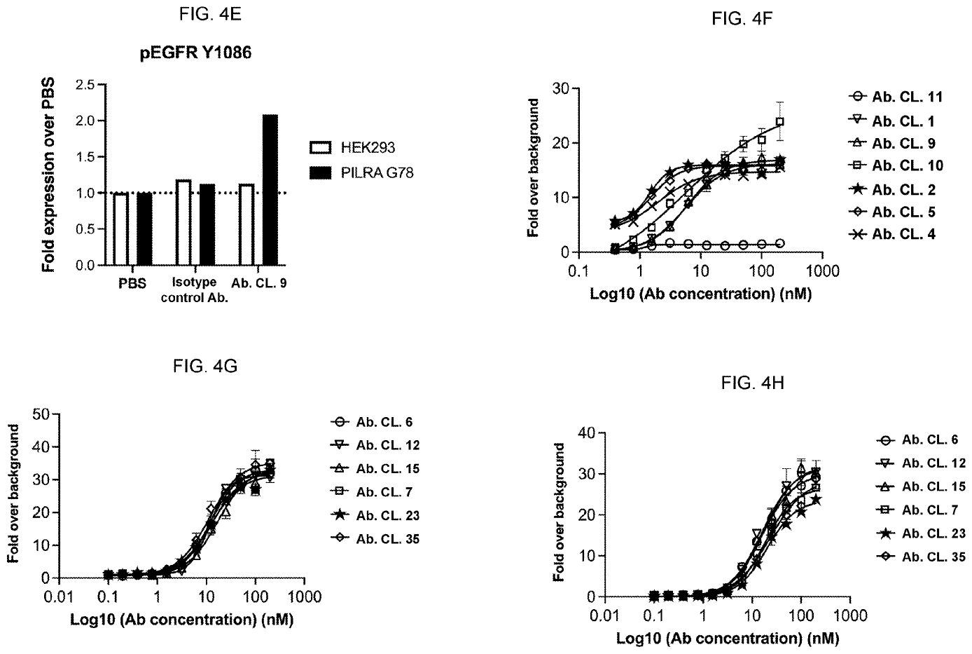

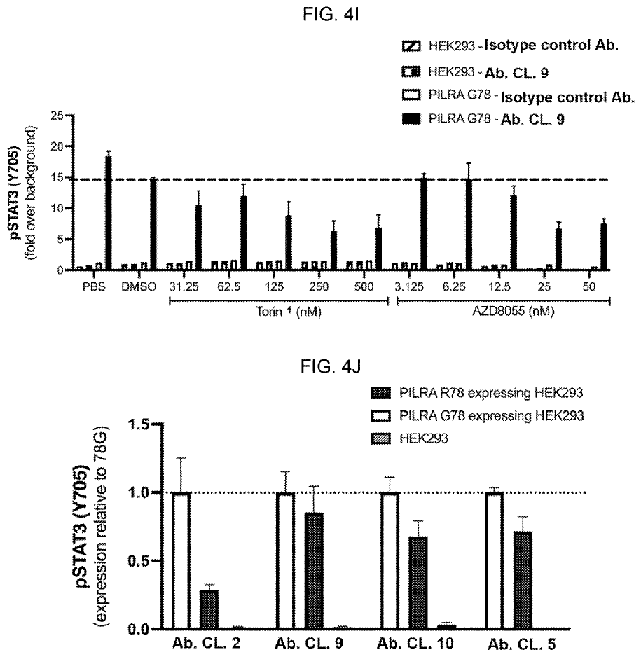

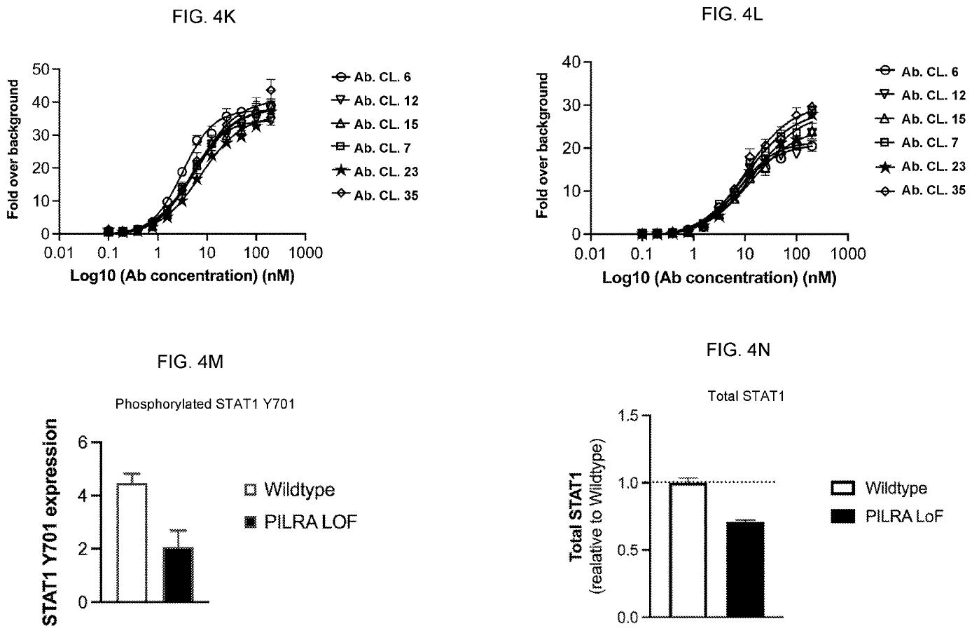

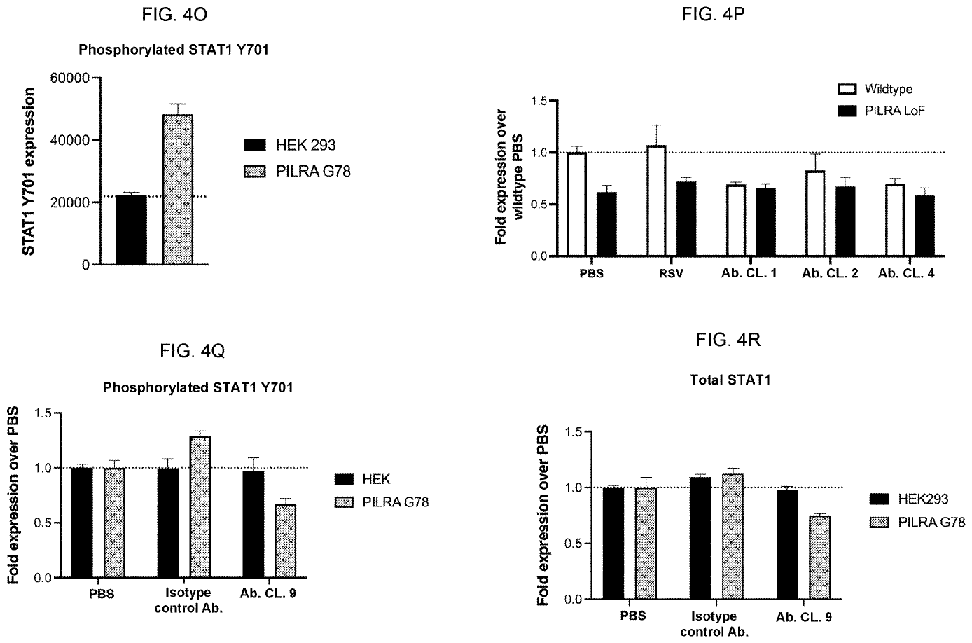

Understanding PILRA downstream signaling is crucial to identifying antagonistic antibodies. Wild-type human iMicroglia and PILRA LoF iMicroglia were plated at DIV 72 in serum containing media. Media was changed after 24 hours to remove serum and cells were lysed after 72 hours. Phosphokinase levels pEGFR Y1086 (FIG. 4A) and pSTAT3 Y705 (FIG. 4B) were measured using Proteome Profiler Human Phospho-Kinase Array Kit (ARY003C, R&D Systems) per manual instructions.

Evaluation of Anti-PILRA Antibody Downstream Signaling in HEK293 Cells Expressing hPILRA G78 or hPILRA R78—STAT3 Y705, STAT3 S727, and EGFR

Parental HEK293 cells or HEK293 cells expressing hPILRA G78 (PILRA with Gly at position 78) or hPILRA R78 (PILRA with Arg at position 78) were dosed with 100 nM anti-PILRA mAb or isotype control for 30-60 minutes in low serum condition (1% fetal bovine serum). Cells were fixed with 4% ice-cold paraformaldehyde and stained for pSTAT3 Y705 (dosed for 30 minutes; FIG. 4C), pSTAT3 S727 (dosed for 60 minutes; FIG. 4D), and pEGFR Y1086 (dosed for 60 minutes; FIG. 4E), or using standard immunocytochemistry protocol. Cells were imaged with a confocal microscope and images were analyzed in Harmony Software to calculate mean fluorescent spot area and intensity per cell.

To assess anti-PILRA mAb dose responsiveness, hPILRA G78-expressing HEK293 cells were dose titrated with anti-PILRA antibodies (<200 nM) for 30 minutes in low serum conditions (FIG. 4F). Table 3 lists the fold over background (fold induction of pSTAT3 Y705 for each antibody compared to isotype control antibody) and EC50 values showing nM potency for induction of pSTAT3 Y705 for each antibody. The dose-responsive induction of pSTAT3 Y705 in hPILRA G78-expressing HEK293 cells can be leveraged to rank order antagonistic antibodies based on potency and maximum effect. Induction of phosphorylated STAT3 Y705, STAT3 S727, and EGFR Y1086 in hPILRA G78-expressing HEK293 cells, but not in parental HEK293 cells, demonstrated specific PILRA-dependent downstream signaling. Further, the lack of signaling induction with isotype control antibody demonstrated PILRA selectivity and specificity.

TABLE 3

| Anti-PILRA Antibody | | |
|---|---|---|
| Clone | Fold Over Background | EC50 (nM) |
| 11 | 1.423 | N/A |
| 1 | 16.23 | 5.660 |
| 9 | 16.90 | 5.931 |
| 10 | 26.74 | 5.690 |
| 2 | 15.96 | 1.454 |
| 5 | 15.82 | 1.510 |
| 4 | 14.71 | 1.979 |

Humanized anti-PILRA antibodies were also tested for induction of phospho-STAT signaling. As shown in FIGS. 4G and 4H, anti-PILRA antibodies dose titrated on human PILRA 78G expressing HEK cells and induced pSTAT3 Y705 (FIG. 4G) or pSTAT3 S727 (FIG. 4H) after 30 minutes. EC50 values (Table 4) show nM potency for induction of pSTAT3 Y705 or pSTAT3 S727 for each antibody. Data is presented as mean+/−SEM fold expression over isotype control, n=2 biological replicates (FIG. 4G), n=2 technical replicates (FIG. 4H).

TABLE 4

| | Induction of | | Induction of | |
|---|---|---|---|---|
| Anti-PILRA | phospho-STAT3 (Y705) | | phospho-STAT3 (S727) | |
| Antibody | Fold Over | | Fold Over | |
| Clone | Background | EC50 (nM) | Background | EC50 (nM) |
| 6 | 33.12 | 12.07 | 30.06 | 16.00 |
| 12 | 31.31 | 11.67 | 31.60 | 16.04 |
| 15 | 33.47 | 16.95 | 32.19 | 17.53 |
| 7 | 32.53 | 12.98 | 26.59 | 18.62 |
| 23 | 31.39 | 12.19 | 23.57 | 20.11 |
| 35 | 35.53 | 11.39 | 28.89 | 23.01 |

Further, as shown in FIGS. 4K and 4L, anti-PILRA antibodies dose titrated on human PILRA 78R expressing HEK293 cells and induced pSTAT3 Y705 (FIG. 4K) or pSTAT3 S727 (FIG. 4L) after 30 minutes. EC50 values (Table 5) show nM potency for induction of pSTAT3 Y705 for each antibody. Data is presented as mean+/−SEM fold expression over isotype control, n=3 biological replicates (FIG. 4K), n=2 technical replicates (FIG. 4L).

TABLE 5

| | Induction of | | Induction of | |
|---|---|---|---|---|
| Anti-PILRA | phospho-STAT3 (Y705) | | phospho-STAT3 (S727) | |
| Antibody | Fold Over | | Fold Over | |
| Clone | Background | EC50 (nM) | Background | EC50 (nM) |
| 6 | 37.64 | 3.177 | 21.01 | 7.133 |
| 12 | 34.64 | 4.533 | 21.45 | 7.412 |
| 15 | 38.21 | 5.655 | 24.78 | 12.65 |
| 7 | 38.30 | 5.378 | 28.85 | 10.70 |
| 23 | 36.18 | 7.547 | 28.49 | 14.57 |
| 35 | 41.03 | 6.193 | 30.05 | 10.57 |

The dose-responsive induction of pSTAT3 (Y705) and/or pSTAT3 (S727) in hPILRA 78R or 78G expressing HEK293 cells could be leveraged to rank order antagonistic antibodies based on potency and maximum effect.

To assess if pSTAT3 Y705 induction is mTOR dependent, cells were dosed with mTOR inhibitors Torin 1 (31.25-500 nM) or AZD8055 (3.125-50 nM) for 2 hours before spiking in 100 nM anti-PILRA mAb or isotype control for 30 mins. FIG. 4I shows that pSTAT3 Y705 induced by anti-PILRA antibodies was partially blocked by mTOR inhibitors.

Further, FIG. 4J shows that anti-PILRA antibodies induced pSTAT3 Y705 in AD-protective PILRA R78 expressing HEK293 cells. Anti-PILRA antibody that binds to PILRA G78 (Ab. Clone 2) partially blocked induction of pSTAT3 Y705 in HEK293 cells expressing the AD-protective PILRA R78. Anti-PILRA antibodies that bind to non-G78 epitopes (Ab. Clones 9, 10, and 5) showed similar pSTAT3 Y705 induction in PILRA G78 and PILRA R78. There was no pSTAT3 induction in parental HEK293 cells. The AD-protective PILRA variant R78 has reduced ligand binding capacity and likely also less affinity to antibodies that bind to G78 (e.g., Ab. Clones 2 and 4). The frequency of this AD-protective PILRA variant R78 varies around the world. It is the minor allele in African (10%) and European (38%) populations, but the major allele (65%) in East Asian populations. Anti-PILRA antibodies that bind to PILRA R78 could be associated with loss-of-affinity in a large fraction of people. However, Ab. Clone 5, which binds to a different epitope, induced robust downstream signaling (pSTAT3 Y705) in cells expressing the R78 variant of PILRA. Such anti-PILRA antibodies could help to de-risk the loss-of-potency for PILRA R78 expressing cells seen with antibodies that bind to G78, e.g., Ab. Clone 2.

Evaluation of STAT1 in Human iMicroglia, PILRA LoF iMicroglia, and HEK293 Cells

Wild-type human iMicroglia and PILRA LoF iMicroglia were plated at DIV 45 in serum containing media. Media was changed after 24 hours to remove serum. Cells were dosed with 100 nM anti-PILRA antibody 4 days post plating and lysed after 30 minutes. Phosphorylated STAT1 Y701 levels (FIG. 4M) were measured using AlphaLisa assay per manual instructions.

Wild-type human iMicroglia and PILRA LoF iMicroglia were plated at DIV 58 in serum containing media. Media was changed after 24 hours to remove serum and cells were lysed after 72 hours. Total STAT1 levels (FIG. 4N) were measured using AlphaLisa assay per manual instructions. Phospho-STAT1 Y701 levels (FIG. 4O) were also measured in lysed parental or hPILRA G78-expressing HEK293 cells using AlphaLisa assay per manual instructions.

Anti-PILRA antibodies were used to dose wild-type human iMicroglia and PILRA LoF iMicroglia. As shown in FIG. 4P, only 30 minutes of dosing with anti-PILRA antibodies at 100 nM reduced phosphylated STAT1 Y701 levels, mimicking the phenotype of PILRA LoF iMicroglia. Anti-PILRA antibodies were also used to dose parental HEK293 cells and HEK293 cells expressing PILRA G78. As shown in FIGS. 4Q and 4R, anti-PILRA mAb reduced phosphorylated STAT1 Y701 and total STAT1 levels in HEK293 cells expressing PILRA G78. There was no reduction in parental HEK293 cells or by isotype control antibody.

Overall, as the results showed, basal changes in the phosphorylation states of EGFR, STAT3, and STAT1 in anti-PILRA antibody dosed wild-type and PILRA LoF iMicroglia suggested that these pathways are downstream of PILRA, which is a significant discovery related to PILRA biology that was never realized before.

### Example 4—Evaluation of PILRA-Dependent iMicroglial Migration

Understanding PILRA dependent functions inhuman IPSC-derived microglia in vitro could help predicting function in vivo. Enhanced microglial motility could be beneficial in neurodegenerative diseases.

Wild-type human iMicroglia, PILRA LoF iMicroglia, and PILRA LoF iMicroglia expressing hPILRA (PILRA LoF OE) at DIV 53 were plated at 20,000 cells per well in 96-well plates with a rubber stopper creating a central cell-free detection zone. Anti-PILRA antibodies (100 nM), isotype control (100 nM), or PBS (Wild-type human iMicroglia and PILRA LoF iMicroglia samples) was added in fresh media and rubber stopper removed on day 2. NucBlue was added on day 6 and the cells were imaged using confocal microscopy. Images were analyzed in Harmony Software to calculate mean area of nuclear labeling in detection zone.

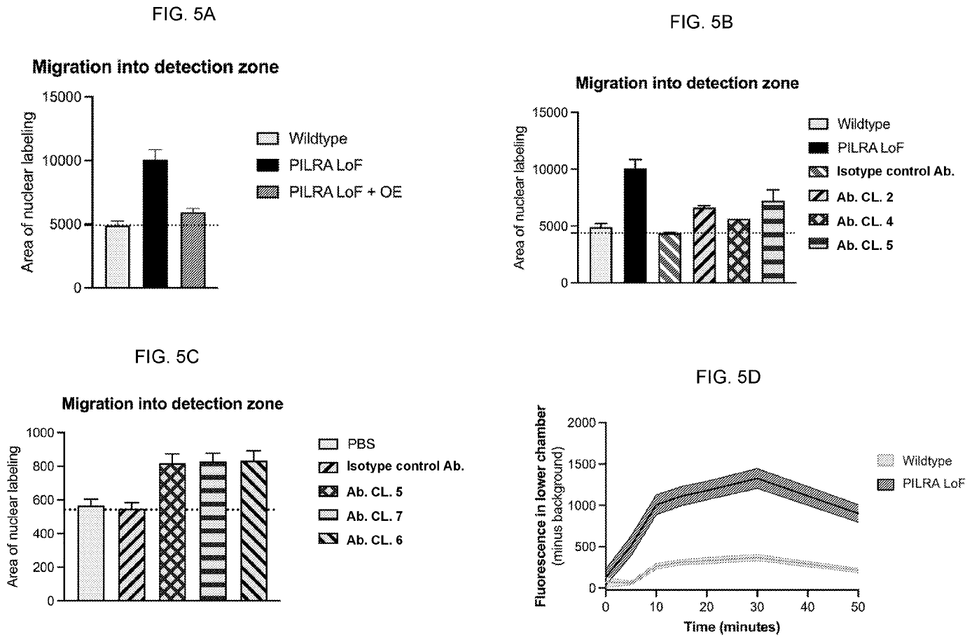

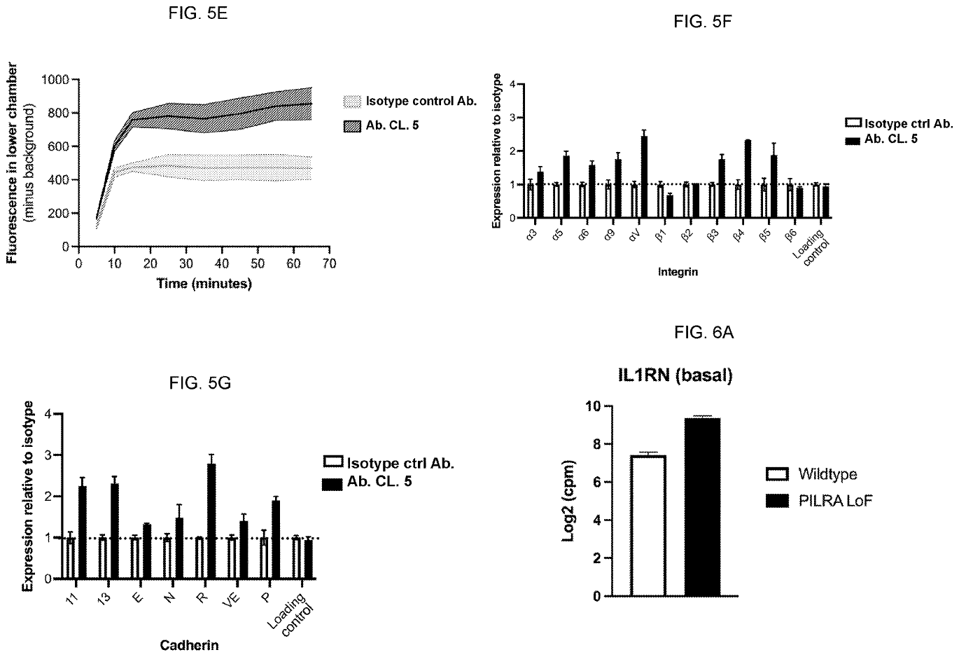

Re-expression of hPILRA in PILRA LoF iMicroglia reversed the migration phenotype back to wild-type levels, demonstrating that migration is a PILRA dependent and specific endpoint (FIG. 5A). Anti-PILRA antibodies that phenocopy PILRA LoF iMicroglia functions are classified as functional antagonists. As shown in FIG. 5B and FIG. 5C, anti-PILRA antibodies enhanced wild-type iMicroglial migration to cell-free detection zone 120 hours after stopper removal, similar to PILRA LoF iMicroglia cells. The lack of migration phenotype with isotype control antibody demonstrated specificity. PILRA LoF iMicroglia and wild-type iMicroglia were both only dosed with vehicle (PBS).

Further, anti-PILRA antibodies were shown to enhance cell migration to chemoattractant complement 5a (C5a). Wildtype and PILRA LoF iMicroglia cells at DIV 62 (FIG. 5D) or at DIV 110 (FIG. 5E) were harvested and subsequently labeled with calcein-AM dye for transwell assay. Cells in FIG. 5E were pre-treated with anti-PILRA antibody or isotype control (100 nM) for 4 days before harvest. Human Complement 5a (10 ng/ml) was added as chemoattractant to lower chamber at time 0. Data is presented as mean+/−SEM, n=3 technical replicates. FIGS. 5D and 5E show that PILRA LoF enhanced iMicroglial migration to chemoattractant complement 5a (C5a) (FIG. 5D) and anti-PILRA antibodies enhanced chemotaxis of iMicroglia to C5a, similar to PILRA LoF iMicroglia cells (FIG. 5E).

Anti-PILRA antibodies also enhanced microglial secretion of motile proteins. Human iMicroglia derived from wildtype (PILRA R78/G78 heterozygous) IPSCs were plated in serum containing media at DIV 42. Media was changed after 24 hours to remove serum and cells were dosed with anti-PILRA antibody clone 5 (100 nM) or isotype control for 4 days. Soluble analytes in the supernatants dosed with anti-PILRA antibody clone 5 or isotype control were analyzed with Proteome profiler kit; Human Soluble Receptor Array Kit—Non-Hematopoietic Panel (R&D ARY012). Data is presented as mean+/−SD expression relative to isotype, n=2 technical replicates. FIGS. 5F and 5G show that anti-PILRA antibodies enhanced iMicroglial secretion of integrins (FIG. 5F) and cadherins (FIG. 5G) into the supernatant after 4 days of treatment.

This example demonstrates that anti-PILRA antibodies acted as functional antagonists to PILRA and phenocopied PILRA LoF iMicroglia functions by enhancing cell migration. The antibodies also increased cell secretion of cadherins and integrins, which are motile proteins associated with enhanced cell migration.

### Example 5—Effect of PILRA on Anti-Inflammatory Phenotype in iMicroglia

Understanding PILRA-dependent functions in human iMicroglia in vitro could help to predict function in vivo. Modulation of inflammation state could be beneficial in neurodegenerative diseases. To assess PILRA-dependent transcriptional changes, wildtype iMicroglia and PILRA LoF iMicroglia were plated in serum containing media. Media was changed after 24 hours to remove serum. LPS (10 ng/ml) or vehicle was added 72 hours later to stimulate cytokine response. Cells were collected at 24 h post LPS treatment and RNA was isolated from 5 independent harvests (DIV 59, 63, 70, 73, 77) obtained from the same differentiation batch of wildtype iMicroglia or PILRA LoF iMicroglia. Two technical reps were pooled for each condition per harvest.

To assess PILRA-dependent IL1RA cytokine secretion, wild-type iMicroglia and PILRA LoF iMicroglia were plated in serum-containing media at DIV70. Media was changed after 24 hours to remove serum and dose cells with antibodies (100 nM). Supernatants were collected 72 hours after media change and run on human IL1RA MSD.

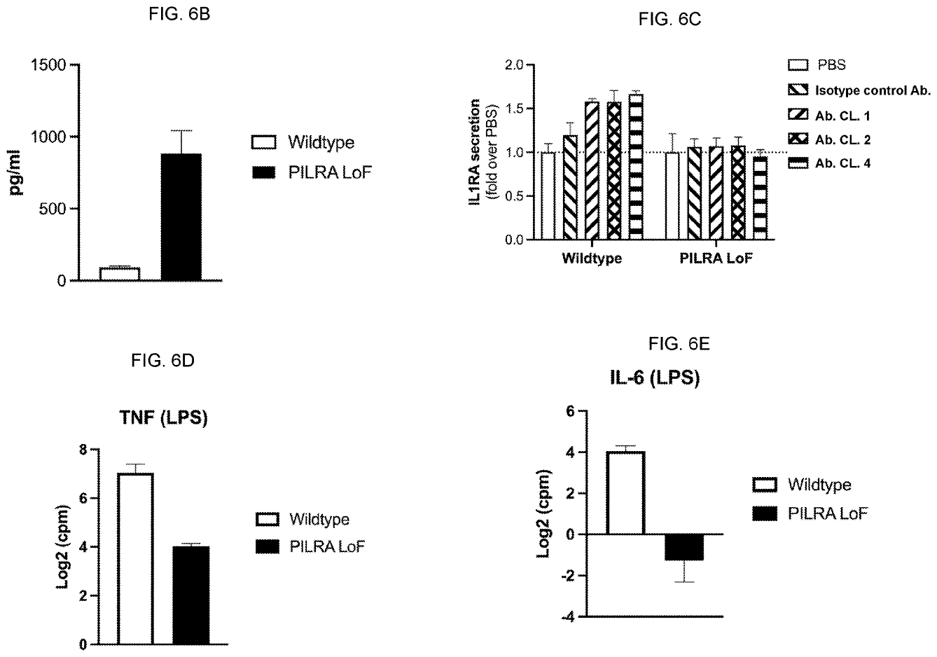

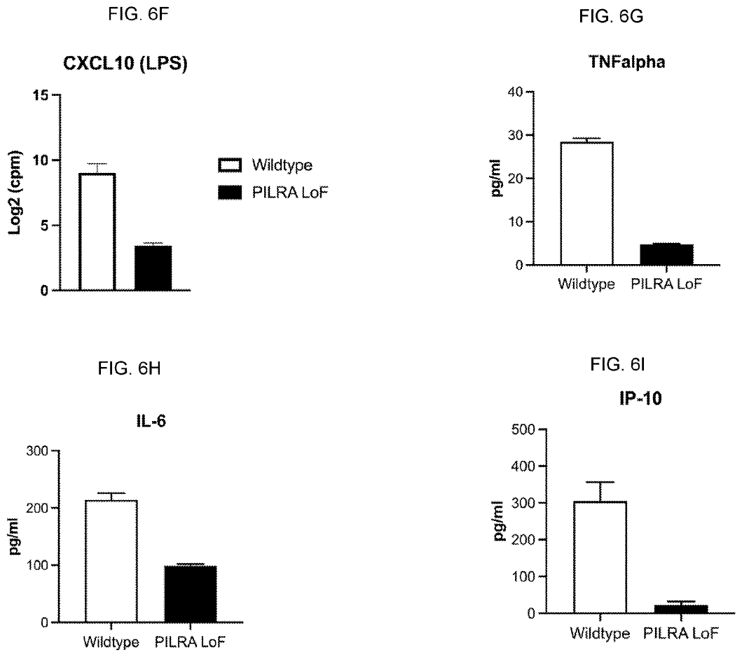

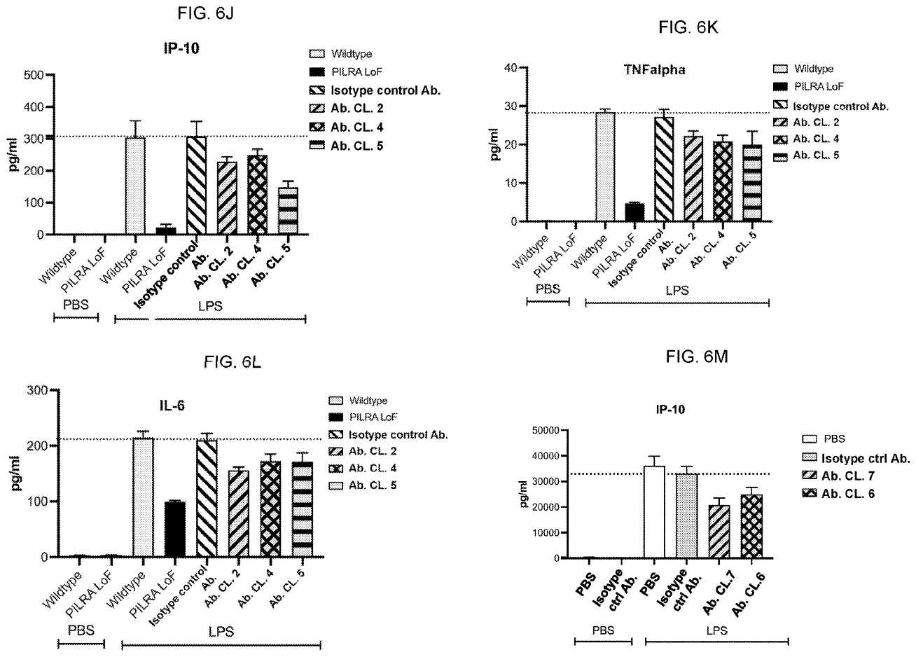

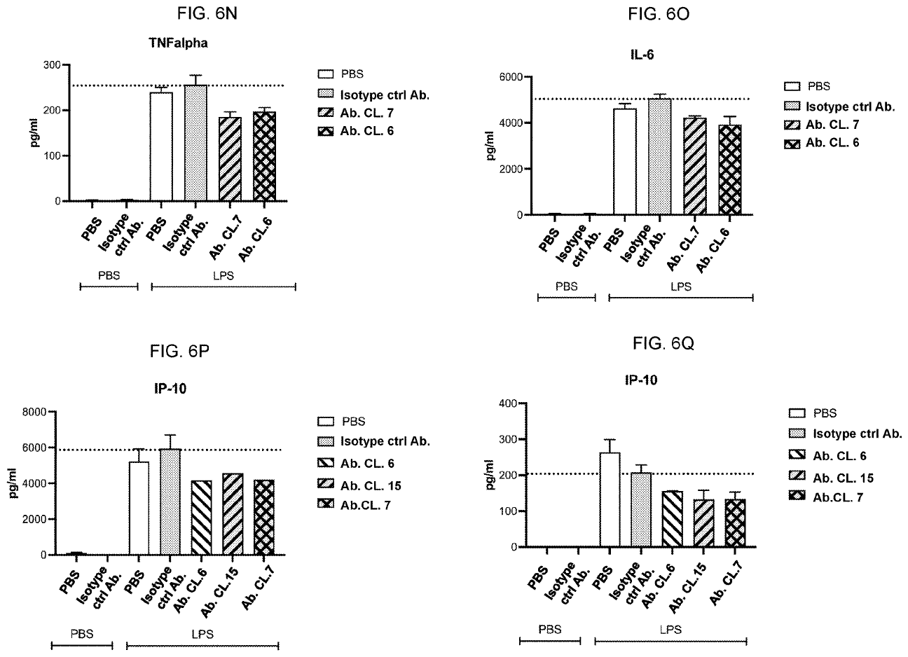

For IL1RN transcriptional change and IL1RA cytokine secretion, PILRA LoF promoted IL1RN gene expression (FIG. 6A) and IL1RA cytokine secretion (FIG. 6B) in PILRA LoF iMicroglia compared to wild-type iMicroglia in serum-free media. Further, anti-PILRA antibodies (100 nM, 72 hours) stimulated IL1RA cytokine secretion in wild-type iMicroglia in serum-free media, mimicking the phenotype observed in PILRA LoF iMicroglia (FIG. 6C). Anti-PILRA antibodies did not increase IL1RA secretion in PILRA LoF iMicroglia.

To assess LPS-induced PILRA-dependent pro-inflammatory cytokine secretion, wild-type iMicroglia and PILRA LoF iMicroglia were plated in serum containing media at DIV53. Media was changed after 24 hours to remove serum and wild-type cells were dosed with antibodies (100 nM). LPS (10 ng/ml) was added 72 hours later to stimulate cytokine response. Supernatants were collected at 24 h post LPS treatment and run on human pro-inflammatory (4-plex) MSD and human IP-10 MSD.

For LPS-induced transcriptional changes, PILRA LoF suppressed LPS-induced TNF, IL-6, and CXCL10 gene expressions in PILRA LoF iMicroglia relative to wild-type iMicroglia (FIGS. 6D-6F). For LPS-induced changes in cytokine secretion, PILRA LoF suppressed LPS-induced secretions of TNFalpha, IL-6, and IP-10 in PILRA LoF iMicroglia relative to wild-type iMicroglia (FIGS. 6G-6I).

Moreover, anti-PILRA antibodies were able to attenuate LPS-induced IP-10, TNFalpha, and IL-6 cytokine secretion in wild-type iMicroglia, mimicking the phenotype observed in PILRA LoF iMicroglia (FIGS. 6J-6O).

Further, homozygous G78 or R78 PILRA expressing iMicroglia cells were also used to test the anti-inflammatory effects of the antibodies. CRISPR mediated KI lines were generated to determine antibody impact on homozygous G78R PILRA (AD-protective; R78) and R78G PILRA (normal AD-risk; G78) genetic variants. Microglia were plated in serum-containing media at DIV54. Media was changed after 24 hours to remove serum and iMicroglia cells were dosed with antibodies (100 nM). LPS (10 ng/ml) was added 72 hours later to stimulate cytokine response. Supernatants were collected at 24 h post LPS treatment and run on human IP-10 MSD. Data is presented as mean+/−SEM. FIGS. 6P and 6Q show anti-PILRA antibodies (100 nM) attenuated LPS-induced IP-10 cytokine secretion in homozygous G78 (FIG. 6P) and R78 (FIG. 6Q) PILRA expressing IPSC-derived iMicroglia in serum-free media. Anti-PILRA antibodies promoted anti-inflammatory phenotype in IPSC-derived Microglia with either PILRA allele combination (R78/R78, R78/G78, or G78/G78) which demonstrated antagonistic function in a CNS-relevant cell type with endogenous cell surface levels of hPILRA receptor.

This example demonstrates that anti-PILRA antibodies stimulated IL1RA cytokine secretion in wild-type iMicroglia, mimicking the phenotype observed in PILRA LoF iMicroglia. Further, anti-PILRA antibodies attenuated the LPS-induced cytokine secretions in wild-type iMicroglia and IPSC-derived iMicroglia with endogenous level of hPILRA. Overall, the anti-PILRA antibodies promoted anti-inflammatory phenotype.

### Example 6—Effect of PILRA on Mitochondrial Respiration

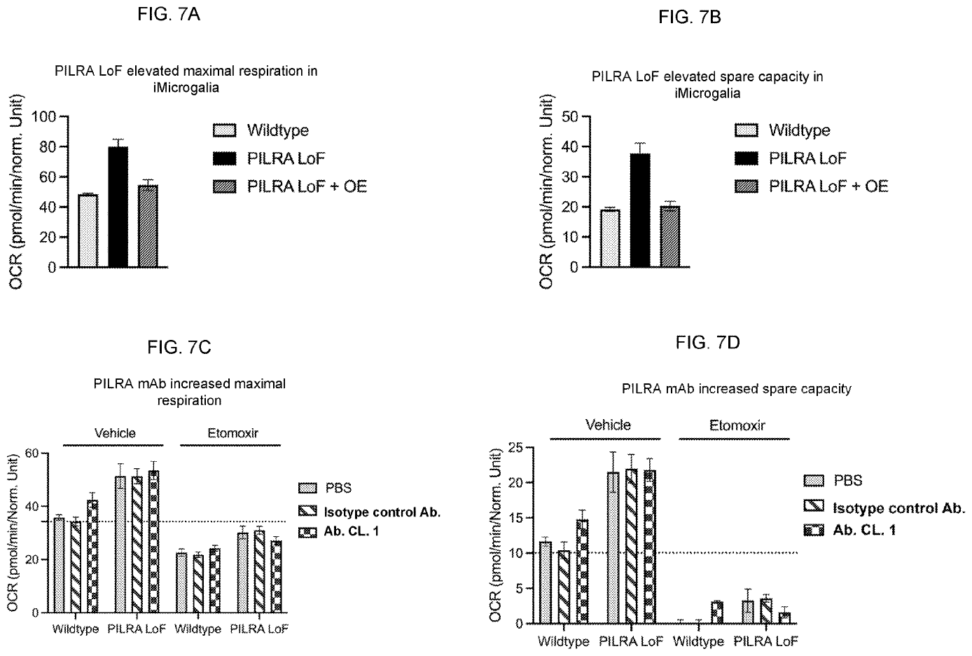

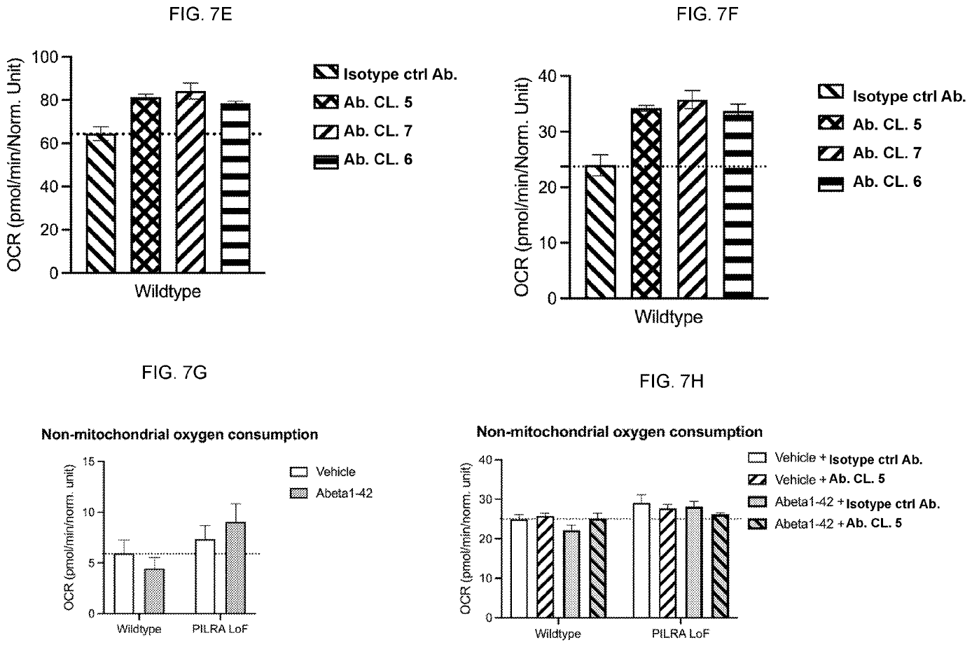

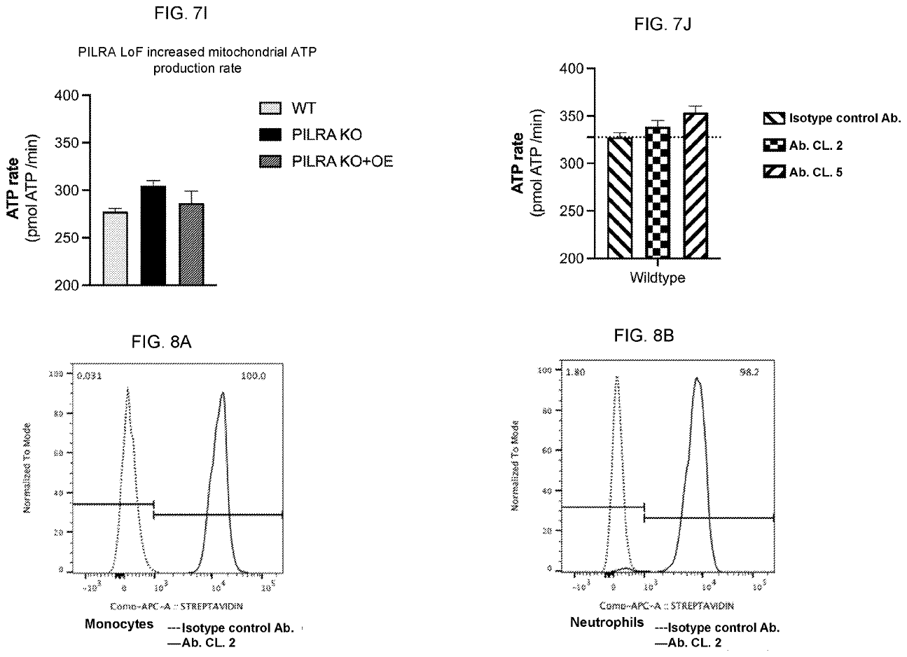

Metabolic dysfunction in microglia are pathological hallmarks of neurodegenerative disease. To assess PILRA-dependent effects on mitochondrial respiration at basal conditions, wild-type iMicroglia, PILRA LoF iMicroglia, and PILRA LoF iMicroglia expressing hPILRA (DIV 51) were plated at a density of 20 k/well in a pre-coated 96-well plate suited for Seahorse XF assays. Serum-containing media (C+++) was replaced with substrate limited media (SLM) to prime cells for fat metabolism 72 h-post plating for an overnight period. On the day of assay, the Seahorse long-chain fatty acid oxidation kit was performed according to manufacturer instructions, with sequential injections of oligomycin (1.5 μM), FCCP (2 μM), and rotenone/antimycin (0.5 μM) for the determination of mitochondrial fitness and capacity. As shown in FIGS. 7A and 7B, PILRA LoF iMicroglia displayed elevated maximal respiration and spare mitochondrial capacity. Re-expression of PILRA in PILRA LoF iMicroglia expressing hPILRA (PILRA LoF+OE) restored mitochondrial respiration to wild-type levels.

To assess anti-PILRA mAb-dependent effects on mitochondrial respiration, wildtype and PILRA LoF iMicroglia cells (DIV 67) were plated at a density of 20 k/well in a pre-coated 96-well plate suited for Seahorse XF assays. Serum-containing media (C+++) was replaced with substrate limited media (SLM) to prime cells for fat metabolism 24 h-post plating. Anti-PILRA antibodies were dosed at 100 nM during substrate limitation for a treatment duration of 72 h. On the day of assay, the Seahorse long-chain fatty acid oxidation kit was performed according to manufacturer instructions, with sequential injections of etomoxir (4 μM), oligomycin (1.5 μM), FCCP (2 μM), and rotenone/antimycin (0.5 μM) for the determination of mitochondrial fitness and capacity. Anti-PILRA antibodies (100 nM) increased maximal respiration (FIG. 7C and FIG. 7E) and spare mitochondrial respiratory capacity (FIG. 7D and FIG. 7F) in wildtype iMicroglia relative to isotype control. There was no additional impact of antibodies on PILRA LoF iMicroglia, which indicates antibody specificity. Antibody treatment and genotype effects were mitigated by carnitine palmitoyltransferase 1 (CPT1) inhibition, suggesting that fatty acid oxidation is a significant driver of improved mitochondrial function.

To assess the effect of PILRA LoF on Abeta1-42 fibril-induced reduction in non-mitochondrial oxygen consumption rate, wild-type and PILRA LoF iMicroglia (DIV 67) were plated at a density of 20 k/well in a pre-coated 96-well plate suited for Seahorse XF assays. Serum-containing media (C+++) was replaced with glutamine-free DMEM XF assay media containing Abeta1-42 fibrils (100 nM). 10-min sonication of fibrils prior to overnight cell treatment was performed. One day of assay, the Seahorse mitostress kit was performed according to manufacturer instructions, with sequential injections of oligomycin (1.5 μM), FCCP (1 μM), and rotenone/antimycin (0.5 μM) for the determination of mitochondrial fitness and capacity. To assess the effect of anti-PILRA mAb, wild-type and PILRA LoF iMicroglia (DIV 80) were plated at a density of 20 k/well. Anti-PILRA antibodies were dosed 24 h post-plating at 100 nM and supplemented during change to glutamine-free DMEM XF media. As shown in FIGS. 7G and 7H, Abeta1-42 fibril-induced reduction in non-mitochondrial oxygen consumption rate in wild-type iMicroglia (gray bar in FIG. 7G) can be rescued with anti-PILRA antibody (striped gray bar in FIG. 7H).

To assess PILRA-dependent effects on mitochondrial ATP production rate, wild-type iMicroglia, PILRA LoF iMicroglia, and PILRA LoF iMicroglia expressing hPILRA (DIV 61) were plated at a density of 20 k/well in a pre-coated 96-well plate suited for Seahorse XF assays. Cells were incubated in serum-containing media for 72 hours and subsequently, Seahorse ATP rate kit was performed according to manufacturer instructions in DMEM XF assay media. Sequential injections of oligomycin (1.5 μM) and rotenone/antimycin (0.5 μM) were applied for the determination of ATP production rate (FIG. 7I). To assess the effect of anti-PILRA mAb, wild-type iMicroglia (DIV40) were plated at a density of 20 k/well. Anti-PILRA antibodies were dosed 24 h post-plating at 100 nM and supplemented during change to DMEM XF media. PILRA LoF iMicroglia exhibited higher mitochondrial OXPHOS activity with increased ATP production (FIG. 7I). Anti-PILRA antibodies recapitulated PILRA LoF in wild-type iMicroglia with enhanced rate ATP generation (FIG. 7J).

Overall, anti-PILRA antibodies enhanced mitochondrial activity in wild-type iMicroglia, including maximal respiration capacity, spare reserve, and rate of ATP production. Anti-PILRA antibodies also enhanced fatty acid oxidation in wild-type iMicroglia, mimicking the phenotype observed in PILRA LoF iMicroglia, suggesting a potentially greater capacity to metabolize accumulating lipid substrates in a disease context. Further, non-mitochondrial respiration was also enhanced by PILRA LoF and anti-PILRA antibodies. Non-mitochondrial respiration is primarily via NOX2 superoxide production, which could have detrimental effects, such as lipid and protein oxidation, if left uncontrolled.

### Example 7—Effect of PILRA on Peripheral Immune Cells

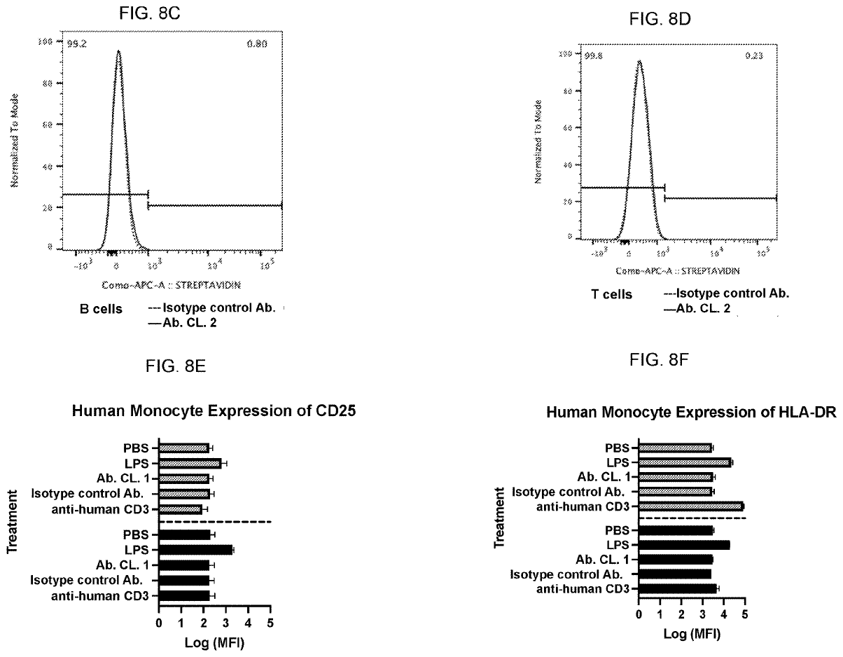

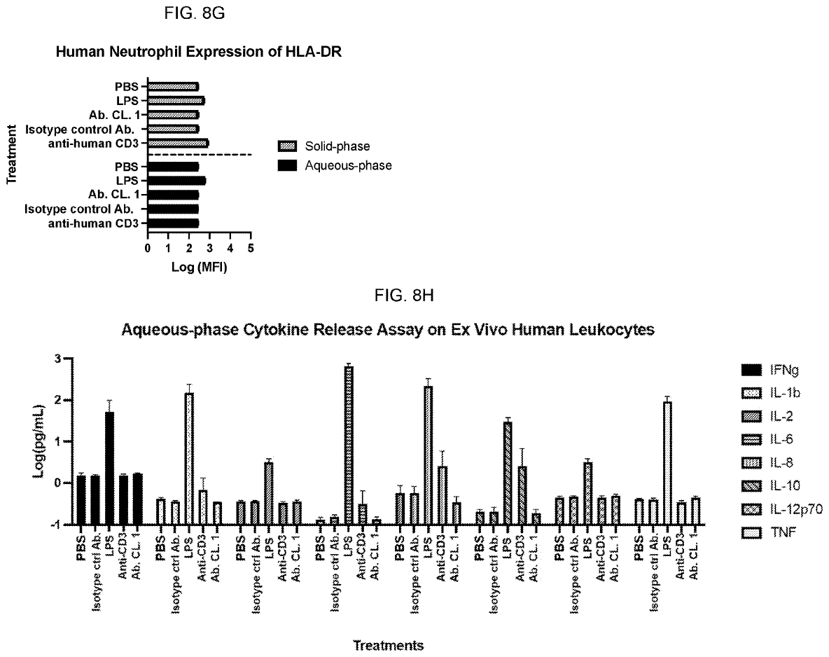

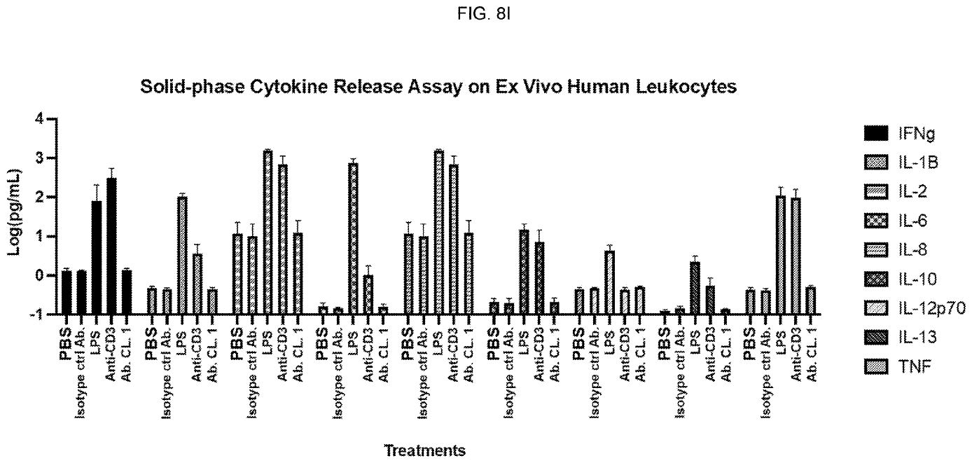

Human PILRA is expressed on neutrophils and monocytes. Binding of anti-PILRA antibodies to these peripheral immune cells allows for peripheral evaluation of therapeutic equivalence. To assess anti-PILRA mAb binding, human leukocytes were enriched from heparinized whole blood by hypotonic lysis of erythrocytes and resuspended in cold PBS containing 0.5% BSA and 2 mM EDTA. Fc receptors were blocked with Human TruStain FcX. Cells were then labelled with fluorescent antibodies against CD3, CD14, CD19, CD45, CD66b, and PILRA. Fluorescence intensity was quantified by flow cytometry. Anti-PILRA antibody was able to bind ex-vivo to monocytes (FIG. 8A) and neutrophils (FIG. 8B). There was no binding to B-cells and T-cells (FIGS. 8C and 8D).

To assess if anti-PILRA mAb activate neutrophils and monocytes, human leukocytes were enriched from heparinized whole blood by hypotonic lysis of erythrocytes and resuspended in complete RPMI 1640 cell culture media. Cells were treated with antibodies at 100 nM or LPS at 10 ng/mL in both aqueous-phase and solid-phase for 24 hours. They were then washed and labeled with fluorescent antibodies against CD11b, CD14, CD25, CD66b, and HLA-DR. Fluorescence intensity was quantified by flow cytometry. Anti-PILRA antibodies did not activate ex vivo human neutrophils and monocytes. Anti-PILRA antibody-treated cells did not show elevated CD25 (FIG. 8E) or HLA-DR (FIGS. 8F and 8G). Expressions of CD25 and HLA-DR were compared to positive (i.e., LPS-treated and anti-CD3 antibody-treated) and negative (i.e., isotype control-treated and PBS-treated) controls.

To assess if anti-PILRA mAb activate ex vivo human leukocytes, human leukocytes were enriched from heparinized whole blood by hypotonic lysis of erythrocytes and resuspended in complete RPMI 1640 cell culture media. Cells were treated with antibodies at 100 nM or LPS at 10 ng/mL in both aqueous-phase and solid-phase for 24 hours. Supernatants were collected after centrifugation at 1000 g for 20 minutes and stored at 80° C. Soluble proteins were quantified using the MSD Human Proinflammatory Panel I kit. As shown in FIGS. 8H and 8I, ex vivo human leukocytes did not increase production of proinflammatory cytokines after treatment with aqueous-phase (FIG. 8H) or solid-phase (FIG. 8I) anti-PILRA antibodies at 100 nM for 24 hours.

Overall, anti-PILRA antibodies did not have any major impact on myeloid cell activation ex vivo based on absence of key cell surface markers and secreted pro-inflammatory cytokines.

### Example 8—Anti-PILRA Antibody Epitope Bins

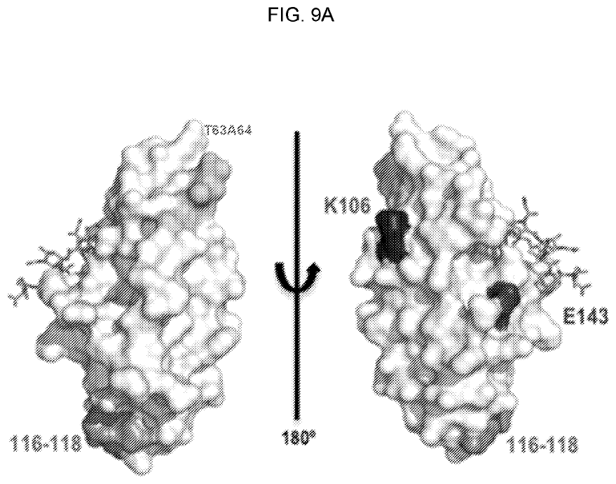

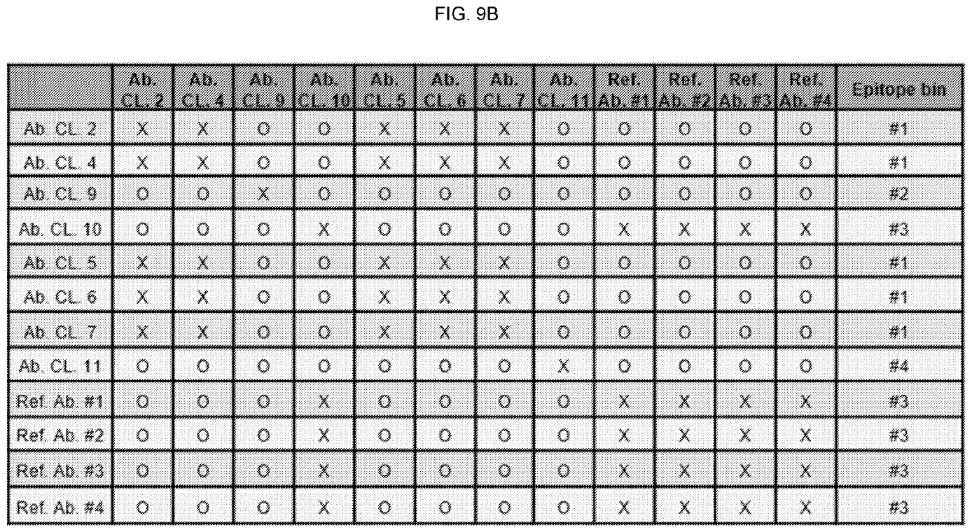

PILRA binding epitopes of anti-PILRA antibodies described herein and four reference anti-PILRA antibodies were identified by SPR using a Biacore 8K instrument. Anti-PILRA antibodies were captured on Biacore™ Series S CM5 sensor chips immobilized with mouse anti-human Fab (human Fab capture kit from GE Healthcare) followed by injections of various PILRA to PILRB mutant variants (hPILRA M1-hPILRA M11 (SEQ ID NOS:109-119)) at 1 μM concentration. FIG. 9A is a molecular structure showing hPILRA epitopes of anti-PILRA antibodies. PILRA binding epitopes were identified by the lack of antibody binding with a specific PILRA to PILRB mutation (FIG. 9B). As shown in FIG. 9B, Reference Antibodies #1-#4 were identified to bind epitope bin #3 (amino acids 116-118 of hPILRA). The heavy chain and light chain sequences of each of Reference Antibodies #1-#4 are provided in SEQ ID NOS:128-135. In each light chain and heavy chain sequences, sequences of the CDR1-3 are in bold and sequences of the V_{L} and V_{H} are underlined. Of the anti-PILRA antibodies described herein, only clone 10 binds to epitope bin #3, while the rest of the antibodies all bind to epitopes different from Reference antibodies #1-#4.

We were able identify distinct binding epitopes for our anti-PILRA antibodies and the results also demonstrated the diversity of epitopes covered by our antibodies.

### Example 9—Evaluation of Reference Anti-PILRA Antibodies

The binding affinities of four reference anti-PILRA antibodies to hPILRA, hPILRB, and cynoPILRA were measured by SPR using a Biacore 8K instrument (described in Example 2). The EC50 binding values of the four reference antibodies were also measured in HEK293 cells expressing hPILRA G78.

TABLE 6

| | | | | | HEK |
|---|---|---|---|---|---|
| Reference | hPILRA | hPILRB | cynoPILRA | Epitope | PILRA |
| mAbs | K_{D} | K_{D} | K_{D} | (bin) | G78 EC50 |
| Reference | 18 nM | No binding | No binding | 116-118 | 639 pM |
| Antibody #1 | | | | (Bin #3) | |
| Reference | 5.6 nM | No binding | No binding | 116-118 | 1.33 nM |
| Antibody #2 | | | | (Bin #3) | |
| Reference | 3.8 nM | Very weak | Very weak | 116-118 | 1.56 nM |
| Antibody #3 | | binding | binding | (Bin #3) | |
| Reference | 86 nM | No binding | No binding | 116-118 | 2.06 nM |
| Antibody #4 | | | | (Bin #3) | |

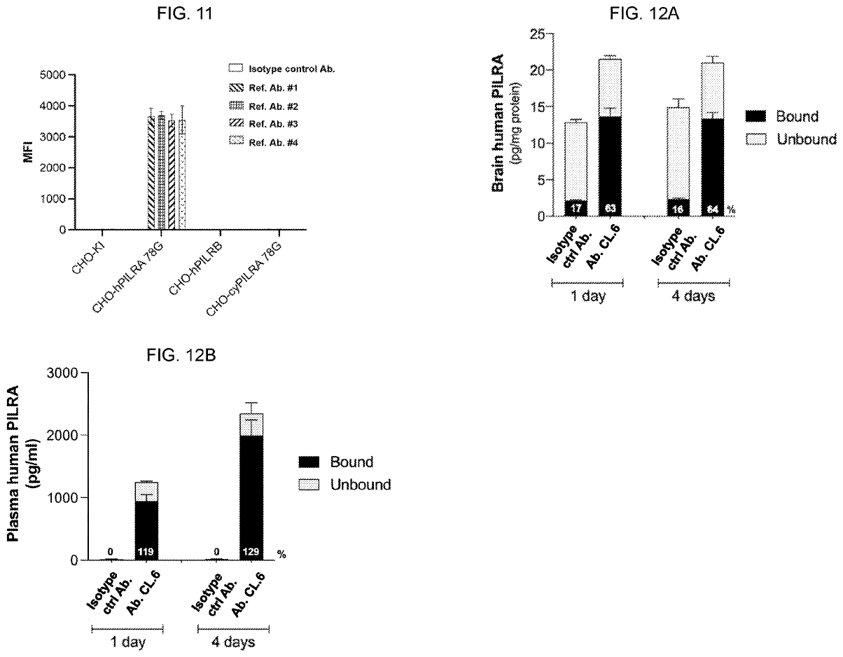

As shown by both Table 6 below and FIG. 11, Reference Antibodies #1-#4 did not bind to cynoPILRA and thus lack cross-reactivity between hPILRA and cynoPILRA.

### Example 10—Humanized Anti-PILRA Antibodies and Their Characterization

Exemplary anti-PILRA antibodies were humanized. Characterization for binding affinities of the humanized antibodies to hPILRA ECD were measured by SPR using a Biacore 8K instrument (Table 7). Each of the antibodies listed in Table 7 contains two wild-type Fc polypeptides (e.g., SEQ ID NO:94) forming an Fc domain in the antibody.

TABLE 7

| | SPR Binding Affinity to | |
|---|---|---|
| Anti-PILRA Antibody Clone | hPILRA | |
| 12 | 1.58 | nM |
| 13 | 655 | pM |
| 14 | 1.63 | nM |
| 15 | 917 | pM |
| 16 | 11.0 | nM |
| 17 | 1.29 | nM |
| 18 | 7.53 | nM |
| 19 | 2.00 | nM |
| 20 | 1.73 | nM |
| 21 | 667 | pM |
| 22 | 515 | pM |
| 23 | 495 | pM |
| 24 | 1.45 | nM |
| 25 | 794 | pM |
| 26 | 511 | pM |
| 27 | 536 | pM |
| 28 | 1.25 | nM |
| 29 | 1.28 | nM |
| 30 | 878 | pM |
| 31 | 915 | pM |
| 32 | 2.80 | nM |
| 33 | 2.04 | nM |
| 34 | 1.80 | nM |
| 35 | 1.71 | nM |

Further, humanized antibody clones 36-39 were generated. The binding affinities of these clones for hPILRA G78, hPILRA R78, hPILRB, and cyno PILRA were measured by SPR (Table 8).

TABLE 8

| Anti-PILRA | SPR Binding Affinities (nM) | | | |
|---|---|---|---|---|
| Antibody Clone | hPILRA G78 | hPILRA R78 | hPILRB | Cyno PILRA |
| 36 | 0.4-0.5 | 7.1-11 | 42-97 | 0.2-0.3 |
| 37 | 1.6-2 | 33-34 | 100-150 | 5.1-6.1 |
| 38 | 0.3-0.7 | 11 | 35-53 | 0.2-0.3 |
| 39 | 0.2-0.4 | 6.2-6.3 | 38-56 | 0.4 |

Moreover, EC50 binding values of the humanized antibodies with modified Fe polypeptides were measured in HEK293 cells expressing hPILRA G78 or hPILRA R78 (Table 9). In addition, EC50 values to induce phospho-pSTAT3 Y705 were also measured.

TABLE 9

| | | | EC50 (nM) to induce | |
|---|---|---|---|---|
| Anti-PILRA | | | phospho-STAT3 (Y705) in | |
| Antibody | EC50 (nM) in HEK293 cells | | HEK293 cells | |
| Clone | hPILRA G78 | hPILRA R78 | hPILRA G78 | hPILRA R78 |
| 36 | 4-13 | 1-4 | 12-13 | 3-4 |
| 37 | 8-17 | 3 | 12-13 | 3-5 |
| 38 | 13-14 | 2-4 | 7-8 | 2-3 |
| 39 | 7-8 | 1-2 | 6-9 | 3-4 |

Table 10 below further shows the binding affinities of selected humanized anti-PILRA antibodies to hPILRA G78, hPILRA R78, hPILRB, and cynoPILRA, the EC50 binding values measured in HEK293 cells expressing hPILRA G78, and the antibodies' binding epitopes of hPILRA.

TABLE 10

| Anti- | SPR Binding Affinities | | | | | | | Human |
|---|---|---|---|---|---|---|---|---|
| PILRA | Human | | Human | | | | Cell Binding | PILRA |
| Antibody | PILRA | | PILRA | Human | Cyno | | EC50 (HEK293 | Binding |
| Clone | G78 | | R78 | PILRB | PILRA | | hPILRA G78) | Epitope |
| 12 | 1.2 | nM | 17 nM | 263 nM | 1.8 | nM | 12.0 nM | G78, K106, |
| | | | | | | | | E143 |
| 15 | 650 | pM | 10 nM | 95 nM | 720 | pM | 11.2 nM | G78, K106, |
| | | | | | | | | E143 |
| 23 | 2.8 | nM | 47 nM | 117 nM | 7.3 | nM | 21.4 nM | G78, K106, |
| | | | | | | | | E143 |
| 35 | 1.4 | nM | 25 nM | 150 nM | 5.3 | nM | 12.8 nM | G78, K106, |
| | | | | | | | | E143 |

We successfully generated humanized anti-PILRA antibodies that maintained strong binding affinities to hPILRA (both G78 and R78 variants) and cynoPILRA, and much weaker binding to hPILRB.

### Example 11—Cell Binding of Humanized Anti-PILRA Antibodies

PILRA G78 or R78 overexpressing HEK293 cells were stained with NucBlue Live cell stain for 30 mins at RT. Parental HEK293 and HEK hPILRA G78 cells were mixed, washed, and incubated with various concentrations of anti-PILRA or isotype control antibodies for 30 mins at 290 rpm in 4° C. using FACS diluent (PBS 2% FBS 1 mM EDTA). Cells were washed twice with FACS diluent, incubated with Alexa Fluor 647-conjugated anti-human IgG at 290 rpm in 4C for 30 mins. Antibody binding to cells was detected via BD FACS Canto II and median fluorescence intensity (MFI) was derived after analyzing results via FLOJO and PRISM software. Cell lines were run in duplicate to account for intra-variability, for a total of N=3. The same protocol was used for CHO-K1 cells expressing hPILRA G78. FIGS. 1D and 1E show that anti-PILRA antibodies bound to PILRA G78 expressing HEK293 cells but not to parental cells. The antibodies have the following EC50 values on PILRA G78 expressing HEK293 cells: clone 6: 9.3 nM; clone 7: 12.95 nM; clone 12: 12.04 nM; clone 15: 11.2 nM; clone 23: 21.4 nM; and clone 35: 12.8 nM. FIG. 1F further shows that the humanized antibodies also bound to PILRA R78 expressing HEK293 cells. The antibodies have the following EC50 values on PILRA R78 expressing HEK293 cells: clone 2 5.1 nM; clone 6 2 nM; clone 7: 2.5 nM; clone 12 2.7 nM; clone 15 2.7 nM; clone 23 3.7 nM; and clone 35 3.1 nM. FIGS. 1H and 1I further show that anti-PILRA antibodies bound to PILRA G78 expressing CHO-K1 cells but not to parental cells. The antibodies have the following EC50 values on PILRA G78 expressing CHO-K1 cells: clone 6: 7.6 nM; clone 7: 10 nM; clone 12: 10.8 nM; clone 15: 9 nM; clone 23: 18.2 nM; and clone 35.

The antibodies were also tested for binding to iMicroglia. CRISPR mediated knock-in lines were generated to determine antibody binding to cells with homozygous G78R PILRA (AD-protective; R78) or R78G PILRA (normal AD-risk; G78) genetic variants. Human iMicroglia heterozygous for PILRA G78 or PILRA R78 were dosed with 100 nM biotinylated PILRA or isotype control antibodies for 45 minutes on ice. Cells were washed with PBS followed by 30 minutes incubation with Alexa Fluor 488-conjugated streptavidin for 30 minutes on ice. Cells were image using confocal microscopy after several PBS washes. Harmony Software was used to calculate mean fluorescent spot area per cell. Data is presented as fold expression over background signal in Alexa Fluor 488-conjugated streptavidin-only treated control wells (mean+/−SD). As shown in FIGS. 1L and 1M, anti-PILRA antibodies bound to human IPSC-derived iMicroglia homozygous for PILRA G78 (FIG. 1L) or PILRA R78 (FIG. 1M). Clone 2, which binds G78 epitope, showed no specific binding to iMicroglia expressing R78 PILRA. The AD-protective PILRA variant R78 has reduced ligand binding capacity and less affinity to clone 2. The frequency of this AD-protective variant varies around the world. It is the minor allele in African (10%) and European (38%) populations, but the major allele (65%) in East Asian populations. Anti-PILRA antibodies that bind to this epitope could be associated with loss-of-affinity in a large fraction of people, thus we sought to develop antibodies that bind to both variants.

Binding of anti-PILRA antibodies to iMicroglia homozygous for the protective R78 PILRA or to human R78 PILRA expressed on HEK293 demonstrated cell surface target engagement. Binding of anti-PILRA antibodies to human IPSC-derived Microglia with either PILRA allele combination (R78/R78, R78/G78, or G78/G78) demonstrated binding to a CNS-relevant cell type with endogenous cell surface levels of hPILRA receptor. This result predicts high affinity antibody binding to PILRA in humans regardless of allele.

The antibodies also demonstrated binding to cyno PILRA, but not to hPILRB, which is desirable. CHO-K1 and cyno PILRA expressing CHO cells were stained with NucBlue Live cell stain for 30 mins at RT. Cells were mixed, washed, and incubated with various concentrations of PILRA or isotype control antibodies for 30 mins at 290 rpm in 4° C. using FACS diluent (PBS 2% FBS 1 mM EDTA). Cells were washed twice with FACS diluent, incubated with Alexa Fluor 647-conjugated anti-human IgG at 290 rpm in 4° C. for 30 mins. Antibody binding to cells was detected via BD FACS Canto II and median fluorescence intensity (MFI) was derived after analyzing results via FLOJO and PRISM software. Cell lines were run in duplicates to account for intra-variability, for a total of N=3 (B) or N=1 (C). FIGS. 2B and 2C show that anti-PILRA antibodies bound to CHO cells expressing cyno PILRA (FIG. 2B) and did not bind to CHO cells expressing hPILRB (FIG. 2C).

Further, the antibodies were shown to not bind hPILRB. Human PILRB-DAP12 OE HEK293 cells were stained with NucBlue Live cell stain for 30 mins at RT. Cells were mixed, washed, and incubated with various concentrations of PILRA, isotype control antibodies, or a PILRB binding positive control antibody (HC SEQ ID NO:156; and LC SEQ ID NO:157; EC50 on hPILRB-DAP12 OE HEK293 cells is 1.2 nM) for 30 mins at 290 rpm in 4° C. using FACS diluent (PBS 2% FBS 1 mM EDTA). Cells were washed twice with FACS diluent, incubated with Alexa Fluor 647-conjugated anti-human IgG at 290 rpm in 4° C. for 30 mins. Antibody binding to cells was detected via BD FACS Canto II and median fluorescence intensity (MFI) was derived after analyzing results via FLOJO and PRISM software. Cell lines were run in duplicates to account for intra-variability, for a total of N=3. FIG. 2D shows anti-PILRA antibodies did not bind to HEK293 cells expressing hPILRB-DAP12.

This example further demonstrated that the humanized anti-PILRA antibodies maintained the desirable selective binding profile with strong binding for hPILRA and cynoPILRA and very weak binding for hPILRB on various cell types, e.g., HEK293 cells expressing PILRA G78 or R78, CHO-K1 cells expressing hPILRA G78 or cynoPILRA, and human IPSC-derived iMicroglia expressing PILRA G78 or R78.

### Example 12—Comparison to Reference Antibodies

We sought to compare our antibodies to reference PILRA antibodies to determine the binding profiles against hPILRA, hPILRB, and cynoPILRA. To do this, CHO-K1, CHO-hPILRA, CHO-hPILRB, or CHO-cyPILRA cells were incubated with anti-PILRA or isotype control antibodies at a single concentration of 100 nM for 30 minutes on ice. Cells were washed twice with FACS diluent, then incubated with Alexa Fluor 647-conjugated anti human IgG for 30 minutes on ice and washed once in FACS diluent. Antibody binding to the cells was detected by FACS and median fluorescence intensity (MFI) was derived from data analysis performed with the FLOJO software. Data is precented as fold signal over background (isotype control). Mean+/−SD, n=2 technical replicates.

FIGS. 2E and 2F show that Reference Antibodies bound CHO cells expressing hPILRA but, critically, did not bind to CHO cells expressing cynoPILRA. Our antibodies, by contrast, bound to both hPILRA and cynoPILRA and did not bind to hPILRB.

### Example 13—Sialidase Treatment of PILRA G78 or R78 HEK Cells

PILRA 78G or PILRA 78R expressing HEK293 cells were treated with SialEXO (Genovis). SialEXO is used for removal of sialic acids on native glycoproteins, and it works on both O- and N-linked glycans. It is a combination of two sialidases acting on α2-3, α2-6 and α2-8 linkages. Cells were incubated with 400 nM sialidase for 1 hour at 37° C. in serum-free DMEM to remove sialic acids on native glycoproteins (i.e., PILRA ligands). Cells were then washed twice and incubated and stained with NucBlue Live Stain for 30 mins at RT. Both the Parental Hek293 and HEK human PILRA 78G (or 78R) cells were mixed, washed and incubated with various concentrations of Anti-PILRA or isotype control antibodies for 30 mins. Cells were washed twice with FACS diluent (PBS with 2% FBS and 1 mM EDTA) and incubated with Alexa Fluor 647 conjugated anti human IgG at 290 rpm in 4° C. for 30 mins. Antibody binding to cells was detected via BD FACS Canto II and Median fluorescence Intensity (MFI) was derived after analyzing results via FLOJO and Prism software. The antibodies have the following EC50 values on HEK293 PILRA G78 cells without sialidase treatment: clone 5: 17 nM; clone 6: 13 nM; clone 7; 9.5 nM; and clone 1: 6.8 nM. The antibodies have the following EC50 values on HEK293 PILRA G78 cells with sialidase treatment: clone 5: 9.8 nM; clone 6: 4.8 nM; clone 7: 3.2 nM; and clone 1: 1.8 nM. FIGS. 3C-3F show that sialidase treatment in PILRA G78 HEK cells enhanced anti-PILRA antibody binding.

Following the same protocol, sialidase treatment was also performed on HEK cells expressing PILRA R78. The antibodies have the following EC50 values on HEK293 PILRA R78 cells without sialidase treatment: clone 5: 1.3M; clone 6: 0.7 nM; clone 7: 0.7 nM; and clone 1: 14 nM. The antibodies have the following EC50 values on HEK293 PILRA R78 cells with sialidase treatment: clone 5: 0.9 nM; clone 6: 0.3 nM; clone 7: 0.3 nM; and clone 1: 5.6 nM. FIGS. 3G-3J show that sialidase treatment of PILRA R78 HEK cells had minimal effect on anti-PILRA antibody binding.

Removal of cell surface sialylated ligands increased anti-PILRA antibody binding to PILRA G78 overexpressing cells, indicating that our antibodies compete for PILRA binding with endogenous cis-ligands. This is further supported by the minimal effect of sialidase treatment on anti-PILRA antibody binding to PILRA R78 cells, which displayed reduced ligand binding.

### Example 14—Anti-PILRA Antibodies Showed Target Engagement In Vivo

To test anti-PILRA antibodies in vivo, we generated BAC transgenic (BACtg) containing the human genomic sequence including the PILRA gene. These mice were generated by microinjecting BAC clone CTD-2110B7 into embryos from C57BL/6J (JAX Stock #000664) strain. This BAC clone contains the entire human PILRA (R78 version) and PILRB coding region and its regulatory elements. We confirmed expression of human PILRA in this model. These mice were administered clone 6 at 50 mg/kg 1 day and 4 days prior to sacrifice. Mice were later anesthetized with Avertin and perfused with PBS and brains were extracted and frozen on dry ice. The brain tissue was homogenized to produce brain lysates and total protein concentration was determined by BCA. Mesoscale Discovery streptavidin-coated plates were further coated with biotinylated PILRA capture antibody (R&D Systems AF6484) for 1 hour at room temperature at 800 rpm then washed 3 times with TBST. Brain lysate samples were split into two aliquots of 50 μl each. The first aliquot received a spike in of clone 6 (10 μg/ml final) to saturate all PILRA present while the second aliquot received only assay buffer to quantify the amount of PILRA that was bound by dosing in vivo. These aliquots were then applied to the plate and incubated 1 hour at room temperature at 800 rpm followed by washing 3 times with TBST. Finally, anti-human suflotag antibody was added as the detector at 0.25 μg/ml and incubated 1 hour at room temperature at 800 rpm followed by washing 3 times with TBST. Fluorescence was measured using Meso SECTOR S plate reader and PILRA concentrations were normalized to total protein level.

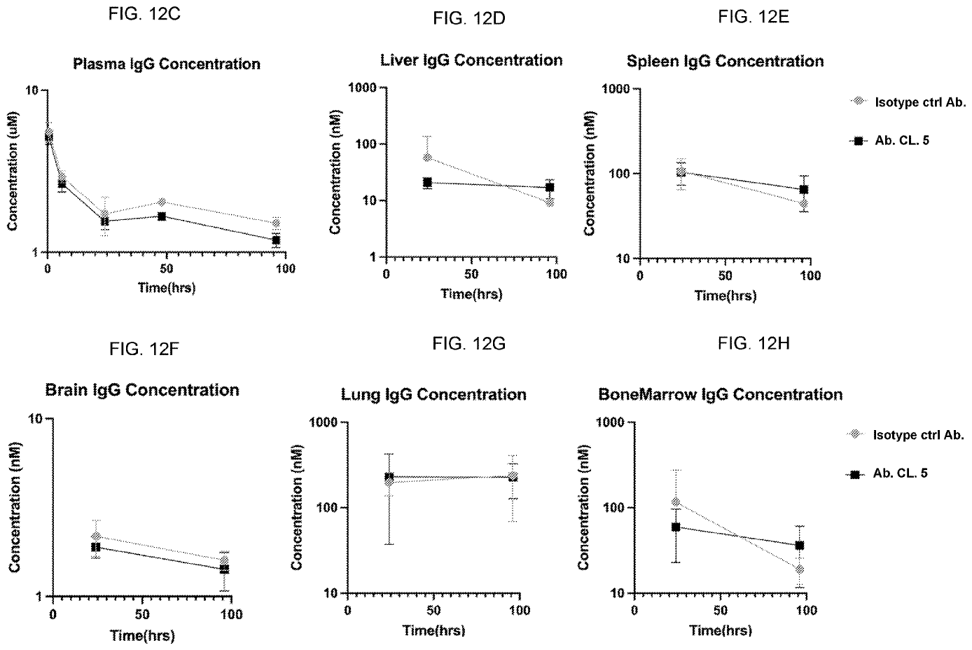

FIGS. 12A and 12B show that anti-PILRA antibody achieved target engagement in brain and plasma at 1 day and 4 days after 50 mg/kg dosing in human PILRA expressing BACtg mice. Anti-PILRA antibody increased total receptor levels of full length (brain) and soluble (plasma) human PILRA receptor compared to isotype antibody dosed animals.

Concentrations of the antibodies in various organs were also investigated using anti-human IgG ELISA (Capture antibody: Donkey anti-hu IgG (Fab′)2-minimal cross-reactivity (JIR #709-006-098); Detect antibody: Goat anti-hu IgG (Fab′)2-minimal cross-reactivity (JIR #109-036-098)). FIGS. 12C-12H show that anti-PILRA antibody demonstrated an IgG-like pharmacokinetics in brain, plasma, liver, lung, spleen, and bone marrow at 1 day and 4 days after 50 mg/kg IV administration in human PILRA expressing BACtg mice.

Pharmacokinetic profiles of anti-PILRA antibody or control (untargeted) antibody in a PILRA/PILRB BACtg mouse model resulted in comparable drug exposure profiles in plasma and other tissues tested. These data indicate that anti-PILRA mAb behaved as a typical antibody, without target engagement in this animal model.

ADDITIONAL SEQUENCES

| SEQ ID | | |
|---|---|---|
| NO | Sequence | Description |
| 94 | APELLGGPSVFLFPPKPKDTLMISRTPEVTCVVVDVSHEDPEVKFNWYV | Wild-type human Fc sequence |
| | DGVEVHNAKTKPREEQYNSTYRVVSVLTVLHQDWLNGKEYKCKVSNK | |
| | ALPAPIEKTISKAKGQPREPQVYTLPPSRDELTKNQVSLTCLVKGFYPSDI | |
| | AVEWESNGQPENNYKTTPPVLDSDGSFFLYSKLTVDKSRWQQGNVFSC | |
| | SVMHEALHNHYTQKSLSLSPGK | |
| 95 | APELLGGPSVFLFPPKPKDTLMISRTPEVTCVVVDVSHEDPEVKFNWYV | CH2 domain sequence |
| | DGVEVHNAKTKPREEQYNSTYRVVSVLTVLHQDWLNGKEYKCKVSNK | |
| | ALPAPIEKTISKAK | |
| 96 | GQPREPQVYTLPPSRDELTKNQVSLTCLVKGFYPSDIAVEWESNGQPEN | CH3 domain sequence |
| | NYKTTPPVLDSDGSFFLYSKLTVDKSRWQQGNVFSCSVMHEALHNHYT | |
| | QKSLSLSPGK | |
| 97 | EPKSCDKTHTCPPCP | human IgG1 hinge |
| 98 | DKTHTCPPCP | human IgG1 partial hinge |
| 99 | APEAAGGPSVFLFPPKPKDTLMISRTPEVTCVVVDVSHEDPEVKFNWYV | Fc sequence with LALA |
| | DGVEVHNAKTKPREEQYNSTYRVVSVLTVLHQDWLNGKEYKCKVSNK | |
| | ALPAPIEKTISKAKGQPREPQVYTLPPSRDELTKNQVSLTCLVKGFYPSDI | |
| | AVEWESNGQPENNYKTTPPVLDSDGSFFLYSKLTVDKSRWQQGNVFSC | |
| | SVMHEALHNHYTQKSLSLSPGK | |
| 100 | APEAAGGPSVFLFPPKPKDTLMISRTPEVTCVVVDVSHEDPEVKFNWYV | Fc sequence with LALA PG |
| | DGVEVHNAKTKPREEQYNSTYRVVSVLTVLHQDWLNGKEYKCKVSNK | |
| | ALGAPIEKTISKAKGQPREPQVYTLPPSRDELTKNQVSLTCLVKGFYPSD | |
| | IAVEWESNGQPENNYKTTPPVLDSDGSFFLYSKLTVDKSRWQQGNVFS | |
| | CSVMHEALHNHYTQKSLSLSPGK | |
| 101 | APEAAGGPSVFLFPPKPKDTLMISRTPEVTCVVVDVSHEDPEVKFNWYV | Fc sequence with LALA PS |
| | DGVEVHNAKTKPREEQYNSTYRVVSVLTVLHQDWLNGKEYKCKVSNK | |
| | ALSAPIEKTISKAKGQPREPQVYTLPPSRDELTKNQVSLTCLVKGFYPSDI | |
| | AVEWESNGQPENNYKTTPPVLDSDGSFFLYSKLTVDKSRWQQGNVFSC | |
| | SVMHEALHNHYTQKSLSLSPGK | |
| 102 | APELLGGPSVFLFPPKPKDTLMISRTPEVTCVVVDVSHEDPEVKFNWYV | Fc sequence with LS |
| | DGVEVHNAKTKPREEQYNSTYRVVSVLTVLHQDWLNGKEYKCKVSNK | |
| | ALPAPIEKTISKAKGQPREPQVYTLPPSRDELTKNQVSLTCLVKGFYPSDI | |
| | AVEWESNGQPENNYKTTPPVLDSDGSFFLYSKLTVDKSRWQQGNVFSC | |
| | SVLHEALHSHYTQKSLSLSPGK | |
| 103 | APEAAGGPSVFLFPPKPKDTLMISRTPEVTCVVVDVSHEDPEVKFNWYV | Fc sequence with LALA LS |
| | DGVEVHNAKTKPREEQYNSTYRVVSVLTVLHQDWLNGKEYKCKVSNK | |
| | ALPAPIEKTISKAKGQPREPQVYTLPPSRDELTKNQVSLTCLVKGFYPSDI | |
| | AVEWESNGQPENNYKTTPPVLDSDGSFFLYSKLTVDKSRWQQGNVFSC | |
| | SVLHEALHSHYTQKSLSLSPGK | |
| 104 | APEAAGGPSVFLFPPKPKDTLMISRTPEVTCVVVDVSHEDPEVKFNWYV | Fc sequence with LALA PG LS |
| | DGVEVHNAKTKPREEQYNSTYRVVSVLTVLHQDWLNGKEYKCKVSNK | |
| | ALGAPIEKTISKAKGQPREPQVYTLPPSRDELTKNQVSLTCLVKGFYPSD | |
| | IAVEWESNGQPENNYKTTPPVLDSDGSFFLYSKLTVDKSRWQQGNVFS | |
| | CSVLHEALHSHYTQKSLSLSPGK | |
| 105 | APEAAGGPSVFLFPPKPKDTLMISRTPEVTCVVVDVSHEDPEVKFNWYV | Fc sequence with LALA PS LS |
| | DGVEVHNAKTKPREEQYNSTYRVVSVLTVLHQDWLNGKEYKCKVSNK | |
| | ALSAPIEKTISKAKGQPREPQVYTLPPSRDELTKNQVSLTCLVKGFYPSDI | |
| | AVEWESNGQPENNYKTTPPVLDSDGSFFLYSKLTVDKSRWQQGNVFSC | |
| | SVLHEALHSHYTQKSLSLSPGK | |
| 106 | QPSGSTGSGPSYLYGVTQPKHLSASMGGSVEIPFSFYYPWELATAPDVRI | Portion of hPILRA protein- |
| | SWRRGHFHGQSFYSTRPPSIHKDYVNRLFLNWTEGQKSGFLRISNLQKQ | linker- |
| | DQSVYFCRVELDTRSSGRQQWQSIEGTKLSITQAVTTTTQRPSSMTTTW | His tag |
| | RLSSTTTTTGLRVTQGKRRSDSWHISLETGGGSGGGSHHHHHHHH | |
| 107 | QPGGSAGSGPSGPYGVTQRKHLSAPMGGSVEIPFSFYHPWELAAAPNM | Portion of cynoPILRA protein- |
| | KISWRRGNFHGEFFYRTRPAFIHEDYSNRLLLNWTEGQDRGLLRIWNLR | linker-His tag |
| | KEDQSVYFCRVELDTRRSGRQRWQSIEGTKLTITQAVTTTTQRPSSMTT | |
| | TRRPSSATTTAGLRVTQGKRHSDSWHLSLKTGGGSGGGSHHHHHHHH | |
| 108 | QPGGSTGSGPSYLYGVTQPKHLSASMGGSVEIPFSFYYPWELAIVPNVRI | Portion of hPILRB protein- |
| | SWRRGHFHGQSFYSTRPPSIHKDYVNRLFLNWTEGQESGFLRISNLRKE | linker- |
| | DQSVYFCRVELDTRRSGRQQLQSIKGTKLTITQAVTTTTTWRPSSTTTIA | His tag |
| | GLRVTESKGHSESWHLSLDTGGGSGGGSHHHHHHHH | |
| 109 | QPSGSTGSGPSYLYGVTQPKHLSASMGGSVEIPFSFYYPWELATAPDVRI | PILRA-STEM-Truncated-His |
| | SWRRGHFHGQSFYSTRPPSIHKDYVNRLFLNWTEGQKSGFLRISNLQKQ | (hPILRA M1) |
| | DQSVYFCRVELDTRSSGRQQWQSIEGTKLSITQAVTTTTTWRLSSTTTTT | |
| | GLRVTQGKRRSDSWHISLETGGGSGGGSHHHHHHHH | |
| 110 | QPSGSTGSGPSYLYGVTQPKHLSASMGGSVEIPFSFYYPWELAIVPNVRI | PILRA-TAPD63IVPN (hPILRA |
| | SWRRGHFHGQSFYSTRPPSIHKDYVNRLFLNWTEGQKSGFLRISNLQKQ | M2) |
| | DQSVYFCRVELDTRSSGRQQWQSIEGTKLSITQAVTTTTQRPSSMTTTW | |
| | RLSSTTTTTGLRVTQGKRRSDSWHISLETGGGSGGGSHHHHHHHH | |
| 111 | QPSGSTGSGPSYLYGVTQPKHLSASMGGSVEIPFSFYYPWELATAPDVRI | PILRA-G78R-His (hPILRA M3) |
| | SWRRGHFHRQSFYSTRPPSIHKDYVNRLFLNWTEGQKSGFLRISNLQKQ | |
| | DQSVYFCRVELDTRSSGRQQWQSIEGTKLSITQAVTTTTQRPSSMTTTW | |
| | RLSSTTTTTGLRVTQGKRRSDSWHISLETGGGSGGGSHHHHHHHH | |
| 112 | QPSGSTGSGPSYLYGVTQPKHLSASMGGSVEIPFSFYYPWELATAPDVRI | PILRA-K106E-His (hPILRA M4) |
| | SWRRGHFHGQSFYSTRPPSIHKDYVNRLFLNWTEGQESGFLRISNLQKQ | |
| | DQSVYFCRVELDTRSSGRQQWQSIEGTKLSITQAVTTTTQRPSSMTTTW | |
| | RLSSTTTTTGLRVTQGKRRSDSWHISLETGGGSGGGSHHHHHHHH | |
| 113 | QPSGSTGSGPSYLYGVTQPKHLSASMGGSVEIPFSFYYPWELATAPDVRI | PILRA-QKQ116RKE-His |
| | SWRRGHFHGQSFYSTRPPSIHKDYVNRLFLNWTEGQKSGFLRISNLRKE | (hPILRA M5) |
| | DQSVYFCRVELDTRSSGRQQWQSIEGTKLSITQAVTTTTQRPSSMTTTW | |
| | RLSSTTTTTGLRVTQGKRRSDSWHISLETGGGSGGGSHHHHHHHH | |
| 114 | QPSGSTGSGPSYLYGVTQPKHLSASMGGSVEIPFSFYYPWELATAPDVRI | PILRA-S133R-His (hPILRA M6) |
| | SWRRGHFHGQSFYSTRPPSIHKDYVNRLFLNWTEGQKSGFLRISNLQKQ | |
| | DQSVYFCRVELDTRRSGRQQWQSIEGTKLSITQAVTTTTQRPSSMTTTW | |
| | RLSSTTTTTGLRVTQGKRRSDSWHISLETGGGSGGGSHHHHHHHH | |
| 115 | QPSGSTGSGPSYLYGVTQPKHLSASMGGSVEIPFSFYYPWELATAPDVRI | PILRA-W139L-His (hPILRA |
| | SWRRGHFHGQSFYSTRPPSIHKDYVNRLFLNWTEGQKSGFLRISNLQKQ | M7) |
| | DQSVYFCRVELDTRSSGRQQLQSIEGTKLSITQAVTTTTQRPSSMTTTWR | |
| | LSSTTTTTGLRVTQGKRRSDSWHISLETGGGSGGGSHHHHHHHH | |
| 116 | QPSGSTGSGPSYLYGVTQPKHLSASMGGSVEIPFSFYYPWELATAPDVRI | PILRA-E143K-His (hPILRA M8) |
| | SWRRGHFHGQSFYSTRPPSIHKDYVNRLFLNWTEGQKSGFLRISNLQKQ | |
| | DQSVYFCRVELDTRSSGRQQWQSIKGTKLSITQAVTTTTQRPSSMTTTW | |
| | RLSSTTTTTGLRVTQGKRRSDSWHISLETGGGSGGGSHHHHHHHH | |
| 117 | QPSGSTGSGPSYLYGVTQPKHLSASMGGSVEIPFSFYYPWELATAPDVRI | PILRA-L169P-His (hPILRA M9) |
| | SWRRGHFHGQSFYSTRPPSIHKDYVNRLFLNWTEGQKSGFLRISNLQKQ | |
| | DQSVYFCRVELDTRSSGRQQWQSIEGTKLSITQAVTTTTQRPSSMTTTW | |
| | RPSSTTTTTGLRVTQGKRRSDSWHISLETGGGSGGGSHHHHHHHH | |
| 118 | QPSGSTGSGPSYLYGVTQPKHLSASMGGSVEIPFSFYYPWELATAPDVRI | PILRA-TT175IA-His (hPILRA |
| | SWRRGHFHGQSFYSTRPPSIHKDYVNRLFLNWTEGQKSGFLRISNLQKQ | M10) |
| | DQSVYFCRVELDTRSSGRQQWQSIEGTKLSITQAVTTTTQRPSSMTTTW | |
| | RLSSTTTIAGLRVTQGKRRSDSWHISLETGGGSGGGSHHHHHHHH | |
| 119 | QPSGSTGSGPSYLYGVTQPKHLSASMGGSVEIPFSFYYPWELATAPDVRI | PILRA-CT-swap-His (hPILRA |
| | SWRRGHFHGQSFYSTRPPSIHKDYVNRLFLNWTEGQKSGFLRISNLQKQ | M11) |
| | DQSVYFCRVELDTRSSGRQQWQSIEGTKLSITQAVTTTTQRPSSMTTTW | |
| | RLSSTTTTTGLRVTESKGHSESWHLSLDTGGGSGGGSHHHHHHHH | |
| 120 | ASTKGPSVFPLAPSSKSTSGGTAALGCLVKDYFPEPVTVSWNSGALTSG | Heavy chain constant domain 1 |
| | VHTFPAVLQSSGLYSLSSVVTVPSSSLGTQTYICNVNHKPSNTKVDKKV | (CH1) |
| | EPKSCDKTHTCPPCP | |
| 121 | RTVAAPSVFIFPPSDEQLKSGTASVVCLLNNFYPREAKVQWKVDNALQS | light chain constant domain |
| | GNSQESVTEQDSKDSTYSLSSTLTLSKADYEKHKVYACEVTHQGLSSPV | (CL) |
| | TKSFNRGEC | |
| 122 | EVQLVESGGGLVQPGRSLRLSCAVSGFTFDDYAMHWVRQAPGKGLEW | Heavy chain sequence of Ab.2 |
| | VSGFSWNSGSIGYPDSVKGRFTISRDNAKNSLYLQMNSLRAEDTAFYYC | |
| | AKDKSISAAGRFDYWGQGTLVTVSSASTKGPSVFPLAPSSKSTSGGTAA | |
| | LGCLVKDYFPEPVTVSWNSGALTSGVHTFPAVLQSSGLYSLSSVVTVPS | |
| | SSLGTQTYICNVNHKPSNTKVDKKVEPKSCDKTHTCPPCPAPEAAGGPS | |
| | VFLFPPKPKDTLMISRTPEVTCVVVDVSHEDPEVKFNWYVDGVEVHNA | |
| | KTKPREEQYNSTYRVVSVLTVLHQDWLNGKEYKCKVSNKALGAPIEKT | |
| | ISKAKGQPREPQVYTLPPSRDELTKNQVSLTCLVKGFYPSDIAVEWESN | |
| | GQPENNYKTTPPVLDSDGSFFLYSKLTVDKSRWQQGNVFSCSVMHEAL | |
| | HNHYTQKSLSLSPGK | |
| 123 | DIQMTQSPSSLSASVGDRVTITCQASRRINNYLNWYQQKPGKAPKLLIY | Light chain sequence of Ab.2 |
| | DASNLETGVPSRFTGSGSGTDFTLTISSLQPEDIATYYCQQYDNLPLTFG | |
| | GGTKVEIKRTVAAPSVFIFPPSDEQLKSGTASVVCLLNNFYPREAKVQW | |
| | KVDNALQSGNSQESVTEQDSKDSTYSLSSTLTLSKADYEKHKVYACEV | |
| | THQGLSSPVTKSFNRGEC | |
| 124 | EVQLVESGGGLVQPGRSLRLSCAASGFTFDDYAIHWVRQAPGKGLEWV | Heavy chain sequence of Ab.4 |
| | SGMSWNSGSIGYGDSVKGRFTISRDNAKNSLYLQMNSLRAEDTAFYYC | |
| | AKDKSIGAAGRFDSWGQGTLVTVSSASTKGPSVFPLAPSSKSTSGGTAA | |
| | LGCLVKDYFPEPVTVSWNSGALTSGVHTFPAVLQSSGLYSLSSVVTVPS | |
| | SSLGTQTYICNVNHKPSNTKVDKKVEPKSCDKTHTCPPCPAPEAAGGPS | |
| | VFLFPPKPKDTLMISRTPEVTCVVVDVSHEDPEVKFNWYVDGVEVHNA | |
| | KTKPREEQYNSTYRVVSVLTVLHQDWLNGKEYKCKVSNKALGAPIEKT | |
| | ISKAKGQPREPQVYTLPPSRDELTKNQVSLTCLVKGFYPSDIAVEWESN | |
| | GQPENNYKTTPPVLDSDGSFFLYSKLTVDKSRWQQGNVFSCSVMHEAL | |
| | HNHYTQKSLSLSPGK | |
| 125 | DIQMTQSPSSLSASVGDRVTITCQASQGINNYLNWYQQKPGKAPKLLIY | Light chain sequence of Ab.4 |
| | DASNLETGVPSRFSGSGSGTDFTFTISSLQPEDIATYYCQQYDNLPLTFGG | |
| | GTKVEIKRTVAAPSVFIFPPSDEQLKSGTASVVCLLNNFYPREAKVQWK | |
| | VDNALQSGNSQESVTEQDSKDSTYSLSSTLTLSKADYEKHKVYACEVT | |
| | HQGLSSPVTKSFNRGEC | |
| 126 | QVQLVQSGAEVKKPGASVRVSCKASGYTFIGFYIHWVRQAPGQGLEW | Heavy chain sequence of Ab.5 |
| | MGWINPESGDTTYAQKFQGRVTMTTDTSINTAYMDLNRLRSDDSAVYF | |
| | CARGNWNFPDTFDFWGQGTMVIVSSASTKGPSVFPLAPSSKSTSGGTAA | |
| | LGCLVKDYFPEPVTVSWNSGALTSGVHTFPAVLQSSGLYSLSSVVTVPS | |
| | SSLGTQTYICNVNHKPSNTKVDKKVEPKSCDKTHTCPPCPAPEAAGGPS | |
| | VFLFPPKPKDTLMISRTPEVTCVVVDVSHEDPEVKFNWYVDGVEVHNA | |
| | KTKPREEQYNSTYRVVSVLTVLHQDWLNGKEYKCKVSNKALGAPIEKT | |
| | ISKAKGQPREPQVYTLPPSRDELTKNQVSLTCLVKGFYPSDIAVEWESN | |
| | GQPENNYKTTPPVLDSDGSFFLYSKLTVDKSRWQQGNVFSCSVMHEAL | |
| | HNHYTQKSLSLSPGK | |
| 127 | DIQMTQSPSSLSASVGDRVTITCRSSQSISIYLNWHQQIPGKAPKLLIYVA | Light chain sequence of Ab.5 |
| | SSLQSGIPSRFSGRGSGTEFTLTISSLQPEDFATYYCQQSYSAPFTFGPGTK | |
| | VDIKRTVAAPSVFIFPPSDEQLKSGTASVVCLLNNFYPREAKVQWKVDN | |
| | ALQSGNSQESVTEQDSKDSTYSLSSTLTLSKADYEKHKVYACEVTHQGL | |
| | SSPVTKSFNRGEC | |
| 128 | QVTLQESGPGILQPSQTLSLTCSFSGFSLSTFGMGVGWIRQPSGKGLEW | Heavy chain sequence of |
| | LAHIWWDDDKYYNPALKSRLTISKDTSKNQVFLKIASVDTADIATYYC | Reference Antibody #1 |
| | ARVEDYGNPFDYWGQGTTLTVSSASTKGPSVFPLAPSSKSTSGGTAAL | |
| | GCLVKDYFPEPVTVSWNSGALTSGVHTFPAVLQSSGLYSLSSVVTVPSS | |
| | SLGTQTYICNVNHKPSNTKVDKKVEPKSCDKTHTCPPCPAPEAAGGPSV | |
| | FLFPPKPKDTLMISRTPEVTCVVVDVSHEDPEVKFNWYVDGVEVHNAK | |
| | TKPREEQYNSTYRVVSVLTVLHQDWLNGKEYKCKVSNKALGAPIEKTI | |
| | SKAKGQPREPQVYTLPPSRDELTKNQVSLTCLVKGFYPSDIAVEWESNG | |
| | QPENNYKTTPPVLDSDGSFFLYSKLTVDKSRWQQGNVFSCSVMHEALH | |
| | NHYTQKSLSLSPGK | |
| 129 | DIQMNQSPSSLSASLGDTITITCHASQNIHVWLNWYQQKPGNIPKLLIY | Light chain sequence of |
| | KASNLHTGVPSRFSGSGSGTGFTVTISSLQPEDIATYYCQQGQSYPLTF | Reference |
| | GAGTKLELKRTVAAPSVFIFPPSDEQLKSGTASVVCLLNNFYPREAKVQ | Antibody #1 |
| | WKVDNALQSGNSQESVTEQDSKDSTYSLSSTLTLSKADYEKHKVYACE | |
| | VTHQGLSSPVTKSFNRGEC | |
| 130 | QVILKESGPGILQSSQTLSLTCSFSGFSLSTFGMGVGWIRQPSGKGLESL | Heavy chain sequence of |
| | AHIWWDDDKFYNPALKSRLTISKDTSKSQVFLKIANVDTADIATYYCT | Reference Antibody #2 |
| | RIEDYGSYFAYWGQGTTLTVSSASTKGPSVFPLAPSSKSTSGGTAALGC | |
| | LVKDYFPEPVTVSWNSGALTSGVHTFPAVLQSSGLYSLSSVVTVPSSSLG | |
| | TQTYICNVNHKPSNTKVDKKVEPKSCDKTHTCPPCPAPEAAGGPSVFLF | |
| | PPKPKDTLMISRTPEVTCVVVDVSHEDPEVKFNWYVDGVEVHNAKTKP | |
| | REEQYNSTYRVVSVLTVLHQDWLNGKEYKCKVSNKALGAPIEKTISKA | |
| | KGQPREPQVYTLPPSRDELTKNQVSLTCLVKGFYPSDIAVEWESNGQPE | |
| | NNYKTTPPVLDSDGSFFLYSKLTVDKSRWQQGNVFSCSVMHEALHNHY | |
| | TQKSLSLSPGK | |
| 131 | DVQMNQSPSSLSASLGDPITITCHASQNIHVWLNWYQQRPGNIPRLLIY | Light chain sequence of |
| | KASNLHTGVPSRFSGSGSGTGFTLTISSLQPEDIATYYCQQGQSYPYTF | Reference |
| | GGGTKLEIKRTVAAPSVFIFPPSDEQLKSGTASVVCLLNNFYPREAKVQ | Antibody #2 |
| | WKVDNALQSGNSQESVTEQDSKDSTYSLSSTLTLSKADYEKHKVYACE | |
| | VTHQGLSSPVTKSFNRGEC | |
| 132 | QVTLKESGPGMLQPSQTLSLACSFSGFSLNSFGVAVGWIRQPSGKGLEW | Heavy chain sequence of |
| | LAHIWWDDDKSYNPALKSRLTISKDTSKNQVFLKLANVDTADTATYY | Reference Antibody #3 |
| | CTRIADYGNHFDYWGQGTALTVSSASTKGPSVFPLAPSSKSTSGGTAAL | |
| | GCLVKDYFPEPVTVSWNSGALTSGVHTFPAVLQSSGLYSLSSVVTVPSS | |
| | SLGTQTYICNVNHKPSNTKVDKKVEPKSCDKTHTCPPCPAPEAAGGPSV | |
| | FLFPPKPKDTLMISRTPEVTCVVVDVSHEDPEVKFNWYVDGVEVHNAK | |
| | TKPREEQYNSTYRVVSVLTVLHQDWLNGKEYKCKVSNKALGAPIEKTI | |
| | SKAKGQPREPQVYTLPPSRDELTKNQVSLTCLVKGFYPSDIAVEWESNG | |
| | QPENNYKTTPPVLDSDGSFFLYSKLTVDKSRWQQGNVFSCSVMHEALH | |
| | NHYTQKSLSLSPGK | |
| 133 | DIQMNQSPSSLSASLGDTITITCHASQNSHVWLSWYQQKPGNIPKLLIY | Light chain sequence of |
| | KASNLHTGVPSRFSGSGSGTGFTLTISGLQPEDIATYYCQQGQTYPFTF | Reference |
| | GSGTKLEIKRTVAAPSVFIFPPSDEQLKSGTASVVCLLNNFYPREAKVQW | Antibody #3 |
| | KVDNALQSGNSQESVTEQDSKDSTYSLSSTLTLSKADYEKHKVYACEV | |
| | THQGLSSPVTKSFNRGEC | |
| 134 | QVTLKESGPGILQPSQTLSLTCSFSGFSLTTFGMGVGWIRQPSGKGLEW | Heavy chain sequence of |
| | LAHIWWDDDKYYNPALKSRLTISKDISKNQVFLKIANVDTADTATYYC | Reference Antibody #4 |
| | ARIEDYGNPFDYWGQGTTLTVSSASTKGPSVFPLAPSSKSTSGGTAALG | |
| | CLVKDYFPEPVTVSWNSGALTSGVHTFPAVLQSSGLYSLSSVVTVPSSSL | |
| | GTQTYICNVNHKPSNTKVDKKVEPKSCDKTHTCPPCPAPEAAGGPSVFL | |
| | FPPKPKDTLMISRTPEVTCVVVDVSHEDPEVKFNWYVDGVEVHNAKTK | |
| | PREEQYNSTYRVVSVLTVLHQDWLNGKEYKCKVSNKALGAPIEKTISK | |
| | AKGQPREPQVYTLPPSRDELTKNQVSLTCLVKGFYPSDIAVEWESNGQP | |
| | ENNYKTTPPVLDSDGSFFLYSKLTVDKSRWQQGNVFSCSVMHEALHNH | |
| | YTQKSLSLSPGK | |
| 135 | DIQMNQSPSSLSASLGDTITITCHASQNIHVWLSWYQQKPGNIPKLLIYK | Light chain sequence of |
| | ASNLHTGVPSRFSGSGSGTGFTLTISSLQPEDIATYYCQQGQSYPLTFGA | Reference |
| | GTKLELKRTVAAPSVFIFPPSDEQLKSGTASVVCLLNNFYPREAKVQWK | Antibody #4 |
| | VDNALQSGNSQESVTEQDSKDSTYSLSSTLTLSKADYEKHKVYACEVT | |
| | HQGLSSPVTKSFNRGEC | |
| 136 | MGRPLLLPLLPLLLPPAFLQPSGSTGSGPSYLYGVTQPKHLSASMGGSVE | Human PILRA protein (R78) |
| | IPFSFYYPWELATAPDVRISWRRGHFHRQSFYSTRPPSIHKDYVNRLFLN | |
| | WTEGQKSGFLRISNLQKQDQSVYFCRVELDTRSSGRQQWQSIEGTKLSI | |
| | TQAVTTTTQRPSSMTTTWRLSSTTTTTGLRVTQGKRRSDSWHISLETAV | |
| | GVAVAVTVLGIMILGLICLLRWRRRKGQQRTKATTPAREPFQNTEEPYE | |
| | NIRNEGQNTDPKLNPKDDGIVYASLALSSSTSPRAPPSHRPLKSPQNETL | |
| | YSVLKA | |
| 150 | EVQLVQSGAEVKKPGASVKVSCKASGYTFTEYYMYWVRQAPGQGLEL | Heavy chain sequence of Ab.12 |
| | IGRIDPEDGGTDYIEKFKNRVTITADTSTSTAYLELSSLRSEDTAVYYCAT | |
| | TIRGTVFAFWGQGTLVTVSSASTKGPSVFPLAPSSKSTSGGTAALGCLVK | |
| | DYFPEPVTVSWNSGALTSGVHTFPAVLQSSGLYSLSSVVTVPSSSLGTQT | |
| | YICNVNHKPSNTKVDKKVEPKSCDKTHTCPPCPAPEAAGGPSVFLFPPK | |
| | PKDTLMISRTPEVTCVVVDVSHEDPEVKFNWYVDGVEVHNAKTKPREE | |
| | QYNSTYRVVSVLTVLHQDWLNGKEYKCKVSNKALGAPIEKTISKAKGQ | |
| | PREPQVYTLPPSRDELTKNQVSLTCLVKGFYPSDIAVEWESNGQPENNY | |
| | KTTPPVLDSDGSFFLYSKLTVDKSRWQQGNVFSCSVMHEALHNHYTQK | |
| | SLSLSPGK | |
| 151 | DIQMTQSPSSLSASVGDRVTITCRASEDIFNGLAWYQQKPGKSPKLLIYN | Light chain sequence of Ab.12, |
| | AKTLHTGVPSRFSGSGSGSDYTLTISSLQPEDFATYFCQQYYDYPLTFGQ | and Ab.15 |
| | GTKVEIKRTVAAPSVFIFPPSDEQLKSGTASVVCLLNNFYPREAKVQWK | |
| | VDNALQSGNSQESVTEQDSKDSTYSLSSTLTLSKADYEKHKVYACEVT | |
| | HQGLSSPVTKSFNRGEC | |
| 152 | EVQLVQSGAEVKKPGASVKVSCKASGYTFTEYYMYWVRQAPGQGLEL | Heavy chain sequence of Ab.15 |
| | IGRIDPEDGGTDYIEKFKNRSTLTADTSTSTAYLELSSLRSEDTAVYFCAT | |
| | TIRGTVFAFWGQGTLVTVSSASTKGPSVFPLAPSSKSTSGGTAALGCLVK | |
| | DYFPEPVTVSWNSGALTSGVHTFPAVLQSSGLYSLSSVVTVPSSSLGTQT | |
| | YICNVNHKPSNTKVDKKVEPKSCDKTHTCPPCPAPEAAGGPSVFLFPPK | |
| | PKDTLMISRTPEVTCVVVDVSHEDPEVKFNWYVDGVEVHNAKTKPREE | |
| | QYNSTYRVVSVLTVLHQDWLNGKEYKCKVSNKALGAPIEKTISKAKGQ | |
| | PREPQVYTLPPSRDELTKNQVSLTCLVKGFYPSDIAVEWESNGQPENNY | |
| | KTTPPVLDSDGSFFLYSKLTVDKSRWQQGNVFSCSVMHEALHNHYTQK | |
| | SLSLSPGK | |
| 153 | EVQLVQSGAEVKKPGASVKVSCKASGYTFTEYYMYWVRQAPGQGLEL | Heavy chain sequence of Ab.23, |
| | IGRIDPEDGGTDYVEKFKNRATLTADTSTSTAYLELSSLRSEDTAVYFCA | and Ab.35 |
| | STIRGTVFVYWGQGTLVTVSSASTKGPSVFPLAPSSKSTSGGTAALGCLV | |
| | KDYFPEPVTVSWNSGALTSGVHTFPAVLQSSGLYSLSSVVTVPSSSLGTQ | |
| | TYICNVNHKPSNTKVDKKVEPKSCDKTHTCPPCPAPEAAGGPSVFLFPP | |
| | KPKDTLMISRTPEVTCVVVDVSHEDPEVKFNWYVDGVEVHNAKTKPRE | |
| | EQYNSTYRVVSVLTVLHQDWLNGKEYKCKVSNKALGAPIEKTISKAKG | |
| | QPREPQVYTLPPSRDELTKNQVSLTCLVKGFYPSDIAVEWESNGQPENN | |
| | YKTTPPVLDSDGSFFLYSKLTVDKSRWQQGNVFSCSVMHEALHNHYTQ | |
| | KSLSLSPGK | |
| 154 | DIQMTQSPSSLSASVGDRVTITCRPSEDIYNGLAWYQQKPGKAPKLLIYN | Light chain sequence of Ab.23 |
| | ANSLHTGVPSRFSGSGSGTDYTLTISSLQPEDFATYYCQQYYDYPLTFGQ | |
| | GTKVEIKRTVAAPSVFIFPPSDEQLKSGTASVVCLLNNFYPREAKVQWK | |
| | VDNALQSGNSQESVTEQDSKDSTYSLSSTLTLSKADYEKHKVYACEVT | |
| | HQGLSSPVTKSFNRGEC | |
| 155 | DIQMTQSPSSLSASVGDRVTITCRPSEDIYNGLAWYQQKPGKSPKLLIYN | Light chain sequence of Ab.35 |
| | ANSLHTGVPSRFSGSGSGTDYTLTISSLQPEDFATYFCQQYYDYPLTFGQ | |
| | GTKVEIKRTVAAPSVFIFPPSDEQLKSGTASVVCLLNNFYPREAKVQWK | |
| | VDNALQSGNSQESVTEQDSKDSTYSLSSTLTLSKADYEKHKVYACEVT | |
| | HQGLSSPVTKSFNRGEC | |
| 156 | QVQLQESGPGLVKPSGTLSLTCAVSGGSISSNHWWSWVRQPPGKGLEW | Heavy chain sequence of PILRB |
| | IGEIYHYGTTDYNPSLQSRVTISVDKSKNQFSLKLTSVTAADTAVYFCA | binding positive control |
| | RGLRDYYYYMDVWGKGTTVTVSSASTKGPSVFPLAPSSKSTSGGTAAL | antibody |
| | GCLVKDYFPEPVTVSWNSGALTSGVHTFPAVLQSSGLYSLSSVVTVPSS | (CDRS bold and underlined) |
| | SLGTQTYICNVNHKPSNTKVDKKVEPKSCDKTHTCPPCPAPEAAGGPSV | |
| | FLFPPKPKDTLMISRTPEVTCVVVDVSHEDPEVKFNWYVDGVEVHNAK | |
| | TKPREEQYNSTYRVVSVLTVLHQDWLNGKEYKCKVSNKALGAPIEKTI | |
| | SKAKGQPREPQVYTLPPSRDELTKNQVSLTCLVKGFYPSDIAVEWESNG | |
| | QPENNYKTTPPVLDSDGSFFLYSKLTVDKSRWQQGNVFSCSVMHEALH | |
| | NHYTQKSLSLSPGK | |
| 157 | DIQMTQSPSSLSASVGDRVTITCRASQTISSYLNWYQQKPGKAPKLLIY | Light chain sequence of PILRB |
| | AASSLQSGVPSRFSGSGFGTDFTLTISSLQPEDFATYYCQQSYSIPLTFGG | binding positive control |
| | GTKVEIKRTVAAPSVFIFPPSDEQLKSGTASVVCLLNNFYPREAKVQWK | antibody |
| | VDNALQSGNSQESVTEQDSKDSTYSLSSTLTLSKADYEKHKVYACEVT | (CDRS bold and underlined) |
| | HQGLSSPVTKSFNRGEC | |

1-5. (canceled)

6. An isolated antibody or antigen-binding fragment thereof that binds to a human paired immunoglobulin-like type 2 receptor alpha (hPILRA), wherein the antibody or antigen-binding fragment thereof binds to the amino acid(s) at:
(a) positions 78, 106, and 143 of SEQ ID NO:1; or
(b) positions 63 and 64 of SEQ ID NO: 1; or
(c) position 106 of SEQ ID NO:1; or
(d) positions 116-118 of SEQ ID NO: 1; or
(e) positions 182-186 of SEQ ID NO:1.

7. (canceled)

8. The isolated antibody or antigen-binding fragment thereof of claim 6, wherein the antibody or antigen-binding fragment thereof binds to:
(a) G78, K106, and E143 of SEQ ID NO:1 or R78, K106, and E143 of SEQ ID NO:136; or
(b) T63 and A64 of SEQ ID NO:1; or
(c) K106 of SEQ ID NO:1; or
(d) Q116, K117, and Q118 of SEQ ID NO:1; or
(e) Q182, G183, K184, R185, and R186 of SEQ ID NO:1.

9-15. (canceled)

16. The isolated antibody or antigen-binding fragment thereof of claim 6, wherein the antibody or antigen-binding fragment thereof comprises:
(a) a heavy chain CDR1 (CDR-H1) sequence having at least 90% sequence identity to the amino acid sequence of any one of SEQ ID NOS:4-11, or having up to two amino acid substitutions relative to the amino acid sequence of any one of SEQ ID NOS:4-11;
(b) a heavy chain CDR2 (CDR-H2) sequence having at least 80% sequence identity to the amino acid sequence of any one of SEQ ID NOS:12-19, or having up to two amino acid substitutions relative to the amino acid sequence of any one of SEQ ID NOS:12-19;
(c) a heavy chain CDR3 (CDR-H3) sequence having at least 80% sequence identity to the amino acid sequence of any one of SEQ ID NOS:20-29, or having up to two amino acid substitutions relative to the amino acid sequence of any one of SEQ ID NOS:20-29;
(d) a light chain CDR1 (CDR-L1) sequence having at least 90% sequence identity to the amino acid sequence of any one of SEQ ID NOS:30-38, or having up to two amino acid substitutions relative to the amino acid sequence of any one of SEQ ID NOS:30-38;
(e) a light chain CDR2 (CDR-L2) sequence having at least 80% sequence identity to the amino acid sequence of SEQ ID NO:39-46, or having up to two amino acid substitutions relative to the amino acid sequence of SEQ ID NO:39-46; and
(f) a light chain CDR3 (CDR-L3) sequence having at least 80% sequence identity to the amino acid sequence of any one of SEQ ID NOS:47-53, or having up to two amino acid substitutions relative to the amino acid sequence of any one of SEQ ID NOS:47-53.

17. (canceled)

18. The isolated antibody or antigen-binding fragment of claim 17, wherein the antibody or antigen-binding fragment comprises:
(i) a CDR-H1 comprising the sequence of SEQ ID NO:4 or one or more conservative substitutions relative to the sequence of SEQ ID NO:4; a CDR-H2 comprising the sequence of SEQ ID NO:12 or one or more conservative substitutions relative to the sequence of SEQ ID NO:12; a CDR-H3 comprising the sequence of SEQ ID NO:20 or one or more conservative substitutions relative to the sequence of SEQ ID NO:20; a CDR-L1 comprising the sequence of SEQ ID NO:30 or one or more conservative substitutions relative to the sequence of SEQ ID NO:30; a CDR-L2 comprising the sequence of SEQ ID NO:39 or one or more conservative substitutions relative to the sequence of SEQ ID NO:39; and a CDR-L3 comprising the sequence of SEQ ID NO:47 or one or more conservative substitutions relative to the sequence of SEQ ID NO:47; or
(ii) a CDR-H1 comprising the sequence of SEQ ID NO:5 or one or more conservative substitutions relative to the sequence of SEQ ID NO:5; a CDR-H2 comprising the sequence of SEQ ID NO:13 or one or more conservative substitutions relative to the sequence of SEQ ID NO:13; a CDR-H3 comprising the sequence of SEQ ID NO:22 or one or more conservative substitutions relative to the sequence of SEQ ID NO:22; a CDR-L1 comprising the sequence of SEQ ID NO:31 or one or more conservative substitutions relative to the sequence of SEQ ID NO:31; a CDR-L2 comprising the sequence of SEQ ID NO:39 or one or more conservative substitutions relative to the sequence of SEQ ID NO:39; and a CDR-L3 comprising the sequence of SEQ ID NO:47 or one or more conservative substitutions relative to the sequence of SEQ ID NO:47; or
(iii) a CDR-H1 comprising the sequence of SEQ ID NO:6 or one or more conservative substitutions relative to the sequence of SEQ ID NO:6; a CDR-H2 comprising the sequence of SEQ ID NO:14 or one or more conservative substitutions relative to the sequence of SEQ ID NO:14; a CDR-H3 comprising the sequence of SEQ ID NO:23 or one or more conservative substitutions relative to the sequence of SEQ ID NO:23; a CDR-L1 comprising the sequence of SEQ ID NO:32 or one or more conservative substitutions relative to the sequence of SEQ ID NO:32; a CDR-L2 comprising the sequence of SEQ ID NO:40 or one or more conservative substitutions relative to the sequence of SEQ ID NO:40; and a CDR-L3 comprising the sequence of SEQ ID NO:48 or one or more conservative substitutions relative to the sequence of SEQ ID NO:48;
(iv) a CDR-H1 comprising the sequence of SEQ ID NO:7 or one or more conservative substitutions relative to the sequence of SEQ ID NO:7; a CDR-H2 comprising the sequence of SEQ ID NO:15 or one or more conservative substitutions relative to the sequence of SEQ ID NO:15; a CDR-H3 comprising the sequence of SEQ ID NO:24 or one or more conservative substitutions relative to the sequence of SEQ ID NO:24; a CDR-L1 comprising the sequence of SEQ ID NO:33 or one or more conservative substitutions relative to the sequence of SEQ ID NO:33; a CDR-L2 comprising the sequence of SEQ ID NO:41 or one or more conservative substitutions relative to the sequence of SEQ ID NO:41; and a CDR-L3 comprising the sequence of SEQ ID NO:49 or one or more conservative substitutions relative to the sequence of SEQ ID NO:49; or
(v) a CDR-H1 comprising the sequence of SEQ ID NO:7 or one or more conservative substitutions relative to the sequence of SEQ ID NO:7; a CDR-H2 comprising the sequence of SEQ ID NO:15 or one or more conservative substitutions relative to the sequence of SEQ ID NO:15; a CDR-H3 comprising the sequence of SEQ ID NO:25 or one or more conservative substitutions relative to the sequence of SEQ ID NO:25; a CDR-L1 comprising the sequence of SEQ ID NO:34 or one or more conservative substitutions relative to the sequence of SEQ ID NO:34; a CDR-L2 comprising the sequence of SEQ ID NO:42 or one or more conservative substitutions relative to the sequence of SEQ ID NO:42; and a CDR-L3 comprising the sequence of SEQ ID NO:49 or one or more conservative substitutions relative to the sequence of SEQ ID NO:49; or
(vi) a CDR-H1 comprising the sequence of SEQ ID NO:8 or one or more conservative substitutions relative to the sequence of SEQ ID NO:8; a CDR-H2 comprising the sequence of SEQ ID NO:16 or one or more conservative substitutions relative to the sequence of SEQ ID NO:16; a CDR-H3 comprising the sequence of SEQ ID NO:26 or one or more conservative substitutions relative to the sequence of SEQ ID NO:26; a CDR-L1 comprising the sequence of SEQ ID NO:35 or one or more conservative substitutions relative to the sequence of SEQ ID NO:35; a CDR-L2 comprising the sequence of SEQ ID NO:43 or one or more conservative substitutions relative to the sequence of SEQ ID NO:43; and a CDR-L3 comprising the sequence of SEQ ID NO:50 or one or more conservative substitutions relative to the sequence of SEQ ID NO:50.

19-21. (canceled)

22. The isolated antibody or antigen-binding fragment of claim 6, comprising a heavy chain variable region (V_{H}) sequence that has at least 85% sequence identity to any one of SEQ ID NOS:54-63.

23. (canceled)

24. The isolated antibody or antigen-binding fragment of claim 6, comprising a heavy chain variable region (V_{H}) sequence that has at least 85% sequence identity to any one of SEQ ID NOS:137-144 and 158.

25. (canceled)

26. The isolated antibody or antigen-binding fragment of claim 6, comprising a light chain variable region (V_{L}) sequence that has at least 85% sequence identity to any one of SEQ ID NOS:64-73.

27. (canceled)

28. The isolated antibody or antigen-binding fragment of any one of claim 6, comprising a light chain variable region (V_{L}) sequence that has at least 85% sequence identity to any one of SEQ ID NOS:145-149.

29. (canceled)

30. The isolated antibody or antigen-binding fragment of claim 6, wherein the antibody or antigen-binding fragment comprises:
(i) a V_{H} sequence comprising SEQ ID NO:54 and a V_{L} sequence comprising SEQ ID NO:65; or
(ii) a V_{H} sequence comprising SEQ ID NO:56 and a V_{L} sequence comprising SEQ ID NO:66; or
(iii) a V_{H} sequence comprising SEQ ID NO:57 and a V_{L} sequence comprising SEQ ID NO:67; or
(iv) a V_{H} sequence comprising SEQ ID NO:58 and a V_{L} sequence comprising SEQ ID NO:68; or
(v) a V_{H} sequence comprising SEQ ID NO:59 and a V_{L} sequence comprising SEQ ID NO:69; or
(vi) a V_{H} sequence comprising SEQ ID NO:60 and a V_{L} sequence comprising SEQ ID NO:70; or
(vii) a V_{H} sequence comprising SEQ ID NO:137 and a V_{L} sequence comprising SEQ ID NO:145; or
(viii) a V_{H} sequence comprising SEQ ID NO:140 and a V_{L} sequence comprising SEQ ID NO:145; or
(ix) a V_{H} sequence comprising SEQ ID NO:143 and a V_{L} sequence comprising SEQ ID NO:146; or
(x) a V_{H} sequence comprising SEQ ID NO:143 and a V_{L} sequence comprising SEQ ID NO:149.

31-32. (canceled)

33. The isolated antibody or antigen-binding fragment of claim 6, wherein the antibody comprises two Fc polypeptides forming an Fc domain.

34. The isolated antibody or antigen-binding fragment of claim 33, wherein one or both Fc polypeptides comprise a sequence having at least 85% identity to the sequence of SEQ ID NO:94.

35. The isolated antibody or antigen-binding fragment of claim 6, wherein the antibody is an IgG1, a full-length antibody, a monoclonal antibody, a humanized antibody, and a chimeric antibody or a fully human antibody.

36-38. (canceled)

39. The isolated antibody or antigen-binding fragment thereof of claim 6, wherein the antibody or antigen-binding fragment thereof:
(a) antagonizes hPILRA activity; and/or
(b) blocks binding of a sialyated protein to hPILRA; and/or
(c) enhances phosphorylation of EGFR or STAT3, or decreases phosphorylation of STAT1; and/or
(d) enhances cell migration; and/or
(e) enhances anti-inflammatory gene or protein expression; and/or
(f) reduces pro-inflammatory cytokine protein expression or secretion; and/or
(g) increases cellular respiration; and/or
(h) increases ATP production; and/or
(i) increases fatty acid metabolism; and/or
(j) does not activate peripheral immune cells.

40-60. (canceled)

61. A pharmaceutical composition comprising the isolated antibody or antigen-binding fragment thereof of claim 6 and a pharmaceutically acceptable carrier.

62. A polynucleotide comprising a nucleic acid sequence encoding the isolated antibody or antigen-binding fragment thereof of claim 6.

63. A vector or a host cell comprising the polynucleotide of claim 62.

64. (canceled)

65. A method for producing an isolated antibody or antigen-binding fragment thereof, comprising culturing a host cell under conditions in which the isolated antibody or antigen-binding fragment thereof encoded by the polynucleotide of claim 62 is expressed.

66. A kit comprising:
the isolated antibody or antigen-binding fragment thereof of any one of claim 6; and
instructions for use thereof.

67. A method of treating a neurodegenerative disease in a subject, comprising administering to the subject the isolated antibody or antigen-binding fragment thereof of claim 6.

68. The method of claim 67, wherein the neurodegenerative disease is selected from the group consisting of: Alzheimer's disease, primary age-related tauopathy, progressive supranuclear palsy (PSP), frontotemporal dementia, frontotemporal dementia with parkinsonism linked to chromosome 17, argyrophilic grain dementia, amyotrophic lateral sclerosis, amyotrophic lateral sclerosis/parkinsonism-dementia complex of Guam (ALS-PDC), corticobasal degeneration, chronic traumatic encephalopathy, Creutzfeldt-Jakob disease, dementia pugilistica, diffuse neurofibrillary tangles with calcification, Down's syndrome, familial British dementia, familial Danish dementia, Gerstmann-Straussler-Scheinker disease, globular glial tauopathy, Guadeloupean parkinsonism with dementia, Guadelopean PSP, Hallevorden-Spatz disease, hereditary diffuse leukoencephalopathy with spheroids (HDLS), Huntington's disease, inclusion-body myositis, multiple system atrophy, myotonic dystrophy, Nasu-Hakola disease, neurofibrillary tangle-predominant dementia, Niemann-Pick disease type C, pallido-ponto-nigral degeneration, Parkinson's disease, Pick's disease, postencephalitic parkinsonism, prion protein cerebral amyloid angiopathy, progressive subcortical gliosis, subacute sclerosing panencephalitis, and tangle only dementia.

69-109. (canceled)
